SEQUENCE LISTING (102 sequences)

SEQ ID NO: 1 - DNA, 1779 bp, Homo sapiens
  source 1..1779: mol_type = genomic DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaagaatataacaccagcaatcctgacttgttgctagtcatatttcaagtgacaggcatcagcctcctgccaccactgggagttgccatatctgtcatcatcatcttctactgctaccgcgttaaccggcagcagaagctgagttcaacctgggaaaccggcaagacgcggaagctcatggagttcagcgagcactgtgccatcatcctggaagatgaccgctctgacatcagctccacgtgtgccaacaacatcaaccacaacacagagctgctgcccattgagctggacaccctggtggggaaaggtcgctttgctgaggtctataaggccaagctgaagcagaacacttcagagcagtttgagacagtggcagtcaagatctttccctatgaggagtatgcctcttggaagacagagaaggacatcttctcagacatcaatctgaagcatgagaacatactccagttcctgacggctgaggagcggaagacggagttggggaaacaatactggctgatcaccgccttccacgccaagggcaacctacaggagtacctgacgcggcatgtcatcagctgggaggacctgcgcaagctgggcagctccctcgcccgggggattgctcacctccacagtgatcacactccatgtgggaggcccaagatgcccatcgtgcacagggacctcaagagctccaatatcctcgtgaagaacgacctaacctgctgcctgtgtgactttgggctttccctgcgtctggaccctactctgtctgtggatgacctggctaacagtgggcaggtgggaactgcaagatacatggctccagaagtcctagaatccaggatgaatttggagaatgttgagtccttcaagcagaccgatgtctactccatggctctggtgctctgggaaatgacatctcgctgtaatgcagtgggagaagtaaaagattatgagcctccatttggttccaaggtgcgggagcacccctgtgtcgaaagcatgaaggacaacgtgttgagagatcgagggcgaccagaaattcccagcttctggctcaaccaccagggcatccagatggtgtgtgagacgttgactgagtgctgggaccacgacccagaggcccgtctcacagcccagtgtgtggcagaacgcttcagtgagctggagcatctggacaggctctcggggaggagctgctcggaggagaagattcctgaagacggctccctaaacactaccaaatag

SEQ ID NO: 2 - DNA, 561 bp, Homo sapiens
  source 1..561: mol_type = genomic DNA
atgggtcgggggctgctcaggggcctgtggccgctgcacatcgtcctgtggacgcgtatcgccagcacgatcccaccgcacgttcagaagtcggatgtggaaatggaggcccagaaagatgaaatcatctgccccagctgtaataggactgcccatccactgagacatattaataacgacatgatagtcactgacaacaacggtgcagtcaagtttccacaactgtgtaaattttgtgatgtgagattttccacctgtgacaaccagaaatcctgcatgagcaactgcagcatcacctccatctgtgagaagccacaggaagtctgtgtggctgtatggagaaagaatgacgagaacataacactagagacagtttgccatgaccccaagctcccctaccatgactttattctggaagatgctgcttctccaaagtgcattatgaaggaaaaaaaaagcctggtgagactttcttcatgtgttcctgtagctctgatgagtgcaatgacaacatcatcttctcagaagaatataacaccagcaatcctgacttgttgctag

SEQ ID NO: 3 - DNA, 457 bp, Homo sapiens
  source 1..457: mol_type = genomic DNA
tgcgaatgctggagaacaggaaccagctgccgttgttaggaacaacttcatgaaggaaaagtattccagattgcctttctgtctggaggccatattattcatttattctctttctctctctccctctcccctcgcttccaatgaatctcttcactctaggagaaagaatgacgagaacataacactagagacagtttgccatgaccccaagctcccctaccatgactttattctggaagatgctgcttctccaaagtgcattatgaaggaaaaaaaaaagcctggtgagactttcttcatgtgttcctgtagctctgatgagtgcaatgacaacatcatcttctcagaaggtgagttttcttctcttaagggtgtgggacctgagatctgtgccaattttttgtatccttggtctgcagtgtcatagagcacattcctcctgtggtggattgcatac

SEQ ID NO: 4 - DNA, 456 bp, Homo sapiens
  source 1..456: mol_type = genomic DNA
tgcgaatgctggagaacaggaaccagctgccgttgttaggaacaacttcatgaaggaaaagtattccagattgcctttctgtctggaggccatattattcatttattctctttctctctctccctctcccctcgcttccaatgaatctcttcactctaggagaaagaatgacgagaacataacactagagacagtttgccatgaccccaagctcccctaccatgactttattctggaagatgctgcttctccaaagtgcattatgaaggaaaaaaaaagcctggtgagactttcttcatgtgttcctgtagctctgatgagtgcaatgacaacatcatcttctcagaaggtgagttttcttctcttaagggtgtgggacctgagatctgtgccaattttttgtatccttggtctgcagtgtcatagagcacattcctcctgtggtggattgcatac

SEQ ID NO: 5 - DNA, 1737 bp, Homo sapiens
  source 1..1737: mol_type = genomic DNA
atggcccattccctttctgttggtgggagtgggtacgtatgtcccttactcatccgggaagtattcatacaggttttgatcaagctgcgggtgtgttttgtccagtgcttttcagaagcagatcgggacattatgacacttgctaaccattggaattgccctgtgttatcatcagatagtgacttttgcatttttgacctgaaaactgggttttgcccattgaatagctttcagtggagaaatatgaacactattaagggcacacaaaactatatccctgccaaatgcttttcccttgatgcattctgccatcacttcagcaatatgaataaagctctactacctctctttgcggtgctatgtggaaatgaccatgttaatctacccatcatggagacattcttaagtaaagcgcgtcttcctcttggagctaccagttctaaagggaggagacaccaccgaatcctgggacttctgaattggttgtctcattttgccaaccctaccgaagcactagataatgttctgaaatacctcccaaaaaaggatcgagaaaatgttaaggaacttctctgctgttccatggaagaataccaacagtcccaggtgaagctacaggacttcttccagtgtggtacttatgtctgtccagatgccttgaatcttggtttaccagaatgggtattagtggctttagctaaaggccagctatctcctttcatcagtgatgctttggtcctaagacggaccattcttcccacacaggtggaaaacatgcagcaaccaaatgcccacagaatatctcagcccatcaggcaaatcatctatgggcttcttttaaatgcctcaccacatctggacaagacatcctggaatgcattgcctcctcagcctctagctttcagtgaagtggaaaggattaataaaaatatcagaacctcaatcattgatgcagtagaactggccaaggatcattctgacttaagcagattgactgagctctccttgaggaggcggcagatgcttctgttagaaaccctgaaggtgaaacagaccattctggagccaatccctacttcactgaagttgcccattgctgtcagttgctactggttgcagcacaccgagaccaaagcaaagctacatcatctacaatccttactgctcacaatgctagtggggcccttgattgccataatcaacagccctggtaaggaagagctgcaggaagatggtgctaagatgttgtatgcagagttccaaagagtgaaggcgcagacacggctgggcacaagactggacttagacacagctcacatcttctgtcagtggcagtcctgtctccagatggggatgtatctcaaccagctgctgtccactcctctcccagagccagacctaactcgactgtacagtggaagcctggtgcacggactatgccagcaactgctagcatcgacctctgtagaaagtctcctgagcatatgtcctgaggctaagcaactttatgaatatctattcaatgccacaaggtcatatgcccccgctgaaatattcctaccaaaaggtagatcaaattcaaaaaaaaaaaggcagaagaaacagaataccagctgttctaagaacagagggagaaccactgcacacaccaagtgttggtatgagggaaacaaccggtttgggttgttaatggttgaaaacttagaggaacatagtgaggcctccaacattgaataa

SEQ ID NO: 6 - DNA, 1689 bp, Homo sapiens
  source 1..1689: mol_type = genomic DNA
atggcccattccctttctgttggtgggagtgggtacgtatgtcccttactcatccgggaagtattcatacaggttttgatcaagctgcgggtgtgttttgtccagtgcttttcagaagcagatcgggacattatgacacttgctaaccattggaattgccctgtgttatcatcagatagtgacttttgcatttttgacctgaaaactgggttttgcccattgaatagctttcagtggagaaatatgaacactattaagggcacacaaaactatatccctgccaaatgcttttcccttgatgcattctgccatcacttcagcaatatgaataaagctctactacctctctttgcggtgctatgtggaaatgaccatgttaatctacccatcatggagacattcttaagtaaagcgcgtcttcctcttggagctaccagttctaaagggaggagacaccaccgaatcctgggacttctgaattggttgtctcattttgccaaccctaccgaagcactagataatgttctgaaatacctcccaaaaaaggatcgagaaaatgttaaggaacttctctgctgttccatggaagaataccaacagtcccaggtgaagctacaggacttcttccagtgtggtacttatgtctgtccagatgccttgaatcttggtttaccagaatgggtattagtggctttagctaaaggccagctatctcctttcatcagtgatgctttggtcctaagacggaccattcttcccacacaggtggaaaacatgcagcaaccaaatgcccacagaatatctcagcccatcaggcaaatcatctatgggcttcttttaaatgcctcaccacatctggacaagacatcctggaatgcattgcctcctcagcctctagctttcagtgaagtggaaaggattaataaaaatatcagaacctcaatcattgatgcagtagaactggccaaggatcattctgacttaagcagattgactgagctctccttgaggaggcggcagatgcttctgttagaaaccctgaaggtgaaacagaccattctggagccaatccctacttcactgaagttgcccattgctgtcagttgctactggttgcagcacaccgagaccaaagcaaagctacatcatctacaatccttactgctcacaatgctagtggggcccttgattgccataatcaacagccctggtaaggaagagctgcaggaagatggtgctaagatgttgtatgcagagttccaaagagtgaaggcgcagacacggctgggcacaagactggacttagacacagctcacatcttctgtcagtggcagtcctgtctccagatggggatgtatctcaaccagctgctgtccactcctctcccagagccagacctaactcgactgtacagtggaagcctggtgcacggactatgccagcaactgctagcatcgacctctgtagaaagtctcctgagcatatgtcctgaggctaagcaactttatgaatatctattcaatgccacaaggtcatatgcccccgctgaaatattcctaccaaaaggtagatcaaattcaaaaaaaaaaggcagaagaaacagaataccagctgttctaagaacagagggagaaccactgcacacaccaagtgttggtatgagggaaacaaccggtttgggttgttaa

SEQ ID NO: 7 - DNA, 495 bp, Homo sapiens
  source 1..495: mol_type = genomic DNA
agagttccaaagagtgaaggcgcagacacggctgggcacaagactggacttagacacagctcacatcttctgtcagtggcagtcctgtctccagatggggatgtatctcaaccagctgctgtccactcctctcccagagccagacctaactcgactgtacagtggaagcctggtgcacggactatgccagcaactgctagcatcgacctctgtagaaagtctcctgagcatatgtcctgaggctaagcaactttatgaatatctattcaatgccacaaggtcatatgcccccgctgaaatattcctaccaaaaggtagatcaaattcaaaaaaaaaaaggcagaagaaacagaataccagctgttctaagaacagagggagaaccactgcacacaccaagtgttggtatgagggaaacaaccggtttgggttgttaatggttgaaaacttagaggaacatagtgaggcctccaacattgaataaaactcagtttg

SEQ ID NO: 8 - DNA, 494 bp, Homo sapiens
  source 1..494: mol_type = genomic DNA
agagttccaaagagtgaaggcgcagacacggctgggcacaagactggacttagacacagctcacatcttctgtcagtggcagtcctgtctccagatggggatgtatctcaaccagctgctgtccactcctctcccagagccagacctaactcgactgtacagtggaagcctggtgcacggactatgccagcaactgctagcatcgacctctgtagaaagtctcctgagcatatgtcctgaggctaagcaactttatgaatatctattcaatgccacaaggtcatatgcccccgctgaaatattcctaccaaaaggtagatcaaattcaaaaaaaaaaggcagaagaaacagaataccagctgttctaagaacagagggagaaccactgcacacaccaagtgttggtatgagggaaacaaccggtttgggttgttaatggttgaaaacttagaggaacatagtgaggcctccaacattgaataaaactcagtttg

SEQ ID NO: 9 - DNA, 1767 bp, Homo sapiens
  source 1..1767: mol_type = genomic DNA
atggacctcgaggaggcggaagagtttaaagaacgctgtactcagtgtgctgctgtctcatggggtcttactgatgaaggcaaatattattgcacttcttgccacaatgttacagagagatatcaggaagttacaaacactgatcttattcctaatacccaaataaaagccctcaaccgggggcttaaaaaaaaaaacaatactgaaaaaggctgggattggtatgtgtgtgaaggtttccagtatattctttatcaacaagcagaagccttaaagaaccttggagtaggcccagagttaaagaacgatgttttacataatttttggaagcgctaccttcagaagagcaagcaggcatattgtaagaacccagtttataccactggaaggaaacctacggtattagaagataatctaagtcattcagactgggctagtgagcctgagctgctaagtgatgtcagctgtcctccttttcttgaaagtggagcggagtctcagtctgacatccacactcgaaaacctttccccgtcagcaaagcatcacaatcagaaacgtctgtctgctctggatctctggatggagttgaatactcacaacgaaaggagaagggaatcgtgaagatgaccatgccacagacacttgccttctgttatctgtccttactttggcagagagaagcaataacactttcagatcttttgaggtttgttgaagaggaccatattccttacataaatgcttttcagcattttccagaacagatgaaattatatggacgtgacagaggaatctttggtatagagtcttggcctgactacgaggacatctacaaaaaaacagtagaagttggaacatttttagatttgcctcgttttccagacataactgaagactgctatcttcatcccaacatactgtgtatgaaatacttgatggaagtcaacctccctgatgaaatgcatagcttaacttgccacgtggtaaaaatgactggaatgggagaagtggattttctgacatttgatcctatagctaaaatggcaaaaactgttaagtacgatgtacaagctgtagctatcattgtggtggtattgaaactgctctttctattggatgacagtttcgagtggtctttgtctaatcttgctgaaaagcataatgaaaagaacaaaaaagataagccatggtttgatttcagaaagtggtaccaaattatgaagaaagcttttgatgagaaaaaacaaaaatgggaagaagcaagggccaagtacctgtggaaaagtgaaaagccactctactactcatttgtcgacaaaccagtagcatataaaaaaagagaaatggtggtgaatctacagaaacaatttagcacactggtcgagtcaacagcaactgctggaaaaaaaagcccttcaagttttcagttcaactggactgaagaggacactgatagaacgtgtttccatggacacagccttcagggagtcctgaaagagaaaggccaatcactgctgactaagaattcattatattggcttagtacacagaaattctgcagatgctattgtacacatgtgacaacctatgaagaatcaaattattctctgagttatcagtttatactaaatctcttctccttcctgctcagaataaagacttcccttctccatgaagaagtgagcttagttgagaagaaactttttgagaaaaaatacagtgtaaaaagaaagaaatcaagatccaagaaagtgagacgacattga

SEQ ID NO: 10 - DNA, 1766 bp, Homo sapiens
  source 1..1766: mol_type = genomic DNA
atggacctcgaggaggcggaagagtttaaagaacgctgtactcagtgtgctgctgtctcatggggtcttactgatgaaggcaaatattattgcacttcttgccacaatgttacagagagatatcaggaagttacaaacactgatcttattcctaatacccaaataaaagccctcaaccgggggcttaaaaaaaaaacaatactgaaaaaggctgggattggtatgtgtgtgaaggtttccagtatattctttatcaacaagcagaagccttaaagaaccttggagtaggcccagagttaaagaacgatgttttacataatttttggaagcgctaccttcagaagagcaagcaggcatattgtaagaacccagtttataccactggaaggaaacctacggtattagaagataatctaagtcattcagactgggctagtgagcctgagctgctaagtgatgtcagctgtcctccttttcttgaaagtggagcggagtctcagtctgacatccacactcgaaaacctttccccgtcagcaaagcatcacaatcagaaacgtctgtctgctctggatctctggatggagttgaatactcacaacgaaaggagaagggaatcgtgaagatgaccatgccacagacacttgccttctgttatctgtccttactttggcagagagaagcaataacactttcagatcttttgaggtttgttgaagaggaccatattccttacataaatgcttttcagcattttccagaacagatgaaattatatggacgtgacagaggaatctttggtatagagtcttggcctgactacgaggacatctacaaaaaaacagtagaagttggaacatttttagatttgcctcgttttccagacataactgaagactgctatcttcatcccaacatactgtgtatgaaatacttgatggaagtcaacctccctgatgaaatgcatagcttaacttgccacgtggtaaaaatgactggaatgggagaagtggattttctgacatttgatcctatagctaaaatggcaaaaactgttaagtacgatgtacaagctgtagctatcattgtggtggtattgaaactgctctttctattggatgacagtttcgagtggtctttgtctaatcttgctgaaaagcataatgaaaagaacaaaaaagataagccatggtttgatttcagaaagtggtaccaaattatgaagaaagcttttgatgagaaaaaacaaaaatgggaagaagcaagggccaagtacctgtggaaaagtgaaaagccactctactactcatttgtcgacaaaccagtagcatataaaaaaagagaaatggtggtgaatctacagaaacaatttagcacactggtcgagtcaacagcaactgctggaaaaaaaagcccttcaagttttcagttcaactggactgaagaggacactgatagaacgtgtttccatggacacagccttcagggagtcctgaaagagaaaggccaatcactgctgactaagaattcattatattggcttagtacacagaaattctgcagatgctattgtacacatgtgacaacctatgaagaatcaaattattctctgagttatcagtttatactaaatctcttctccttcctgctcagaataaagacttcccttctccatgaagaagtgagcttagttgagaagaaactttttgagaaaaaatacagtgtaaaaagaaagaaatcaagatccaagaaagtgagacgacattga

SEQ ID NO: 11 - DNA, 499 bp, Homo sapiens
  source 1..499: mol_type = genomic DNA
ctttcccggaagctgcgctcgctacccgggtaacgggtcccggctgtggaagctcccgcggcgccgcgatggacctcgaggaggcggaagagtttaaagaacgctgtactcagtgtgctgctgtctcatggggtcttactgatgaaggcaaatattattgcacttcttgccacaatgttacagagagatatcaggaagttacaaacactgatcttattcctaatacccaaataaaagccctcaaccgggggcttaaaaaaaaaaacaatactgaaaaaggctgggattggtatgtgtgtgaaggtttccagtatattctttatcaacaagcagaagccttaaagaaccttggagtaggcccagagttaaagaacgatgttttacataatttttggaagcgctaccttcagaagagcaagcaggcatattgtaagaacccagtttataccactggaaggaaacctacggtattagaagataatctaagtcattcagactg

SEQ ID NO: 12 - DNA, 498 bp, Homo sapiens
  source 1..498: mol_type = genomic DNA
ctttcccggaagctgcgctcgctacccgggtaacgggtcccggctgtggaagctcccgcggcgccgcgatggacctcgaggaggcggaagagtttaaagaacgctgtactcagtgtgctgctgtctcatggggtcttactgatgaaggcaaatattattgcacttcttgccacaatgttacagagagatatcaggaagttacaaacactgatcttattcctaatacccaaataaaagccctcaaccgggggcttaaaaaaaaaacaatactgaaaaaggctgggattggtatgtgtgtgaaggtttccagtatattctttatcaacaagcagaagccttaaagaaccttggagtaggcccagagttaaagaacgatgttttacataatttttggaagcgctaccttcagaagagcaagcaggcatattgtaagaacccagtttataccactggaaggaaacctacggtattagaagataatctaagtcattcagactg

SEQ ID NO: 13 - DNA, 22 bp, synthetic construct
  source 1..22: mol_type = other DNA; note = synthetic primer
cagtgtcatagagcacattcct

SEQ ID NO: 14 - DNA, 19 bp, synthetic construct
  source 1..19: mol_type = other DNA; note = synthetic primer
agacagtttgccatgaccc

SEQ ID NO: 15 - DNA, 23 bp, synthetic construct
  source 1..23: mol_type = other DNA; note = synthetic primer
gtctcaccaggctttttttttcc

SEQ ID NO: 16 - DNA, 22 bp, synthetic construct
  source 1..22: mol_type = other DNA; note = synthetic primer
caccaggctttttttttccttc

SEQ ID NO: 17 - DNA, 23 bp, synthetic construct
  source 1..23: mol_type = other DNA; note = synthetic primer
gtctcaccaggctttttttttcg

SEQ ID NO: 18 - DNA, 23 bp, synthetic construct
  source 1..23: mol_type = other DNA; note = synthetic primer
gtctcaccaggcttttttttccg

SEQ ID NO: 19 - DNA, 23 bp, synthetic construct
  source 1..23: mol_type = other DNA; note = synthetic primer
gtctcaccaggctttttttaccg

SEQ ID NO: 20 - DNA, 23 bp, synthetic construct
  source 1..23: mol_type = other DNA; note = synthetic primer
gtctcaccaggctttttttttcc

SEQ ID NO: 21 - DNA, 23 bp, synthetic construct
  source 1..23: mol_type = other DNA; note = synthetic primer
gtctcaccaggcttttttttacg

SEQ ID NO: 22 - DNA, 22 bp, synthetic construct
  source 1..22: mol_type = other DNA; note = synthetic primer
caccaggctttttttatcgttc

SEQ ID NO: 23 - DNA, 22 bp, synthetic construct
  source 1..22: mol_type = other DNA; note = synthetic primer
caccaggctttttttatcgtcc

SEQ ID NO: 24 - DNA, 23 bp, synthetic construct
  source 1..23: mol_type = other DNA; note = synthetic primer
gtctcaccaggctttttttttcd

SEQ ID NO: 25 - DNA, 23 bp, synthetic construct
  source 1..23: mol_type = other DNA; note = synthetic primer
gtctcaccaggcttttttttvcd

SEQ ID NO: 26 - DNA, 23 bp, synthetic construct
  source 1..23: mol_type = other DNA; note = synthetic primer
gtctcaccaggctttttttvvcd

SEQ ID NO: 27 - DNA, 22 bp, synthetic construct
  source 1..22: mol_type = other DNA; note = synthetic primer
caccaggctttttttvtcdttc

SEQ ID NO: 28 - DNA, 22 bp, synthetic construct
  source 1..22: mol_type = other DNA; note = synthetic primer
caccaggctttttttvtcdtvc

SEQ ID NO: 29 - DNA, 17 bp, synthetic construct
  source 1..17: mol_type = other DNA; note = synthetic primer
atgtcctgaggctaagc

SEQ ID NO: 30 - DNA, 17 bp, synthetic construct
  source 1..17: mol_type = other DNA; note = synthetic primer
cactgcacacaccaagt

SEQ ID NO: 31 - DNA, 18 bp, synthetic construct
  source 1..18: mol_type = other DNA; note = synthetic primer
cactgcacacaccaagtg

SEQ ID NO: 32 - DNA, 23 bp, synthetic construct
  source 1..23: mol_type = other DNA; note = synthetic primer
gtttcttctgccttttttttttg

SEQ ID NO: 33 - DNA, 24 bp, synthetic construct
  source 1..24: mol_type = other DNA; note = synthetic primer
cttttttttttgaatttgatctac

SEQ ID NO: 34 - DNA, 23 bp, synthetic construct
  source 1..23: mol_type = other DNA; note = synthetic primer
gtttcttctgcctttttttatcg

SEQ ID NO: 35 - DNA, 23 bp, synthetic construct
  source 1..23: mol_type = other DNA; note = synthetic primer
gtttcttctgcctttttttgtcg

SEQ ID NO: 36 - DNA, 23 bp, synthetic construct
  source 1..23: mol_type = other DNA; note = synthetic primer
gtttcttctgccttttttttacg

SEQ ID NO: 37 - DNA, 23 bp, synthetic construct
  source 1..23: mol_type = other DNA; note = synthetic primer
gtttcttctgccttttttaatcg

SEQ ID NO: 38 - DNA, 23 bp, synthetic construct
  source 1..23: mol_type = other DNA; note = synthetic primer
gtttcttctgccttttttactcg

SEQ ID NO: 39 - DNA, 23 bp, synthetic construct
  source 1..23: mol_type = other DNA; note = synthetic primer
gtttcttctgcctttttttagtg

SEQ ID NO: 40 - DNA, 24 bp, synthetic construct
  source 1..24: mol_type = other DNA; note = synthetic primer
cttttttttctgaatgtgatctcc

SEQ ID NO: 41 - DNA, 23 bp, synthetic construct
  source 1..23: mol_type = other DNA; note = synthetic primer
gtttcttctgccttttttatacg

SEQ ID NO: 42 - DNA, 23 bp, synthetic construct
  source 1..23: mol_type = other DNA; note = synthetic primer
gtttcttctgcctttttaattcg

SEQ ID NO: 43 - DNA, 23 bp, synthetic construct
  source 1..23: mol_type = other DNA; note = synthetic primer
gtttcttctgcctttttttvtvg

SEQ ID NO: 44 - DNA, 23 bp, synthetic construct
  source 1..23: mol_type = other DNA; note = synthetic primer
gtttcttctgccttttttttvvg

SEQ ID NO: 45 - DNA, 23 bp, synthetic construct
  source 1..23: mol_type = other DNA; note = synthetic primer
gtttcttctgccttttttvvtvg

SEQ ID NO: 46 - DNA, 23 bp, synthetic construct
  source 1..23: mol_type = other DNA; note = synthetic primer
gtttcttctgcctttttttvvtg

SEQ ID NO: 47 - DNA, 24 bp, synthetic construct
  source 1..24: mol_type = other DNA; note = synthetic primer
cttttttttvtgaatvtgatctbc

SEQ ID NO: 48 - DNA, 23 bp, synthetic construct
  source 1..23: mol_type = other DNA; note = synthetic primer
gtttcttctgccttttttvtvvg

SEQ ID NO: 49 - DNA, 23 bp, synthetic construct
  source 1..23: mol_type = other DNA; note = synthetic primer
gtttcttctgcctttttvvttvg

SEQ ID NO: 50 - DNA, 19 bp, synthetic construct
  source 1..19: mol_type = other DNA; note = synthetic primer
tgcacttcttgccacaatg

SEQ ID NO: 51 - DNA, 22 bp, synthetic construct
  source 1..22: mol_type = other DNA; note = synthetic primer
aggcttctgcttgttgataaag

SEQ ID NO: 52 - DNA, 21 bp, synthetic construct
  source 1..21: mol_type = other DNA; note = synthetic primer
ccgggggcttaaaaaaaaaac

SEQ ID NO: 53 - DNA, 21 bp, synthetic construct
  source 1..21: mol_type = other DNA; note = synthetic primer
ccgggggcttaaaaaaaaatc

SEQ ID NO: 54 - DNA, 21 bp, synthetic construct
  source 1..21: mol_type = other DNA; note = synthetic primer
ccgggggcttaaaaaaaaagc

SEQ ID NO: 55 - DNA, 21 bp, synthetic construct
  source 1..21: mol_type = other DNA; note = synthetic primer
ccgggggcttaaaaaaaattc

SEQ ID NO: 56 - DNA, 21 bp, synthetic construct
  source 1..21: mol_type = other DNA; note = synthetic primer
ccgggggcttaaaaaaaagtc

SEQ ID NO: 57 - DNA, 21 bp, synthetic construct
  source 1..21: mol_type = other DNA; note = synthetic primer
ccgggggcttaaaaaatattc

SEQ ID NO: 58 - DNA, 21 bp, synthetic construct
  source 1..21: mol_type = other DNA; note = synthetic primer
ccgggggcttaaaaaagattc

SEQ ID NO: 59 - DNA, 21 bp, synthetic construct
  source 1..21: mol_type = other DNA; note = synthetic primer
ccgggggcttaaaaaagagtc

SEQ ID NO: 60 - DNA, 21 bp, synthetic construct
  source 1..21: mol_type = other DNA; note = synthetic primer
ccgggggcttaaaaaaaaabc

SEQ ID NO: 61 - DNA, 21 bp, synthetic construct
  source 1..21: mol_type = other DNA; note = synthetic primer
ccgggggcttaaaaaaaabbc

SEQ ID NO: 62 - DNA, 21 bp, synthetic construct
  source 1..21: mol_type = other DNA; note = synthetic primer
ccgggggcttaaaaaababbc

SEQ ID NO: 63 - DNA, 24 bp, Homo sapiens
  source 1..24: mol_type = genomic DNA
ggaaaaaaaaaagcctggtgagac

SEQ ID NO: 64 - DNA, 23 bp, Homo sapiens
  source 1..23: mol_type = genomic DNA
gaaggaaaaaaaaaagcctggtg

SEQ ID NO: 65 - DNA, 23 bp, Homo sapiens
  source 1..23: mol_type = genomic DNA
ggaaaaaaaaagcctggtgagac

SEQ ID NO: 66 - DNA, 22 bp, Homo sapiens
  source 1..22: mol_type = genomic DNA
gaaggaaaaaaaaagcctggtg

SEQ ID NO: 67 - DNA, 24 bp, Homo sapiens
  source 1..24: mol_type = genomic DNA
caaaaaaaaaaaggcagaagaaac

SEQ ID NO: 68 - DNA, 25 bp, Homo sapiens
  source 1..25: mol_type = genomic DNA
gtagatcaaattcaaaaaaaaaaag

SEQ ID NO: 69 - DNA, 23 bp, Homo sapiens
  source 1..23: mol_type = genomic DNA
caaaaaaaaaaggcagaagaaac

SEQ ID NO: 70 - DNA, 24 bp, Homo sapiens
  source 1..24: mol_type = genomic DNA
gtagatcaaattcaaaaaaaaaag

SEQ ID NO: 71 - DNA, 22 bp, Homo sapiens
  source 1..22: mol_type = genomic DNA
gtttttttttttaagcccccgg

SEQ ID NO: 72 - DNA, 21 bp, Homo sapiens
  source 1..21: mol_type = genomic DNA
gttttttttttaagcccccgg

SEQ ID NO: 73 - DNA, 204 bp, Homo sapiens
  source 1..204: mol_type = genomic DNA
ttggcacagatctcaggtcccacacccttaagagaagaaaactcaccttctgagaagatgatgttgtcattgcactcatcagagctacaggaacacatgaagaaagtctcaccaggctttttttttccttcataatgcactttggaaaagcagcatcttccaaaataaagtcatggtaggggagcttggggtcatggcaaactg

SEQ ID NO: 74 - DNA, 203 bp, Homo sapiens
  source 1..203: mol_type = genomic DNA
tggcacagatctcaggtcccacacccttaagagaagaaaactcaccttctgagaagatgatgttgtcattgcactcatcagagctacaggaacacatgaagaaagtctcaccaggctttttttttccttcataatgcactttggagaaccagcatcttccaaaataaagtcatggaaggggagcttggggtcagggcaaactg

SEQ ID NO: 75 - DNA, 219 bp, Homo sapiens
  source 1..219: mol_type = genomic DNA
ccggaaaaaattggcacagatctcaggtcccacacccttaagagaagaaaactcaccttctgagaagatgatgttgtcattgcactcatcagagctacaggaacacatgaagaaagtctcaccaggctttttttttccttcataatgcactttggagaagcagcatcttccagaataaagtcatggtaggggagcttggggtcatggcaaactgtctaa

SEQ ID NO: 76 - DNA, 194 bp, Homo sapiens
  source 1..194: mol_type = genomic DNA
acagatctcaggtcccacacccttaagagaagaaaactcaccttctgagaagatgatgttgtcattgcactcatcagagctacaggaacacatgaagaaagtctcaccaggctttttttttccttcataatgcactttggaaaagcagcatcttccaaaataaagtcatggtaggggagcttggggtcatggca

SEQ ID NO: 77 - DNA, 151 bp, Homo sapiens
  source 1..151: mol_type = genomic DNA
tggcacagatctcaggtcccacacccttaagagaagaaaactcaccttctgagaagatgatgttgtcattgcactcatcagagctacaggaacacatgaagaaagtctcaccaggctttttttttccttcataatgcactttggagaagca

SEQ ID NO: 78 - DNA, 135 bp, Homo sapiens
  source 1..135: mol_type = genomic DNA
attggcacagatctcaggtcccacacccttaagagaagaaaactcaccttctgagaagatgatgttgtcattgcactcatcagagctacaggaacacatgaagaaagtctcaccaggcttttttttttccttcaa

SEQ ID NO: 79 - DNA, 136 bp, Homo sapiens
  source 1..136: mol_type = genomic DNA
tggcacagatctcaggtcccacacccttaagagaagaaaactcaccttctgagaagatgatgttgtcattgcactcatcagagctacaggaacacatgaagaaagtctcaccaggcttttttttttccttcataag

SEQ ID NO: 80 - DNA, 130 bp, Homo sapiens
  source 1..130: mol_type = genomic DNA
acagatctcaggtcccacacccttaagagaagaaaactcaccttctgagaagatgatgttgtcattgcactcatcagagctacaggaacacatgaagaaagtctcaccaggcttttttttttccttctta

SEQ ID NO: 81 - DNA, 135 bp, Homo sapiens
  source 1..135: mol_type = genomic DNA
tggcacagatctcaggtcccacacccttaagagaagaaaactcaccttctgagaagatgatgttgtcattgcactcatcagagctacaggaacacatgaagaaagtctcaccaggcttttttttttccttcataa

SEQ ID NO: 82 - DNA, 129 bp, Homo sapiens
  source 1..129: mol_type = genomic DNA
agatctcaggtcccacacccttaagagaagaaaactcaccttctgagaagatgatgttgtcattgcactcatcagagctacaggaacacatgaagaaagtctcaccaggcttttttttttccttcataa

SEQ ID NO: 83 - DNA, 23 bp, synthetic construct
  source 1..23: mol_type = other DNA
gtttcttctgccttttttvvvtg

SEQ ID NO: 84 - DNA, 23 bp, synthetic construct
  source 1..23: mol_type = other DNA
gtttcttctgccttttttgatgg

SEQ ID NO: 85 - DNA, 23 bp, synthetic construct
  source 1..23: mol_type = other DNA
gtttcttctgccttttttcatcg

SEQ ID NO: 86 - DNA, 23 bp, synthetic construct
  source 1..23: mol_type = other DNA
gtttcttctgccttttttaggtg

SEQ ID NO: 87 - DNA, 23 bp, synthetic construct
  source 1..23: mol_type = other DNA
gtttcttctgccttttttaagtg

SEQ ID NO: 88 - DNA, 23 bp, synthetic construct
  source 1..23: mol_type = other DNA
gtttcttctgccttttttacctg

SEQ ID NO: 89 - DNA, 22 bp, synthetic construct
  source 1..22: mol_type = other DNA
agttgttgtgcataaaabbbga

SEQ ID NO: 90 - DNA, 22 bp, synthetic construct
  source 1..22: mol_type = other DNA
agttgttgtgcataaaatttga

SEQ ID NO: 91 - DNA, 22 bp, synthetic construct
  source 1..22: mol_type = other DNA
agttgttgtgcataaaacccga

SEQ ID NO: 92 - DNA, 22 bp, synthetic construct
  source 1..22: mol_type = other DNA
gaatatcctgtgtgaagatcac

SEQ ID NO: 93 - DNA, 20 bp, synthetic construct
  source 1..20: mol_type = other DNA
gttccttatgtcctctgtgc

SEQ ID NO: 94 - DNA, 5214 bp, Homo sapiens
  source 1..5214: mol_type = genomic DNA
gtctgggcttccgaatatgttttatgacggttgattttacaccaggaggtttgtctccgaggaagacccagggaactggatatctagcgagaacttcctccggattccccggcgcctcgggaaaatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacgg

SEQ ID NO: 95 - DNA, 5213 bp, Homo sapiens
  source 1..5213: mol_type = genomic DNA
gtctgggcttccgaatatgttttatgacggttgattttacaccaggaggtttgtctccgaggaagacccagggaactggatatctagcgagaacttcctccggattccccggcgcctcgggaaaatgggagctgctgcaaagttggcgtttgccgtctttcttatctcctgttcttcaggtgctatacttggtagatcagaaactcaggagtgtcttttctttaatgctaattgggaaaaagacagaaccaatcaaactggtgttgaaccgtgttatggtgacaaagataaacggcggcattgttttgctacctggaagaatatttctggttccattgaaatagtgaaacaaggttgttggctggatgatatcaactgctatgacaggactgattgtgtagaaaaaaaagacagccctgaagtatatttttgttgctgtgagggcaatatgtgtaatgaaaagttttcttattttccggagatggaagtcacacagcccacttcaaatccagttacacctaagccaccctattacaacatcctgctctattccttggtgccacttatgttaattgcggggattgtcatttgtgcattttgggtgtacaggcatcacaagatggcctaccctcctgtacttgttccaactcaagacccaggaccacccccaccttctccattactaggtttgaaaccactgcagttattagaagtgaaagcaaggggaagatttggttgtgtctggaaagcccagttgcttaacgaatatgtggctgtcaaaatatttccaatacaggacaaacagtcatggcaaaatgaatacgaagtctacagtttgcctggaatgaagcatgagaacatattacagttcattggtgcagaaaaacgaggcaccagtgttgatgtggatctttggctgatcacagcatttcatgaaaagggttcactatcagactttcttaaggctaatgtggtctcttggaatgaactgtgtcatattgcagaaaccatggctagaggattggcatatttacatgaggatatacctggcctaaaagatggccacaaacctgccatatctcacagggacatcaaaagtaaaaatgtgctgttgaaaaacaacctgacagcttgcattgctgactttgggttggccttaaaatttgaggctggcaagtctgcaggcgatacccatggacaggttggtacccggaggtacatggctccagaggtattagagggtgctataaacttccaaagggatgcatttttgaggatagatatgtatgccatgggattagtcctatgggaactggcttctcgctgtactgctgcagatggacctgtagatgaatacatgttgccatttgaggaggaaattggccagcatccatctcttgaagacatgcaggaagttgttgtgcataaaaaaagaggcctgttttaagagattattggcagaaacatgctggaatggcaatgctctgtgaaaccattgaagaatgttgggatcacgacgcagaagccaggttatcagctggatgtgtaggtgaaagaattacccagatgcagagactaacaaatattattaccacagaggacattgtaacagtggtcacaatggtgacaaatgttgactttcctcccaaagaatctagtctatgatggttgcgccatctgtgcacactaagaaatgggactctgaactggagctgctaagctaaagaaactgcttacagtttattttctgtgtaaaatgagtaggatgtctcttggaaatgttaagaaagaagaccctttgttgaaaaatgttgctctgggagacttactgcattgccgacagcacagatgtgaaggacatgagactaagagaaaccttgcaaactctataaagaaacttttgaaaaagtgtacatgaagaatgtagccctctccaaatcaaggatcttttggacctggctaatggagtgtttgaaaactgacatcagatttcttaatgtctgtcagaagacactaattccttaaatgaactactgctattttttttaaatcaaaaacttttcatttcagattttaaaaagggtaacttgtttttattgcatttgctgttgtttctataaatgactattgtaatgccaatatgacacagcttgtgaatgtttagtgtgctgctgttctgtgtacataaagtcatcaaagtggggtacagtaaagaggcttccaagcattactttaacctccctcaacaaggtatacctcagttccacggttgctaaattataaaattgaaaacactaacaaaatttgaataataaatcgatccatgttttgtaacaaattcactgtgttatttaaggaaaaaaaggtaagctatgcttagtgccaacaataagtggccattcgtaaagcagtgttttagcatttcttgtgctggcttgtaatgtagggaaaaaaagtgctgttttttgaaaagatggtgtcatttcccccttcttcccatgttttaaagccccatcttatatccagttcccaaaatttgcatacttacctaagtattttttttaggtgtgctgtgtttggggaatatttgaaaatttaaagcatgatttaaaattttttaaagtgagctgtgacactggaaagctcttcattttatcttttaaaatagagttttttctatttatatatgtaaaattgtagtgtatttcttttcaccaaacagtgtgtgggacattctttatcactgttttaggatcacctcaggaagtgtcgttacccagaattccccactgtctgctatgagacttgtaactttatcactatacttctgcttggtgccatcttgtcagagtaatatttgatgtctgtgatatgtaaagaattatcctaggataaagatattaaactttaagcagatttcagatgttactgctttaaaacaaatcagggataacaaattaaacgtataacttaaaatatgcaatgacatttagaggtaaccaatgttgatataggtagcatagcctagcctcctccccaaaattgcttttacaactaacactgatactaatttaggatagttcatgccttatccttgctaagaaaatggaattgatggtaggcaggtgctaaagtgcttttcaaaacaatattacgttagaatacaattggattcttcctcaaatttatacaggccaaaaagtaaaacattaattttctgaatttccagattaccaatcaattaatcaacaaatagccagtattatgctgtgtatttctgtcaggtcattttaaaatccatgttaattttataaaagaattttttacatgtcactgtcaggagctcactgtgaatgtgttgtcttcaaatggttatttaaccacacagtacactacattttacatatatgtacgtaatctctgggaatagtaaattaattatgttatttataaacaatacataggtcaacagactttaagcagggaggaaaagaagagtaatagcgtctgtgtgctgcagaccattcagaactgtcacgtgtgtccccatggtctcattcattgtattcctagcaattcccttttcaatgttgagttcacctctttatttcacaaagtacttggtctctcaatttcttgatctggttttgcttccatttaaaaactaatcaagaagggaaaatattgagaatgtgcatacaagaaaatcattaatttcctgaagatgaatttctacctgttgtgaacatttaactttctttttaaaagttaaacaaaaataaacaagggatattatgatgaatgtttggcttatgtgagtactagagataaaatttttaaacccagttattcacaatataaaatgttttcaagttagaaaaaatttttagaaatcctgggtattgtatttaactgtagctaaccaattttaaaacttgtattcttttgagaactattattaatagaaaaactttttataagcagtaaaataagaatgttccagtgactacctgtccttatacctagtcttgttaaaactttcttttgcagggtatttagtgtttggtttacagtcagtgcagagtgggcaagttaacagaaagtttgagctagagatactggaaaaaaaaaagatcaaagaatgagaaaaatggtgatccattttggggcaaactgagaccccccaaataactctttcctcatgtgtatggtgctcctcatgactcgtcttgtattttgcctttctgatacccatcagaactgctgctgctctaacttatactctttaccttgcccagatctccgcgtaaggaatgctttatgatcaacttgccataggactgatggattaaccagtgttcggctttatttgaagtctatgccctgcacagctcttgtatgtattttagatgctagaagtttttttagcatgtgatgtgtgattcttgtttgaattctaggtaccttgtgaattccagaaaaagagactgtgcttcacgattgttagtcccatgaacttgcactatctatctttcatggtgatgttttgaaaatacaatcaggaaaaaacccaacacctttggaatttaaaatagaatcatatcatgaaatttaaaaagaatctcttctgttgcatttcctcacccctaagtaacagctacatttaagtaaaatgcaggtggtaggggaaaaaaaaccatggcgagatggtggtttagtggaataaactgattactggtttttttgtttttttttttttttttaaagaaagaagcttcatcacagatactttccagtttctcttttatacttttttgaaagattactttttaggaacatttggtatgatatgcataaaattatttatccatttatgggcaaaatgatacaagtagcatcttgattgaacatcatttacctcagatattcaaccagcagtacgttttttatgcagtctcaacccatatcccatttgttacctctcagaatattggtaagcagttattttcgctttactctgtatttcttgtgttttgggcacaggttattgtactactgtcaaatcgtacttgctattttttctgcaagtatttaacagaaagcttaaaatccccataaaaccccaccttggataagtgattgttaaatattgtacaaataaaatgtatgctatccccattccatccccaagttaaataaaaaaatgaatacgg

SEQ ID NO: 96 - DNA, 992 bp, Homo sapiens
  source 1..992: mol_type = genomic DNA
catggttataatagattgaacctagagcctccagagctggaagcaccactgttaacattctgatctgaattgtaaggaatgggtaccttaaagggatattatataacgttaatttacaaatactgattgttccttatgtcctctgtgcagatggaaataggcatcctttatacctagataagaaagccccttatgcaatctttaagggaattacatgccaaattataggccttttcatttcccatacattagtttggtcacactgtggtataagtacagttgagagtctgtttctcttctgtcctcatagcatgtaaacagttgggaataggtgacagagtatattttagaaagtttgtaccagtttgaaagtcaggaggattttaatgaaaatgatttattttacttttcttacttttcaggacctgtagatgaatacatgttgccatttgaggaggaaattggccagcatccatctcttgaagacatgcaggaagttgttgtgcataaaaaaaagaggcctgttttaagagattattggcagaaacatgctgtaagttatccagttagcttttcatttgaaattccaataaaacacttttcagaggaattatttatctctgcacatttctctttcttctgcaagtattttctggaaggtgatcttcacacaggatattctagagttctagaggcagaattagggctatgtctgtatacccctgaaggtgattgtaaagtaatagagctttagagggcttttgtctcaatggtcctgtgcagaagattgtgtcatctatttagaaagtttcccagggaaaaggcatgccagactcggaaactgtggatagttgggtaactttgctgatgaccacttccaatatgacaacattttaaaaagtttatgaagcaccatgttttctcctctccatgggagtttgttgtagccttttaatttcggcttagactttcaagtcttaatagtgctttaa

SEQ ID NO: 97 - DNA, 991 bp, Homo sapiens
  source 1..991: mol_type = genomic DNA
catggttataatagattgaacctagagcctccagagctggaagcaccactgttaacattctgatctgaattgtaaggaatgggtaccttaaagggatattatataacgttaatttacaaatactgattgttccttatgtcctctgtgcagatggaaataggcatcctttatacctagataagaaagccccttatgcaatctttaagggaattacatgccaaattataggccttttcatttcccatacattagtttggtcacactgtggtataagtacagttgagagtctgtttctcttctgtcctcatagcatgtaaacagttgggaataggtgacagagtatattttagaaagtttgtaccagtttgaaagtcaggaggattttaatgaaaatgatttattttacttttcttacttttcaggacctgtagatgaatacatgttgccatttgaggaggaaattggccagcatccatctcttgaagacatgcaggaagttgttgtgcataaaaaaagaggcctgttttaagagattattggcagaaacatgctgtaagttatccagttagcttttcatttgaaattccaataaaacacttttcagaggaattatttatctctgcacatttctctttcttctgcaagtattttctggaaggtgatcttcacacaggatattctagagttctagaggcagaattagggctatgtctgtatacccctgaaggtgattgtaaagtaatagagctttagagggcttttgtctcaatggtcctgtgcagaagattgtgtcatctatttagaaagtttcccagggaaaaggcatgccagactcggaaactgtggatagttgggtaactttgctgatgaccacttccaatatgacaacattttaaaaagtttatgaagcaccatgttttctcctctccatgggagtttgttgtagccttttaatttcggcttagactttcaagtcttaatagtgctttaa

SEQ ID NO: 98 - DNA, 23 bp, synthetic construct
  source 1..23: mol_type = other DNA
gtttcttctgccttttttagctg

SEQ ID NO: 99 - DNA, 23 bp, synthetic construct
  source 1..23: mol_type = other DNA
gtttcttctgccttttttggatg

SEQ ID NO: 100 - DNA, 23 bp, synthetic construct
  source 1..23: mol_type = other DNA
gtttcttctgcctttttvvvttg

SEQ ID NO: 101 - DNA, 23 bp, synthetic construct
  source 1..23: mol_type = other DNA
gtttcttctgcctttttaccttg

SEQ ID NO: 102 - DNA, 17 bp, synthetic construct
  source 1..17: mol_type = other DNA
acttggtgtgtgcagtg

(19) United States
(12) Patent Application Publication
(10) Pub. No.: US 2026/0218308 A1
(43) Pub. Date: Jul. 30, 2026

(54) TAF1b and ASTE1 Frameshift Primers

(71) Applicant: Hubro Bioscience AS, Oslo (NO)

(72) Inventors: Henrik Kvalheim ERIKSEN, Oslo (NO); Brage Milch JOHANNESEN, Oslo (NO); Jasmin Victoria NICOLAYSEN, Oslo (NO)

(73) Assignee: Hubro Bioscience AS, Oslo (NO)

(21) Appl. No.: 19/147,219

(22) PCT Filed: Jan. 10, 2024
(86) PCT No.: PCT/EP2024/050461

§ 371 Date: Jul. 10, 2025

(30) Foreign Application Priority Data
Jan. 11, 2023 (LU) LU503335

(51) Int. Cl. C12Q 1/6886; C12Q 1/6806; C12Q 1/6827; C12Q 1/686

(52) U.S. Cl. CPC C12Q 1/6886; C12Q 1/6806; C12Q 1/6827; C12Q 1/686; C12Q 2600/156

(57) ABSTRACT

A primer for detection of a mutation in a microsatellite contained in a target sequence of a double stranded DNA molecule, wherein the mutation is a frameshift of the microsatellite as compared with the corresponding wild type microsatellite sequence. The primer comprises a region of at least 10 nucleotides that is complementary to the target sequence of the antisense or the sense strand of the DNA molecule containing the microsatellite having a frameshift mutation, except that the primer includes between one and four nucleotides which are mismatched to the target sequence containing the mutation in the microsatellite and which are also mismatched to a corresponding sequence containing the wild type microsatellite, wherein the primer is configured to anneal across the length of the microsatellite having a frameshift mutation and to anneal to at least one nucleotide flanking the 5′ end of the microsatellite having a frameshift mutation and at least one nucleotide flanking the 3′ end of the microsatellite having a frameshift mutation.

### FIELD OF INVENTION

The present invention relates to a primer and, more specifically, a primer for detection of a mutation in a polynucleotide sequence. The present invention also relates to a kit comprising the primer and a method of detecting a mutation in a microsatellite sequence.

### BACKGROUND OF INVENTION

Frameshift mutations can cause disease by disrupting normal protein translation. Amongst other pathologies, frameshift mutations have been linked with cancer, and in particular cancers that are linked to microsatellite instability. Microsatellite instability (MSI) is a hypermutable state of cells caused by an impairment in DNA mismatch repair (MMR). In other words, cells affected by MSI do not have proper functioning repair mechanisms and therefore accrue spontaneous mutations during DNA replications, which can cause frameshift mutations. These errors accumulate particularly in microsatellites, which are repeated sequences of DNA (most commonly a dinucleotide repeat of the nucleotides C and A). Therefore, MSI results in insertion and deletion mutations in these repeated sequences called microsatellites.

MSI has been linked to many cancers, including colon, gastric, endometrium, ovarian, hepatobiliary tract, urinary tract, brain and skin cancers (Cortes-Ciriano et al. 2017). Frameshift mutations have been found in TGFβR2, ASTE1, TAF1β, KIAA2018, SLC22A9 and ACVR2A. In particular, frameshift mutation of TGFβR2 is present in large numbers of colorectal cancers (CRC) and gastric cancers (GC) caused by microsatellite instability (MSI). Frameshift mutation of TGFβR2 is also known to be associated with Lynch Syndrome. MSI consists of insertion and deletion mutations in stretches of short tandem DNA repeats (microsatellites) throughout the genome. Over 95% of the frameshift mutations in CRC are reported to be single nucleotide deletions (Maby et al. 2015).

In view of the above, frameshift mutations offer a therapeutic target for treatment of diseases associated with such mutations, including many cancers. However, not all patients having such a disease will have this mutation, particularly for cancer patients where the disease is known to be highly heterogeneous. Thus, it is important to be able to detect the subset of patients within a disease population that have a frameshift mutation, in order to find the patients who would respond to therapy targeting the frameshift mutation.

For example, the cancer vaccine FMPV-1, which consists of a mutant immunogenic peptide, targets frameshift mutant TGFβR2 as described in WO2020/239937. Not all patients with MSI-CRC (approximately 75%) and MSI-GC (approximately 80%) have frameshift mutated TGFβR2. In order to ensure recruitment of only eligible patients to clinical studies with FMPV-1 it would be highly useful to screen patients for detection of such a mutant TGFβR2 to find the patients who will benefit from such therapy. In other words, the detection of frameshift mutations may help to provide a personalised medicine approach for diseases where this mutation may or may not be present. In addition, detecting such mutations can assist with recruiting suitable candidates to a clinical trial for testing therapies that target such frameshift mutations.

This screening and personalised medicine approach would also be useful for patients with a single nucleotide deletion in the ASTE1, TAF1β or ACVR2A gene. In particular, the cancer vaccine FMPV-2 (also referred to as “fsp8”) is a mutant immunogenic peptide targeting frameshift mutant ASTE1, as described in WO2021/239980. WO2021/239980 also describes a cancer vaccine being immunogenic peptides targeting frameshift mutant TAF1β gene. A single nucleotide deletion is the most dominant frameshift mutation in each of the ASTE1 and TAF1β genes, but does not occur in all MSI-H cancers. Therefore, it would be useful to screen patients for detection of such mutant ASTE1 or mutant TAF1β, to find the patients who will benefit from such therapy, as mentioned above in respect of TGFβR2.

Additionally, FMPV-3 is a cancer vaccine which comprises a cocktail of immunogenic peptides targeting one or more of TGFβR2, ASTE1 and TAF1β, as described in Luxembourg Patent Application No. LU502776. The screening of patients for detection of frameshift mutant TGFβR2, frameshift mutant ASTE1 and/or frameshift mutant TAF1β, would therefore also be useful to identify the patients who will benefit from such a therapy.

Activin Receptor type 2A (ACVR2A) is within the TGF-β superfamily of structurally related signalling proteins. It shares a 37% homology with TGFβR2, a gene that is frequently mutated in MSI-H cancers. ACVR2A is also frequently mutated in MSI-H cancers. ACVR2A contains two polyadenine (A8) tracts in the coding region that are hotspot mutation sites in MSI-H cancers, namely in exon 3 and in exon 10. The frameshift mutation in exon 3 leads to a functional gene inactivation through nonsense-mediated decay of the mutant mRNA, while the frameshift mutation in exon 10 results in a truncated version of the ACVR2A gene. The frameshift mutation in exon 10 is the most frequent of the two and leaves behind mRNA. ACVR2A is frequently mutated in MSI-H cancers such as stomach adenocarcinoma (STAD), uterine corpus endometrial cancer (UCEC) and colon adenocarcinoma (COAD). For both COAD and STAD, ACVR2A is more frequently mutated than TGFβR2 and TAF1β. ACVR2A is also frequently mutated in UCEC, where approximately 30% of cancers are MSI-H.

Currently, it is necessary either to sequence the genome of the patient to understand their TGFβR2, ASTE1, TAF1β and ACVR2A status, or to rely on DNA amplification followed by sequencing to detect the presence or absence of the frameshift mutation. These sequencing methods take around 2 days to complete and typically must be conducted by an external laboratory, as hospitals generally do not have the equipment available to carrying out such sequencing. This therefore requires samples to be transported to such a testing lab. Therefore, there is a need to provide an easier assay for specific detection of frameshift mutations, particularly for the purposes of personalised medicine.

Polymerase chain reaction (PCR) is a well-established technology for analysing DNA sequences. The DNA specificity of a PCR test is determined by the primers designed to interact with the target DNA sequence. However, PCR primers cannot normally target a region with more than four nucleotide repeats, meaning the use of primers to detect a change in a microsatellite region is not trivial.

Guanine (G) and cytosine (C), and adenine (A) and thymine (T), are complementary nucleotides, and thus will hybridise e.g. during DNA replication. The GC base pair is held together by three hydrogen bonds, whilst the AT base pair is held together by two hydrogen bonds. Therefore, double-stranded DNA with a higher proportion of GC base pairs will be more strongly hybridised, more stable, and will have a higher melting temperature. Target DNA sequences with a high GC content may have higher melting temperature, require higher annealing temperatures and have a higher probability of mismatches of primer-dimer formation in the complementary primer. Primers may therefore be designed taking into account the GC content of the target DNA sequence this ensure stable annealing of the primer to the sequence. Preferably, the GC content of the corresponding primer and target DNA sequence is 40% to 60%. In contrast, target DNA sequences with a low GC content may have low stability and require lower annealing temperatures with the complementary primer, resulting in a low PCR efficiency. To put it in another way, a low GC content in the target DNA sequences makes it difficult to design a primer that will anneal. In particular, the ASTE1 gene has a low GC content in the regions flanking the microsatellite, meaning that the design of useful primers targeting this region of the ASTE1 gene is challenging.

In addition, repeats and/or low variation in the target DNA sequence make primer design difficult due to low specificity of the primer to the desired target DNA sequence and therefore the primer may have a higher probability of non-specific binding. In particular, the TAF1β gene comprises repeats in both the upstream and downstream regions flanking the microsatellite, as well as low variation in the sequences in these flanking regions, which further makes the design of primers targeting this region of the TAF1β gene a challenge.

Thus, there is a need to develop a technique for specific detection in tumour biopsies of frameshift mutations that may be relevant in disease, in particular in TGFβR2, ASTE1, TAF1β and ACVR2A. Solid tumour biopsies are not representative of the entire tumour, since tumours tends to be heterogeneous and as such tumour biopsies are representative only of the part of tumour that the biopsy is taken from. In contrast, liquid biopsies are much more representative of whole cancer, and are easier to work on. It is therefore desirable for this technique to be suitable for detection of cell free DNA (cfDNA) in liquid biopsies (e.g. plasma).

### SUMMARY OF INVENTION

The present invention solves the needs and objectives discussed above through the design of primers, and DNA amplification assays using such primers, that are able to distinguish between a microsatellite with a frameshift mutation, and a microsatellite with no frameshift mutation (i.e. a wild type microsatellite). In particular, these primers allow for the use of PCR to distinguish between a frameshift mutation and a wild type microsatellite even though they differ by only as little as a single nucleotide, by for example, conducting the PCR under suboptimal conditions. These suboptimal conditions increase the stringency of the reaction so that the difference between the amplification efficiency between the frameshift mutant and the wild type microsatellite is magnified, providing a more sensitive test for the frameshift mutant microsatellite. These suboptimal conditions can be achieved in a number of different ways. Therefore, this is a useful invention in the detection of frameshift mutations, diagnosis of MSI-related disease, and/or the screening of subjects for suitability for treatment against such frameshift mutations.

In one aspect of the invention, there is provided a primer for detection of a mutation in a microsatellite contained in a target sequence of a double stranded DNA molecule, wherein the mutation is a frameshift of the microsatellite as compared with the corresponding wild type microsatellite sequence, and wherein the primer comprises a region of at least 10 nucleotides that is complementary to the target sequence of the antisense or the sense strand of the DNA molecule containing the microsatellite having a frameshift mutation, except that the primer includes between one and four nucleotides which are mismatched to the target sequence containing the mutation in the microsatellite and which are also mismatched to a corresponding sequence containing the wild type microsatellite, wherein the primer is configured to anneal across the length of the microsatellite having a frameshift mutation and to anneal to at least one nucleotide flanking the 5′ end of the microsatellite having a frameshift mutation and at least one nucleotide flanking the 3′ end of the microsatellite having a frameshift mutation.

In some embodiments, the primer consists of between 16 and 30 nucleotides.

10 In some embodiments, the primer comprises a region of at least 11 or at least 12 nucleotides that is complementary to the target sequence of the antisense or the sense strand of the DNA molecule containing the microsatellite having a frameshift mutation, except that the primer includes between one and four nucleotides which are mismatched to the target sequence containing the mutation in the microsatellite and which are also mismatched to a corresponding sequence containing the wild type microsatellite.

In some embodiments, at least one mismatched nucleotide is located in a position of the primer that is configured to anneal 3′ downstream of the microsatellite or within the microsatellite.

In some embodiments, at least one mismatched nucleotide is within five nucleotides 5′ upstream of the 3′ end of the microsatellite or within 12 nucleotides 3′ downstream of the 3′ end of the microsatellite.

In some embodiments, the target sequence is in a TGFβR2 gene, an ASTE1 gene, a TAF1β gene or an ACVR2A gene.

In some embodiments, the target sequence is in an ASTE1 gene, a TAF1β gene or an ACVR2A gene.

In some embodiments, the first nucleotide 3′ downstream of the 3′ end of the microsatellite is not a mismatched nucleotide.

In some embodiments, the primer comprises between 1 and 13, or between 1 and 12, nucleotides flanking the 5′ end of the microsatellite.

In some embodiments, the primer comprises between 4 and 13 nucleotides flanking the 5′ end of the microsatellite.

In some embodiments, the primer comprises 9, 10, 11, 12 or 13 nucleotides flanking the 5′ end of the microsatellite.

In some embodiments, the primer comprises 10, 12 or 13 nucleotides flanking the 5′ end of the microsatellite.

In some embodiments, the primer comprises between 1 and 13 nucleotides flanking the 3′ end of the microsatellite.

In some embodiments, the primer has 1 or 2 nucleotides flanking the 3′ end of the microsatellite.

In some embodiments, all of the at least one mismatched nucleotides are within the microsatellite.

In some embodiments, the primer comprises at least two nucleotides which are mismatched to the target sequence containing the mutation in the microsatellite and which are also mismatched to a corresponding sequence containing the wild type microsatellite (i.e. at least two mismatched nucleotides), wherein at least one of the mismatched nucleotides is within the microsatellite and at least one of the mismatched nucleotides is located in a position of the primer that is configured to anneal 3′ downstream of the microsatellite.

In some embodiments, at least one mismatched nucleotide is located at the first nucleotide 5′ upstream of the 3′ end of the microsatellite and/or at the second nucleotide 5′ upstream of the 3′ end of the microsatellite.

In some embodiments, at least one mismatched nucleotide is a substitution of a thymine (T) with an adenine (A), a substitution of a thymine (T) with a cytosine (C), or a substitution of a cytosine (C) with a guanine (G).

In some embodiments, at least one mismatched nucleotide is a substitution of a thymine (T) with a cytosine (C), a substitution of a thymine (T) with an adenine (A), a substitution of a thymine (T) with a guanine (G), or a substitution of an adenine (A) with a cytosine (C).

In some embodiments, at least one mismatched nucleotide is a substitution of an adenine (A) with a guanine (G), a substitution of an adenine (A) with a thymine (T) or a substitution of an adenine (A) with a cytosine (C).

In some embodiments, the primer comprises at least two mismatched nucleotides, and the at least two mismatched nucleotides comprise:
a substitution of a two pyrimidine nucleotides with a purine nucleotide and a pyrimidine nucleotide, or
a substitution of two purine nucleotides with two pyrimidine nucleotides or with a purine nucleotide and a pyrimidine nucleotide.

In some embodiments, at least one mismatched nucleotide is located at the first nucleotide 5′ upstream of the 3′ end of the microsatellite and at the second nucleotide 5′ upstream of the 3′ end of the microsatellite, wherein the at least one mismatched nucleotide located at the first nucleotide 5′ upstream of the 3′ end of the microsatellite is a substitution of an adenine (A) with a thymine (T) and the at least one mismatched nucleotide located at the second nucleotide 5′ upstream of the 3′ end of the microsatellite is a substitution of an adenine (A) with a guanine (G) or thymine (T).

In some embodiments, at least one mismatched nucleotide is located at the first, second or third nucleotide 5′ upstream of the 3′ end of the microsatellite, and wherein the at least one mismatched nucleotide is a substitution of a thymine (T) with a guanine (G) or a cytosine (C).

In some embodiments, at least one mismatched nucleotide is located at the fifth nucleotide 5′ upstream of the 3′ end of the microsatellite.

In some embodiments, the mismatched nucleotide at the fifth nucleotide 5′ upstream of the 3′ end of the microsatellite is a substitution of a thymine (T) with an adenine (A).

In some embodiments, the primer has a mismatched nucleotide at the second nucleotide 5′ upstream of the 3′ end of the microsatellite and at the third nucleotide 5′ upstream of the 3′ end of the microsatellite, and wherein the frameshift mutation is preferably in a microsatellite in ASTE1.

In some embodiments, the primer has a mismatched nucleotide at the first nucleotide 5′ upstream of the 3′ end of the microsatellite, at the second nucleotide 5′ upstream of the 3′ end of the microsatellite and at the fourth nucleotide 5′ upstream of the 3′ end of the microsatellite, and wherein the frameshift mutation is preferably in a microsatellite in ASTE1.

In some embodiments, the primer has a mismatched nucleotide at the first nucleotide 5′ upstream of the 3′ end of the microsatellite, at the fourth nucleotide 5′ upstream of the 3′ end of the microsatellite and at the fifth nucleotide 5′ upstream of the 3′ end of the microsatellite, and wherein the frameshift mutation is preferably in a microsatellite in ASTE1.

In some embodiments, the primer has a mismatched nucleotide at the first nucleotide 5′ upstream of the 3′ end of the microsatellite, at the third nucleotide 5′ upstream of the 3′ end of the microsatellite and at the fourth nucleotide 5′ upstream of the 3′ end of the microsatellite, and wherein the frameshift mutation is preferably in a microsatellite in ASTE1.

In some embodiments, the primer has a mismatched nucleotide at the second nucleotide 5′ upstream of the 3′ end of the microsatellite, at the third nucleotide 5′ upstream of the 3′ end of the microsatellite and at the fourth nucleotide 5′ upstream of the 3′ end of the microsatellite, and wherein the frameshift mutation is preferably in a microsatellite in ASTE1.

In some embodiments, the primer has a mismatched nucleotide at the third 5′ upstream of the 3′ end of the microsatellite, at the fourth nucleotide 5′ upstream of the 3′ end of the microsatellite and at the fifth nucleotide 5′ upstream of the 3′ end of the microsatellite, and wherein the frameshift mutation is preferably in a microsatellite in ASTE1.

In some embodiments, the primer has a mismatched nucleotide at the first nucleotide 5′ upstream of the 3′ end of the microsatellite, and wherein the frameshift mutation is preferably in a microsatellite in TAF1β.

In some embodiments, the primer has a mismatched nucleotide at the first nucleotide 5′ upstream of the 3′ end of the microsatellite and at the second nucleotide 5′ upstream of the 3′ end of the microsatellite, and wherein the frameshift mutation is preferably in a microsatellite in TAF1β.

In some embodiments, the primer has a mismatched nucleotide at the first nucleotide 5′ upstream of the 3′ end of the microsatellite, at the second nucleotide 5′ upstream of the 3′ end of the microsatellite, and at the fourth nucleotide 5′ upstream of the 3′ end of the microsatellite, and wherein the frameshift mutation is preferably in a microsatellite in TAF1β.

In some embodiments, the primer has a mismatched nucleotide at the first nucleotide 5′ upstream of the 3′ end of the microsatellite, at the second nucleotide 5′ upstream of the 3′ end of the microsatellite and at the third nucleotide 5′ upstream of the 3′ end of the microsatellite, and wherein the frameshift mutation is preferably in a microsatellite in ACVR2A.

In some embodiments, the primer includes between one and three nucleotides which are mismatched to the target sequence containing the mutation in the microsatellite and which are also mismatched to a corresponding sequence containing the wild type microsatellite.

In another aspect of the invention, there is provided a primer for DNA amplification comprising the sequence defined by any one of SEQ ID NO: 24 to 28, by any one of SEQ ID NO: 45, 47, 83 or 100, by any one of SEQ ID NO: 43, 44, 46, 48 or 49, by any one of SEQ ID NO: 60 to 62, or by SEQ ID NO: 89, and wherein X defines a substitution to any nucleotide which is mismatched to the corresponding nucleotide in the target sequence and the wild type sequence.

In another aspect of the invention, there is provided a primer for DNA amplification comprising the sequence defined by any one of SEQ ID NO: 17 to 19 or 21 to 23, by any one of SEQ ID NO: 34 to 38 or 40, by any one of SEQ ID NO: 39, 41, 42, 84 to 88, 98, 99 or 101, by any one of SEQ ID NO: 53 to 59, or by SEQ ID NO: 90 or 91.

In another aspect of the invention there is provided a kit for detection of a mutation in a microsatellite contained in a target sequence of a double stranded DNA molecule, wherein the mutation is a frameshift of the microsatellite as compared with the corresponding wild type microsatellite sequence, and wherein the kit comprises a first primer of the invention, and optionally a second primer, wherein the second primer is configured to anneal to the target sequence 3′ downstream of the microsatellite on the opposite strand of the DNA molecule to the strand on which the first primer is configured to anneal.

In some embodiments, the frameshift mutation is in a microsatellite in a TGFβR2 gene, an ASTE1 gene, a TAF1β gene or an ACVR2A gene.

In some embodiments, the frameshift mutation is in a microsatellite in the TGFβR2 gene and comprises the sequence according to residues 270 to 278 of SEQ ID NO: 3, and wherein the first primer comprises the sequence defined by any one of SEQ ID NO: 24 to 28, and/or, when present, the second primer comprises the sequence defined by SEQ ID NO: 14, and wherein X defines a substitution to any nucleotide which is mismatched to the corresponding nucleotide in the target sequence and the wild type sequence.

In some embodiments, the frameshift mutation is in a microsatellite in the ASTE1 gene and comprises the sequence according to residues 328 to 337 of SEQ ID NO: 7, and wherein the first primer comprises the sequence defined by SEQ ID NO: 45, 47, 83 or 100, and/or, when present, the second primer comprises the sequence defined by SEQ ID NO: 29, and wherein X defines a substitution to any nucleotide which is mismatched to the corresponding nucleotide in the target sequence and the wild type sequence.

In some embodiments, the frameshift mutation is in a microsatellite in the ASTE1 gene and comprises the sequence according to residues 328 to 337 of SEQ ID NO: 7, and wherein the first primer comprises the sequence defined by SEQ ID NO: 43, 44, 46, 48 or 49, and/or, when present, the second primer comprises the sequence defined by SEQ ID NO: 29, and wherein X defines a substitution to any nucleotide which is mismatched to the corresponding nucleotide in the target sequence and the wild type sequence.

In some embodiments, the frameshift mutation is in a microsatellite in the TAF1β gene and comprises the sequence according to residues 255 to 264 of SEQ ID NO: 11, and wherein the first primer comprises the sequence defined by any one of SEQ ID NOS: 60 to 62, and/or, when present, the second primer comprises the sequence defined by SEQ ID NO: 51, and wherein X defines a substitution to any nucleotide which is mismatched to the corresponding nucleotide in the target sequence and the wild type sequence.

In some embodiments, the frameshift mutation is in a microsatellite in the ACVR2A gene and comprises the sequence according to residues 509 to 515 of SEQ ID NO: 96, and wherein the first primer comprises the sequence defined by SEQ ID NO: 89, and/or, when present, the second primer comprises the sequence defined by SEQ ID NO: 92, and wherein X defines a substitution to any nucleotide which is mismatched to the corresponding nucleotide in the target sequence and the wild type sequence.

In some embodiments, the frameshift mutation is in a microsatellite in the TGFβR2 gene and comprises the sequence according to residues 270 to 278 of SEQ ID NO: 3, and wherein the first primer comprises the sequence defined by any one of SEQ ID NO: 17 to 19 or 21 to 23, and/or, when present, the second primer comprises the sequence defined by SEQ ID NO: 14.

In some embodiments, the frameshift mutation is in a microsatellite in the ASTE1 gene and comprises the sequence according to residues 328 to 337 of SEQ ID NO: 7, and wherein the first primer comprises the sequence defined by any one of SEQ ID NO: 34 to 38 or 40, and/or the second primer comprises the sequence defined by SEQ ID NO: 29.

In some embodiments, the frameshift mutation is in a microsatellite in the ASTE1 gene and comprises the sequence according to residues 328 to 337 of SEQ ID NO: 7, and wherein the first primer comprises the sequence defined by any one of SEQ ID NO: 39, 41, 42, 84 to 88, 98, 99 and 101, and/or, when present, the second primer comprises the sequence defined by SEQ ID NO: 29.

In some embodiments, the frameshift mutation is in a microsatellite in the TAF1β gene and comprises the sequence according to residues 255 to 264 of SEQ ID NO: 11, and wherein the first primer comprises the sequence defined by SEQ ID NOS: 53 to 59, and/or, when present, the second primer comprises the sequence defined by SEQ ID NO: 51.

In some embodiments, the frameshift mutation is in a microsatellite in the ACVR2A gene and comprises the sequence according to residues 509 to 515 of SEQ ID NO: 96, and wherein primer comprises the sequence defined by SEQ ID NO: 90 or 91, and/or, when present, the second primer comprises the sequence defined by SEQ ID NO: 92.

In some embodiments, the kit comprises the second primer and the kit further comprises: a third primer, wherein the third primer is configured to anneal 5′ upstream of the region to which the second primer is configured to anneal, and wherein the third primer is configured to anneal to the same strand as the first primer and the opposite strand to the second primer, or another control primer pair.

In some embodiments, the microsatellite is in a TGFβR2 gene, the sequence containing the wild type microsatellite comprises the sequence defined in SEQ ID NO: 3, and preferably the third primer comprises the sequence defined by SEQ ID NO: 13.

In some embodiments, the microsatellite is in an ASTE1 gene, the sequence containing the wild type microsatellite comprises the sequence defined in SEQ ID NO: 7, and preferably the third primer comprises the sequence defined in SEQ ID NO: 30 or SEQ ID NO: 31.

In some embodiments, the microsatellite is in a TAF1β gene, the sequence containing the wild type microsatellite comprises the sequence defined in SEQ ID NO: 11, and preferably the third primer comprises the sequence defined in SEQ ID NO: 50.

In some embodiments, the microsatellite is in an ACVR2A gene, the sequence containing the wild type microsatellite comprises the sequence defined in SEQ ID NO: 96, and preferably the third primer comprises the sequence defined in SEQ ID NO: 93.

In some embodiments, the kit further comprises the components for carrying out DNA amplification, preferably wherein the components comprise at least one of: a buffer, dNTPs, and

Taq-polymerase.

In another aspect of the invention there is provided a method for detecting a mutation in a microsatellite contained in a target sequence of a double stranded DNA molecule, wherein the mutation is a frameshift of the microsatellite as compared with the corresponding wild type microsatellite sequence, and the method comprising:
a) providing a first aliquot of a sample comprising human DNA,
b) adding to the first aliquot the necessary components for DNA amplification and a first primer to form a first reaction mix,
wherein the first primer is suitable for detection of a mutation in a microsatellite contained in a target sequence of a double stranded DNA molecule and comprises a region of nucleotides that is complementary to the target sequence containing the microsatellite having a frameshift mutation except for between one and four nucleotides which are mismatched to the target sequence containing the microsatellite having a frameshift mutation and which are also mismatched to a corresponding sequence containing the wild type microsatellite, and wherein the first primer is configured to anneal across the length of the microsatellite having a frameshift mutation and to anneal to at least one nucleotide flanking the 3′ end of the microsatellite having a frameshift mutation and at least one nucleotide flanking the 5′ end of the microsatellite having a frameshift mutation,
c) carrying out DNA amplification on the first reaction mix,
d) detecting the presence of the frameshift mutation in the sample when an amplification product is produced from the DNA amplification of the first reaction mix.

In some embodiments, the method comprises:
a) providing a first aliquot of a sample comprising human DNA,
b) adding to the first aliquot the necessary components for DNA amplification, a first primer and a second primer; wherein the first primer is suitable for detection of a mutation in a microsatellite contained in a target sequence of a double stranded DNA molecule and comprises a region of nucleotides that is complementary to the target sequence containing the microsatellite having a frameshift mutation except for between one and four nucleotides which are mismatched to the target sequence containing the microsatellite having a frameshift mutation and which are also mismatched to a corresponding sequence containing the wild type microsatellite and wherein the first primer is configured to anneal across the length of the microsatellite having a frameshift mutation and to anneal to at least one nucleotide flanking the 3′ end of the microsatellite having a frameshift mutation and at least one nucleotide flanking the 5′ end of the microsatellite having a frameshift mutation; and the second primer is configured to anneal to the target sequence 3′ downstream of the microsatellite, to form a first reaction mix;
wherein the first primer is configured to anneal to the sense strand of the DNA molecule and the second primer is configured to anneal to the antisense strand of the DNA molecule, or the first primer is configured to anneal to the antisense strand of the DNA molecule and the second primer is configured to anneal to the sense strand of the DNA molecule,
c) carrying out DNA amplification on the first reaction mix,
d) detecting the presence of the frameshift mutation in the sample when an amplification product is produced from the DNA amplification of the first reaction mix. In some embodiments, the primer comprises between one and three nucleotides which are mismatched to the target sequence containing the microsatellite having a frameshift mutation and which are also mismatched to a corresponding sequence containing the wild type microsatellite.

In some embodiments, the first primer is a primer according to the invention, and as defined above.

In some embodiments, the method further comprises:
a) also adding to the first aliquot either:
   I) the second primer and a third primer, wherein the third primer is configured to anneal 5′ upstream of the region to which the second primer is configured to anneal, and wherein the third primer is configured to anneal to the same strand as the first primer and the opposite strand to the second primer or
   II) a control primer pair, to form the first reaction mix or
b) providing a second aliquot of the sample comprising human DNA and adding to the second aliquot the necessary components for DNA amplification and either:
   I) the second primer and a third primer, wherein the third primer is configured to anneal 5′ upstream of the region to which the second primer is configured to anneal, and wherein the third primer is configured to anneal to the same strand as the first primer and the opposite strand to the second primer or
   II) a control primer pair, to form a second reaction mix
      carrying out DNA amplification on the reaction mix, and detecting that DNA amplification has been carried out successfully when an amplification product is produced from the DNA amplification of the second reaction mix.

In some embodiments, DNA amplification is polymerase chain reaction (PCR), wherein the PCR comprises a plurality of cycles of denaturation, annealing and extension.

In some embodiments, the reaction mix(es) comprises 1× or 0.5× buffer, 0.4 mM dNTPs, 0.2 μM forward primer, and 0.2 μM reverse primer. In some embodiments, the reaction mix(es) comprises a final concentration of dNTPs of 200 μM. In some embodiments, the reaction mix(es) comprises, as a final concentration, 1× or 0.5× buffer, 200 μM dNTPs, 0.2 μM forward primer and 0.2 μM reverse primer.

In some embodiments, step d) further comprises running the product of the PCR reaction on a gel and visualising a band to confirm that DNA amplification has been successful.

In some embodiments, the method further comprises step f) cutting out the band for DNA sequencing.

In some embodiments, the frameshift mutation is in a microsatellite in a TGFβR2 gene, an ASTE1 gene, a TAF1β gene or an ACVR2A gene.

In some embodiments, the frameshift mutation is in a microsatellite in the TGFβR2 gene and comprises the sequence according to residues 270 to 278 of SEQ ID NO: 3, and wherein the first primer comprises the sequence defined by any one of SEQ ID NO: 24 to 28, and/or the second primer comprises the sequence defined by SEQ ID NO: 14, and wherein X defines a substitution to any nucleotide which is mismatched to the corresponding nucleotide in the target sequence and the wild type sequence.

In some embodiments, the frameshift mutation is in a microsatellite in the ASTE1 gene and comprises the sequence according to residues 328-337 of SEQ ID NO: 7, and wherein the first primer comprises the sequence defined by SEQ ID NO: 45, 47, 83 or 100, and/or, when present, the second primer comprises the sequence defined by SEQ ID NO: 29, and wherein X defines a substitution to any nucleotide which is mismatched to the corresponding nucleotide in the target sequence and the wild type sequence.

In some embodiments, the frameshift mutation is in a microsatellite in the ASTE1 gene and comprises the sequence according to residues 328-337 of SEQ ID NO: 7, and wherein the first primer comprises the sequence defined by SEQ ID NO: 43, 44, 46, 48 or 49, and/or, when present, the second primer comprises the sequence defined by SEQ ID NO: 29, and wherein X defines a substitution to any nucleotide which is mismatched to the corresponding nucleotide in the target sequence and the wild type sequence.

In some embodiments, the frameshift mutation is in a microsatellite in the TAF1β gene and comprises the sequence according to residues 255 to 264 of SEQ ID NO: 11, and wherein the first primer comprises the sequence defined by SEQ ID NOS: 60 to 62, and/or, when present, the second primer comprises the sequence defined by SEQ ID NO: 51, and wherein X defines a substitution to any nucleotide which is mismatched to the corresponding nucleotide in the target sequence and the wild type sequence.

In some embodiments, the frameshift mutation is in a microsatellite in the ACVR2A gene and comprises the sequence according to residues 509 to 515 of SEQ ID NO: 96, and wherein the first primer comprises the sequence defined by SEQ ID NO: 89, and/or, when present, the second primer comprises the sequence defined by SEQ ID NO: 92, and wherein X defines a substitution to any nucleotide which is mismatched to the corresponding nucleotide in the target sequence and the wild type sequence.

In some embodiments, the frameshift mutation is in a microsatellite in the TGFβR2 gene and comprises the sequence according to residues 270 to 278 of SEQ ID NO: 3, and wherein the first primer comprises the sequence defined by any one of SEQ ID NO: 17 to 19 or 21 to 23, and/or, when present, the second primer comprises the sequence defined by SEQ ID NO: 14.

In some embodiments, the frameshift mutation is in a microsatellite in the ASTE1 gene and comprises the sequence according to residues 328-337 of SEQ ID NO: 7, and wherein the first primer comprises the sequence defined by any one of SEQ ID NO: 34 to 38, and/or, when present, the second primer comprises the sequence defined by SEQ ID NO: 29.

In some embodiments, the frameshift mutation is in a microsatellite in the ASTE1 gene and comprises the sequence according to residues 328-337 of SEQ ID NO: 7, and wherein the first primer comprises the sequence defined by any one of SEQ ID NO: 39, 41, 42, 84 to 88, 98, 99 and 101, and/or, when present, the second primer comprises the sequence defined by SEQ ID NO: 29.

In some embodiments, the frameshift mutation is in a microsatellite in the TAF1β gene and comprises the sequence according to residues 255 to 264 of SEQ ID NO: 11, and wherein the first primer comprises the sequence defined by SEQ ID NOS: 53 to 59, and/or the second primer comprises the sequence defined by SEQ ID NO: 51.

In some embodiments, the frameshift mutation is in a microsatellite in the ACVR2A gene and comprises the sequence according to residues 509 to 515 of SEQ ID NO: 96, and wherein the first primer comprises the sequence defined by SEQ ID NO: 90 or 91, and/or, when present, the second primer comprises the sequence defined by SEQ ID NO: 92

In some embodiments, the microsatellite is in a TGFβR2 gene, the sequence comprising the wild type microsatellite consists of the sequence according to SEQ ID NO: 3, and wherein the third primer comprises the sequence defined by SEQ ID NO: 13.

In some embodiments, the microsatellite is in an ASTE1 gene, the sequence comprising the wild type microsatellite comprises the sequence according to SEQ ID NO: 7, and wherein the third primer comprises the sequence defined by SEQ ID NO: 30 or SEQ ID NO: 31.

In some embodiments, the microsatellite is in a TAF1β gene, the sequence comprising the wild type microsatellite comprises the sequence according to SEQ ID NO: 11, and wherein the third primer comprises the sequence defined by SEQ ID NO: 50.

In some embodiments, the microsatellite is in an ACVR2A gene, the sequence comprising the wild type microsatellite comprises the sequence according to SEQ ID NO: 96, and wherein the third primer comprises the sequence defined by SEQ ID NO: 93.

In another aspect of the invention, there is provided a method of diagnosing a disease associated with a frameshift mutation in a microsatellite, comprising carrying out the method for detecting a mutation in a microsatellite according to the invention.

In some embodiments, the method further comprises determining that a patient suffering from a disease or disorder associated with a frameshift mutation is suitable for a treatment targeting said frameshift mutation if the frameshift mutation is detected in step d) in a sample from the patient.

In some embodiments, the disease or disorder associated with a frameshift mutation is a cancer.

In some embodiments, the disease or disorder is colorectal cancer or gastric cancer, and the treatment targeting the frameshift mutation is FMPV-1 or FMPV-3, wherein the frameshift mutation is in a microsatellite in the TGFβR2 gene and comprises the sequence according to residues 270 to 278 of SEQ ID NO: 4; or the disease or disorder is endometrial cancer or gastric cancer and the treatment targeting the frameshift mutation is FMPV-2 or FMPV-3, and the frameshift mutation is in a microsatellite in the ASTE1 gene and comprises the sequence according to residues 328 to 337 of SEQ ID NO: 8; or the disease or disorder is endometrial cancer or gastric cancer and the treatment targeting the frameshift mutation is an immunogenic fragment of the TAF1β-1a frameshift mutant protein or FMPV-3, and the frameshift mutation is in a microsatellite in the TAF1β gene and comprises the sequence according to residues 255 to 264 of SEQ ID NO: 12; or the disease or disorder is colon adenocarcinoma, stomach adenocarcinoma or uterine corpus endometrial cancer, and the frameshift mutation is in a microsatellite in the ACVR2A gene and comprises the sequence according to residues 509 to 515 of SEQ ID NO: 97.

In some embodiments, the method further comprises step e) of treating the patient with FMPV-1, FMPV-2, an immunogenic fragment of the TAF1β-1a frameshift mutant protein, or FMPV-3.

In some embodiments, the microsatellite is in a TGFβR2 gene, the sequence comprising the wild type microsatellite comprises the sequence according to SEQ ID NO: 3, and the third primer comprises the sequence defined by SEQ ID NO: 13.

In some embodiments, the microsatellite is in an ASTE1 gene, the sequence comprising the wild type microsatellite comprises the sequence according to SEQ ID NO: 7, and the third primer comprises the sequence defined by SEQ ID NO: 30 or SEQ ID NO: 31.

In some embodiments, the microsatellite is in a TAF1β gene, the sequence comprising the wild type microsatellite comprises the sequence according to SEQ ID NO: 11, and wherein the third primer comprises the sequence defined by SEQ ID NO: 50.

In some embodiments, the microsatellite is in an ACVR2A gene, the sequence comprising the wild type microsatellite comprises the sequence according to SEQ ID NO: 96, and the third primer comprises the sequence defined by SEQ ID NO: 93.

In some embodiments, the first primer comprises the sequence defined by any one of SEQ ID NO: 24 to 28, and/or, when present, the second primer comprises the sequence defined by SEQ ID NO: 14; or the first primer comprises the sequence defined by SEQ ID NO: 45, 47, 83 or 100, and/or, when present, the second primer comprises the sequence defined by SEQ ID NO: 29; or the first primer comprises the sequence defined by SEQ ID NO: 43, 44, 46, 48 or 49, and/or, when present, the second primer comprises the sequence defined by SEQ ID NO: 29; or the first primer comprises the sequence defined by SEQ ID NO: 60 to 62, and/or, when present, the second primer comprises the sequence defined by SEQ ID NO: 51; or the first primer comprises the sequence defined by SEQ ID NO: 89, and/or, when present, the second primer comprises the sequence defined by SEQ ID NO: 92; and wherein X defines a substitution to any nucleotide which is mismatched to the corresponding nucleotide in the target sequence and the wild type sequence.

In some embodiments, the first primer comprises the sequence defined by any one of SEQ ID NO: 17 to 19 or 21 to 23, and/or, when present, the second primer comprises the sequence defined by SEQ ID NO: 14; or the first primer comprises the sequence defined by SEQ ID NO: 34 to 38 or 40, and/or, when present, the second primer comprises the sequence defined by SEQ ID NO: 29; or the first primer comprises the sequence defined by any one of SEQ ID NO: 39, 41, 42, 84 to 88, 98, 99 and 101, and/or, when present, the second primer comprises the sequence defined by SEQ ID NO: 29; or the first primer comprises the sequence defined by SEQ ID NO: 53 to 59, and/or, when present, the second primer comprises the sequence defined by SEQ ID NO: 51; or the first primer comprises the sequence defined by SEQ ID NO: 90 or 91, and/or, when present, the second primes comprises the sequence defined by SEQ ID NO: 92.

In some embodiments, the sample is a liquid biopsy comprising cell free DNA, preferably wherein the liquid biopsy is plasma.

In some embodiments, the DNA amplification is PCR, and the PCR is carried out in high stringency conditions, optionally wherein the high stringency conditions comprise at least one of:
a) carrying out the annealing step of PCR at a temperature that is at least 1° C., 2° C., 3° C., 4° C., 5° C., 6° C., 7° C., 8° C., 9° C. or 10° C. higher than the recommended annealing temperature of the reaction;
b) carrying out the annealing step of PCR for only 30 seconds, preferably 15 seconds, per cycle;
c) carrying out the DNA amplification in a buffer concentration that is less than 0.1×, 0.2×, 0.3×, 0.4×, 0.5×, 0.6×, or 0.75×;
d) carrying out the DNA amplification in a buffer comprising ammonium ions;
e) performing 25, 20, 15, or 10 cycles of PCR, or fewer than 25, fewer than 20, fewer than 15 or fewer than 10 cycles of PCR; and
f) carrying out the DNA amplification using a template DNA concentration of 0.2 ng or less, or 0.05 ng or less.

In some embodiments, the buffer includes ammonium (NH₄⁺) ions.

In some embodiments, the buffer includes ammonium (NH₄⁺) ions and is at a concentration of 1×.

In some embodiments, the buffer is 1× Key buffer.

### Definitions

In this specification, the following terms may be understood as follows:

The terms “gene”, “polynucleotides”, and “nucleic acid molecules” are used interchangeably herein to refer to a polymer of multiple nucleotides. The nucleic acid molecules may comprise naturally occurring nucleic acids (i.e. DNA or RNA) or may comprise artificial nucleic acids such as peptide nucleic acids, morpholin and locked nucleic acids as well as glycol nucleic acids and threose nucleic acids.

The term “nucleotide” as used herein refers to naturally occurring nucleotides and synthetic nucleotide analogues that are recognised by cellular enzymes.

The term “purine nucleotide” as used herein refers to a nucleotide containing a purine base. In particular, the term “purine nucleotide” refers to adenine or guanine.

The term “pyrimidine nucleotide” as used herein refers to a nucleotide containing a pyrimidine base. In particular, the term “pyrimidine nucleotide” refers to cytosine, thymine or uracil, preferably to cytosine or thymine.

The phrase “high stringency conditions” means conditions that only allow for successful DNA amplification where there is a very high level of matching to the target sequence. In other words, the conditions are suboptimal for DNA amplification in order that primers with lower sequence matching to the target sequence are not able to anneal. Such suboptimal conditions can be generated by altering one or more of temperature, cycle number, ionic strength and the presence of certain organic solvents that allow pairing of nucleic acid sequences. Further details on the high stringency conditions are provided below. In particular, the high stringency conditions may include where the annealing step of PCR is carried out at a temperature that is at least 2%, 5%, or 10% higher than the recommended annealing temperature of the reaction. Alternatively, the annealing step of PCR is carried out at a temperature that is at least 1° C., 2° C., 3° C., 4° C., 5° C., 6° C., 7° C., 8° C., 9° C. or 10° C. higher than the recommended annealing temperature of the reaction. In some embodiments, the annealing step of PCR is carried out at a temperature that is 4° C. higher than the recommended annealing temperature of the reaction. For example, the annealing step may be carried out between 42° C. and 61° C. or 62° C., as further detailed below. The annealing step may additionally or alternatively be carried out for a shorter period than is recommended for the reaction, for example, the annealing step may be reduced from 45 seconds to 30 seconds or 15 seconds. Additionally or alternatively, the high stringency conditions may include using a lower buffer concentration than is recommended for the reaction, such as 10%, 20%, 30%, 40%, 50%, 60% or 75% of the recommended concentration of a buffer in a reaction, in particular the buffer concentration may be less than 0.1×, 0.2×, 0.3×, 0.4×, 0.5×, 0.6× or 0.75×. Additionally or alternatively, the high stringency conditions may include using a more stringent buffer in the reaction, such as a buffer including ammonium ions (NH₄⁺), for example, Key buffer. A more stringent buffer may be used at 1× concentration. In some embodiments, 1× Key buffer is used. Additionally or alternatively, the high stringency conditions may include reducing the number of cycles in a PCR, such as carrying out 25 cycles, 20 cycles, 15 cycles or even 10 cycles, compared to 30, 35 or 40 cycles. In some embodiments, 30 or fewer, 25 or fewer, 20 or fewer, 15 or fewer or 10 or fewer cycles of PCR are carried out. Additionally or alternatively, the high stringency conditions may include using a lower template DNA concentration than is recommended for the reaction, for example, the template DNA concentration may be between 0.001 ng/μL and 1.5 ng/μL, 0.002 ng/μL and 1.2 ng/μL, 0.01 ng/μL and 1.0 ng/μL, or 0.02 ng/μL and 0.5 ng/μL, preferably between 0.05 ng/μL and 0.2 ng/μL, as further detailed below. Any one or more of the above conditions may be used.

The term “frameshift” means a genetic mutation caused by a deletion or insertion in a DNA sequence that causes a shift in the sequence, meaning that the nucleotides are grouped into a different series of codons resulting in a different protein being translated from this sequence. In many cases, the frameshift may cause a premature stop codon in the protein sequence, resulting in a truncated protein sequence. For example, one such frameshift mutation in TGFβR2 is the deletion of a single adenine, meaning that a sequence of 10 adenines (a10; which is a microsatellite) is reduced to 9 adenines (a9). The sequence of wild type TGFβR2 is shown in SEQ ID NO: 1 and the sequence of a9 TGFβR2 is shown in SEQ ID NO: 2. Another such frameshift is a deletion of a single adenine in ASTE1, meaning that a sequence of 11 adenines (a11; which is a microsatellite) is reduced to 10 adenines (a10). The sequence of wild type ASTE1 is shown in SEQ ID NO: 5 and the sequence of a10 ASTE1 is shown in SEQ ID NO: 6. Another such frameshift is a deletion of a single adenine in TAF1β, meaning that a sequence of 11 adenines (a11; which is a microsatellite) is reduced to 10 adenines (a10). The sequence of wild type TAF1β is shown in SEQ ID NO: 9 and the sequence of a10 ASTE1 is shown in SEQ ID NO: 10. Another such frameshift is a deletion of a single adenine in exon 10 ACVR2A, meaning that a sequence of 8 adenines (a9; which is a microsatellite) is reduced to 7 adenines (a7). The sequence of wild type ACVR2A is shown in SEQ ID NO: 94 and the sequence of a7 ACVR2A is shown in SEQ ID NO: 95.

The term “microsatellite” means a region of DNA where a sequence is repeated, typically between 5 and 50 times. Microsatellites may also be particularly vulnerable to mutation. Microsatellites may also be known as “short tandem repeats (STRs)”. Microsatellites may be a repeated series of a single nucleotide such as A, G, C or T, or may be a repeated series of a longer motif, such as TA (dinucleotide repeat) or GTC (trinucleotide repeat).

As is well known in molecular biology, guanine (G) and cytosine (C), and adenine (A) and thymine (T), are complementary nucleotides, and thus will pair e.g. during DNA replication. In the current invention, a “mismatch” or a “mismatched” nucleotide may be a deletion of a complementary nucleotide, an addition of a nucleotide, or a substitution of a complementary nucleotide for a non-complementary (i.e. mismatched) nucleotide. Preferably, a “mismatch” or a “mismatched” nucleotide is a substitution of a complementary nucleotide for a non-complementary (i.e. mismatched) nucleotide. In some embodiments, each of the mismatches in the primer of the invention is a nucleotide substitution. For example, a mismatch to a target guanine (G) may be an adenine (A), a thymine (T) or another guanine (G), but not a cytosine (C). Where a frameshift mutation causes an increase in the microsatellite length, then an addition of a nucleotide is particularly useful for distinguishing the frameshift mutated microsatellite from the wild type microsatellite. In contrast, where a frameshift mutation causes a decrease in the microsatellite length, then a deletion of a nucleotide is particularly useful for distinguishing the frameshift mutated microsatellite from the wild type microsatellite. In one example, this deletion is a deletion of a single nucleotide from a microsatellite sequence.

Again, as is well known in the field of molecular biology, in humans DNA is formed of a double stranded DNA helix formed of two strands, a sense and an antisense strand. Thus the term “sense strand” will be known to the skilled person as the coding strand, carrying transcribable nucleotides in a 5′ to 3′ direction and the term “antisense strand” will be known to the skilled person as a strand having a reverse complementary sequence to the coding strand in a 5′ to 3′ direction, and being the template for mRNA transcription.

The term “primer” refers to a short single stranded DNA sequence that is used to initiate targeted DNA amplification. It is typically between 18 and 24 bases in length, but is shorter or longer than this typical length in some embodiments. A “primer pair” is two such primers that cause DNA amplification of a specific target sequence lying between the annealing sites of these two primers.

The term “treating” refers to any partial or complete treatment and includes: inhibiting the disease or symptom, i.e. arresting its development; and relieving the disease or symptom, i.e. causing regression of the disease or symptom.

It is to be understood that where reference is made to “3′ downstream” this is with respect to the (first) primer, i.e. in the direction of DNA polymerase extension of the (first) primer. In other words, the term “3′ downstream” refers to a position 3′ in the primer, which corresponds to a position 5′ in the template sequence. For example, the expression “at least one mismatched nucleotide is located in a position of the primer that is configured to anneal 3′ downstream of the microsatellite”, above, means that the at least one mismatched nucleotide is 3′ downstream, in the primer, of the microsatellite, i.e. 5′ upstream of the microsatellite in the template sequence. Similarly, it is to be understood that where reference is made to the “3′ end of the microsatellite having a frameshift mutation”, this is with respect to the (first) primer. In other words, the term “3′ end” refers to the 3′ end in the primer, which corresponds to the 5′ end in the template sequence.

Similarly, it is to be understood that, where reference is made to “5′ upstream”, this is with respect to the (first) primer. For example, the expression “the primer is configured to anneal across the length of the microsatellite having a frameshift mutation, and to at least two nucleotides flanking the 5′ and/or at least two nucleotides flanking the 3′ end of the microsatellite having a frameshift mutation”, above, means that the primer may anneal to at least two nucleotides flanking the 5′ end of the microsatellite with respect to the primer, i.e. to at least two nucleotides flanking the 3′ end of the microsatellite with respect to the template. Similarly, it is to be understood that where reference is made to the “5′ end of the microsatellite having a frameshift mutation”, this is with respect to the (first) primer. In other words, the term “5′ end” refers to the 5′ end in the primer, which corresponds to the 3′ end in the template sequence.

### BRIEF DESCRIPTION OF THE FIGURES

FIG. 1 shows two electrophoresis gels of the products of PCR reactions carried out on 10a microsatellite TGFβR2 template DNA (i.e. wild type) using primer pair P1 and P2 using different quantities of template DNA. FIG. 1A shows results with increasing amounts of template DNA in nanograms (ng), and FIG. 1B shows results with increasing amounts of template DNA in pictograms (pg).

FIG. 2 shows an electrophoresis gel of the products of PCR reactions carried out on 9a microsatellite TGFβR2 template DNA (i.e. mutant) and 10a microsatellite TGFβR2 template DNA (i.e. wild type) for primers P1 and P4 in combination with primer P2, at a more stringent condition using an annealing temperature of 53° C. and a reduced cycle number (25).

FIG. 3 shows an electrophoresis gel of the products of PCR reactions carried out on 9a microsatellite TGFβR2 template DNA (i.e. mutant) and 10a microsatellite TGFβR2 template DNA (i.e. wild type) for primer pair P4 and P2 at an even more stringent condition using an annealing temperature of 55° C.

FIG. 4 shows an electrophoresis gel of the products of PCR reactions carried out on 9a microsatellite TGFβR2 template DNA (i.e. mutant) and 10a microsatellite TGFβR2 template DNA (i.e. wild type) for primers P6 and P10 in combination with primer P2. In particular, those lanes denoted with an asterisk (*) were those for products of a PCR reaction carried out at a low buffer concentration.

FIG. 5 shows three electrophoresis gels of the products of PCR reactions carried out on 9a microsatellite TGFβR2 template DNA (i.e. mutant) for primers P4, P6 and P10 in combination with primer P2 (as well as primer P1 in combination with primer P2 as a positive control) at increasingly stringent PCR conditions. The reactions in FIG. 5A were carried out at an annealing temperature of 55° C. for 30 cycles. The reactions in FIG. 5B were carried out at an annealing temperature of 56° C. for 25 cycles. The reactions in FIG. 5C were carried out at an annealing temperature of 58° C. for 25 cycles. Again, those lanes denoted with an asterisk (*) were those for products of a PCR reaction carried out at a low buffer concentration.

FIG. 6 shows two electrophoresis gels of the products of PCR reactions carried out on 9a microsatellite TGFβR2 template DNA (i.e. mutant) and 10a microsatellite TGFβR2 template DNA (i.e. wild type) for primers P1, P6 and P10 in combination with primer P2. In particular, those lanes denoted with an asterisk (*) were those for products of a PCR reaction carried out at a low buffer concentration. The reactions in FIG. 6A were carried out with primer pairs and the reactions in FIG. 6B were carried out with three primers.

FIG. 7 shows an electrophoresis gel of the products of PCR reactions carried out on 9a microsatellite TGFβR2 template DNA (i.e. mutant) and 10a microsatellite TGFβR2 template DNA (i.e. wild type) for primers P1, P4 and P10 in combination with primer P2. This includes three primer reactions that include both primers P1 and P2 as well primer P4 or P10.

FIG. 8 shows an electrophoresis gel of the products of PCR reactions carried out on 9a microsatellite TGFβR2 template DNA (i.e. mutant) and 10a TGFβR2 microsatellite template DNA (i.e. wild type) for each of the primer combinations listed, demonstrating that all of these primers are functional.

FIG. 9 shows two electrophoresis gels of the products of PCR reactions carried out on 9a microsatellite TGFβR2 template DNA (i.e. mutant) DNA for primers P1, P4.1, P4.2 and P10.1, where the PCR reactions have either been carried out in standard Taq (KCl) buffer or Key buffer (containing ammonium ions, NH₄⁺). The reactions in FIG. 9A were carried out at an annealing temperature of 56° C. and the reactions in FIG. 9B were carried out at an annealing temperature of 58° C.

FIG. 10 shows a gel electrophoresis of the PCR reactions carried out on 9a microsatellite template TGFβR2 DNA (i.e. mutant) and 10a microsatellite TGFβR2 template DNA (i.e. wild type) for primers P4, P4.1, P4.2 and P6 in combination with primer P2. The reactions in FIG. 10A were carried out using standard Taq buffer, and the reactions in FIG. 10B were carried out using Key buffer (comprising ammonium, NH₄⁺).

FIG. 11 shows a gel electrophoresis of the PCR reactions carried out on 9a microsatellite TGFβR2 template DNA (i.e. mutant) and 10a microsatellite TGFβR2 template DNA (i.e. wild type) for primers P4, P4.1, P4.2 and P6 in combination with primer P2. The reactions in FIG. 11A were carried out using standard Taq buffer (which does not comprise ammonium), and the reactions in FIG. 11B were carried out using Key buffer (comprising ammonium, NH₄⁺).

FIG. 12 shows the results of the detection of TGFβR2 cfDNA from MSI-CRC and microsatellite stable-CRC (MSS-CRC) patients using primers defining a fragment of TGFβR2 comprising the a10/a9 microsatellite. ¹primer designed for detection of TGFβR2 a9 (mutant) microsatellite; ²Control primers for detection of both a9 and a10 TGFβR2 microsatellites.

FIG. 13 shows an electrophoresis gel of the products of PCR reactions carried out on 9a microsatellite TGFβR2 template DNA (i.e. mutant, F1) and 10a microsatellite TGFβR2 template DNA (i.e. wildtype, F2) using primers P2 and P4 at different annealing temperatures.

FIG. 14 shows an electrophoresis gel of the products of PCR reactions carried out on (A) 9a microsatellite TGFβR2 template DNA (i.e. mutant, F1) and (B) 10a microsatellite TGFβR2 template DNA (i.e. wildtype, F2) using primers P2 and P4 at different annealing temperatures.

FIG. 15 shows an electrophoresis gel of the products of PCR reactions carried out using primer P4 on 9a microsatellite TGFβR2 template DNA (i.e. mutant, F1) and 10a microsatellite TGFβR2 template DNA (i.e. wildtype, F2) using standard Taq (KCl) buffer using an annealing temperature of 57° C. Primer P2 was the forward primer for all reactions and primer P1 was the positive control reverse primer.

FIG. 16 shows an electrophoresis gel of the products of PCR reactions carried out with primers P11 and P16 on (A) 10a microsatellite ASTE1 template DNA (i.e. mutant, F3) and (B) 11a microsatellite ASTE1 template DNA (i.e. wild type, F4), using a range of annealing temperatures.

FIG. 17 shows electrophoresis gels of the products of PCR reactions carried out with primer P24 on (A) 10a microsatellite ASTE1 template DNA (i.e. mutant, F3, referred to as “M” in the gels) and (B) 11a microsatellite ASTE1 template DNA (i.e. wild type, F4, referred to as “Wt” in the gels), using a range of annealing temperatures.

FIG. 18 shows electrophoresis gels of the products of PCR reactions carried out with primer P12 on (A) 10a microsatellite ASTE1 template DNA (i.e. mutant, F3) and (B) 11a microsatellite ASTE1 template DNA (i.e. wild type, F4,), using a range of annealing temperatures.

FIG. 19 shows electrophoresis gels of the products of PCR reactions carried out with primer P13 on (A and C) 10a microsatellite ASTE1 template DNA (i.e. mutant, F3) and (B and D) 11a microsatellite ASTE1 template DNA (i.e. wild type, F4), using a range of annealing temperatures. P12 was used as the positive control reverse primer. Replicate experimental results are shown.

FIG. 20 shows electrophoresis gels of the products of PCR reactions carried out with primer P13 on (A) 10a microsatellite ASTE1 template DNA (i.e. mutant, F3) and (B) 11a microsatellite ASTE1 template DNA (i.e. wild type, F4), using a narrow range of annealing temperatures. P12 was used as the positive control reverse primer.

FIG. 21 shows electrophoresis gels of the products of PCR reactions carried out with primer P23 on (A) 10a microsatellite ASTE1 template DNA (i.e. mutant, F3) and (B) 11a microsatellite ASTE1 template DNA (i.e. wild type, F4), using a range of annealing temperatures. P12 was used as the positive control reverse primer.

FIG. 22 shows electrophoresis gels of the products of PCR reactions carried out with primer P23 on (A) 10a microsatellite ASTE1 template DNA (i.e. mutant, F3) and (B) 11a microsatellite ASTE1 template DNA (i.e. wild type, F4), using a narrow range of annealing temperatures. P12 was used as the positive control reverse primer.

FIG. 23 shows electrophoresis gels of the products of PCR reactions carried out with primer P23 in standard Taq buffer on (A) 10a microsatellite ASTE1 template DNA (i.e. mutant, F3) and (B) 11a microsatellite ASTE1 template DNA (i.e. wild type, F4), using a narrow range of annealing temperatures. P12 was used as the positive control reverse primer.

FIG. 24 shows electrophoresis gels of the products of PCR reactions carried out with primer P29 on (A) 10a microsatellite ASTE1 template DNA (i.e. mutant, F3) and (B) 11a microsatellite ASTE1 template DNA (i.e. wild type, F4), using a range of annealing temperatures. P12 was used as the positive control reverse primer.

FIG. 25 shows electrophoresis gels of the products of PCR reactions carried out with primer P31 on (A) 10a microsatellite ASTE1 template DNA (i.e. mutant, F3) and (B) 11a microsatellite ASTE1 template DNA (i.e. wild type, F4), using a range of annealing temperatures. P12 was used as the positive control reverse primer.

FIG. 26 shows an electrophoresis gel of the products of PCR reactions carried out with primers P42 and P43 on 10a microsatellite TAF1β template DNA (i.e. mutant, F5) and 11a microsatellite TAF1β template DNA (i.e. wild type, F6), at 54° C. P37 was used as the positive control forward primer.

FIG. 27 shows an electrophoresis gel of the products of PCR reactions carried out with primers P.ACV.21 and P.ACV.22 on 8a microsatellite ACVR2A template DNA (i.e. wild type, SEQ ID NO: 96) and 7a microsatellite ACVR2A template DNA (i.e. mutant, SEQ ID NO: 97), using a range of annealing temperatures. P.ACV.1 (SEQ ID NO: 92) was used as the positive control reverse primer.

FIG. 28 shows an electrophoresis gel of the products of PCR reactions carried out with primers P42 and P43 on 11a microsatellite TAF1β template DNA (i.e. wild type, SEQ ID NO: 11) and 10a microsatellite TAF1β template DNA (i.e. mutant, SEQ ID NO: 12), using a range of annealing temperatures. P37 (SEQ ID NO: 50) was used as the positive control reverse primer.

FIG. 29 shows an electrolysis gel of the products of PCR reactions carried out with primers P.AST.54 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 30 shows an electrolysis gel of the products of PCR reactions carried out with primers P.AST.55 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 31 shows an electrolysis gel of the products of PCR reactions carried out with primers P.AST.59 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 32 shows an electrolysis gel of the products of PCR reactions carried out with primers P.AST.60 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 33 shows an electrolysis gel of the products of PCR reactions carried out with primers P.AST.61 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 34 shows an electrolysis gel of the products of PCR reactions carried out with primers P.AST.59 and P11 on (A) 10a microsatellite ASTE1 template DNA (i.e. mutant), and on (B) 11a microsatellite ASTE1 template DNA (i.e. wild-type), using a narrow range of annealing temperatures. P.AST.2 (SEQ ID NO: 102) was used as the positive control reverse primer.

FIG. 35 shows an electrolysis gel of the products of PCR reactions carried out with primers P.AST.60 and P11 on (A) 10a microsatellite ASTE1 template DNA (i.e. mutant), and on (B) 11a microsatellite ASTE1 template DNA (i.e. wild-type), using a narrow range of annealing temperatures. P.AST.2 (SEQ ID NO: 102) was used as the positive control reverse primer.

FIG. 36 shows an electrolysis gel of the products of PCR reactions carried out with primers P.AST.61 and P11 on (A) 10a microsatellite ASTE1 template DNA (i.e. mutant), and on (B) 11a microsatellite ASTE1 template DNA (i.e. wild-type), using a narrow range of annealing temperatures. P.AST.2 (SEQ ID NO: 102) was used as the positive control reverse primer.

FIG. 37 shows an electrolysis gel of the products of PCR reactions carried out with primers P.AST.65 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 38 shows an electrolysis gel of the products of PCR reactions carried out with primers P.AST.67 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

FIG. 39 shows an electrolysis gel of the products if PCR reactions carried out with primers P.AST.79 and P11 on (A) 10a microsatellite ASTE1 template DNA (i.e. mutant, F3), and on (B) 11a microsatellite ASTE1 template DNA (i.e. wild-type, F4), using a range of annealing temperatures. P.AST.2 (SEQ ID NO: 102) was used as the positive control reverse primer.

### BRIEF DESCRIPTION OF THE SEQUENCE LISTING

SEQ ID NO: 1 is the full length wild type TGFβR2 (a10) gene sequence.

SEQ ID NO: 2 is the full length a9 frameshift mutant TGFβR2 gene sequence.

SEQ ID NO: 3 is a peptide of the wild type TGFβR2 (a10) gene sequence (referred to as “F2” herein).

SEQ ID NO: 4 is a peptide of the a9 frameshift mutant TGFβR2 gene sequence (referred to herein as “F1”).

SEQ ID NO: 5 is the full length wild type ASTE1 (a11) gene sequence.

SEQ ID NO: 6 is the full length a10 frameshift mutant ASTE1 gene sequence.

SEQ ID NO: 7 is a peptide of the wild type ASTE1 (a11) gene sequence (referred to herein as “F4”).

SEQ ID NO: 8 is a peptide of the a10 frameshift mutant ASTE1 gene sequence (referred to herein as “F3”).

SEQ ID NO: 9 is the full length wild type TAF1β (a11) gene sequence.

SEQ ID NO: 10 is the full length a10 frameshift mutant TAF1β gene sequence.

SEQ ID NO: 11 is a peptide of the wild type TAF1β (a11) gene sequence (referred to herein as “F6”).

SEQ ID NO: 12 is a peptide of the a10 frameshift mutant TAF1β gene sequence (referred to herein as “F5”).

SEQ ID NO: 13 is a primer referred to herein as “P1”.

SEQ ID NO: 14 is a primer referred to herein as “P2”.

SEQ ID NO: 15 is a primer referred to herein as “Pwt”.

SEQ ID NO: 16 is a primer referred to herein as “Pwt2”.

SEQ ID NO: 17 is a primer referred to herein as “P4”.

SEQ ID NO: 18 is a primer referred to herein as “P4.1”.

SEQ ID NO: 19 is a primer referred to herein as “P4.2”.

SEQ ID NO: 20 is a primer referred to herein as “P5”.

SEQ ID NO: 21 is a primer referred to herein as “P6”.

SEQ ID NO: 22 is a primer referred to herein as “P10”.

SEQ ID NO: 23 is a primer referred to herein as “P10.1”.

SEQ ID NO: 24 is a primer referred to herein as “P4x”.

SEQ ID NO: 25 is a primer referred to herein as “P4.1x/P6x”.

SEQ ID NO: 26 is a primer referred to herein as “P4.2”.

SEQ ID NO: 27 is a primer referred to herein as “P10”.

SEQ ID NO: 28 is a primer referred to herein as “P10.1”.

SEQ ID NO: 29 is a primer referred to herein as “P11”.

SEQ ID NO: 30 is a primer referred to herein as “P12”.

SEQ ID NO: 31 is a primer referred to herein as “P12.1”.

SEQ ID NO: 32 is a primer referred to herein as “Pwt3”.

SEQ ID NO: 33 is a primer referred to herein as “Pwt4”.

SEQ ID NO: 34 is a primer referred to herein as “P13”.

SEQ ID NO: 35 is a primer referred to herein as “P14”.

SEQ ID NO: 36 is a primer referred to herein as “P15”.

SEQ ID NO: 37 is a primer referred to herein as “P16”.

SEQ ID NO: 38 is a primer referred to herein as “P17”.

SEQ ID NO: 29 is a primer referred to herein as “P23”.

SEQ ID NO: 40 is a primer referred to herein as “P24”.

SEQ ID NO: 41 is a primer referred to herein as “P29”.

SEQ ID NO: 42 is a primer referred to herein as “P31”.

SEQ ID NO: 43 is a primer referred to herein as “P13x/P14x”.

SEQ ID NO: 44 is a primer referred to herein as “P15”.

SEQ ID NO: 45 is a primer referred to herein as “P16x/P17x”.

SEQ ID NO: 46 is a primer referred to herein as “P23”.

SEQ ID NO: 47 is a primer referred to herein as “P24x”.

SEQ ID NO: 48 is a primer referred to herein as “P29x”.

SEQ ID NO: 49 is a primer referred to herein as “P31”.

SEQ ID NO: 50 is a primer referred to herein as “P37”.

SEQ ID NO: 51 is a primer referred to herein as “P38”.

SEQ ID NO: 52 is a primer referred to herein as “P39”.

SEQ ID NO: 53 is a primer referred to herein as “P40”.

SEQ ID NO: 54 is a primer referred to herein as “P41”.

SEQ ID NO: 55 is a primer referred to herein as “P42”.

SEQ ID NO: 56 is a primer referred to herein as “P43”.

SEQ ID NO: 57 is a primer referred to herein as “P44”.

SEQ ID NO: 58 is a primer referred to herein as “P45”.

SEQ ID NO: 59 is a primer referred to herein as “P46”.

SEQ ID NO: 60 is a primer referred to herein as “P40x/P41x”.

SEQ ID NO: 61 is a primer referred to herein as “P42x/P43x”.

SEQ ID NO: 62 is a primer referred to herein as “P44x/P45x/P46x”.

SEQ ID NO: 63 is the corresponding target sequence in the wild type TGFβR2 (a10) gene for primers Pwt, P4, P4.1, P4.2, P5, P6, P4x, P4.1x/P6x, and P4.2x.

SEQ ID NO: 64 is the corresponding target sequence in the wild type TGFβR2 (a10) gene for primers Pwt2, P10, P10.1, P10x, and P10.1x.

SEQ ID NO: 65 is the corresponding target sequence in the a9 frameshift mutant TGFβR2 gene for primers Pwt, P4, P4.1, P4.2, P5, P6, P4x, P4.1x/P6x, and P4.2x.

SEQ ID NO: 66 is the corresponding target sequence in the a9 frameshift mutant TGFβR2 gene for primers Pwt2, P10, P10.1, P10x, and P10.1x.

SEQ ID NO: 67 is the corresponding target sequence in the wild type ASTE1 (a11) gene for primers Pwt3, P13, P14, P15, P16, P17, P23, P29, P31, P13x/P14x, P15x, P16x/P17x, P23x, P29x, and P31x.

SEQ ID NO: 68 is the corresponding target sequence in the wild type ASTE1 (a11) gene for primers Pwt4, P24, and P24x.

SEQ ID NO: 69 is the corresponding target sequence in the a10 frameshift mutant ASTE1 gene for primers Pwt3, P13, P14, P15, P16, P17, P23, P29, P31, P13x/P14x, P15x, P16x/P17x, P23x, P29x, and P31x.

SEQ ID NO: 70 is the corresponding target sequence in the a10 frameshift mutant ASTE1 gene for primers Pwt4, P24, and P24x.

SEQ ID NO: 71 is the corresponding target sequence in the wild type TAF1β (a11) gene for primers P39, P40, P41, P42, P43, P44, P45, P46, P40x/P41x, P42x/P43x, and P44x/P45x/P46x.

SEQ ID NO: 72 is the corresponding target sequence in the a10 frameshift mutant TAF1β gene for primers P39, P40, P41, P42, P43, P44, P45, P46, P40x/P41x, P42x/P43x, and P44x/P45x/P46x.

SEQ ID NO: 73 is the sequence of the cfDNA from patient BD_001 (MSI-H).

SEQ ID NO: 74 is the sequence of the cfDNA from patient BD_002 (MSI-H).

SEQ ID NO: 75 is the sequence of the cfDNA from patient BD_003 (MSI-H).

SEQ ID NO: 76 is the sequence of the cfDNA from patient BD_004 (MSI-H).

SEQ ID NO: 77 is the sequence of the cfDNA from patient BD_005 (MSI-H).

SEQ ID NO: 78 is the sequence of the cfDNA from patient BD_006 (MSI-H).

SEQ ID NO: 79 is the sequence of the cfDNA from patient BD_007 (MSI-H).

SEQ ID NO: 80 is the sequence of the cfDNA from patient BD_008 (MSI-H).

SEQ ID NO: 81 is the sequence of the cfDNA from patient BD_009 (MSI-H).

SEQ ID NO: 82 is the sequence of the cfDNA from patient BD_010 (MSI-H).

SEQ ID NO: 83 is a primer referred to herein as P.AST.X1.

SEQ ID NO: 84 is a primer referred to herein as P.AST.54.

SEQ ID NO: 85 is a primer referred to herein as P.AST.55.

SEQ ID NO: 86 is a primer referred to herein as P.AST.59.

SEQ ID NO: 87 is a primer referred to herein as P.AST.60.

SEQ ID NO: 88 is a primer referred to herein as P.AST.61.

SEQ ID NO: 89 is a primer referred to herein as P.ACV.X.

SEQ ID NO: 90 is a primer referred to herein as P.ACV.21.

SEQ ID NO: 91 is a primer referred to herein as P.ACV.22.

SEQ ID NO: 92 is a primer referred to herein as P.ACV.1.

SEQ ID NO: 93 is a primer referred to herein as P.ACV.2.

SEQ ID NO: 94 is the full length wild type ACVR2A (a8) gene sequence.

SEQ ID NO: 95 is the full length a7 exon 10 frameshift mutant ACVR2A gene sequence.

SEQ ID NO: 96 is a peptide of the wild type ACVR2A (a8) gene sequence.

SEQ ID NO: 97 is a peptide of the a7 frameshift mutant ACVR2A gene sequence. SEQ ID NO: 98 is a primer referred to herein as P.AST.65.

SEQ ID NO: 99 is a primer referred to herein as P.AST.67.

SEQ ID NO: 100 is a primer referred to herein as P.AST.X2.

SEQ ID NO: 101 is a primer referred to herein as P.AST.79.

SEQ ID NO: 102 is a primer referred to herein as P.AST.2.

Pwt, Pwt2, P5, Pwt3, Pwt4, P39 are reference primers which comprises sequences which correspond to the primers of the invention but do not contain any mismatches to the target sequence containing the frameshift mutation in the microsatellite.

### DETAILED DESCRIPTION OF THE INVENTION

In view of the above, there is a need to develop a tool for specific detection of frameshift mutations that may be relevant in disease, such as a frameshift mutation in the microsatellite of TGFβR2, ASTE1, TAF1β or ACVR2A. There is a further need for this tool to be suitable for detection of cell free DNA (cfDNA) in liquid biopsies (e.g. plasma).

Thus, in a first aspect of the invention there is provided a primer for detection of a mutation in a microsatellite contained in a target sequence of a double stranded DNA molecule, wherein the mutation is a frameshift of the microsatellite as compared with the corresponding wild type microsatellite sequence, and wherein the primer comprises a region of at least 10 nucleotides that is complementary to the target sequence of the antisense or the sense strand of the DNA molecule containing the microsatellite having a frameshift mutation, except that the primer includes between one and four nucleotides which are mismatched to the target sequence containing the mutation in the microsatellite and which are also mismatched to a corresponding sequence containing the wild type microsatellite. In some embodiments, the primer includes between one and three nucleotides which are mismatched to the target sequence containing the mutation in the microsatellite and which are also mismatched to a corresponding sequence containing the wild type microsatellite.

As is disclosed herein, there are many microsatellites in which disease-relevant frameshift mutations can occur. For example, the frameshift may be in a microsatellite in any gene selected from ASTE1, ACVR22 (also known as ACVR2A), TAF1β, KIAA2018, SLC22A9 and TGFβR2. In some embodiments, the frameshift is in a microsatellite in TGFβR2, ASTE1, TAF1β or ACVR2A. When the frameshift mutation is in ACVR2A, the frameshift mutation in is exon 10 of ACVR2A.

In some embodiments, the primer is configured to anneal across the length of the microsatellite having a frameshift mutation, and to at least one nucleotide, or at least two nucleotides, flanking the 5′ end of the microsatellite and/or at least one nucleotide flanking the 3′ end of the microsatellite having a frameshift mutation. In some embodiments, the primer is configured to anneal across the length of the microsatellite having a frameshift mutation, and to at least one nucleotide flanking the 5′ end of the microsatellite having a frameshift mutation and/or at least one nucleotide flanking the 3′ end of the microsatellite having a frameshift mutation. The primer may anneal to at least two, three, four, or five nucleotides flanking one or both ends of the microsatellite having a frameshift mutation.

It is to be understood that where reference is made to “anneal across the length of the microsatellite having a frameshift mutation”, this means that the primer anneals to the target sequence at least for the full length of the microsatellite in the target sequence (which has a frameshift mutation). Preferably, the target sequence is in a TGFβR2 gene, an ASTE1 gene, a TAF1β gene or an ACVR2A gene. For example, when the target sequence is a9 TGFβR2, the primer anneals to the target sequence for the whole length of the a9 microsatellite. When the target sequence is a10 ASTE1, the primer anneals to the target sequence for the whole length of the a10 microsatellite. When the target sequence is a10 TAF1β, the primer anneals to the target sequence for the whole length of the a10 microsatellite. When the target sequence is a7 ACVR2A, the primer anneals to the target sequence for the whole length of the a7 microsatellite.

In some embodiments, the primer does not anneal across the length of the corresponding wild-type microsatellite sequence, and the 3′ end of the primer does not anneal to the corresponding sequence containing the wild-type microsatellite. This provides the advantage that the primer anneals to the sequence containing the microsatellite having a frameshift mutation, but does not anneal to the corresponding sequence having the wild-type microsatellite.

In some embodiments, the primer anneals to between 1 and 15, preferably between 1 and 13, nucleotides flanking the 3′ end of the microsatellite having a frameshift mutation (i.e. the primer comprises between 1 and 15, preferably between 1 and 13, nucleotides 3′ of the microsatellite, with respect to the primer, which anneal to the corresponding sequence comprising the microsatellite having a frameshift mutation). In some embodiments, the primer anneals to one, two, three, five or thirteen, preferably one, two, five or thirteen, and more preferably one or two, nucleotides flanking the 3′ end of the microsatellite having a frameshift mutation (i.e. the primer comprises one, two, three, five or thirteen, preferably one, two, five or thirteen, more preferably one or two, nucleotides 3′ of the microsatellite, with respect to the primer, which anneal to the corresponding sequence comprising the microsatellite having a frameshift mutation). In some embodiments, the primer anneals to one, two or three nucleotides flanking the 3′ end of the microsatellite having a frameshift mutation and in some embodiments the two or three nucleotides form a GC-clamp. In some embodiments, the primer anneals across the length of the microsatellite having the frameshift mutation and, 3′ of the microsatellite having the frameshift mutation, with respect to the primer, the primer comprises or consists of one, two, three, five or thirteen, preferably one, two, five or thirteen, more preferably one or two, nucleotides which anneal to the nucleotides flanking the microsatellite having the frameshift mutation. This provides the advantage that the primer annealed to the sequence containing the microsatellite having a frameshift mutation can be extended in the 5′-to-3′ direction, thereby allowing replication of the sequence containing the microsatellite having the frameshift mutation. In some embodiments, the primer anneals to one, two, three or five nucleotides flanking the 3′ end of the microsatellite having a frameshift mutation and the frameshift mutation is in a microsatellite in TGFβR2. In some embodiments, the primer anneals to one, two, three or thirteen, preferably one, two or thirteen, more preferably one, nucleotides flanking the 3′ end of the microsatellite having a frameshift mutation and the frameshift mutation is in a microsatellite in ASTE1. In some embodiments, the primer anneals to one nucleotide flanking the 3′ end of the microsatellite having a frameshift mutation and the frameshift mutation is in a microsatellite in TAF1β. In some embodiments, the primer anneals to two nucleotides flanking the 3′ end of the microsatellite having a frameshift mutation and the frameshift mutation is in a microsatellite in ACVR2A.

In some embodiments, the primer anneals to at least 1, 2, 3, 4, 5, 6, 7, 8, 9, 10, 11 or 12 nucleotides flanking the 5′ end of the microsatellite having a frameshift mutation, with respect to the primer. In some embodiments, the primer anneals to at least 10, 12 or 13 nucleotides flanking the 5′ end of the microsatellite having a frameshift mutation. In some embodiments, the primer anneals to 8, 9, 10, 11 or 12 nucleotides, preferably 8 or 12 nucleotides, flanking the 5′ end of the microsatellite having a frameshift mutation, with respect to the primer, and preferably the frameshift mutation is in a microsatellite in TGFβR2. In some embodiments, the primer anneals to 1, 2, 3, 4, 5, 6, 7, 8, 9, 10, 11 or 12 nucleotides, preferably 1 or 12 nucleotides, flanking the 5′ end of the microsatellite having a frameshift mutation, with respect to the primer, and preferably the frameshift mutation is in a microsatellite in ASTE1. In some embodiments, the primer anneals to 12 nucleotides flanking the 5′ end of the microsatellite having a frameshift mutation, with respect to the primer, and preferably the frameshift mutation is in a microsatellite in ASTE1. In some embodiments, the primer anneals to 9, 10, 11 nucleotides, preferably 9 or 10 nucleotides, more preferably 10 nucleotides, flanking the 5′ end of the microsatellite having a frameshift mutation, with respect to the primer, and preferably the frameshift mutation is in a microsatellite in TAF1β. In some embodiments, the primer anneals to 13 nucleotides flanking the 5′ end of the microsatellite having a frameshift mutation, with respect to the primer, and preferably the frameshift mutation is in a microsatellite in ACVR2A. In some embodiments, the primer anneals to 1, 8, 10, 12 or 13 nucleotides flanking the 5′ end of the microsatellite having a frameshift mutation, with respect to the primer. The primer may anneal to any of the above-detailed number of nucleotides flanking the 3′ end of the microsatellite having a frameshift mutation.

In some embodiments, the primer is configured to anneal to at least 1, at least 2, at least 3, at least 5, at least 8, at least 10, at least 12 or at least 13 nucleotides flanking the 5′ end of the microsatellite having a frameshift mutation and to at least 1, at least 2, at least 3, at least 5 or at least 13 nucleotides flanking the 3′ end of the microsatellite having a frameshift mutation. In some embodiments, the primer is configured to anneal to 1, 8, 10, 12 or 13 nucleotides flanking the 5′ end of the microsatellite having a frameshift mutation and to anneal to at least 1, at least 2, at least 3, at least 5 or at least 13, preferably to 1, 2, 3, 5 or 13, nucleotides flanking the 3′ end of the microsatellite having a frameshift mutation. In some embodiments, the primer is configured to anneal to at least 10, 12 or 13 nucleotides flanking the 5′ end of the microsatellite having a frameshift mutation and to anneal to at least 1 or at least 2 nucleotides flanking the 3′ end of the microsatellite having a frameshift mutation. In some embodiments, the primer is configured to anneal to 8, 9, 10, 11 or 12 nucleotides, preferably 8 or 12 nucleotides, flanking the 5′ end of the microsatellite having a frameshift mutation and to anneal to at least 1, at least 2, at least 3, at least 5 or at least 13 nucleotides, preferably to 2 or 5 nucleotides, flanking the 3′ end of the microsatellite having a frameshift mutation, and preferably the frameshift mutation is in a microsatellite in TGFβR2. In some embodiments, the primer anneals to 1, 2, 3, 4, 5, 6, 7, 8, 9, 10, 11 or 12 nucleotides, preferably 1 or 12 nucleotides, flanking the 5′ end of the microsatellite having a frameshift mutation and to anneal to at least 1, at least 2, at least 3, at least 5 or at least 13 nucleotides, preferably to 1 or 13 nucleotides, flanking the 3′ end of the microsatellite having a frameshift mutation, and preferably the frameshift mutation is in a microsatellite in ASTE1. In some embodiments, the primer anneals to 1, 2, 3, 4, 5, 6, 7, 8, 9, 10, 11 or 12 nucleotides, preferably 12 nucleotides, flanking the 5′ end of the microsatellite having a frameshift mutation and to anneal to at least 1, at least 2, at least 3, at least 5 or at least 13 nucleotides, preferably to 1 nucleotide, flanking the 3′ end of the microsatellite having a frameshift mutation, and preferably the frameshift mutation is in a microsatellite in ASTE1. In some embodiments, the primer is configured to anneal to 9, 10 or 11 nucleotides, preferably 9 or 10 nucleotides, more preferably 10 nucleotides, flanking the 5′ end of the microsatellite having a frameshift mutation and to anneal to at least 1, at least 2, at least 3, at least 5 or at least 13 nucleotides, preferably to 1 nucleotide, flanking the 3′ end of the microsatellite having a frameshift mutation, and preferably the frameshift mutation is in a microsatellite in TAF1β. In some embodiments, the primer is configured to anneal to 11, 12, 13 or 14 nucleotides, preferably 13 nucleotides, flanking the 5′ end of the microsatellite having a frameshift mutation and to anneal to at least 1, at least 2, at least 3, at least 4 or at least 5 nucleotides, preferably to 1 nucleotide, flanking the 3′ end of the microsatellite having a frameshift mutation, and preferably the frameshift mutation is in a microsatellite in ACVR2A. In some embodiments, the primer is configured to anneal to 12 nucleotides flanking the 5′ end of the microsatellite having a frameshift mutation and to 2 nucleotides flanking the 3′ end of the microsatellite having a frameshift mutation, to 8 nucleotides flanking the 5′ end of the microsatellite having a frameshift mutation and to 5 nucleotides flanking the 3′ end of the microsatellite having a frameshift mutation, to 12 nucleotides flanking the 5′ end of the microsatellite having a frameshift mutation and to 1 nucleotide flanking the 3′ end of the microsatellite having a frameshift mutation, to 1 nucleotide flanking the 5′ end of the microsatellite having a frameshift mutation and to 13 nucleotides flanking the 3′ end of the microsatellite having a frameshift mutation, to 13 nucleotides flanking the 5′ end of the microsatellite having a frameshift mutation and to 2 nucleotides flanking the 3′ end of the microsatellite having a frameshift mutation, or to 10 nucleotides flanking the 5′ end of the microsatellite having a frameshift mutation and to 1 nucleotide flanking the 3′ end of the microsatellite having a frameshift mutation. Preferably, the primer is configured to anneal to 12 nucleotides flanking the 5′ end of the microsatellite having a frameshift mutation and to 1 nucleotide flanking the 3′ end of the microsatellite having a frameshift mutation, to 13 nucleotides flanking the 5′ end of the microsatellite having a frameshift mutation and to 2 nucleotides flanking the 3′ end of the microsatellite having a frameshift mutation, or to 10 nucleotides flanking the 5′ end of the microsatellite having a frameshift mutation and to 1 nucleotide flanking the 3′ end of the microsatellite having a frameshift mutation.

In some embodiments, the primer is configured to anneal to 12 nucleotides flanking the 5′ end of the microsatellite having a frameshift mutation and to 2 nucleotides flanking the 3′ end of the microsatellite having a frameshift mutation, or to 8 nucleotides flanking the 5′ end of the microsatellite having a frameshift mutation and to 5 nucleotides flanking the 3′ end of the microsatellite having a frameshift mutation, preferably wherein the frameshift mutation is in a microsatellite in TGFβR2. In some embodiments, the primer is configured to anneal to 12 nucleotides flanking the 5′ end of the microsatellite having a frameshift mutation and to 1 nucleotide flanking the 3′ end of the microsatellite having a frameshift mutation, or to 1 nucleotide flanking the 5′ end of the microsatellite having a frameshift mutation and to 13 nucleotides flanking the 3′ end of the microsatellite having a frameshift mutation, preferably wherein the frameshift mutation is in a microsatellite in ASTE1. In some embodiments, the primer is configured to anneal to 12 nucleotides flanking the 5′ end of the microsatellite having a frameshift mutation and to 1 nucleotide flanking the 3′ end of the microsatellite having a frameshift mutation, preferably wherein the frameshift mutation is in a microsatellite in ASTE1. In some embodiments, the primer is configured to anneal to 10 nucleotides flanking the 5′ end of the microsatellite having a frameshift mutation and to 1 nucleotide flanking the 3′ end of the microsatellite having a frameshift mutation, preferably wherein the frameshift mutation is in a microsatellite in TAF1β. In some embodiments, the primer is configured to anneal to 13 nucleotides flanking the 5′ end of the microsatellite having a frameshift mutation and to 2 nucleotides flanking the 3′ end of the microsatellite having a frameshift mutation, preferably wherein the frameshift mutation is in a microsatellite in ACVR2A.

In some embodiments, at least 30%, 35%, 40%, 45%, 50%, 55%, 60% or 65% of the nucleotides of the primer anneal to nucleotides flanking the 5′ end of the microsatellite having a frameshift mutation, with respect to the primer. In other words, at least 30%, 35%, 40%, 45%, 50%, 55%, 60% or 65% of the nucleotides making up the primer anneal to nucleotides flanking the 5′ end of the microsatellite having a frameshift mutation with respect to the primer (i.e. flanking the 3′ end of the microsatellite having a frameshift mutation with respect to the target sequence). In some embodiments, the part of the primer which anneals to the nucleotides flanking the 5′ end the microsatellite having a frameshift mutation, with respect to the primer, also anneals to the corresponding sequence containing the wild-type microsatellite. This provides the advantage that the primer is specific for the desired target sequence. In some embodiments, at least 50% of the nucleotides of the primer anneal to nucleotides flanking the 5′ end of the microsatellite having a frameshift mutation with respect to the primer (i.e. anneal to nucleotides flanking the 3′ end of the microsatellite having a frameshift mutation with respect to the target sequence). In some embodiments, at least 30%, 35%, 40%, 45%, 50%, 55%, 60% or 65%, preferably at least 35% or at least 50%, of the nucleotides of the primer anneal to nucleotides flanking the 5′ end of the microsatellite having a frameshift mutation, with respect to the primer, and the frameshift mutation is in a microsatellite in TGFβR2. In some embodiments, at least 30%, 35%, 40%, 45%, 50%, 55%, 60% or 65%, preferably at least 50%, of the nucleotides of the primer anneal to nucleotides flanking the 5′ end of the microsatellite having a frameshift mutation, with respect to the primer, and the frameshift mutation is in a microsatellite in ASTE1. In some embodiments, at least 30%, 35%, 40%, 45%, 50%, 55%, 60% or 65%, preferably at least 45%, of the nucleotides of the primer anneal to nucleotides flanking the 5′ end of the microsatellite having a frameshift mutation, with respect to the primer, and the frameshift mutation is in a microsatellite in TAF1β. In some embodiments, at least 30%, 35%, 40%, 45%, 50%, 55%, 60% or 65%, preferably at least 55%, of the nucleotides of the primer anneal to nucleotides flanking the 5′ end of the microsatellite having a frameshift mutation, with respect to the primer, and the frameshift mutation is in a microsatellite in ACVR2A.

In some embodiments, less than 35%, 30%, 25%, 20%, 15%, 10% or 5% of the nucleotides of the primer anneal to nucleotides flanking the 5′ end of the microsatellite having a frameshift mutation, with respect to the primer. In other words, less than 35%, 30%, 25%, 20%, 15%, 10% or 5% of the nucleotides making up the primer anneal to nucleotides flanking the 5′ end of the microsatellite having a frameshift mutation with respect to the primer (i.e. flanking the 3′ end of the microsatellite having a frameshift mutation with respect to the target sequence). In the embodiments of this paragraph, the frameshift mutation is preferably in a microsatellite in ASTE1. In some embodiments, less than 5% of the nucleotides of the primer anneal to nucleotides flanking the 5′ end of the microsatellite having a frameshift mutation, with respect to the primer, preferably wherein the frameshift mutation is in a microsatellite in ASTE1. Correspondingly, at least 30%, 35%, 40%, 45%, 50%, 55%, 60% or 65% of the nucleotides of the primer anneal to nucleotides flanking the 3′ end of the microsatellite having a frameshift mutation, with respect to the primer.

In some embodiments, the nucleotide(s) of the primer which anneal to nucleotide(s) flanking the 5′ end of the microsatellite having a frameshift mutation, with respect to the primer, are 100% complementary to the corresponding nucleotide(s) in the target sequence having the frameshift mutation in the microsatellite. This provides the advantage that the primer is specific to the target sequence.

In some embodiments, the primer consists of between 16 and 30 nucleotides. Preferably, the primer consists of between 16 and 25 nucleotides or between 16 and 24 nucleotides. Preferably still, the primer consists of between 17 and 24 nucleotides, between 17 and 23 nucleotides, between 19 and 22 nucleotides, or between 19 and 21 nucleotides.

In some embodiments, the primer is isolated or recombinant. In some embodiments, the primer is less than 50, 30, 25 or 20 nucleotides in length.

In some embodiments, the primer comprises a region of at least 10, at least 11, at least 12, at least 13, at least 14, at least 15, at least 16, at least 17, at least 18, at least 19 or at least 20 nucleotides that is complementary to the target sequence of the antisense or the sense strand of the DNA molecule containing the microsatellite having a frameshift mutation, except that the primer includes between one and four nucleotides which are mismatched to the target sequence containing the mutation in the microsatellite and which are also mismatched to a corresponding sequence containing the wild type microsatellite. Preferably, the primer comprises a region of at least 10, at least 11 or at least 12 nucleotides that is complementary to the target sequence of the antisense or the sense strand of the DNA molecule containing the microsatellite having a frameshift mutation.

In some embodiments, the primer consists of 21, 22, 23 or 24 nucleotides. In some embodiments, the primer consists of 21, 22 or 23 nucleotides. In some embodiments, the primer consists of 23 nucleotides and the frameshift mutation is in a microsatellite in ASTE1, the primer consists of 21 nucleotides and the frameshift mutation is in a microsatellite in TAF1β, or the primer consists of 22 nucleotides and the frameshift mutation is in a microsatellite in ACVR2A.

In some embodiments, the wild type microsatellite sequence is a sequence from human genomic DNA.

In some embodiments, the primer comprises 1, 2, 3 or 4 nucleotides which are mismatched to the target sequence containing the mutation in the microsatellite and which are also mismatched to a corresponding sequence containing the wild type microsatellite. This means that the primer contains between 1 and 4 such mismatches, but no more than 4 such mismatches. Preferably, the primer comprises between 1 and 3 mismatches, i.e. between 1 and 3 mismatches but no more than 3 mismatches, and more preferably the primer has 2 or 3 mismatches (i.e. exactly 2 or exactly 3 mismatches). In some embodiments, at least one mismatched nucleotide is located in a position of the primer that anneals 3′ downstream of the microsatellite or within the microsatellite. In some embodiments, at least one mismatched nucleotide is located in a position of the primer that anneals 3′ downstream of the microsatellite and at least one mismatched nucleotide is located within the microsatellite, with respect to the primer. In some embodiments, the primer has only one mismatched nucleotide, which is located in a position of the primer that anneals 3′ downstream of the microsatellite. In some embodiments, the primer has one mismatched nucleotide located in a position of the primer that anneals 3′ downstream of the microsatellite and two mismatched nucleotides located within the microsatellite. In some embodiments, the primer has one mismatched nucleotide located in a position of the primer that anneals 3′ downstream of the microsatellite and one mismatched nucleotide located within the microsatellite, with respect to the primer. In some embodiments, the primer has one mismatched nucleotides located within the microsatellite, with respect to the primer. In some embodiments, the primer has two mismatched nucleotides located within the microsatellite, with respect to the primer. In some embodiments, the primer has three mismatched nucleotides located within the microsatellite, with respect to the primer. In some embodiments, the primer has one mismatched nucleotide located within the microsatellite and two mismatched nucleotides located in a position of the primer that anneals 3′ downstream of the microsatellite, with respect to the primer. In some embodiments, all of the mismatched nucleotides are located in a position of the primer that anneals 3′ downstream of the microsatellite or within the microsatellite. In some embodiments, all of the mismatched nucleotides are located in a position of the primer that anneals within the microsatellite. In some embodiments, the primer has 2 or 3 mismatches, which are all located within the microsatellite, with respect to the primer, preferably wherein the frameshift mutation is in a microsatellite in ASTE1, TAF1β or ACVR2A.

In some embodiments, the primer has one mismatched nucleotide, which is located in a position of the primer that anneals 3′ downstream of the microsatellite, with respect to the primer, or the primer has one mismatched nucleotide located in a position of the primer that anneals 3′ downstream of the microsatellite and one mismatched nucleotide located within the microsatellite, with respect to the primer, or the primer has one mismatched nucleotide located in a position of the primer that anneals 3′ downstream of the microsatellite and two mismatched nucleotides located within the microsatellite, with respect to the primer, or the primer has one mismatched nucleotide located within the microsatellite and two mismatched nucleotides located in a position of the primer that anneals 3′ downstream of the microsatellite, with respect to the primer, and the frameshift mutation is in a microsatellite in TGFβR2. In some embodiments, the primer has two mismatched nucleotides located within the microsatellite, with respect to the primer, or the primer has three mismatched nucleotides located within the microsatellite, with respect to the primer, or the primer has one mismatched nucleotide located within the microsatellite and two mismatched nucleotides located in a position of the primer that anneals 3′ downstream of the microsatellite, with respect to the primer, and the frameshift mutation is in a microsatellite in ASTE1. In some embodiments, the primer has one mismatched nucleotides located within the microsatellite, with respect to the primer, or the primer has two mismatched nucleotides located within the microsatellite, with respect to the primer, or the primer has three mismatched nucleotides located within the microsatellite, with respect to the primer, and the frameshift mutation is in a microsatellite in TAF1β. In some embodiments, the primer has three mismatched nucleotides located within the microsatellite, with respect to the primer, and the frameshift mutation is in a microsatellite in ACVR2A.

In some embodiments, at least one mismatched nucleotide is within three or five nucleotides 5′ upstream or within 12 nucleotides 3′ downstream of the 3′ end of the microsatellite with respect to the primer. In some embodiments, at least one mismatched nucleotide in the primer is within 1, 2, 3, 4 or 5 nucleotides 5′ upstream of the 3′ end of the microsatellite or within 2, 4, 5 or 12 nucleotides 3′ downstream of the 3′ end of the microsatellite. In some embodiments, at least one mismatched nucleotide is within 12 nucleotides 3′ downstream of the 3′ end of the microsatellite, with respect to the primer. Preferably, all of the mismatched nucleotides are within two, three, four or five nucleotides 5′ upstream or within 12 nucleotides 3′ downstream of the 3′ end of the microsatellite with respect to the primer. Preferably, all of the mismatched nucleotides are within two, three, four or five nucleotides 5′ upstream of the 3′ end of the microsatellite with respect to the primer.

In some embodiments, the first nucleotide 3′ downstream of the 3′ end of the microsatellite is not a mismatched nucleotide.

Each of the mismatches in the primer may be a nucleotide substitution to any nucleotide (e.g. A, T, C or G) which is mismatched to the corresponding nucleotide in the target sequence and the wild type sequence. In some embodiments, at least one mismatched nucleotide is a substitution of a thymine (T) with an adenine (A), a substitution of a thymine (T) with a guanine (G), a substitution of a thymine (T) with a cytosine (C), a substitution of an adenine (A) with a guanine (G), a substitution of an adenine (A) with a thymine (T), a substitution of an adenine (A) with a cytosine (C), a substitution of a cytosine (C) with a guanine (G), a substitution of a cytosine (C) with a thymine (T), a substitution of a cytosine (C) with an adenine (A), a substitution of a guanine (G) with a thymine (T), a substitution of a guanine (G) with an adenine (A), or a substitution of a guanine (G) with a cytosine (C). In some embodiments, at least one mismatched nucleotide is a substitution of a thymine (T) with an adenine (A), a substitution of a thymine (T) with a cytosine (C), a substitution of a thymine (T) with a guanine (G), a substitution of an adenine (A) with a cytosine (C), a substitution of an adenine (A) with a guanine (G), a substitution of an adenine (A) with a thymine (T), or a substitution of a cytosine (C) with a guanine (G).

In some embodiments, the primer comprises at least two mismatched nucleotides, and the at least two mismatched nucleotides comprise a substitution of a two pyrimidine nucleotides with a purine nucleotide and a pyrimidine nucleotide, or comprise a substitution of two purine nucleotides with two pyrimidine nucleotides or with a purine nucleotide and a pyrimidine nucleotide.

In some embodiments, at least one mismatched nucleotide is a substitution of a thymine (T) with an adenine (A), a substitution of a thymine (T) with a cytosine (C), or a substitution of a cytosine (C) with a guanine (G), preferably wherein the frameshift mutation is in a microsatellite in TGFβR2. In some embodiments, at least one mismatched nucleotide is a substitution of a thymine (T) with a cytosine (C), a substitution of a thymine (T) with an adenine (A), a substitution of a thymine (T) with a guanine (G), or a substitution of an adenine (A) with a cytosine (C), preferably wherein the frameshift mutation is in a microsatellite in ASTE1. In some embodiments, at least one mismatched nucleotide is a substitution of a thymine (T) with a cytosine (C), a substitution of a thymine (T) with an adenine (A) or a substitution of a thymine (T) with a guanine (G), preferably wherein the frameshift mutation is in a microsatellite in ASTE1. In some embodiments, at least one mismatched nucleotide is a substitution of an adenine (A) with a guanine (G) or a substitution of an adenine (A) with a thymine (T), preferably wherein the frameshift mutation is in a microsatellite in TAF1β. In some embodiments, at least one mismatched nucleotide is a substitution of a thymine (T) with a cytosine (C) or an adenine (A) or a guanine (G), preferably wherein the frameshift mutation is in a microsatellite in TGFβR2 or ASTE1. In some embodiments, at least one mismatched nucleotide is a substitution of an adenine (A) with a guanine (G) or a thymine (T), preferably wherein the frameshift mutation is in a microsatellite in TAF1β. In some embodiments, at least one mismatched nucleotide is a substitution of an adenine (A) with a thymine (T) or a cytosine (C), preferably wherein the frameshift mutation is in a microsatellite in ACVR2A.

In some embodiments, the primer has a mismatched nucleotide at the second nucleotide 3′ downstream of the 3′ end of the microsatellite, with respect to the primer. The mismatch is a substitution to any nucleotide which is mismatched to the corresponding nucleotide in the target sequence and the wild type sequence, and, in some embodiments, the mismatched nucleotide is G, A or T, preferably G. In some embodiments, the mismatched nucleotide at the second nucleotide 3′ downstream of the 3′ end of the microsatellite of the primer is the only mismatch in the primer. In the embodiments of this paragraph, the frameshift mutation is preferably in a microsatellite in TGFβR2. In some embodiments, the primer comprises or consists of the sequence of SEQ ID NO: 24, wherein X₁ is A, G or T. In some embodiments, the primer comprises or consists of the sequence of SEQ ID NO: 17.

In some embodiments, the primer has a mismatched nucleotide at the second nucleotide 3′ downstream of the 3′ end of the microsatellite and at the first nucleotide 5′ upstream of the 3′ end of the microsatellite, with respect to the primer. In these embodiments, the second nucleotide 3′ downstream of the 3′ end of the microsatellite may be G, A or T, preferably G, and the first nucleotide 5′ upstream of the 3′ end of the microsatellite may be G, C or A, preferably C or A. In some embodiments, the mismatched nucleotide at the second nucleotide 3′ downstream of the 3′ end of the microsatellite and the mismatched nucleotide at the first nucleotide 5′ upstream of the 3′ end of the microsatellite of the primer are the only mismatches in the primer. In the embodiments of this paragraph, the frameshift mutation is preferably in a microsatellite in TGFβR2. In some embodiments, the primer comprises or consists of the sequence of SEQ ID NO: 25, wherein X₃ is A, G or T, and X₂ is A, C or G. In some embodiments, the primer comprises or consists of the sequence of SEQ ID NO: 18 or SEQ ID NO: 21.

In some embodiments, the primer has a mismatched nucleotide at the second nucleotide 3′ downstream of the 3′ end of the microsatellite, at the first nucleotide 5′ upstream of the 3′ end of the microsatellite and at the second nucleotide 5′ upstream of the 3′ end of the microsatellite, with respect to the microsatellite. In these embodiments, the second nucleotide 3′ downstream of the 3′ end of the microsatellite may be G, A or T, preferably G, the first nucleotide 5′ upstream of the 3′ end of the microsatellite may be G, C or A, preferably C and the second nucleotide 5′ upstream of the 3′ end of the microsatellite may be G, C or A, preferably A. In some embodiments, the mismatched nucleotide at the second nucleotide 3′ downstream of the 3′ end of the microsatellite, at the first nucleotide 5′ upstream of the 3′ end of the microsatellite and at the second nucleotide 5′ upstream of the 3′ end of the microsatellite of the primer are the only mismatches in the primer. In the embodiments of this paragraph, the frameshift mutation is preferably in a microsatellite in TGFβR2. In some embodiments, the primer comprises or consists of the sequence of SEQ ID NO: 26, wherein X₆ is A, G or T, and each of X₄ and X₅, independently, is A, C or G. In some embodiments, the primer comprises or consists of the sequence of SEQ ID NO: 19.

In some embodiments, the primer has a mismatched nucleotide at the second nucleotide 3′ downstream of the 3′ end of the microsatellite and at the second nucleotide 5′ upstream of the 3′ end of the microsatellite, with respect to the primer. In these embodiments, the second nucleotide 3′ downstream of the 3′ end of the microsatellite may be G, A or T, preferably G, and the second nucleotide 5′ upstream of the 3′ end of the microsatellite may be G, C or A, preferably A. In some embodiments, the mismatched nucleotide at the second nucleotide 3′ downstream of the 3′ end of the microsatellite and at the second nucleotide 5′ upstream of the 3′ end of the microsatellite of the primer are the only mismatches in the primer. In the embodiments of this paragraph, the frameshift mutation is preferably in a microsatellite in TGFβR2. In some embodiments, the primer comprises or consists of the sequence of SEQ ID NO: 27, wherein X₈ is A, G or T, and X₇ is A, C or G. In some embodiments, the primer comprises or consists of the sequence of SEQ ID NO: 22.

In some embodiments, the primer has a mismatched nucleotide at the second nucleotide 3′ downstream of the 3′ end of the microsatellite, at the second nucleotide 5′ upstream of the 3′ end of the microsatellite and at fourth nucleotide 3′ downstream of the 3′ end of the microsatellite, with respect to the primer. In these embodiments, the second nucleotide 3′ downstream of the 3′ end of the microsatellite may be G, A or T, preferably G, the second nucleotide 5′ upstream of the 3′ end of the microsatellite may be G, C or A, preferably A, and the fourth nucleotide 3′ downstream of the 3′ end of the microsatellite may be A, G or C, preferably C. In some embodiments, the mismatched nucleotide at the second nucleotide 3′ downstream of the 3′ end of the microsatellite, at the second nucleotide 5′ upstream of the 3′ end of the microsatellite and at fourth nucleotide 3′ downstream of the 3′ end of the microsatellite of the primer are the only mismatches in the primer. In the embodiments of this paragraph, the frameshift mutation is preferably in a microsatellite in TGFβR2. In some embodiments, the primer comprises or consists of the sequence of SEQ ID NO: 28, wherein X₁₀ is A, G or T, X₉ is A, C or G, and X₁₁ is A, C or G. In some embodiments, the primer comprises or consists of the sequence of SEQ ID NO: 23.

In some embodiments, the primer has a mismatched nucleotide at the first nucleotide 5′ upstream of the 3′ end of the microsatellite and at the third nucleotide 5′ upstream of the 3′ end of the microsatellite, with respect to the primer. In these embodiments, the first nucleotide 5′ upstream of the 3′ end of the microsatellite may be A, C or G, preferably C, and the third nucleotide 5′ upstream of the 3′ end of the microsatellite may be A, C or G, preferably A or G. In some embodiments, the mismatched nucleotide at the first nucleotide 5′ upstream of the 3′ end of the microsatellite and at the third nucleotide 5′ upstream of the 3′ end of the microsatellite of the primer are the only mismatches in the primer. In the embodiments of this paragraph, the frameshift mutation is preferably in a microsatellite in ASTE1. In some embodiments, the primer comprises or consists of the sequence of SEQ ID NO: 43, wherein X₁₂ is A, C or G, and X₁₃ is A, C or G. In some embodiments, the primer comprises or consists of the sequence of SEQ ID NO: 34 or SEQ ID NO: 35.

In some embodiments, the primer has a mismatched nucleotide at the first nucleotide 5′ upstream of the 3′ end of the microsatellite and at the second nucleotide 5′ upstream of the 3′ end of the microsatellite, with respect to the primer. In these embodiments, the first nucleotide 5′ upstream of the 3′ end of the microsatellite may be A, C or G, preferably C, and the second nucleotide 5′ upstream of the 3′ end of the microsatellite may be A, C or G, preferably A or G. In some embodiments, the mismatched nucleotide at the first nucleotide 5′ upstream of the 3′ end of the microsatellite and at the second nucleotide 5′ upstream of the 3′ end of the microsatellite of the primer are the only mismatches in the primer. In the embodiments of this paragraph, the frameshift mutation is preferably in a microsatellite in ASTE1. In some embodiments, the primer comprises or consists of the sequence of SEQ ID NO: 44, wherein X₁₄ is A, C or G, and X₁₅ is A, C or G. In some embodiments, the primer comprises or consists of the sequence of SEQ ID NO: 36.

In some embodiments, the primer has a mismatched nucleotide at the first nucleotide 5′ upstream of the 3′ end of the microsatellite, at the third nucleotide 5′ upstream of the 3′ end of the microsatellite and at the fourth nucleotide 5′ upstream of the 3′ end of the microsatellite, with respect to the primer. In these embodiments, the first nucleotide 5′ upstream of the 3′ end of the microsatellite may be A, C or G, preferably C or G, the third nucleotide 5′ upstream of the 3′ end of the microsatellite may be A, C or G, preferably A or C, more preferably A, and the fourth nucleotide 5′ upstream of the 3′ end of the microsatellite may be A, C or G, preferably A, more preferably G or C. In some embodiments, the mismatched nucleotide at the first nucleotide 5′ upstream of the 3′ end of the microsatellite, at the third nucleotide 5′ upstream of the 3′ end of the microsatellite and at the fourth nucleotide 5′ upstream of the 3′ end of the microsatellite of the primer are the only mismatches in the primer. In some embodiments, the primer has only three mismatched nucleotides, which are at the first, third and fourth nucleotides 5′ upstream of the 3′ end of the microsatellite, with respect to the primer. In these embodiments, it is preferred that the first nucleotide 5′ upstream of the 3′ end of the microsatellite is C, the third nucleotide 5′ upstream of the 3′ end of the microsatellite is A, and the fourth nucleotide 5′ upstream of the 3′ end of the microsatellite is A or C. In other preferred embodiments, the first nucleotide 5′ upstream of the 3′ end of the microsatellite is G, the third nucleotide 5′ upstream of the 3′ end of the microsatellite is A, and the fourth nucleotide 5′ upstream of the 3′ end of the microsatellite is G. In the embodiments of this paragraph, the frameshift mutation is preferably in a microsatellite in ASTE1. In some embodiments, the primer comprises or consists of the sequence of SEQ ID NO: 45, wherein each of X₁₆, X₁₇ and X₁₈, independently, is A, C or G. In some embodiments, the primer comprises or consists of the sequence of SEQ ID NO: 37, SEQ ID NO: 38, 84 or 85.

In some embodiments, the primer has a mismatched nucleotide at the second nucleotide 5′ upstream of the 3′ end of the microsatellite, at the third nucleotide 5′ upstream of the 3′ end of the microsatellite, with respect to the primer, and at the fourth nucleotide 5′ upstream of the 3′ end of the microsatellite, with respect to the primer. In these embodiments, the second nucleotide 5′ upstream of the 3′ end of the microsatellite may be A, C or G, preferably G or C, the third nucleotide 5′ upstream of the 3′ end of the microsatellite may be A, C or G, and the fourth nucleotide 5′ upstream of the 3′ end of the microsatellite may be A, C or G, preferably A. In some embodiments, the mismatched nucleotide at the second nucleotide 5′ upstream of the 3′ end of the microsatellite, at the third nucleotide 5′ upstream of the 3′ end of the microsatellite of the primer and at the fourth nucleotide 5′ upstream of the 3′ end of the microsatellite of the primer are the only mismatches in the primer are the only mismatches in the primer. In the embodiments of this paragraph, the frameshift mutation is preferably in a microsatellite in ASTE1. In some embodiments, the primer comprises or consists of the sequence of SEQ ID NO: 83, wherein X₃₆ is A, C or G, X₃₇ is A, C or G and X₃₈ is A, C or G. In some embodiments, the primer comprises or consists of the sequence of SEQ ID NO: 86, 87, 88, 98 or 99.

In some embodiments, the primer has a mismatched nucleotide at the third nucleotide 5′ upstream of the 3′ end of the microsatellite, with respect to the primer, at the fourth nucleotide 5′ upstream of the 3′ end of the microsatellite, with respect to the primer, and at the fifth nucleotide 5′ upstream of the 3′ end of the microsatellite. In these embodiments, the third nucleotide 5′ upstream of the 3′ end of the microsatellite may be A, C or G, preferably C, the fourth nucleotide 5′ upstream of the 3′ end of the microsatellite may be A, C or G, preferably C, and the fifth nucleotide 5′ upstream of the 3′ end of the microsatellite may be A, C or G, preferably A. In some embodiments, the mismatched nucleotide at the third nucleotide 5′ upstream of the 3′ end of the microsatellite, at the fourth nucleotide 5′ upstream of the 3′ end of the microsatellite of the primer and at the fifth nucleotide 5′ upstream of the 3′ end of the microsatellite of the primer are the only mismatches in the primer are the only mismatches in the primer. In the embodiments of this paragraph, the frameshift mutation is preferably in a microsatellite in ASTE1. In some embodiments, the primer comprises or consists of the sequence of SEQ ID NO: 100, wherein X₄₂ is A, C or G, X₄₃ is A, C or G and X₄ is A, C or G. In some embodiments, the primer comprises or consists of the sequence of SEQ ID NO: 101.

In some embodiments, the primer has a mismatched nucleotide at the second nucleotide 5′ upstream of the 3′ end of the microsatellite and at the third nucleotide 5′ upstream of the 3′ end of the microsatellite, with respect to the primer. In these embodiments, the second nucleotide 5′ upstream of the 3′ end of the microsatellite may be A, C or G, preferably G, and the third nucleotide 5′ upstream of the 3′ end of the microsatellite may be A, C or G, preferably A. In some embodiments, the mismatched nucleotide at the second nucleotide 5′ upstream of the 3′ end of the microsatellite and at the third nucleotide 5′ upstream of the 3′ end of the microsatellite of the primer are the only mismatches in the primer. In the embodiments of this paragraph, the frameshift mutation is preferably in a microsatellite in ASTE1. In some embodiments, the primer comprises or consists of the sequence of SEQ ID NO: 46, wherein X₁₉ is A, C or G, and X₂₀ is A, C or G. In some embodiments, the primer comprises or consists of the sequence of SEQ ID NO: 39.

In some embodiments, the primer has mismatched nucleotides at the second nucleotide 5′ upstream of the 3′ end of the microsatellite, with respect to the primer, and at the fifth and twelfth nucleotides 3′ downstream of the 3′ end of the microsatellite, with respect to the primer. In some embodiments, the primer has only three mismatched nucleotides, which are at the second nucleotide 5′ upstream of the 3′ end of the microsatellite and the fifth and twelfth nucleotides 3′ downstream of the 3′ end of the microsatellite. In these embodiments, it is preferred that the second nucleotide 5′ upstream of the 3′ end of the microsatellite is C, that the fifth nucleotide 3′ downstream of the 3′ end of the microsatellite is G, and that the twelfth nucleotide 3′ downstream of the 3′ end of the microsatellite is C. In some embodiments, the mismatched nucleotides at the second nucleotide 5′ upstream of the 3′ end of the microsatellite and at the fifth and twelfth nucleotides 3′ downstream of the 3′ end of the microsatellite are the only mismatches in the primer. In the embodiments of this paragraph, the frameshift mutation is preferably in a microsatellite in ASTE1. In some embodiments, the primer comprises or consists of the sequence of SEQ ID NO: 47, wherein X₂₁ is A, C or G, X₂₂ is A, C or G and X₂₃ is C, G or T. In some embodiments, the primer comprises or consists of the sequence of SEQ ID NO: 40.

In some embodiments, the primer has a mismatched nucleotide at the first nucleotide 5′ upstream of the 3′ end of the microsatellite, at the second nucleotide 5′ upstream of the 3′ end of the microsatellite and at the fourth nucleotide 5′ upstream of the 3′ end of the microsatellite, with respect to the primer. In these embodiments, the first nucleotide 5′ upstream of the 3′ end of the microsatellite may be A, C or G, preferably C, the second nucleotide 5′ upstream of the 3′ end of the microsatellite may be A, C or G, preferably A, and the fourth nucleotide 5′ upstream of the 3′ end of the microsatellite may be A, C or G, preferably A. In some embodiments, the mismatched nucleotide at the first nucleotide 5′ upstream of the 3′ end of the microsatellite, at the second nucleotide 5′ upstream of the 3′ end of the microsatellite and at the fourth nucleotide 5′ upstream of the 3′ end of the microsatellite of the primer are the only mismatches in the primer. In the embodiments of this paragraph, the frameshift mutation is preferably in a microsatellite in ASTE1. In some embodiments, the primer comprises or consists of the sequence of SEQ ID NO: 48, wherein each of X₂₄, X₂₅ and X₂₆, independently, is A, C or G. In some embodiments, the primer comprises or consists of the sequence of SEQ ID NO: 41.

In some embodiments, the primer has at least one mismatched nucleotide located at the fifth nucleotide 5′ upstream of the 3′ end of the microsatellite. In some embodiments, the primer has a mismatched nucleotide at the first nucleotide 5′ upstream of the 3′ end of the microsatellite, at the fourth nucleotide 5′ upstream of the 3′ end of the microsatellite and at the fifth nucleotide 5′ upstream of the 3′ end of the microsatellite, with respect to the primer. In these embodiments, the first nucleotide 5′ upstream of the 3′ end of the microsatellite may be A, C or G, preferably C, the fourth nucleotide 5′ upstream of the 3′ end of the microsatellite may be A, C or G, preferably A, and the fifth nucleotide 5′ upstream of the 3′ end of the microsatellite may be A, C or G, preferably A. In some embodiments, the mismatched nucleotide at the first nucleotide 5′ upstream of the 3′ end of the microsatellite, at the fourth nucleotide 5′ upstream of the 3′ end of the microsatellite and at the fifth nucleotide 5′ upstream of the 3′ end of the microsatellite of the primer are the only mismatches in the primer. In the embodiments of this paragraph, the frameshift mutation is preferably in a microsatellite in ASTE1. In some embodiments, the primer comprises or consists of the sequence of SEQ ID NO: 49, wherein each of X₂₇, X₂₈ and X₂₉, independently, is A, C or G. In some embodiments, the primer comprises or consists of the sequence of SEQ ID NO: 42.

In some embodiments, the primer has a mismatched nucleotide at the first nucleotide 5′ upstream of the 3′ end of the microsatellite, with respect to the primer. In these embodiments, the first nucleotide 5′ upstream of the 3′ end of the microsatellite may be C, G or T, preferably G or T. In some embodiments, the mismatched nucleotide at the first nucleotide 5′ upstream of the 3′ end of the microsatellite of the primer is the only mismatch in the primer. In the embodiments of this paragraph, the frameshift mutation is preferably in a microsatellite in TAF1β. In some embodiments, the primer comprises or consists of the sequence of SEQ ID NO: 60, wherein each of X₃₀ is C, G or T. In some embodiments, the primer comprises or consists of the sequence of SEQ ID NO: 53 or SEQ ID NO: 54.

In some embodiments, the primer has a mismatched nucleotide at the first nucleotide 5′ upstream of the 3′ end of the microsatellite and at the second nucleotide 5′ upstream of the 3′ end of the microsatellite, with respect to the primer. In these embodiments, the first nucleotide 5′ upstream of the 3′ end of the microsatellite may be C, G or T, preferably G or T, more preferably T, and the second nucleotide 5′ upstream of the 3′ end of the microsatellite may be C, G or T, preferably G or T. In some embodiments, the mismatched nucleotide at the first nucleotide 5′ upstream of the 3′ end of the microsatellite and at the second nucleotide 5′ upstream of the 3′ end of the microsatellite of the primer are the only mismatches in the primer. In the embodiments of this paragraph, the frameshift mutation is preferably in a microsatellite in TAF1β. In some embodiments, the primer comprises or consists of the sequence of SEQ ID NO: 61, wherein X₃₁ is C, G or T, and X₃₂ is C, G or T. In some embodiments, the primer comprises or consists of the sequence of SEQ ID NO: 55 or SEQ ID NO: 56.

In some embodiments, the primer has a mismatched nucleotide at the first nucleotide 5′ upstream of the 3′ end of the microsatellite, at the second nucleotide 5′ upstream of the 3′ end of the microsatellite, and at the fourth nucleotide 5′ upstream of the 3′ end of the microsatellite, with respect to the primer. In these embodiments, the first nucleotide 5′ upstream of the 3′ end of the microsatellite may be C, G or T, preferably G or T, the second nucleotide 5′ upstream of the 3′ end of the microsatellite may be C, G or T, preferably G or T, and the fourth nucleotide 5′ upstream of the 3′ end of the microsatellite may be C, G or T, preferably G or T. In some embodiments, the mismatched nucleotide at the first nucleotide 5′ upstream of the 3′ end of the microsatellite, at the second nucleotide 5′ upstream of the 3′ end of the microsatellite, and at the fourth nucleotide 5′ upstream of the 3′ end of the microsatellite of the primer are the only mismatches in the primer. In the embodiments of this paragraph, the frameshift mutation is preferably in a microsatellite in TAF1β. In some embodiments, the primer comprises or consists of the sequence of SEQ ID NO: 62, wherein each of X₃₃, X₃₄ and X₃₅, independently, is C, G or T. In some embodiments, the primer comprises or consists of the sequence of SEQ ID NO: 57, 58 or 59.

In some embodiments, the primer has a mismatched nucleotide at the first nucleotide 5′ upstream of the 3′ end of the microsatellite, at the second nucleotide 5′ upstream of the 3′ end of the microsatellite, and at the third nucleotide 5′ upstream of the 3′ end of the microsatellite, with respect to the primer. In these embodiments, the first nucleotide 5′ upstream of the 3′ end of the microsatellite may be C, G or T, preferably C or T, the second nucleotide 5′ upstream of the 3′ end of the microsatellite may be C, G or T, preferably C or T, and the third nucleotide 5′ upstream of the 3′ end of the microsatellite may be C, G or T, preferably C or T. In some embodiments, the mismatched nucleotide at the first nucleotide 5′ upstream of the 3′ end of the microsatellite, at the second nucleotide 5′ upstream of the 3′ end of the microsatellite and at the third nucleotide 5′ upstream of the 3′ end of the microsatellite of the primer are the only mismatches in the primer. In the embodiments of this paragraph, the frameshift mutation is preferably in a microsatellite in ACVR2A. In some embodiments, the primer comprises or consists of the sequence of SEQ ID NO: 89, wherein each of X₃₉, X₄₀ and X₄₁, independently, is C, G or T, preferably C or T. In some embodiments, the primer comprises or consists of the sequence of SEQ ID NO: 90 or 91.

As explained above, the primer includes one, two, three or four nucleotides which are mismatched to the target sequence containing the mutation in the microsatellite and which are also mismatched to a corresponding sequence containing the wild type microsatellite. This means that the primer includes at least one, but no more than four, nucleotides which are mismatched to the target sequence containing the mutation in the microsatellite and which are also mismatched to a corresponding sequence containing the wild type microsatellite.

The successful use of such primers in the context of a frameshift mutation in a microsatellite as disclosed herein came as a surprise to the present inventors. Before this invention, it was thought that it would not be possible to use a primer that anneals across the microsatellite in the target sequence to distinguish a frameshift mutation in said microsatellite due to the highly repeated nature of these regions. It was thought that such primers could not be used to distinguish between the frameshift mutated and the wild type microsatellite sequence, since a primer designed to anneal to the frameshifted microsatellite would be thought also to anneal to the wild type sequence, even if the binding affinity is lower.

This primer approach allows for a distinction between the frameshift mutant and wild type microsatellite by using primers that are complementary to a target sequence containing a microsatellite having a frameshift mutation, except that the primer has at least one mismatch (and up to four mismatches) to this sequence, as well as being mismatched to a corresponding sequence containing the wild type microsatellite in order that this primer has an additional mismatch to the wild type target sequence and, thus, further reduced affinity to the wild type target sequence. In other words, the primers are modelled on the sequence containing the microsatellite having a frameshift mutation, such that they comprise mismatches to the corresponding sequence having the wild-type microsatellite. The additional mismatch(es) between the primer and the microsatellite having a frameshift mutation results in further destabilisation and repulsion between the primer and the sequence containing the wild-type microsatellite.

This distinction between a frameshifted microsatellite and a wild type microsatellite offers a very useful detection tool, where detecting such a frameshift is important, such as in cancers where such frameshifts may be a disease driver and/or a possible therapeutic target. As is disclosed herein, there are many microsatellites in which disease-relevant frameshift mutations can occur. For example, the frameshift may be in a microsatellite in any gene selected from ASTE1, ACVR22 (also known as ACVR2A), TAF1β, KIAA2018, SLC22A9 and TGFβR2. Preferably, the frameshift is in a microsatellite in ASTE1, TAF1β, TGFβR2 or ACVR2A.

In another aspect of the invention, there is provided a kit for detection of a mutation in a microsatellite contained in a target sequence of a double stranded DNA molecule, wherein the mutation is a frameshift of the microsatellite as compared with the corresponding wild type microsatellite sequence, and wherein the kit comprises a first primer of the invention. The kit may optionally also comprise a second primer, wherein the second primer is configured to anneal to the target sequence 3′ downstream of the microsatellite on the opposite strand of the DNA molecule to the strand on which the first primer is configured to anneal. In particular, techniques to amplify a nucleic acid molecule using only one primer are known, for example, Single Specific Primer-Polymerase Chain Reaction (SSP-PCR). In SSP-PCR, the first primer is one of the primers of the invention and is used to produce single stranded amplicons (i.e. ssDNA). After one or more rounds of SSP-PCR, a second primer can be added to form a primer pair, or the single stranded amplicon could be analysed directly by other molecular methods after isolation. The first primer may be any of the primers of the invention described above.

In other words, this provides a primer pair for the detection method of the invention.

In some embodiments, the frameshift mutation may be in a microsatellite in the TGFβR2 gene, and the first primer may comprise the sequence defined by any one of SEQ ID NOs: 17 to 19 or 21 to 28 and/or, when present, the second primer comprises the sequence defined by SEQ ID NO: 14. In some embodiments, the frameshift mutation is in a microsatellite in the TGFβR2 gene and comprises the sequence according to residues 270 to 278 of SEQ ID NO: 3 or SEQ ID NO: 4, wherein the first primer comprises the sequence defined by any one of SEQ ID NOs: 24 to 28 and/or, when present, the second primer comprises the sequence defined by SEQ ID NO: 14, and wherein X defines a substitution to any nucleotide which is mismatched to the corresponding nucleotide in the target sequence and the wild type sequence. Preferably, the first primer comprises the sequence defined by SEQ ID NO: 24. In some embodiments, the frameshift mutation is in a microsatellite in the TGFβR2 gene and comprises the sequence according to residues 270 to 278 of SEQ ID NO: 3 or SEQ ID NO: 4, and wherein the first primer comprises the sequence defined by any one of SEQ ID NO: 17 to 19 or 21 to 23, and/or, when present, the second primer comprises the sequence defined by SEQ ID NO: 14.

Preferably, the first primer comprises or consists of the sequence defined by SEQ ID NO: 17. It will be understood that the use of the term “comprises” also includes primers that consist of the sequences listed above. The data disclosed herein demonstrates that these primers are particularly useful within the scope of the invention for use in the detection of frameshift mutations in a microsatellite within TGFβR2 (also referred to as a9, where a10 is the wild type microsatellite).

However, alternatively, the frameshift mutation may be in a microsatellite in the ASTE1 gene, and the first primer can then comprise the sequence defined by any one of SEQ ID NOs: 34 to 49, 83 to 88 and 98 to 101 and/or, when present, the second primer comprises the sequence defined by SEQ ID NO: 29. In some embodiments, the frameshift mutation is in a microsatellite in the ASTE1 gene and comprises the sequence according to residues 328 to 337 of SEQ ID NO: 7 or SEQ ID NO: 8, wherein the first primer comprises the sequence defined by SEQ ID NO: 45, 47, 83 or 100, and/or, when present, the second primer comprises the sequence defined by SEQ ID NO: 29, and wherein X defines a substitution to any nucleotide which is mismatched to the corresponding nucleotide in the target sequence and the wild type sequence. Preferably, the first primer comprises the sequence defined by SEQ ID NO: 45, 83 or 100. In some embodiments, the frameshift mutation is in a microsatellite in the ASTE1 gene and comprises the sequence according to residues 328 to 337 of SEQ ID NO: 7 or SEQ ID NO: 8, and wherein the first primer comprises the sequence defined by SEQ ID NO: 43, 44, 46, 48 or 49, and/or, when present, the second primer comprises the sequence defined by SEQ ID NO: 29, and wherein X defines a substitution to any nucleotide which is mismatched to the corresponding nucleotide in the target sequence and the wild type sequence. Preferably, the first primer comprises the sequence defined by SEQ ID NO: 48. In some embodiments, the frameshift mutation is in a microsatellite in the ASTE1 gene and comprises the sequence according to residues 328 to 337 of SEQ ID NO: 7 or SEQ ID NO: 8, and wherein the first primer comprises the sequence defined by any one of SEQ ID NO: 34 to 38 or 40, and/or, when present, the second primer comprises the sequence defined by SEQ ID NO: 29. Preferably, the first primer comprises the sequence defined by SEQ ID NO: 37 or 40. In some embodiments, the frameshift mutation is in a microsatellite in the ASTE1 gene and comprises the sequence according to residues 328 to 337 of SEQ ID NO: 7 or SEQ ID NO: 8, and wherein the first primer comprises the sequence defined by SEQ ID NO: 39, 41, 42, 84-88, 98, 99 or 100, and/or, when present, the second primer comprises the sequence defined by SEQ ID NO: 29. Preferably, the first primer comprises or consists of the sequence defined by any one of SEQ ID NOs: 41, 84-88, 98, 99 and 101, more preferably any one of SEQ ID NOs: 84-88, 98, 99 and 101.

Alternatively, the frameshift mutation may be in a microsatellite in the TAF1β gene, and the first primer can then comprise the sequence defined by any one of SEQ ID NOs: 53 to 62 and/or, when present, the second primer comprises the sequence defined by SEQ ID NO: 50. In some embodiments, the frameshift mutation is in a microsatellite in the TAF1β gene and comprises the sequence according to residues 255 to 264 of SEQ ID NO: 11 or SEQ ID NO: 12, and wherein the first primer comprises the sequence defined by any one of SEQ ID NOS: 60 to 62, and/or, when present, the second primer comprises the sequence defined by SEQ ID NO: 51, and wherein X defines a substitution to any nucleotide which is mismatched to the corresponding nucleotide in the target sequence and the wild type sequence. Preferably, the first primer comprises the sequence defined by SEQ ID NO: 61. In some embodiments, the frameshift mutation is in a microsatellite in the TAF1β gene and comprises the sequence according to residues 255 to 264 of SEQ ID NO: 11 or SEQ ID NO: 12, and wherein the first primer comprises the sequence defined by any one of SEQ ID NOS: 53 to 59, and/or, when present, the second primer comprises the sequence defined by SEQ ID NO: 51. Preferably, the first primer comprises or consists of the sequence defined by SEQ ID NO: 55 or 56.

Alternatively, the frameshift mutation may be in a microsatellite in the ACVR2A gene, and the first primer can then comprise the sequence defined by any one of SEQ ID NOs: 89-91, and/or, when present, the second primer comprises the sequence defined by SEQ ID NO: 92. In some embodiments, the frameshift mutation is in a microsatellite in the ACVR2A gene and comprises the sequence according to residues 509 to 515 of SEQ ID NO: 96 or 97, and wherein the first primer comprises the sequence defined by SEQ ID NO: 89, and/or, when present, the second primer comprises the sequence defined by SEQ ID NO: 92, and wherein X defines a substitution to any nucleotide which is mismatched to the corresponding nucleotide in the target sequence and the wild type sequence. In some embodiments, the frameshift mutation is in a microsatellite in the ACVR2A gene and comprises the sequence according to residues 509 to 515 of SEQ ID NO: 96 or 97, and wherein the first primer comprises the sequence defined SEQ ID NO: 90 or 91, and/or, when present, the second primer comprises the sequence defined by SEQ ID NO: 92. Preferably, the first primer comprises or consists of the sequence defined by SEQ ID NO: 90 or 91.

In some embodiments, the kit comprises the second primer and the kit further comprises a third primer, wherein the third primer is configured to anneal 5′ upstream of the region to which the second primer is configured to anneal and wherein the third primer is configured to anneal to the same strand as the first primer and the opposite strand to the second primer, or the kit comprises another control primer pair. For example, when the frameshift mutation is in a microsatellite in the TGFβR2 gene and comprises the sequence according to residues 270 to 278 of SEQ ID NO: 3 or SEQ ID NO: 4, then the third primer may comprise the sequence according to SEQ ID NO: 13. In another example, when the frameshift mutation is in a microsatellite in the ASTE1 gene and comprises the sequence according to residues 328 to 337 of SEQ ID NO: 7 or SEQ ID NO: 8, then the third primer may comprise or consist of the sequence according to SEQ ID NO: 30 or SEQ ID NO: 31. In another example, when the frameshift mutation is in a microsatellite in the TAF1β gene and comprises the sequence according to residues 255 to 264 of SEQ ID NO: 11 or SEQ ID NO: 12, then the third primer may comprise or consist of the sequence according to SEQ ID NO: 50. In another example, when the frameshift mutation is in a microsatellite in the ACVR2A gene and comprises the sequence according to residues 509 to 515 of SEQ ID NO: 96 or 97, then the third primer may comprise the sequence according to SEQ ID NO: 93.

A control primer pair can be understood as being a positive control for any DNA amplification that is carried out with the kit, to ascertain that DNA amplification has been carried out successfully, so that e.g. no detection of a frameshift mutation with the primers of the invention can be confirmed as the absence of a frameshift in the sample, rather than an error or failure in the DNA amplification itself. A control primer pair can be any pair of primers that is able to amplify a target sequence that is known to be present in the sample (e.g. a conserved sequence between a frameshift mutation and a wild type sequence). Instead of a separate control primer pair, a third primer can be used with the second primer of the invention for the same purpose (i.e. a positive control).

Preferably, the sequence containing the wild type microsatellite comprises the sequence defined in SEQ ID NO: 3. As mentioned above, this is a microsatellite in TGFβR2 in which mutations can occur which can be linked with disease, such as gastric cancer (GC) and colorectal cancer (CRC). In some embodiments, the sequence containing the wild type microsatellite comprises the sequence defined in SEQ ID NO: 7. This is a microsatellite in ASTE1 in which mutations can occur which can be linked with disease, such as endometrial cancer and gastric cancer. In some embodiments, the sequence containing the wild type microsatellite comprises the sequence defined in SEQ ID NO: 11. This is a microsatellite in TAF1β in which mutations can occur which can be linked with disease, such as endometrial cancer and gastric cancer. In some embodiments, the sequence containing the wild type microsatellite comprises the sequence defined in SEQ ID NO: 96. This is a microsatellite in ACVR2A in which mutations can occur which can be linked with disease, such as stomach adenocarcinoma (STAD), uterine corpus endometrial cancer (UCEC) and colon adenocarcinoma (COAD).

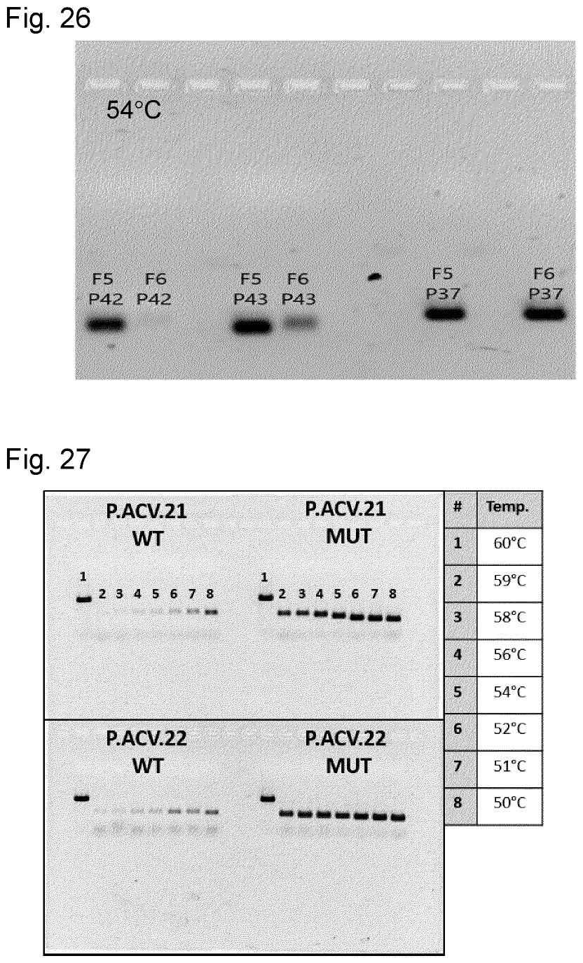

Preferably still, the third primer comprises the sequence defined by SEQ ID NO: 13. Again, it will be understood that the use of the term “comprises” also includes a primer that consist of this sequence. As described herein, this is a suitable example of a primer that can be used as one part of a positive control primer pair for detection of the a10 microsatellite in TGFβR2 and is demonstrated to function successfully as per the experimental data disclosed below. In some embodiments, the primer comprises the sequence defined by SEQ ID NO: 30. This is a suitable example of a primer what can be used as part of a positive control primer pair for detection of the a11 microsatellite in ASTE1, and is demonstrated to function successfully in FIG. 18. In some embodiments, the primer comprises the sequence defined by SEQ ID NO: 50. This is a suitable example of a primer what can be used as part of a positive control primer pair for detection of the a11 microsatellite in TAF1β, and is demonstrated to function successfully in FIG. 26. In some embodiments, the primer comprises the sequence defined by SEQ ID NO: 93. This is a suitable example of a primer what can be used as part of a positive control primer pair for detection of the a8 microsatellite in ACVR2A.

In some embodiments, the target sequence comprising a microsatellite having a frameshift mutation comprises the sequence according to residues 270 to 278 of SEQ ID NO: 3 or SEQ ID NO: 4. As mentioned above, this is the frameshifted microsatellite in TGFβR2 that can be linked with disease, such as gastric cancer (GC) and colorectal cancer (CRC). In some embodiments, the target sequence comprising a microsatellite having a frameshift mutation comprises the sequence according to residues 328 to 337 of SEQ ID NO: 7 or SEQ ID NO: 8. As mentioned above, this is the frameshifted microsatellite in ASTE1 that can be linked with disease, such as gastric cancer (GC) and endometrial cancer. In some embodiments, the target sequence comprising a microsatellite having a frameshift mutation comprises the sequence according to residues 255 to 264 of SEQ ID NO: 11 or SEQ ID NO: 12. As mentioned above, this is the frameshifted microsatellite in TAF1β that can be linked with disease, such as gastric cancer (GC) and endometrial cancer. In some embodiments, the target sequence comprising a microsatellite having a frameshift mutation comprises the sequence according to residues 509 to 515 of SEQ ID NO: 96 or SEQ ID NO: 97. As mentioned above, this is the frameshifted microsatellite in ACVR2A that can be linked with disease, such as stomach adenocarcinoma (STAD), uterine corpus endometrial cancer (UCEC) and colon adenocarcinoma (COAD).

Preferably, the kit further comprises instructions for use.

In some embodiments, the kit further comprises the components for carrying out DNA amplification. Such components will be well known to the skilled person who is well acquainted with techniques for DNA amplification including polymerase chain reaction (PCR), loop mediated isothermal application (LAMP), nucleic acid sequence based amplification (NASBA or 3SR), strand displacement amplification (SDA), rolling circle amplification (RCA), and ligase chain reaction (LCR). The skilled person will know that such components can be provided separately, or can be bought as a commercial product as a “ready mix” of the necessary components required.

Optionally, these components may include a buffer, dNTPs and/or Taq polymerase. For example, this buffer may be provided as a 10× buffer and dNTPs may be provided at a 10 mM concentration. In some embodiments, the buffer includes ammonium (NH₄⁺) ions and, optionally, may be provided as 1× buffer. In some embodiments, the buffer is Key buffer, optionally 1× Key buffer. In some embodiments, the buffer includes potassium ions (K⁺). In some embodiments, the buffer including potassium (K⁺) ions is used at a final concentration of between 0.5× and 1×, preferably at 0.5× or 1×. In some embodiments, the buffer is standard Taq buffer, optionally 1× standard Taq buffer.

In another aspect of the invention, there is provided a primer for DNA amplification comprising or consisting of the sequence defined by any one of SEQ ID NO: 24 to 28, by any one of SEQ ID NO: 45, 47, 83 or 100, by any one of SEQ ID NO: 43, 44, 46, 48 and 49, by any one of SEQ ID NO: 60 to 62, or by SEQ ID NO: 89, and wherein X defines a substitution to any nucleotide which is mismatched to the corresponding nucleotide in the target sequence and the wild type sequence. Preferably, the primer for DNA amplification comprises or consists of the sequence defined by any one of SEQ ID NO: 24 to 28, more preferably, the primer for DNA amplification comprises or consists of the sequence defined by SEQ ID NO: 24. Preferably, the primer for DNA amplification comprises or consists of the sequence defined by any one of SEQ ID NO: 45, 47, 83 and 100, or by any one of SEQ ID NO: 43, 44, 46, 48 and 49, more preferably, the primer for DNA amplification comprises or consists of the sequence defined by any one of SEQ ID NO: 45, 47, 48, 83 and 100, more preferably the primer for DNA amplification comprises or consists of the sequence defined by SEQ ID NO: 45, 83 or 100. Preferably, the primer for DNA amplification comprises or consists of the sequence defined by any one of SEQ ID NO: 60 to 62, more preferably, the primer for DNA amplification comprises or consists of the sequence defined by SEQ ID NO: 61. Preferably, the primer for DNA amplification comprises or consists of the sequence defined by SEQ ID NO: 89. In another aspect of the invention, there is provided a primer for DNA amplification comprising or consisting of the sequence defined by any one of SEQ ID NO: 17 to 19 or 21 to 23, by any one of SEQ ID NO: 34 to 38 and 40, by any one of SEQ ID NO: 39, 41, 42, 84 to 88, 98, 99 and 101, by any one of SEQ ID NO: 53 to 59 or by SEQ ID NO: 90 or 91. Preferably, the primer for DNA amplification comprises or consists of the sequence defined by any one of SEQ ID NO: 17 to 19 or 21 to 23, more preferably, the primer for DNA amplification comprises or consists of the sequence defined by SEQ ID NO: 17. Preferably, the primer for DNA amplification comprises or consists of the sequence defined by any one of SEQ ID NO: 34 to 38 and 40, or by any one of SEQ ID NO: 39, 41, 42, 84 to 88, 98, 99 and 101, more preferably, the primer for DNA amplification comprises or consists of the sequence defined by any one of SEQ ID NO: 37, 40, 41, 84 to 88, 98, 99 and 101, and yet more preferably the primer for DNA amplification comprises or consists of the sequence defined by any one of SEQ ID NO: 84-88, 98, 99 and 101. Preferably, the primer for DNA amplification comprises or consists of the sequence defined by any one of SEQ ID NO: 53 to 59, more preferably, the primer for DNA amplification comprises or consists of the sequence defined by any one of SEQ ID NO: 55 or 56. Preferably, the primer for DNA amplification comprises or consists of the sequence defined by SEQ ID NO: 90 or 91. Again, it will be understood that the use of the term “comprises” also includes primers that consist of the sequences listed above. As described above, the data disclosed herein demonstrates that these primers are particularly useful within the scope of the invention for use in the detection of frameshift mutations in a microsatellite within TGFβR2 (also referred to as a9, where a10 is the wild type microsatellite), ASTE1 (also referred to as a10, where a11 is the wild type microsatellite), TAF1β (also referred to as a10, where a11 is the wild type microsatellite) and ACVR2A (also referred to as a7, where a8 is the wild type microsatellite).

In a further aspect of the invention, there is provided a method for detecting a mutation in a microsatellite contained in a target sequence of a double stranded DNA molecule, wherein the mutation is a frameshift of the microsatellite as compared with the corresponding wild type microsatellite sequence using any of the above-detailed primers. The method comprises:
a) providing a first aliquot of a sample comprising human DNA,
b) adding to the first aliquot the necessary components for DNA amplification, a first primer and optionally a second primer; wherein the first primer is suitable for detection of a mutation in a microsatellite contained in a target sequence of a double stranded DNA molecule and comprises a region of nucleotides that is complementary to the target sequence containing the microsatellite having a frameshift mutation except for between one and four nucleotides which are mismatched to the target sequence containing the microsatellite having a frameshift mutation and which are also mismatched to a corresponding sequence containing the wild type microsatellite; and the second primer, when present, is configured to anneal to the target sequence 3′ downstream of the microsatellite, to form a first reaction mix;
wherein the first primer is configured to anneal to the sense strand of the DNA molecule and the second primer, when present, is configured to anneal to the antisense strand of the DNA molecule, or the first primer is configured to anneal to the antisense strand of the DNA molecule and the second primer, when present, is configured to anneal to the sense strand of the DNA molecule,
c) carrying out DNA amplification on the first reaction mix,
d) detecting the presence of the frameshift mutation in the sample when an amplification product is produced from the DNA amplification of the first reaction mix. In some embodiments, the primer comprises between one and three nucleotides which are mismatched to the target sequence containing the microsatellite having a frameshift mutation and which are also mismatched to a corresponding sequence containing the wild type microsatellite.

As mentioned above, DNA amplification may include polymerase chain reaction (PCR), loop mediated isothermal application (LAMP), nucleic acid sequence based amplification (NASBA or 3SR), strand displacement amplification (SDA), rolling circle amplification (RCA), and ligase chain reaction (LCR). However, preferably the DNA amplification used is PCR.

Preferably, DNA amplification is carried out in high stringency conditions. As mentioned above, high stringency conditions are conditions at which amplification is suboptimal so that the reaction only allows for successful DNA amplification where there is a very high level of matching to the target sequence. In other words, the conditions are suboptimal for DNA amplification in order that primers with lower sequence matching to the target sequence are not able to anneal. Such suboptimal conditions can be generated by altering one or more of temperature, cycle number, ionic strength and the presence of certain organic solvents that allow pairing of nucleic acid sequences. In particular, this may include where the annealing step of PCR is carried out at a temperature that is at least 2%, 5%, or 10% higher than the recommended annealing temperature of the reaction. Alternatively, the annealing step of PCR is carried out at a temperature that is at least 1° C., 2° C., 3° C., 4° C., 5° C., 6° C., 7° C., 8° C., 9° C., 10° C., 11° C. or 12° C. higher, preferably at least 1° C., 2° C., 3° C., 4° C., 5° C., 6° C., 7° C., 8° C., 9° C. or 10° C. higher, than the recommended annealing temperature of the reaction. In some embodiments, the annealing step of PCR is carried out at a temperature that is 4° C., 5° C., 6° C., 7° C., 8° C., 9° C., 10° C., 11° C. or 12° C. higher, preferably 4° C. higher, than the recommended annealing temperature of the reaction.

In some embodiments, the annealing step is carried out at a temperature of 44° C., 44.5° C., 45° C., 45.5° C., 46° C., 46.5° C., 47° C., 47.5° C., 48° C., 48.5° C., 49° C., 49.5° C., 50° C., 50.5° C., 51° C., 51.5° C., 52° C., 52.5° C., 53° C., 53.5° C., 54° C., 54.5° C., 55° C., 55.5° C., 56° C., 56.5° C., 57° C., 57.5° C., 58° C., 58.5° C., 59° C., 59.5° C., 60° C., 60.5° C. or 61° C. In some embodiments, the annealing step is carried out at a temperature between 54° C. and 61° C., preferably between 56° C. and 60° C., more preferably between 56° C. and 59° C. or between 56° and 58° C., and preferably wherein the frameshift is in a microsatellite in a TGFβR2 gene. In some embodiments, the annealing step is carried out at a temperature of 56.0° C., 56.1° C., 56.2° C., 56.3° C., 56.4° C., 56.5° C., 56.7° C., 56.8° C., 56.9° C., 57.0° C., 57.1° C., 57.2° C., 57.3° C., 57.4° C., 57.5° C., 57/6° C., 57.7° C. 57.8° C. 57.9° C. or 58.0° C., and preferably the frameshift is in a microsatellite in a TGFβR2 gene. In some embodiments, the annealing step is carried out at a temperature of 57° C. and preferably the frameshift is in a microsatellite in a TGFβR2 gene.

In some embodiments, the annealing step is carried out at a temperature of between 42° C. and 58° C., preferably between 42° C. and 52° C. or between 45° C. and 58° C. or between 45° C. and 56° C. or between 48° C. and 58° C., more preferably between 45° C. and 54.5° C., and preferably wherein the frameshift is in a microsatellite in a ASTE1 gene. In some embodiments, the annealing step is carried out at 48° C., 48.5° C., 49° C., 49.5° C., 50° C., 50.5° C., 51° C., 51.5° C., 52° C., 52.5° C., 53° C., 53.5° C., 54° C., 54.5° C., 55° C., 55.5° C., 56° C., 56.5° C., 57° C., 57.5° C. or 58° C., preferably wherein the frameshift is in a microsatellite in a ASTE1 gene. In some embodiments, the annealing step is carried out at 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 50.0° C., 50.5° C., 51.0° C., 51.5° C. or 52.0° C., preferably wherein the frameshift is in a microsatellite in a ASTE1 gene. In some embodiments, the annealing step is carried out at 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58° C., preferably wherein the frameshift is in a microsatellite in a ASTE1 gene.

In some embodiments, the annealing step is carried out at a temperature of between 52° C. and 58° C., preferably 54° C., and preferably wherein the frameshift is in a microsatellite in a TAF1β gene. In some embodiments, the annealing step is carried out at 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58.0° C., preferably wherein the frameshift is in a microsatellite in a TAF1β gene. In some embodiments, the annealing step is carried out at 54.0° C., preferably wherein the frameshift is in a microsatellite in a TAF1β gene.

In some embodiments, the annealing step is carried out at a temperature of between 50° C. and 60° C., preferably 56° C., and preferably wherein the frameshift is in a microsatellite in an ACVR2A gene. In some embodiments, the annealing step is carried out at 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5, 57.0° C., 57.5° C., 58.0° C., 58.5° C., 59.0° C., 59.5° C. or 60.0° C., preferably wherein the frameshift is in a microsatellite in an ACVR2A gene.

In some embodiments, the annealing step is carried out at a temperature of 56.0° C., 56.1° C., 56.2° C., 56.3° C., 56.4° C., 56.5° C., 56.7° C., 56.8° C., 56.9° C., 57.0° C., 57.1° C., 57.2° C., 57.3° C., 57.4° C., 57.5° C., 57.6° C., 57.7° C. 57.8° C. 57.9° C. or 58.0° C., and the first primer comprises the sequence defined by any one of SEQ ID NO: 17 to 19 or 21 to 23, SEQ ID NO: 24 and SEQ ID NO: 25 to 28, preferably SEQ ID NO: 17 or 24. In some of these embodiments, the annealing step is carried out at a temperature of 57° C.

In some embodiments, the annealing step is carried out at a temperature of 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58.0° C., and the first primer comprises the sequence defined by any one of SEQ ID NOs: 34 to 38 and 40, SEQ ID NOs: 39, 41 and 42, SEQ ID NOs: 43, 44, 46, 48 and 49, or SEQ ID NOs: 45 and 47, preferably defined by SEQ ID NO: 34 or 43. Preferably, the annealing step is carried out at a temperature of 54° C., 54.5° C., 55° C., 55.5° C., 56° C., 56.5° C., 57° C., 57.5° C. or 58° C.

In some embodiments, the annealing step is carried out at a temperature of 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 50° C., 50.5° C., 51° C., 51.5° C., 52° C., 52.5° C., 53° C., 53.5° C., 54° C., 54.5° C., 55° C., 55.5° C., 56° C., 57.0° C., 57.5° C. or 58.0° C. and the first primer comprises the sequence defined by any one of SEQ ID NOs: 34 to 38 and 40, SEQ ID NOs: 39, 41 and 42, SEQ ID NOs: 43, 44, 46, 48 and 49, or SEQ ID NO: 45 and 47, preferably defined by SEQ ID NO: 37 or 45. Preferably, the annealing step is carried out at a temperature of 48° C., 48.5° C., 49° C., 49.5° C., 50° C., 50.5° C., 51° C., 51.5° C., 52° C., 52.5° C. or 53° C.

In some embodiments, the annealing step is carried out at a temperature of 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58.0° C., and the first primer comprises the sequence defined by any one of SEQ ID NOs: 34 to 38 and 40, SEQ ID NOs: 39, 41 and 42, SEQ ID NOs: 43, 44, 46, 48 and 49, or SEQ ID NOs: 45 and 47, preferably defined by SEQ ID NO: 39 or 46. Preferably, the annealing step is carried out at a temperature of 54° C., 54.5° C., 55° C., 55.5° C., 56° C., 56.5° C., 57° C., 57.5° C. or 58° C.

In some embodiments, the annealing step is carried out at a temperature of 42.0° C., 42.5° C., 43.0° C., 43.5° C., 44.0° C., 44.5° C., 45° C., 45.5° C., 46° C., 46.5° C., 47° C., 47.5° C., 48° C., 48.5° C., 49° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C. 57.5° C. or 58.0° C., and the first primer comprises the sequence defined by any one of SEQ ID NOs: 34 to 38 and 40, SEQ ID NOs: 39, 41 and 42, SEQ ID NOs: 43, 44, 46, 48 and 49, or SEQ ID NOs: 45 and 47, preferably defined by SEQ ID NO: 40 or 47. Preferably, the annealing step is carried out at a temperature of 45° C., 45.5° C., 46° C., 46.5° C., 47° C., 47.5° C. or 48° C.

In some embodiments, the annealing step is carried out at a temperature of 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58.0° C., and the first primer comprises the sequence defined by any one of SEQ ID NOs: 34 to 38 and 40, SEQ ID NOs: 39, 41 and 42, SEQ ID NOs: 43, 44, 46, 48 and 49, or SEQ ID NOs: 45 and 47, preferably defined by SEQ ID NO: 41 or 48. Preferably, the annealing step is carried out at a temperature of 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 50.0° C., 50.5° C., 51.0° C. or 51.5° C.

In some embodiments, the annealing step is carried out at a temperature of 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58.0° C., and the first primer comprises the sequence defined by any one of SEQ ID NOs: 34 to 38 and 40, SEQ ID NOs: 39, 41 and 42, SEQ ID NOs: 43, 44, 46, 48 and 49, or SEQ ID NOs: 45 and 47, preferably defined by SEQ ID NO: 42 or 49. Preferably, the annealing step is carried out at a temperature of 42° C., 42.5° C., 43° C., 43.5° C., 44° C., 44.5° C., 45° C., 45.5° C., 46° C., 46.5° C., 47° C., 47.5° C., 48° C., 48.5° C., 49° C., 49.5° C., 50.0° C., 50.5° C., 51.0° C. or 51.5° C.

In some embodiments, the annealing step is carried out at a temperature of 42.0° C., 42.5° C., 43.0° C., 43.5° C., 44.0° C., 44.5° C., 45.0° C., 45.5° C., 46.0° C., 46.5° C., 47.0° C., 47.5° C., 48.0° C., 48.5° C., 49.0° C., 49.5° C., 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5° C., 57.0° C., 57.5° C. or 58.0° C., and the first primer comprises the sequence defined by any one of SEQ ID NOs: 53 to 59 or SEQ ID NOs: 60 to 62, preferably defined by SEQ ID NO: 55, 56 or 61. Preferably, the annealing step is carried out at a temperature of 54.0° C.

In some embodiments, the annealing step is carried out at a temperature of 50.0° C., 50.5° C., 51.0° C., 51.5° C., 52.0° C., 52.5° C., 53.0° C., 53.5° C., 54.0° C., 54.5° C., 55.0° C., 55.5° C., 56.0° C., 56.5, 57.0° C., 57.5° C., 58.0° C., 58.5° C., 59.0° C., 59.5° C. or 60.0° C., and the first primer comprises the sequence defined by SEQ ID NO: 90 or 91. Preferably, the annealing step is carried out at a temperature of 56.0° C.

Additionally or alternatively, the high stringency conditions may include using a lower buffer concentration than is recommended for the reaction, such as 10%, 20%, 30%, 40%, 50%, 60% or 75% of the recommended concentration of a buffer in a reaction. In some embodiments, the buffer concentration which is lower than the recommended buffer concentration may be less than 0.1×, 0.2×, 0.3×, 0.4×, 0.5×, 0.6× or 0.75×. In some embodiments, the final concentration of buffer in the reaction mix(es) is between 0.1× and 2×, between 0.1× and 1.5×, between 0.1× and 1×, between 0.2× and 1×, between 0.3× and 1×, between 0.4× and 1× or between 0.5× and 1×. In some embodiments, the final concentration of buffer in the reaction mix(es) is 0.5×, 0.6×, 0.7×, 0.8×, 0.9×, 1×, 1.5×, or 2×.

Additionally or alternatively, the high stringency conditions may include using a more stringent buffer in the reaction, such as a buffer including ammonium ions (NH₄⁺). In some embodiments, a buffer including ammonium (NH₄⁺) ions is used at a standard concentration, such as 1×. In some embodiments, a buffer including ammonium (NH₄⁺) ions is used (i.e. is in the reaction mix(es)) at a final concentration of between 1× and 2×, preferably 1×. In some embodiments, the buffer including ammonium (NH₄⁺) ions is Key buffer. In some embodiments, the buffer is 1× Key buffer.

In some embodiments, the buffer includes potassium ions (K⁺). In some embodiments, the buffer including potassium (K⁺) ions is used at a final concentration of between 0.5× and 1×, preferably at 0.5× or 1×. In some embodiments, the buffer is standard Taq buffer (KCl).

In some embodiments, the buffer includes ammonium ions (NH₄⁺) and/or potassium ions (K⁺). In some embodiments, the buffer including ammonium (NH₄⁺) ions and/or potassium (K⁺) ions is used at a final concentration of between 0.5× and 1×, preferably at 0.5× or 1×. In some embodiments, the buffer is ThermoPol® buffer.

In some embodiments, the final concentration of each of the forward and reverse primers in the reaction mix(es) is, independently, between 0.1 μM and 1 μM, between 0.1 μM and 0.5 μM, between 0.1 μM and 0.4 μM, between 0.1 μM and 0.4 μM or between 0.1 μM and 0.3 μM. In some embodiments, the final concentration of each of the forward and reverse primers in the reaction mix(es) is, independently, 0.1 μM, 0.2 μM or 0.3 μM, preferably 0.2 μM.

In some embodiments, the final concentration of the dNTPs in the reaction mix(es) is between 50 μM and 500 μM, between 50 μM and 400 μM, between 50 μM and 300 μM, between 100 μM and 300 μM, between 100 μM and 200 μM, between 150 μM and 300 μM, or between 150 μM and 250 μM. In some embodiments, the final concentration of the dNTPs in the reaction mix(es) is 100 μM, 150 μM, 200 μM, 250 μM or 300 μM, preferably 200 μM.

The high stringency conditions may additionally or alternatively include reducing the number of cycles in a PCR, such as carrying out 25 cycles, 20 cycles, 15 cycles or even 10 cycles, compared to 30, 35 or 40 cycles. In some embodiments, 30 or fewer, 25 or fewer, 20 or fewer, 15 or fewer or 10 or fewer cycles of PCR are carried out. In some embodiments, more than one of these high stringency conditions is used. Preferably, 30 or 25 cycles of PCR are carried out.

In some embodiments, a recommended annealing temperature is provided by the manufacturer of a commercially obtained primer. The skilled person is aware of many publicly available tools for calculating a recommended annealing temperature for a given primer. The skilled person is also aware that a recommended annealing temperature can be calculating by subtracting 3, 4, 5, or 6° C. from the Tₘ (melting temperature) given for a particular primer. In one embodiment, the recommended annealing temperature for a primer is 4° C. below the Tₘ of the primer under the conditions of the reaction. For example the Tₘ calculators are provided by New England BioLabs® (found at http://tmcalculator.neb.com/#!/main) or Thermo Fisher Scientific® (found at https://www.thermofisher.com/uk/en/home/brands/thermo-scientific/molecular-biology/molecular-biology-learning-center/molecular-biology-resource-library/thermo-scientific-web-tools/tm-calculator.html). The Tₘ calculator provided by Thermo Fischer Scientific® will also be known as the modified Allawi and SantaLucia method, as per Allawi and SantaLucia, 1997.

A recommended buffer concentration for a reaction mix is 1×. For example, in some embodiments 5 μl of a 10× buffer is added to a reaction mix to a final volume of 50 μl. In some embodiments, the buffer includes ammonium (NH₄⁺) ions. In some embodiments, the buffer is Key buffer. In some embodiments, the buffer includes potassium (K⁺) ions. In some embodiments, the buffer is standard Taq buffer. In these embodiments, the frameshift mutation in a microsatellite is preferably in a TGFβR2 gene.

A recommended buffer concentration for a reaction mix is 1×. For example, in some embodiments 2.5 μl of a 10× buffer is added to a reaction mix to a final volume of 25 μl, or 5 μl of a 10× buffer is added to a reaction mix to a final volume of 50 μl. In some embodiments, the buffer includes potassium (K⁺) ions. In some embodiments, the buffer is standard Taq buffer. In some embodiments, the buffer includes ammonium (NH₄⁺) ions and/or potassium (K⁺) ions. In some embodiments, the buffer is ThermoPol® buffer. In these embodiments, the frameshift mutation in a microsatellite is preferably in the ASTE1 gene.

A recommended buffer concentration for a reaction mix is 1×. For example, in some embodiments 2.5 μl of a 10× buffer is added to a reaction mix to a final volume of 25 μl. In some embodiments, the buffer includes potassium (K⁺) ions. In some embodiments, the buffer is standard Taq buffer. In these embodiments, the frameshift mutation in a microsatellite is preferably in the TAF1β gene.

In some embodiments, the annealing temperature is at least 57° C. and the buffer includes potassium (K⁺) ions. Preferably the buffer is standard Taq (KCl) buffer. In these embodiments, the frameshift mutation in a microsatellite is preferably in a TGFβR2 gene.

In some embodiments, the annealing temperature is at least 44° C. or 48° C. and the buffer includes potassium (K⁺) ions. Preferably the buffer is standard Taq (KCl) buffer. In these embodiments, the frameshift mutation in a microsatellite is preferably in a ASTE1 gene.

In some embodiments, the annealing temperature is at least 42° C., 50° C., 50.5° C., 52° C. or 54° C. and the buffer includes ammonium (NH₄⁺) ions and/or potassium (K⁺) ions. Preferably the buffer is ThermoPol® buffer. In these embodiments, the frameshift mutation in a microsatellite is preferably in a ASTE1 gene.

In some embodiments, the annealing temperature is 54° C. to 58° C. and the buffer includes ammonium (NH₄⁺) ions and/or potassium (K⁺) ions, preferably the buffer is ThermoPol® buffer.

In some embodiments, the annealing temperature is 54° C. to 55° C. and the buffer includes potassium (K⁺) ions, preferably the buffer is standard Taq (KCl) buffer. In these embodiments, the frameshift mutation in a microsatellite is preferably in a ASTE1 gene.

In some embodiments, the annealing temperature is at least 54° C. and the buffer includes potassium (K⁺) ions. Preferably the buffer is standard Taq (KCl) buffer. In these embodiments, the frameshift mutation in a microsatellite is preferably in a TAF1β gene.

In some embodiments, the annealing temperature is 50° C. to 60° C., preferably 56° C., and the buffer includes ammonium ions (NH₄⁺). Preferably the buffer is Key buffer. In these embodiments, the frameshift mutation in a microsatellite is preferably in an ACVR2A gene.

In some embodiments, the final concentration of the template DNA in the reaction mix(es) is between 0.01 ng/μL and 1.5 ng/μL, 0.02 ng/μL and 1.2 ng/μL, 0.03 ng/μL and 1.0 ng/μL, 0.04 ng/μL and 0.5 ng/μL, or 0.05 ng/μL and 0.2 ng/μL, preferably between 0.05 ng/μL and 0.2 ng/μL. Preferably the minimum concentration of the template DNA in the reaction mix(es) is 0.01 ng/μL, 0.02 ng/μL, 0.03 ng/μL, 0.04 ng/μL, 0.05 ng/L, 0.06 ng/μL, 0.07 ng/L, 0.08 ng/μL, 0.09 ng/μL, 0.1 ng/μL, 0.15 ng/μL or 0.2 ng/μL, preferably 0.05 ng/μL or 0.2 ng/μL. Preferably, the maximum concentration of the template DNA in the reaction mix(es) is 1.0 ng/μL, 0.5 ng/μL, 0.4 ng/μL, 0.3 ng/μL, 0.2 ng/μL, 0.15 ng/μL, 0.1 ng/μL or 0.05 ng/μL, preferably 0.2 ng/μL or 0.05 ng/μL. Preferably, the final concentration of the template DNA in the reaction mix(es) is 0.01 ng/μL, 0.02 ng/μL, 0.03 ng/μL, 0.04 ng/μL or 0.05 ng/μL. Most preferably, the final concentration of the template DNA in the reaction mix(es) is 0.05 ng/μL. A low final concentration of template DNA is beneficial because this means that a lower amount of template DNA is required for the PCR reaction.

A sample comprising human DNA may be any sample obtainable from a patient, for example, the sample may be a bodily fluid, a tissue, or cells. The sample may also be a liquid biopsy, such as plasma, which contains cell free DNA.

In some embodiments, the method further comprises:
a) also adding to the first aliquot at step b) of the method either:
   I) the second primer and a third primer, wherein the third primer is configured to anneal 5′ upstream of the region to which the second primer is configured to anneal, and wherein the third primer is configured to anneal to the same strand as the first primer and the opposite strand to the second primer or
   II) a control primer pair, to form the first reaction mix,
   and detecting that DNA amplification has been carried out successfully when an amplification product is produced from the DNA amplification using the second and third primers or the control primer pair; or
b) providing a second aliquot of the sample comprising human DNA, adding to the second aliquot the necessary components for DNA amplification and either:
   I) the first primer and a third primer, wherein the third primer wherein the third primer is configured to anneal 5′ upstream of the region to which the second primer is configured to anneal, and wherein the third primer is configured to anneal to the same strand as the first primer and the opposite strand to the second primer or
   II) a control primer pair, and
   carrying out DNA amplification on the second reaction mix, and detecting that DNA amplification has been carried out successfully when an amplification product is produced from the DNA amplification of the second reaction mix.

In some embodiments, the first primer is the primer of the invention described above and, therefore, has any of the features described above. For example, in some embodiments, the first primer is configured to anneal across the length of the microsatellite having a frameshift mutation, and to at least one nucleotide flanking the 5′ and/or at least one nucleotide flanking the 3′ end of the microsatellite having a frameshift mutation. The first primer may anneal to at least one, two, three, four, or five nucleotides flanking one or both ends of the microsatellite having a frameshift mutation. In some embodiments, the first primer does not anneal across the length of the corresponding wild-type microsatellite sequence, and the 3′ end of the first primer does not anneal to the corresponding sequence containing the wild-type microsatellite. This provides the advantage that the first primer anneals to the sequence containing the microsatellite having a frameshift mutation, but does not anneal to the corresponding sequence having the wild-type microsatellite. In some embodiments, the first primer anneals to one, two or three, preferably one, two or three, more preferably one, nucleotides flanking the 3′ end of the microsatellite having a frameshift mutation (i.e. the first primer comprises one, two or three nucleotides 3′ of the microsatellite, with respect to the first primer, which anneal to the corresponding sequence comprising the microsatellite having a frameshift mutation). In some embodiments, the first primer comprises one, two or three nucleotides flanking the 3′ end of the microsatellite having a frameshift mutation and these one, two or three nucleotides form a GC-clamp. In some embodiments, the first primer anneals across the length of the microsatellite having the frameshift mutation and, 3′ of the microsatellite having the frameshift mutation, with respect to the first primer, the first primer consists of one, two or three nucleotides which anneal to the nucleotides flanking the microsatellite having the frameshift mutation. This provides the advantage that the first primer annealed to the sequence containing the microsatellite having a frameshift mutation can be extended in the 5′-to-3′ direction, thereby allowing replication of the sequence containing the microsatellite having the frameshift mutation. The nucleotide(s) flanking the 3′ end of the microsatellite having a frameshift mutation are described above.

In some embodiments, the first primer anneals to at least 1, 2, 3, 4, 5, 6, 7, 8, 9, 10, 11, 12 or 13 nucleotides flanking the 5′ end of the microsatellite having a frameshift mutation, with respect to the first primer. In some embodiments, the primer anneals to 9, 10, 11, 12 or 13 nucleotides flanking the 5′ end of the microsatellite having a frameshift mutation, with respect to the primer. In some embodiments, the primer anneals to 1, 8, 10, 12 or 13 nucleotides flanking the 5′ end of the microsatellite having a frameshift mutation, with respect to the primer. In some embodiments, at least 30%, 35%, 40%, 45%, 50%, 55%, 60% or 65% of the nucleotides of the first primer anneal to nucleotides flanking the 5′ end of the microsatellite having a frameshift mutation, with respect to the first primer. In some embodiments, the part of the first primer which anneals to the nucleotides flanking the 5′ end the microsatellite having a frameshift mutation, with respect to the first primer, also anneals to the corresponding sequence containing the wild-type microsatellite. This provides the advantage that the first primer is specific for the desired target sequence. In some embodiments, less than 35%, 30%, 25%, 20%, 15%, 10% or 5% of the nucleotides of the primer anneal to nucleotides flanking the 5′ end of the microsatellite having a frameshift mutation, with respect to the primer. The nucleotides flanking the 5′ end of the microsatellite having the frameshift mutation are further described above.

In some embodiments, each of the primers consists of between 16 and 30 nucleotides. Preferably, the primer consists of between 16 and 25 nucleotides. Preferably still, the primer consists of between 17 and 24 or between 17 and 23 nucleotides. That is to say, each of the primers independently consists of between 16 and 30 nucleotides. Preferably, each of the primers independently consists of between 1 and 25 nucleotides. Preferably still, each of the primers independently consists of between 17 and 24 nucleotides, between 17 and 23 nucleotides, between 19 and 23 nucleotides, between 19 and 22 nucleotides, or between 19 and 21 nucleotides. In some embodiments, the primer consists of 21, 22, 23 or 24 nucleotides, preferably 21, 22 or 23 nucleotides. Other preferred lengths of the primers are described above.

In some embodiments, at least one mismatched nucleotide is located in a position of the first primer that anneals 3′ downstream of the microsatellite or within the microsatellite. Preferably, all of the mismatched nucleotides are located in a position of the first primer that anneals 3′ downstream of the microsatellite or within the microsatellite. In some embodiments, all of the mismatched nucleotides are located in a position of the first primer that anneals within the microsatellite. Other preferred locations of at least one mismatch are described above.

In some embodiments, at least one mismatched nucleotide in the first primer is within five nucleotides 5′ upstream of the 3′ end of the microsatellite or within 12 nucleotides 3′ downstream of the 3′ end of the microsatellite. In some embodiments, at least one mismatched nucleotide in the first primer is within 1, 2, 3, 4 or 5 nucleotides 5′ upstream of the 3′ end of the microsatellite or within 2, 4, 5 or 12 nucleotides 3′ downstream of the 3′ end of the microsatellite. Preferably, all of the mismatched nucleotides in the first primer are within two, three, four or five nucleotides 5′ upstream or within 12 nucleotides 3′ downstream of the 3′ end of the microsatellite. More preferably, all of the mismatched nucleotides in the first primer are within two, three, four or five nucleotides 5′ upstream of the 3′ end of the microsatellite.

In some embodiments, at least one mismatched nucleotide in the first primer is within 1 or 2 nucleotides 5′ upstream of the 3′ end of the microsatellite or within 2 or 4 nucleotides 3′ downstream of the 3′ end of the microsatellite. In some embodiments, at least one mismatched nucleotide in the first primer is within 1 or 2 nucleotides 5′ upstream of the 3′ end of the microsatellite or within 2 nucleotides 3′ downstream of the 3′ end of the microsatellite. In some embodiments, at least one mismatched nucleotide in the first primer is within 1 nucleotide 5′ upstream of the 3′ end of the microsatellite or within 2 nucleotides 3′ downstream of the 3′ end of the microsatellite. In the embodiments of this paragraph, the frameshift mutation is preferably in a microsatellite in the TGFβR2 gene.

In some embodiments, at least one mismatched nucleotide in the first primer is within 1, 2, 3, 4 or 5 nucleotides 5′ upstream of the 3′ end of the microsatellite or within 5 or 12 nucleotides 3′ downstream of the 3′ end of the microsatellite. In some embodiments, at least one mismatched nucleotide in the first primer is within 1, 2, 3, 4 or 5 nucleotides 5′ upstream of the 3′ end of the microsatellite. In the embodiments of this paragraph, the at least one mismatched nucleotide is preferably in a microsatellite in ASTE1. In some embodiments, at least one mismatched nucleotide in the first primer is within 2 nucleotides 5′ upstream of the 3′ end of the microsatellite or within 12 nucleotides 3′ downstream of the 3′ end of the microsatellite. In the embodiments of this paragraph, the frameshift mutation is preferably in a microsatellite in the ASTE1 gene.

In some embodiments, at least one mismatched nucleotide in the first primer is within 1, 2 or 4 nucleotides 5′ upstream of the 3′ end of the microsatellite. In the embodiment of this paragraph, the frameshift mutation is preferably in a microsatellite in the TAF1β gene.

In some embodiments, at least one mismatched nucleotide in the first primer is within 1, 2 or 3 nucleotides 5′ upstream of the 3′ end of the microsatellite. In the embodiment of this paragraph, the frameshift mutation is preferably in a microsatellite in the ACVR2A gene.

In some embodiments, the first nucleotide 3′ downstream of the 3′ end of the microsatellite is not a mismatched nucleotide.

In some embodiments, at least one mismatched nucleotide in the first primer is a substitution of a thymine (T) with an adenine (A), a substitution of a thymine (T) with a cytosine (C), a substitution of a thymine (T) with a guanine (G), a substitution of an adenine (A) with a cytosine (C), a substitution of an adenine (A) with a guanine (G), a substitution of an adenine (A) with a thymine (T) or a substitution of a cytosine (C) with a guanine (G).

In some embodiments, the DNA amplification is polymerase chain reaction (PCR), wherein the PCR comprises a plurality of cycles of denaturation, annealing and extension.

In some embodiments, the reaction mix(s) comprises 1× buffer (or 0.5× buffer), 0.4 mM dNTPs, 0.2 μM forward primer, and 0.2 μM reverse primer. In some embodiments, the buffer includes ammonium (NH₄⁺) ions, and preferably is Key buffer.

In some embodiments, the reaction mix(s) comprises 1× buffer (or 0.5× buffer), 0.2 mM dNTPs, 0.2 μM forward primer, and 0.2 μM reverse primer. In some embodiments, the buffer includes potassium ions (K⁺), and preferably is standard Taq buffer. In some embodiments, the buffer includes ammonium ions (NH₄⁺) and/or potassium ions (K⁺), and preferably is ThermoPol® buffer.

In some embodiments, the method further comprises step d) further comprises running the product of the PCR reaction on a gel and visualising a band to confirm that DNA amplification has been successful.

This approach allows for a visual confirmation as to whether a PCR reaction has successfully generated an amplicon (i.e. amplified a target sequence from the sample). The skilled person is well aware of how to run such a gel, and this will typically involve mixing the PCR reaction with a dye, loading this onto an agarose based gel, carrying out electrophoresis on the gel so as the dyed PCR reaction migrates towards the positive end of the gel forming a band that can be visualised. This provides an advantage over the conventional sequencing approach which can take several days to provide a result, in that this PCR method can be carried out in several hours, or even less than an hour. As such, this method has the advantage that a clinically relevant finding (i.e. presence of a relevant mutation in a given disease) can be provided to a clinician or patient at a greater speed.

In a further embodiment, the method further comprises step f) cutting out the band for DNA sequencing. The advantage of running the PCR reaction on a gel as described above is that a band can be cut out of the gel and processed according to techniques known to the skilled person so that this can be sequenced. Sequencing is not essential, however this can allow for a further confirmation that the frameshift mutation is indeed present in the sample.

As is disclosed herein, there are many microsatellites in which disease-relevant frameshift mutations can occur. For example, the frameshift may be in a microsatellite in any gene selected from ASTE1, ACVR22 (also known as ACVR2A), TAF1β, KIAA2018, SLC22A9 and TGFβR2. In some embodiments, the frameshift mutation is in a microsatellite in TGFβR2, ASTE1, TAF1β or ACVR2A.

In some embodiments, the frameshift mutation is in a microsatellite in the TGFβR2 gene and the target sequence comprises the sequence according to residues 270 to 278 of SEQ ID NO: 3 or SEQ ID NO: 4, and the first primer comprises a sequence defined by any one of SEQ ID NOs: 24 to 28 and/or, when present, the second primer comprises the sequence defined by SEQ ID NO: 14. In some embodiments, the frameshift mutation is in a microsatellite in the ASTE1 gene and the target sequence comprises the sequence according to residues 328 to 337 of SEQ ID NO: 7 or SEQ ID NO: 8, and the first primer comprises a sequence defined by SEQ ID NO: 45, 47, 83 or 100, preferably SEQ ID NO: 45, 83 or 100, and/or, when present, the second primer comprises the sequence defined by SEQ ID NO: 29. In some embodiments, the frameshift mutation is in a microsatellite in the ASTE1 gene and comprises the sequence according to residues 328 to 337 of SEQ ID NO: 7 or SEQ ID NO: 8, and wherein the first primer comprises the sequence defined by SEQ ID NO: 43, 44, 46, 48 or 49, and/or, when present, the second primer comprises the sequence defined by SEQ ID NO: 29. In some embodiments, the frameshift mutation is in a microsatellite in the TAF1β gene and comprises the sequence according to residues 255 to 264 of SEQ ID NO: 11 or SEQ ID NO: 12, and wherein the first primer comprises the sequence defined by SEQ ID NOS: 60 to 62, and/or, when present, the second primer comprises the sequence defined by SEQ ID NO: 51. In some embodiments, the frameshift mutation is in a microsatellite in the ACVR2A gene and comprises the sequence according to residues 509 to 515 of SEQ ID NO: 96 or 97, and wherein the first primer comprises the sequence defined by SEQ ID NO: 89, and/or, when present, the second primer comprises the sequence defined by SEQ ID NO: 92.

In some embodiments, the frameshift mutation is in a microsatellite in the TGFβR2 gene and comprises the sequence according to residues 270 to 278 of SEQ ID NO: 3 or SEQ ID NO: 4, and wherein the first primer comprises the sequence defined by any one of SEQ ID NOs: 17 to 19 or 21 to 23, and/or, when present, the second primer comprises the sequence defined by SEQ ID NO: 14. In some embodiments, the frameshift mutation is in a microsatellite in the ASTE1 gene and comprises the sequence according to residues 328 to 337 of SEQ ID NO: 7 or SEQ ID NO: 8, and wherein the first primer comprises the sequence defined by any one of SEQ ID NOs: 34 to 38, and/or, when present, the second primer comprises the sequence defined by SEQ ID NO: 29. In some embodiments, the frameshift mutation is in a microsatellite in the ASTE1 gene and comprises the sequence according to residues 328 to 337 of SEQ ID NO: 7 or SEQ ID NO: 8, and wherein the first primer comprises the sequence defined by any one of SEQ ID NOs: 39, 41, 42, 84 to 88, 98, 99 and 101, preferably any one of SEQ ID NO: 84 to 88, 98, 99 and 101, and/or, when present, the second primer comprises the sequence defined by SEQ ID NO: 29. In some embodiments, the frameshift mutation is in a microsatellite in the TAF1β gene and comprises the sequence according to residues 255 to 264 of SEQ ID NO: 11 or SEQ ID NO: 12, and wherein the first primer comprises the sequence defined by any one of SEQ ID NOs: 53 to 59, and/or, when present, the second primer comprises the sequence defined by SEQ ID NO: 51. In some embodiments, the frameshift mutation is in a microsatellite in the ACVR2A gene and comprises the sequence according to residues 509 to 515 of SEQ ID NO: 96 or 97, and wherein the first primer comprises the sequence defined by SEQ ID NO: 90 or 91, and/or, when present, the second primer comprises the sequence defined by SEQ ID NO: 92.

In some embodiments, the microsatellite is in a TGFβR2 gene, the sequence comprising the wild type microsatellite comprises the sequence according to SEQ ID NO: 3, and the third primer comprises the sequence defined by SEQ ID NO: 13. In some embodiments, the microsatellite is in a ASTE1 gene, the sequence comprising the wild type microsatellite comprises the sequence according to SEQ ID NO: 7, and the third primer comprises the sequence defined by SEQ ID NO: 30 or SEQ ID NO: 31. In some embodiments, the microsatellite is in a TAF1β gene, the sequence containing the wild type microsatellite comprises the sequence defined in SEQ ID NO: 11, and the third primer comprises the sequence defined in SEQ ID NO: 50. In some embodiments, the microsatellite is in an ACVR2A gene, the sequence containing the wild type microsatellite comprises the sequence defined in SEQ ID NO: 96, and the third primer comprises the sequence defined in SEQ ID NO: 93. As described above, the data disclosed herein demonstrates that these primers are particularly useful within the scope of the invention for use in the detection of frameshift mutations in a microsatellite within TGFβR2 (also referred to as a9, where a10 is the wild type microsatellite), ASTE1 (also referred to as a10, where a11 is the wild type microsatellite), TAF1β (also referred to as a10, where a11 is the wild type microsatellite), or ACVR2A (also referred to as a7, where a8 is the wild type microsatellite).

In some embodiments, the sample is a liquid biopsy comprising cell free DNA, preferably wherein the liquid biopsy is plasma or serum. Alternatively, the sample is a tissue biopsy. Preferably, the sample is obtained from a human. Detection from liquid biopsies is particularly advantageous, as such biopsies are easily obtained from patients and are often easier to extract DNA from than e.g. a tumour tissue biopsy which may be necrotic and/or have variable DNA content making analysis more difficult. As previously mentioned, tumours are often heterogeneous and thus a tumour biopsy may not be representative of the whole tumour, and instead only the part that is sampled. Liquid biopsy overcomes this issue by providing a representative sample.

In some embodiments, the method further comprises determining that a patient suffering from a disease or disorder associated with a frameshift mutation is suitable for a treatment targeting said frameshift mutation if the frameshift mutation is detected in step d) in a sample from the patient. Preferably, the disease or disorder associated with a frameshift mutation is a cancer.

In some embodiments, the disease or disorder is colorectal cancer (CRC), gastric cancer (GC) or Lynch Syndrome, and the treatment targeting the frameshift mutation is FMPV-1 or FMPV-3, wherein the frameshift mutation is in a microsatellite in the TGFβR2 gene and comprises the sequence according to residues 270 to 278 of SEQ ID NO: 3 or SEQ ID NO: 4. In some embodiments, the disease or disorder is endometrial cancer or gastric cancer and the treatment targeting the frameshift mutation is FMPV-2 or FMPV-3, wherein the frameshift mutation is in a microsatellite in the ASTE1 gene and comprises the sequence according to residues 328 to 337 of SEQ ID NO: 7 or SEQ ID NO: 8. In some embodiments, the disease or disorder is endometrial cancer or gastric cancer and the treatment targeting the frameshift mutation is an immunogenic fragment of the TAF1β-1a frameshift mutant protein or FMPV-3, and the frameshift mutation is in a microsatellite in the TAF1β gene and comprises the sequence according to residues 255 to 264 of SEQ ID NO: 11 or SEQ ID NO: 12. In some embodiments, the disease or disorder is endometrial cancer or gastric cancer and the treatment targeting the one or more frameshift mutations is FMPV-3, and the frameshift mutation is in a microsatellite of one or more of the TGFβR2 gene and comprises the sequence according to residues 270 to 278 of SEQ ID NO: 3 or SEQ ID NO: 4, the ASTE1 gene and comprises the sequence according to residues 328 to 337 of SEQ ID NO: 7 or SEQ ID NO: 8, and the TAF1β gene and comprises the sequence according to residues 255 to 264 of SEQ ID NO: 11 or SEQ ID NO: 12. In some embodiments, the disease or disorder is stomach adenocarcinoma (STAD), uterine corpus endometrial cancer (UCEC) or colon adenocarcinoma (COAD).

Preferably, the method further comprises step e) of treating the patient with FMPV-1 or FMPV-2, an immunogenic fragment of the TAF1β-1a frameshift mutant protein or FMPV-3. FMPV-1 is a peptide vaccine as described in WO 2020/239937A1, which is incorporated herein by reference. FPMV-1 is also known as fsp2. FMPV-2 is a peptide vaccine as described in WO2021/239980, which is incorporated herein by reference. FMPV-2 is also known as fsp8. The immunogenic fragment of the TAF1β-1a frameshift mutant protein is a cancer vaccine as described in WO2021/239980. FMPV-3 is a cancer vaccine comprising a mixture of peptides targeting a frameshift mutation in one or more of TGFβR2, ASTE1 and TAF1β, as described in LU502776, which is incorporated herein by reference.

Optionally, a third primer, or another control primer pair, can also be used for DNA amplification from the sample in order to provide a positive control for the detection methods of the invention. In some embodiments, the microsatellite is in a TGFβR2 gene, the sequence comprising the wild type microsatellite comprises or consists of the sequence according to SEQ ID NO: 1 or SEQ ID NO: 3, and the third primer comprises the sequence defined by SEQ ID NO: 13. In some embodiments, the microsatellite is in a ASTE1 gene, the sequence comprising the wild type microsatellite comprises or consists of the sequence according to SEQ ID NO: 5 or SEQ ID NO: 7, and the third primer comprises the sequence defined by SEQ ID NO: 30 or SEQ ID NO: 31. In some embodiments, the microsatellite is in a TAF1β gene, the sequence comprising the wild type microsatellite comprises or consists of the sequence according to SEQ ID NO: 9 or SEQ ID NO: 11, and the third primer comprises the sequence defined by SEQ ID NO: 50. In some embodiments, the microsatellite is in an ACVR2A gene, the sequence comprising the wild type microsatellite comprises or consists of the sequence according to SEQ ID NO: 94 or SEQ ID NO: 96, and the third primer comprises the sequence defined by SEQ ID NO: 93.

In some embodiments, the first primer comprises the sequence defined by any one of SEQ ID NOs: 24 to 28, by any one of SEQ ID NOs: 45, 47, 83 and 100, by any one of SEQ ID NOs: 43, 44, 46, 48 and 49, by any one of SEQ ID NOs: 60 to 62, or by SEQ ID NO: 89, or the first primer comprises the sequence defined by any one of SEQ ID NOs: 17 to 19 or 21 to 23, by any one of SEQ ID NOs: 34 to 38 and 40, by any one of SEQ ID NOs: 39, 41, 42, 84 to 88, 98, 99 and 101, by any one of SEQ ID NOs: 53 to 59, or by SEQ ID NO: 90 or 91, and/or, when present, the second primer comprises the sequence defined by SEQ ID NO: 14, 29, 51 or 92. Preferably, the first primer comprises the sequence defined by any one of SEQ ID NO: 24, 45, 47, 48, 83, 61, 89 and 101, or by any one of SEQ ID NOs: 17, 37, 40, 41, 55, 56, 84 to 88, 90, 91, 98, 99 and 101 and/or, when present, the second primer comprises the sequence defined by SEQ ID NO: 14, 29, 51 or 92. More preferably, the first primer comprises the sequence defined by any one of SEQ ID NO: 45, 61, 83, 89 and 100, preferably by any one of SEQ ID NOs: 55, 56, 84 to 88, 90, 91, 98, 99 and 101, and/or, when present, the second primer comprises the sequence defined by any one of SEQ ID NO: 29, 51 and 92.

In another aspect of the invention, there is provided a method of diagnosing a disease associated with a frameshift mutation in a microsatellite, comprising carrying out any of the methods of the invention. As described above, frameshift mutations in microsatellites have been linked with a number of diseases, including but not limited to: Lynch Syndrome, cystic fibrosis, Crohn's disease, and cancer including colon, gastric, endometrium, ovarian, hepatobiliary tract, urinary tract, brain, skin cancers, stomach adenocarcinoma (STAD), uterine corpus endometrial cancer (UCEC) and colon adenocarcinoma (COAD). In particular cancer-associated frameshift mutations in microsatellites may be detected in any number of genes including but not limited to: ASTE1, ACVR22, TAF1β, KIAA2018, SLC22A9, TGFβR2 and ACVR2A.

In any of the above methods, it is preferable that the sample is a liquid biopsy comprising cell free DNA, preferably wherein the liquid biopsy is plasma.

In any of the above methods, it is preferable that the DNA amplification is PCR, and that the PCR is carried out in high stringency conditions, optionally wherein the high stringency conditions comprise at least one of:
a) carrying out the annealing step of PCR at a temperature that is at least 1° C., 2° C., 3° C., 4° C., 5° C., 6° C., 7° C., 8° C., 9° C. or 10° C. higher than the recommended annealing temperature of the reaction, preferably at a temperature between 53° C. and 60° C.;
b) carrying out the annealing step of PCR for only 30 seconds, preferably 15 seconds, per cycle;
c) carrying out the DNA amplification in a buffer concentration that is less than 0.1×, 0.2×, 0.3×, 0.4×, 0.5×, 0.6× or 0.75×;
d) carrying out the DNA amplification in a buffer comprising ammonium ions;
e) reducing the number of cycles of PCR to 25, 20, 15 or 10 cycles, optionally 30 or fewer, 25 or fewer, 20 or fewer, 15 or fewer, or 10 or fewer cycles;
f) carrying out the DNA amplification using a template DNA concentration of 0.2 ng or less, or 0.05 ng or less. The high stringency conditions may be any of those described above.

For example, in some embodiments, the high stringency conditions comprise carrying out the annealing step at a temperature that is at least 4° C. higher than the recommended annealing temperature, and/or carrying out the DNA amplification in a buffer comprising ammonium (NH₄⁺) ions, and/or carrying out the annealing step of PCR for 15 seconds per cycle, and/or reducing the number of cycles of PCR to 25 or fewer. In some embodiments, the high stringency conditions comprise carrying out the annealing step at a temperature that is at least 4° C. higher than the recommended annealing temperature, preferably at a temperature which is 4° C. higher than the recommended annealing temperature, carrying out the DNA amplification in buffer comprising ammonium (NH₄⁺) ions at 1× concentration, preferably wherein the buffer is Key buffer, carrying out the annealing step of PCR for 15 seconds, and reducing the number of cycles of PCR to 25.

In some embodiments, the high stringency conditions comprise carrying out the annealing step at a temperature that is at least 4° C. higher than the recommended annealing temperature, preferably at a temperature which is 4° C. higher than the recommended annealing temperature, carrying out the DNA amplification in buffer comprising ammonium (NH₄⁺) ions and/or potassium (K⁺) ions at 1× concentration, preferably wherein the buffer is ThermoPol® buffer, carrying out the annealing step of PCR for 15 seconds, and reducing the number of cycles of PCR to 25.

In some embodiments, the high stringency conditions comprise carrying out the annealing step at a temperature that is at least 4° C. higher than the recommended annealing temperature, preferably at a temperature which is 4° C. higher than the recommended annealing temperature, carrying out the DNA amplification in buffer comprising potassium (K⁺) ions at 1× concentration, preferably wherein the buffer is standard Taq (KCl) buffer, carrying out the annealing step of PCR for 15 seconds, and reducing the number of cycles of PCR to 25.

In some embodiments, the high stringency conditions comprise carrying out the annealing step at a temperature of 58° C. in a buffer including potassium (K⁺) ions, preferably standard Taq (KCl) buffer. In these embodiments, it is preferable that the frameshift mutation in the microsatellite is in TGFβR2.

In some embodiments, the high stringency conditions comprise carrying out the annealing step at a temperature of 52° C. to 56° C., 54 to 58° C., 42° C. to 52° C., or 50.3° C. to 52° C. in a buffer including ammonium (NH₄⁺) ions and/or potassium (K⁺) ions, preferably ThermoPol® buffer. In some embodiments, the high stringency conditions comprise carrying out the annealing step at a temperature of 54° C. to 55° C. in a buffer including potassium (K⁺) ions, preferably standard Taq (KCl) buffer, and most preferably, the first primer comprises the sequence defined by SEQ ID NO 39. Preferably, the final concentration of the template DNA in the reaction mix(es) is 0.05 ng/μL or 0.2 ng/μL. In these embodiments, it is preferable that the frameshift mutation in the microsatellite is in ASTE1.

In some embodiments, the high stringency conditions comprise carrying out the annealing step at a temperature of 54° C. in a buffer including potassium (K⁺) ions, preferably standard Taq (KCl) buffer. In these embodiments, it is preferable that the frameshift mutation in the microsatellite is in TAF1β.

In some embodiments, the high stringency conditions comprise carrying out the annealing step at a temperature of 56° C. in a buffer including ammonium (NH₄⁺) ions, preferably Key buffer. In these embodiments, it is preferable that the frameshift mutation in the microsatellite is in ACVR2A.

The invention will now be demonstrated in the below non-limiting experimental examples.

### Example I—Designing Primers for Detection of Frameshifted Mutated TGFβR2

Primer design typically follows particular rules to ensure a desirable yield of a single, specific amplicon (fragment). Most of the time these rules are easily taken in accordance-even when distinguishing between highly similar sequences. However, since the only difference, in the case of distinguishing a frameshift in a microsatellite, is the length of the microsatellite (one nucleotide difference) this is not applicable. The flanking regions of the satellite have the exact same sequence in both variants of DNA, making the affinity of a primer between them close to equal. In turn this is singlehandedly the largest obstacle to overcome in defining the detection test, since the primer design is locked to this exact sequence/area-without a possibility to change the parameters too much. The strategy used in designing mutant detection primers is therefore to create mismatches with the wild type sequence, and in that way induce enough repulsive forces and block primer annealing to the wild type DNA.

In a series of 14 experiments a panel of PCR primers (Table 1) for detection of frameshift mutated TGFβR2 was designed and tested. Initially a first set of primers were designed and tested for amplification of TGFβR2 DNA. Based on the results from the preceding experiment(s), the primers were optimized and re-tested. Synthetic TGFβR2 DNA fragments comprising the wild type and the mutant microsatellites, a10 and a9, respectively, were used for the PCR set up (SEQ ID NOs: 3 and 4 respectively).

TABLE 1

| Primer | SEQ ID NO: | 5′-3′ Direction | Sequence |
|---|---|---|---|
| P1 | 13 | Reverse | CAGTGTCATAGAGCACATTCCT |
| P2 | 14 | Forward | AGACAGTTTGCCATGACCC |
| P4 | 17 | Reverse | GTCTCACCAGGCTTTTTTTTTCG |
| P5 | 20 | Reverse | GTCTCACCAGGCTTTTTTTTTCC |
| P6 | 21 | Reverse | GTCTCACCAGGCTTTTTTTTACG |
| P10 | 22 | Reverse | CACCAGGCTTTTTTTATCGTTC |

### Example II—Testing Functionality of Primers

The first two primers (P1 & P2) act as the positive control regarding the detection test, to ensure reliable results and that all criteria to run the PCR are met (no signal from the control=unsuccessful PCR). In addition, the positive control ensures that the correct length and sequence of cfDNA (circulating free DNA) that is needed to perform the detection/determination of TGFβR2 variant is present. A P1-P2 derived amplicon (249 bp) encompasses all the other designed primer targets (corresponding sequences) and is not mutant specific.

The mutant primers (P4, P5, P6 and P10) were designed with mutant variant of TGFβR2 as primary target and harbours the 9A microsatellite sequence. Just by having a shorter sequence of 9A instead of 10A, there are mismatches between the wild type and the 3′-end of the primers. By introducing a single nucleotide substitution (randomly selected) on this end of primers P4, P6 and P10, the mismatch repulsion can be reinforced. Amplicons (102 bp-169 bp) are produced in conjugation with one of the control primers.

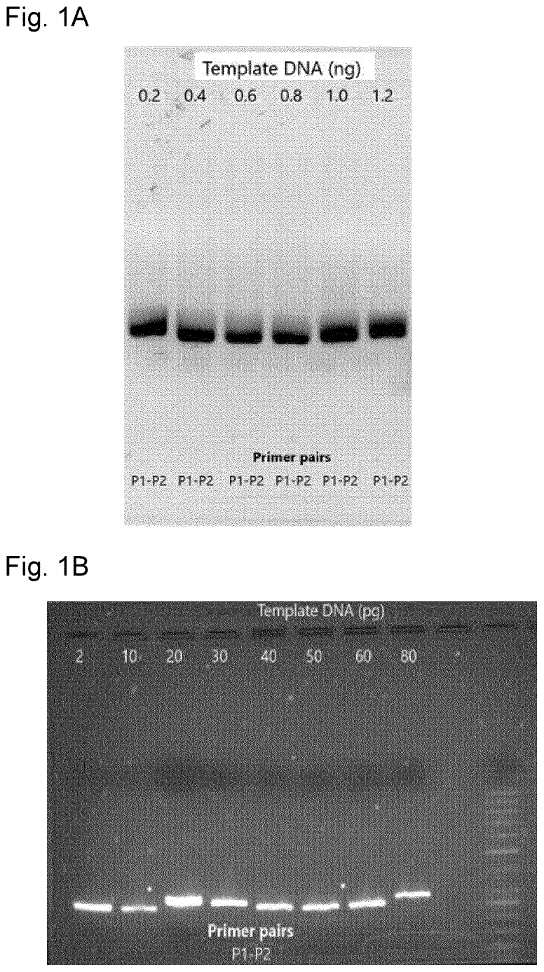

In order to determine the required amount of template DNA for PCR reactions, PCR test runs were conducted under standard PCR conditions, using protocols and calculated temperatures from NEB (New England BioLabs), using different amounts of template DNA. The results of these test runs can be seen in the electrophoresis gels shown in FIG. 1.

In particular, it was found that even a very small amount of template (less than 2 pg, as can be seen in FIG. 1B) achieves a good yield and high resolution bands in the gel. Furthermore, the amplicons have the correct expected length showing high specificity

It was found that all primers were functional, with successful amplification from the 9a microsatellite (mutant) and 10a microsatellite (wild type) template DNA under standard PCR conditions (see Table 2), pairing each primer with P2 (or with P1 in the case of P2). The product of these PCR reactions were stained with DNA staining and run on a pre-made agarose-TAE mixture (2%) by performing electrophoresis at 100V for approximately 20 to 30 minutes until the running front reached the end of the gel. The result of this electrophoresis demonstrated successful amplification from both template DNA types with strong bands visible in all lanes. However, the primers did not differentiate between the microsatellite containing the frameshift mutation (9a) (mutant) and the microsatellite not containing the frameshift mutation (10a) (wild type).

### Example III—Testing P4 in Suboptimal Conditions (High Stringency)

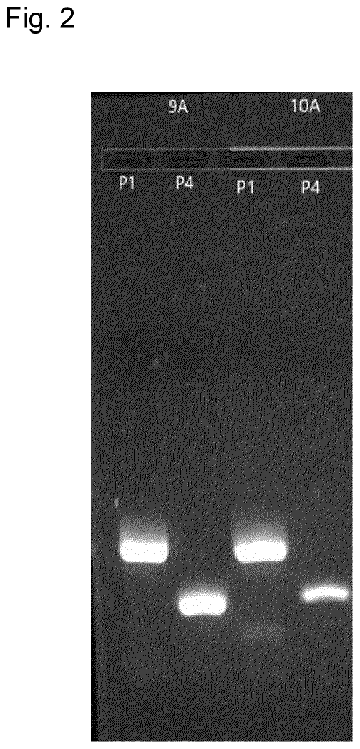

Therefore, this experimental approach was repeated using more stringent PCR conditions, using a slightly higher annealing temperature of 53° C., and fewer cycles of only 25 (see Table 2). The result of this condition change was that there was less amplification of the microsatellite not containing the frameshift mutation (10a) (wild type) which can clearly be seen in FIG. 2 by reduced band sizes for primer P4 where the signal is clearly diminishing in the 10a template.

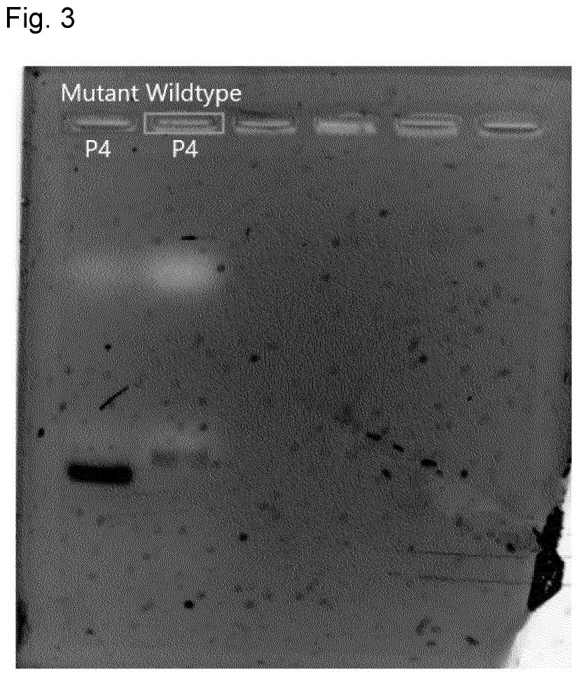

The experiment was then repeated using the P2 and P4 primers under even more stringent conditions than for the experiment shown in FIG. 2, increasing the annealing temperature further to 55° C. and shortening the annealing and extension steps by 15 seconds and 20 seconds respectively. These conditions are shown below in Table 2. These conditions provided even further improved distinction between the 10a template (wild type) and 9a template (mutant) with an extremely weak, almost invisible band for the wild type template, as can be seen in FIG. 3. The same was not found with P5, where no distinction between the 10a template (wild type) and 9a template (mutant) was found using this primer even in stringent conditions (data not shown).

TABLE 2

| | | |
|---|---|---|
| Step | Temperature | Time |

Standard stringency conditions

| | | | | |
|---|---|---|---|---|
| Initial denaturation | 95° | C. | 30 | sec. |
| Denaturation (30 cycles) | 95° | C. | 30 | sec. |
| Annealing (30 cycles) | 52° | C. | 45 | sec. |
| Extension (30 cycles) | 68° | C. | 30 | sec. |
| Final extension | 68° | C. | 5 | min. |

| | | | |
|---|---|---|---|
| Hold | 4° | C. | — |

High stringency conditions used in FIG. 2

| | | | | |
|---|---|---|---|---|
| Initial denaturation | 95° | C. | 30 | sec. |
| Denaturation (25 cycles) | 95° | C. | 30 | sec. |
| Annealing (25 cycles) | 53° | C. | 45 | sec. |
| Extension (25 cycles) | 68° | C. | 30 | sec. |
| Final extension | 68° | C. | 5 | min. |

| | | | |
|---|---|---|---|
| Hold | 4° | C. | — |

High stringency conditions used in FIG. 3

| | | | | |
|---|---|---|---|---|
| Initial denaturation | 95° | C. | 30 | sec. |
| Denaturation (25 cycles) | 95° | C. | 30 | sec. |
| Annealing (25 cycles) | 55° | C. | 35 | sec. |
| Extension (25 cycles) | 68° | C. | 10 | sec. |
| Final extension | 68° | C. | 5 | min. |

| | | | |
|---|---|---|---|
| Hold | 4° | C. | — |

### Example IV—Testing P4, P6 and P10 in Suboptimal Conditions (Higher Stringency)

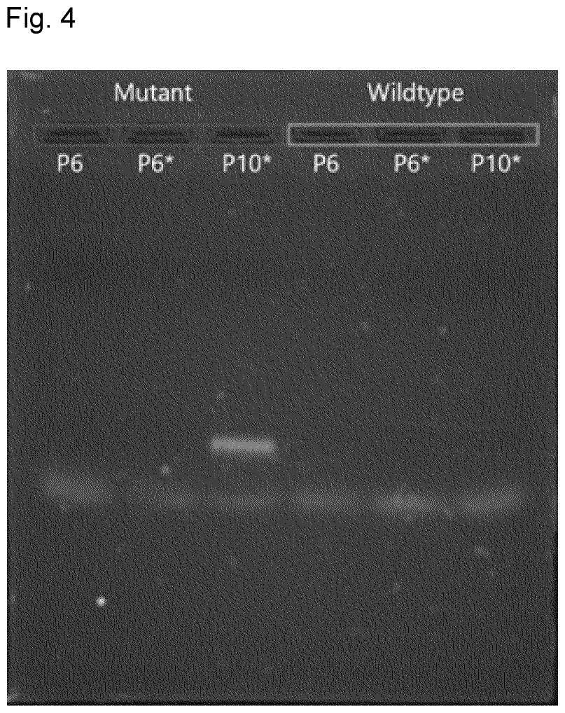

Again, in order to use the primers to distinguish between the mutant (9a) and wild type (10a) template DNA, more stringent PCR conditions were used as per Table 3, with a higher annealing temperature and a shorter annealing step used. In addition, the PCR reaction was carried out twice per primer pair, once at the standard buffer concentration, and once at a low buffer concentration (reduced to 50% of the standard concentration). Only primer pairs using P6 and P10 were examined in this particular experiment. As can be seen in FIG. 4, where low buffer concentration samples are marked with an asterisk (*), this resulted in a clear strong 10 band only in the sample with the low buffer concentration using the P10 primer in the mutant, and not in the wild type template.

TABLE 3

| | | |
|---|---|---|
| Step | Temperature | Time |

Standard stringency conditions

| | | | | |
|---|---|---|---|---|
| Initial denaturation | 95° | C. | 30 | sec. |
| Denaturation (25 cycles) | 95° | C. | 15 | sec. |
| Annealing (25 cycles) | 51° | C. | 30 | sec. |
| Extension (25 cycles) | 68° | C. | 10 | sec. |
| Final extension | 68° | C. | 5 | min. |

| | | | |
|---|---|---|---|
| Hold | 4° | C. | — |

High stringency conditions used in FIG. 4

| | | | | |
|---|---|---|---|---|
| Initial denaturation | 95° | C. | 30 | sec. |
| Denaturation (25 cycles) | 95° | C. | 15 | sec. |
| Annealing (25 cycles) | 56° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

In order to optimise the amplification by the designed primers from the mutant template, further experiments were carried out using P4, P6 and P10 (as well as P1 as a positive control) using the mutant template DNA (9a) at a number of different conditions.

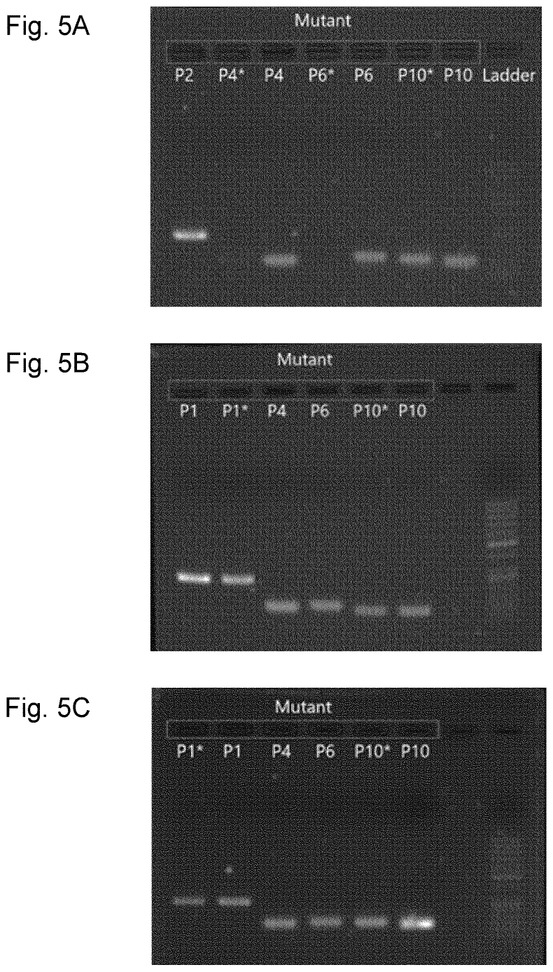

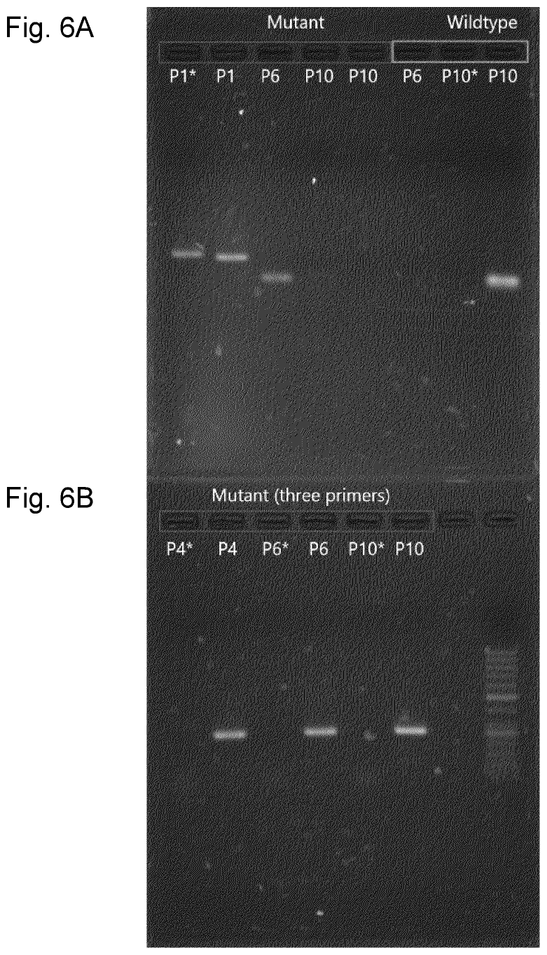

In particular, in the experiments shown in FIGS. 5A-C used annealing temperatures of 55° C., 56° C., and 58° C. respectively as can be seen below in Table 4. Again, a “low” buffer concentration (denoted by an asterisk, *) in the experiments shown in FIGS. 5A-6C comprised a buffer concentration of around 50% of the standard buffer concentration. As can be seen, stronger signals were seen in FIGS. 5B and 5C, particularly at the low buffer concentration. Of note, the P6 primer showed amplification here, confirming the hypothesis that the lack of amplification in the previous experiments was due to human error.

TABLE 4

| | | |
|---|---|---|
| Step | Temperature | Time |

PCR conditions used in FIG. 5A

| | | | | |
|---|---|---|---|---|
| Initial denaturation | 95° | C. | 2 | min. |
| Denaturation (30 cycles) | 95° | C. | 15 | sec. |
| Annealing (30 cycles) | 55° | C. | 15 | sec. |
| Extension (30 cycles) | 68° | C. | 45 | sec. |
| Final extension | 68° | C. | 5 | min. |

| | | | |
|---|---|---|---|
| Hold | 4° | C. | — |

PCR conditions used in FIG. 5B

| | | | | |
|---|---|---|---|---|
| Initial denaturation | 95° | C. | 2 | min. |
| Denaturation (25 cycles) | 95° | C. | 15 | sec. |
| Annealing (25 cycles) | 56° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

PCR conditions used in FIG. 5C

| | | | | |
|---|---|---|---|---|
| Initial denaturation | 95° | C. | 2 | min. |
| Denaturation (25 cycles) | 95° | C. | 15 | sec. |
| Annealing (25 cycles) | 58° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

Further PCR reactions were carried out using primer pairs at an even higher annealing temperature of 60° C. (see Table 5 below) at both standard and low buffer concentrations (FIG. 6). A strong signal was seen in the control (P1) at both the standard and low buffer concentrations.

This higher annealing temperature allowed for the selective amplification of mutant template by primer P6 in combination with primer P2, with no detectable amplification of wild type template, which can be seen in FIG. 6A.

In addition, three primer reactions were also carried out in the same conditions, which can be seen in FIG. 6B. Three primer reactions contained P1, P2 and either P4, P6 or P10, and it was expected that two bands would be seen at 249 bp and 102 bp. No signal was seen in the low buffer concentration reactions, and only a single band was seen in the standard buffer concentration reactions.

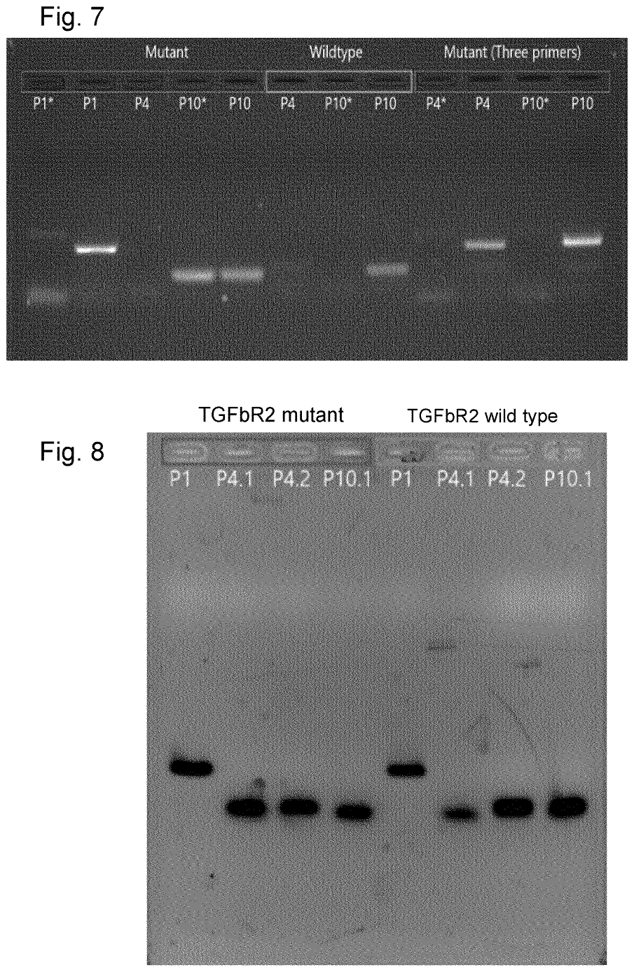

The same conditions were used again (as per Table 5) to perform further PCR reactions, but also including primer P4 (FIG. 7). In these experiments, it was found that a strong band from the positive control (P1) was seen in the standard buffer concentration, and a weaker band was seen in the low buffer concentration. At the standard buffer concentration, amplification with P10 was seen in both the mutant and wild type templates, however in the low buffer concentration, it was seen that amplification with P10 was seen from the mutant template but not the wild type template (FIG. 7). Thus, it was found that P10 in the low buffer concentration had no affinity towards the wild type sequence.

Again, three primer reactions were carried out in the same conditions, and in this experiment, whilst only one weak band was detected at the low buffer concentration, two bands (one strong and one weak) were seen in the standard buffer concentration.

TABLE 5
PCR conditions used in FIG. 6A, 6B and 7

| | | |
|---|---|---|
| Step | Temperature | Time |

| | | | | |
|---|---|---|---|---|
| Initial denaturation | 95° | C. | 2 | min. |
| Denaturation (25 cycles) | 95° | C. | 15 | sec. |
| Annealing (25 cycles) | 60° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

### Example V—Testing Functionality of Primers P4.1, P4.2 and P10.1

As per the above example, further primers P4.1, P4.2 and P10.1, which were further modified from primers P4 and P10 were also tested by pairing with primer P2 and carrying out PCR (conditions are detailed below in Table 6) followed by gel electrophoresis (shown in FIG. 8). This experiment showed that all three of these primers are functional as they showed amplification against both the mutant (9a) and wild type (10a) templates, as can be seen in FIG. 8, which shows strong banding. A significantly reduced band can be seen for P4.1 in the mutant template relative to the equivalent band in the wild type template.

TABLE 6
PCR conditions used in FIG. 8

| | | |
|---|---|---|
| Step | Temperature | Time |

| | | | | |
|---|---|---|---|---|
| Initial denaturation | 95° | C. | 2 | min. |
| Denaturation (25 cycles) | 95° | C. | 15 | sec. |
| Annealing (25 cycles) | 52° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

### Example VI—Testing Candidate Primers in Higher Stringency PCR Conditions

To use the primers to distinguish between the mutant (9a) and wild type (10a) template, PCR reactions were conducted using two different types of buffer: standard Taq buffer which does not comprise ammonium ions, and Key buffer which comprises ammonium ions (NH₄⁺).

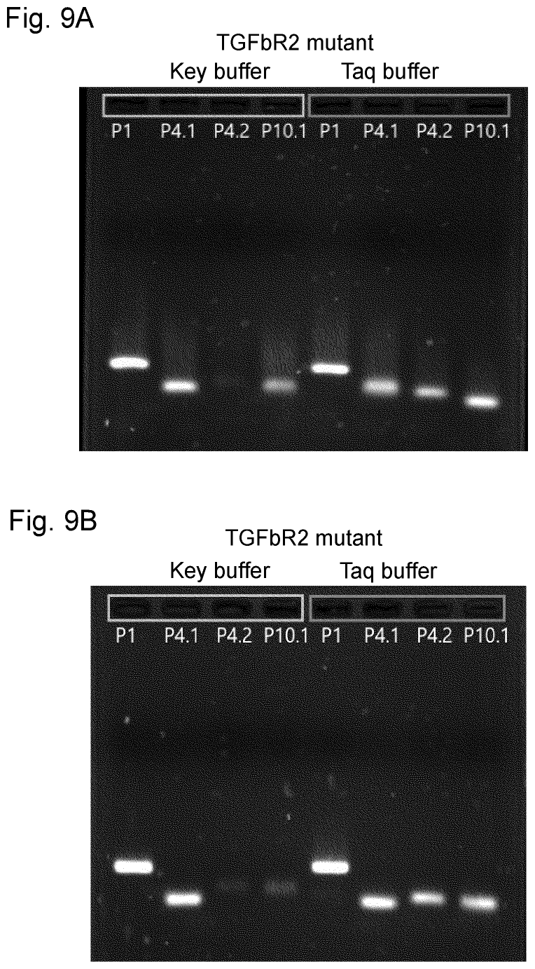

Firstly, PCR reactions on mutant template using Taq buffer or Key buffer were used in the conditions shown in Table 7 and run on an electrophoresis gel as shown in FIG. 9A. It was found that relatively uniform signal strength was seen regardless of which buffer was used, with the exception of primer P4.2 where the reaction in Key buffer showed minimal signal.

These reactions were then repeated with a slightly higher annealing temperature (again shown in Table 7), and run on an electrophoresis gel as shown in FIG. 9B. This experiment showed comparable results as those seen in FIG. 9A, however the signal produced by the reaction with primer P10.1 in Key buffer was significantly reduced. The signal produced by the reaction with primer P4.2 was more distinguishable in this experiment.

The immediate difference between signals produced by some PCR samples containing key buffer and Taq-buffer suggests that stabilization of primer/template interaction is highly affected. Having a higher number of mismatches between designed primers and wildtype DNA could lead to elimination of amplicons produced from these samples using key buffer. From the results it is apparent that several mutant template DNA and primer samples have equal signal strength regardless of buffer variant, and it is anticipated that the interaction remains in these samples and amplicons are produced whereas wild type template have decreased primer annealing. Control primers appear unaffected by the buffer variants and temperatures tested. Since the samples with P4.2 and key buffer were not consistent through the two experiments is indicative that the P4.2 stock were of poor condition.

TABLE 7

| | | |
|---|---|---|
| Step | Temperature | Time |

PCR conditions used in FIG. 9A

| | | | | |
|---|---|---|---|---|
| Initial denaturation | 95° | C. | 2 | min. |
| Denaturation (25 cycles) | 95° | C. | 15 | sec. |
| Annealing (25 cycles) | 56° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

PCR conditions used in FIG. 9B

| | | | | |
|---|---|---|---|---|
| Initial denaturation | 95° | C. | 2 | min. |
| Denaturation (25 cycles) | 95° | C. | 15 | sec. |
| Annealing (25 cycles) | 58° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

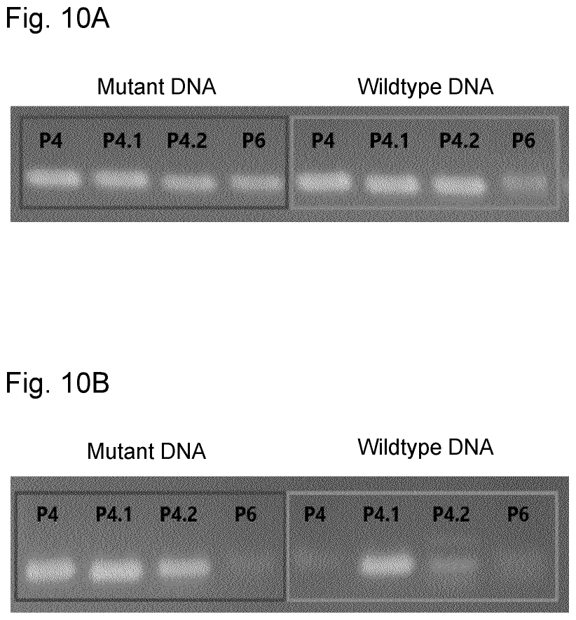

Further experiments were carried out in the same conditions as FIG. 9A shown in Table 7, using primers P4, P4.1, P4.2 and P6 in combination with P2 on both mutant and wild type template DNA. These reactions were also run on electrophoresis gels as can be seen in FIG. 10. Reactions in FIG. 10A were carried out in standard Taq buffer, and reactions in FIG. 10B were carried out in Key buffer, which contains ammonium ions (NH₄⁺).

In samples containing standard Taq-buffer there were sufficient amplicon produce to give strong bands with the various primers and templates (FIG. 10A). Samples containing the Key buffer showed lack of primer annealing and thus product yield as visualized by weak/disappearing bands in some primer/template combinations (FIG. 10B). With mutant template DNA, P6 had heavily reduced functionality. Turning to the wild type template DNA, there were loss of function in P4, P6, and fractioned/reduced performance in P4.2.

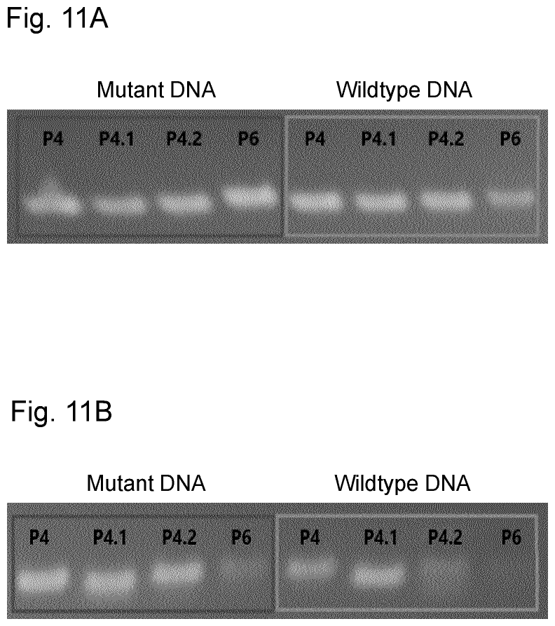

The experiments were then repeated at a slightly higher annealing temperature of 59° C., and run on electrophoresis gels, which can be seen in FIG. 11, with reactions in standard Taq buffer shown in FIG. 11A and reactions in Key buffer shown in FIG. 11B. As seen in the previous experiment, all samples yielded strong signals on the gel regardless of template DNA. Looking at samples with mutated DNA as template and Key buffer, the only primer which did not produce amplicon (or produced a low amount) in this combination is P6. With wild type DNA template and key buffer, weak/disappearing bands were noticed in samples with P4.2 and P6.

Looking at the results from FIGS. 10 and 11, it is evident that key buffer (ammonium, NH₄⁺) has a significant destabilizing effect that prevents the annealing of (selected) primers to template DNA. In addition, slight alterations in temperature plays a significant role in loss- or gain of function (in annealing). Since the designed primers will inherently have more mismatches in combination with wild type sequence as template DNA in comparison to mutant sequence, the influence of Key buffer is significantly higher. Primer/template annealing remain functional with mutant TGFβR2 sequence at a wider area of parameters.

### Example VII—Detection of TGFβR2 a10→a9 Frameshift Mutation in Cell Free DNA (cfDNA) of Liquid Biopsies from MSI-Colorectal Cancer Patients

### Methods

cfDNA was extracted from patient plasma samples using a cfDNA extraction kit (Plasma/Serum Cell-Free Circulating DNA Purification Mini Kit, category number 55100, www.norgenbiotek.com). The patient samples were bought from Indivumed GmbH (www.indivumed.com) and were all taken at the same point in treatment of the individual patient—T0 (baseline). Patients had colorectal cancer (CRC), were MSI-H or MSS, and were aged 42-73.

i) Sequencing of cfDNA

Sequencing of cfDNA fragments present in the plasma samples was done by performing PCR with a high-fidelity polymerase (OneTaq DNA Polymerase, New England Biolabs, www.international.neb.com) and standard conditions, using 1× OneTaq-buffer (KCl). The high-fidelity polymerase had 3′-5′ exonuclease activity and ensured that the sequence of the microsatellite, where polymerases are prone to “slipping” due to the high number of single-nucleotide repeats, was correctly synthesized. Synthetic TGFβR2 template DNA was used as controls (wild-type (same sequence as SEQ ID NO: 3) and 9a mutant variant (same sequence as SEQ ID NO: 4)), in order to verify that the sequencing data of the microsatellite is correct and that the read-out of the electrophoresis gel is consistent compared to known sequences. Primers used in PCR for sequencing purposes were P1 and P2.

The PCR conditions were as set out in Table 8.

TABLE 8
Thermocycling conditions (OneTag)

| | | |
|---|---|---|
| Step | Temperature | Time |

| | | | | |
|---|---|---|---|---|
| Initial denaturation | 94° | C. | 2 | min. |
| Denaturation (30 cycles) | 94° | C. | 15 | sec. |
| Annealing (30 cycles) | 52° | C. | 15 | sec. |
| Extension (30 cycles) | 68° | C. | 20 | sec. |
| Final extension | 68° | C. | 5 | min. |

| | | | |
|---|---|---|---|
| Hold | 4° | C. | — |

The PCR product was purified using gel electrophoresis. The band with fragments lamplicons with the correct length was cut from the gel and extracted using a gel-extraction kit (VWR peqGOLD Gel Extraction Kit, category number 13-2500-01, www.vwr.com). Extracted PCR product was then sent for sequencing, which was performed by Eurofins Genomics (www.eurofinsgenomics.eu).

ii) Detection of Mutated TGFβR2 cfDNA

cfDNA from the patient plasma samples, and synthetic wild-type and a9 mutant TGFβR2 (SEQ ID NOS: 3 and 4) as controls, was amplified by PCR, using the conditions in Table 10. Primers P1 and P2 were used for positive controls, and primers P2 and P4 were used for detection of mutated TGFβR2, as shown in Table 9. In Table 9, the “Primer” column indicates only whether P1 or P4 was used, as primer P2 was used in all tubes. In the “Sample” column”, “synth” indicates that synthetic DNA was used as the template (i.e. the control samples), rather than cfDNA obtained from the patient plasma samples. The buffer used was 1× Key buffer ((NH₄)₂SO₄). The PCR products were subjected to gel electrophoresis, and then sequenced.

TABLE 9

| Tube | Primer | DNA template | Buffer | Sample |
|---|---|---|---|---|
| 1 | P1 | MSI BD_001 | Key | T0 |
| 2 | P4 | MSI BD_001 | Key | T0 |
| 3 | P1 | MSI BD_002 | Key | T0 |
| 4 | P4 | MSI BD_002 | Key | T0 |
| 5 | P1 | MSI BD_003 | Key | T0 |
| 6 | P4 | MSI BD_003 | Key | T0 |
| 7 | P1 | MSI BD_004 | Key | T0 |
| 8 | P4 | MSI BD_004 | Key | T0 |
| 9 | P1 | MSI BD_005 | Key | T0 |
| 10 | P4 | MSI BD_005 | Key | T0 |
| 11 | P1 | MSS BD_006 | Key | T0 |
| 12 | P4 | MSS BD_006 | Key | T0 |
| 13 | P1 | MSS BD_007 | Key | T0 |
| 14 | P4 | MSS BD_007 | Key | T0 |
| 15 | P1 | MSS BD_008 | Key | T0 |
| 16 | P4 | MSS BD_008 | Key | T0 |
| 17 | P1 | MSS BD_009 | Key | T0 |
| 18 | P4 | MSS BD_009 | Key | T0 |
| 19 | P1 | MSS BD_010 | Key | T0 |
| 20 | P4 | MSS BD_010 | Key | T0 |
| 21 | P1 | F1, mutant | Key | Synth. |
| 22 | P1 | F2, wildtype | Key | Synth. |
| 23 | P4 | F1, mutant | Key | Synth. |
| 24 | P4 | F2, wildtype | Key | Synth. |

TABLE 10
Thermocycling conditions

| | | |
|---|---|---|
| Step | Temperature | Time |

| | | | | |
|---|---|---|---|---|
| Initial denaturation | 95° | C. | 30 | sec. |
| Denaturation (25 cycles) | 95° | C. | 15 | sec. |
| Annealing (25 cycles) | 56° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

### Results

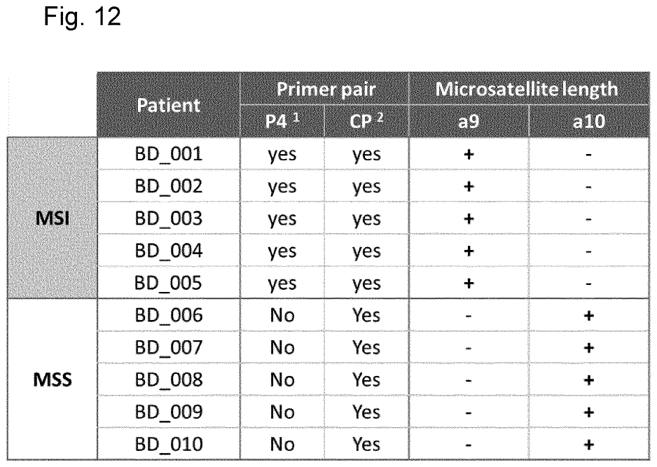

TGFβR2 DNA was present in cfDNA from all (10/10) CRC patients tested. PCR amplification using the primers, followed by gel electrophoresis, showed that a TGFβR2 a10→a9 frameshift was present in all (5/5) MSI-CRC patients and none (0/5) of the MSS-CRC patients tested (FIG. 12). These results were confirmed by sequencing of the PCR products of correct size extracted from the electrophoresis gel. The sequenced PCR products contained the entire a9/a10 microsatellite, as relevant, and the sequencing data was shown to correspond to the PCR results.

PCR using the selected a9 modelled primer (P4) yielded a PCR product that was clearly detectable on the electrophoresis gel only for cfDNA from the MSI-CRC patients (5/5). The TGFβR2 cfDNA fragments were long enough to contain the microsatellite and the regions that the control primer pair anneals to (250 bp).

The results show that TGFβR2 a10→a9 frameshift DNA is present in cfDNA of liquid biopsies from MSI-CRC patients, which establishes cell free TFGbR2 frameshift DNA as a potential biomarker for early detection of hereditary CRC (Lynch Syndrome) as well as for monitoring cancer progression and remission of sporadic MSI-CRC.

The sequences of the cfDNA from each patient, extracted from the electrophoresis gels, are shown in Table 11, with the length of the microsatellite shown in parentheses at the end of each sequence.

TABLE 11

| | SEQ ID | |
|---|---|---|
| Patient | NO: | Sequence |
| BD_001 | 73 | TTGGCACAGATCTCAGGTCCCACACCCTTAAGAGAAGAAAA |
| (MSI-H) | | CTCACCTTCTGAGAAGATGATGTTGTCATTGCACTCATCAG |
| | | AGCTACAGGAACACATGAAGAAAGTCTCACCAGGCTTTTTT |
| | | TTTCCTTCATAATGCACTTTGGAAAAGCAGCATCTTCCAAAA |
| | | TAAAGTCATGGTAGGGGAGCTTGGGGTCATGGCAAACTG |
| | | (A9) |
| BD_002 | 74 | TGGCACAGATCTCAGGTCCCACACCCTTAAGAGAAGAAAA |
| (MSI-H) | | CTCACCTTCTGAGAAGATGATGTTGTCATTGCACTCATCAG |
| | | AGCTACAGGAACACATGAAGAAAGTCTCACCAGGCTTTTTT |
| | | TTTCCTTCATAATGCACTTTGGAGAACCAGCATCTTCCAAAA |
| | | TAAAGTCATGGAAGGGGAGCTTGGGGTCAGGGCAAACTG |
| | | (A9) |
| BD_003 | 75 | CCGGAAAAAATTGGCACAGATCTCAGGTCCCACACCCTTAA |
| (MSI-H) | | GAGAAGAAAACTCACCTTCTGAGAAGATGATGTTGTCATTG |
| | | CACTCATCAGAGCTACAGGAACACATGAAGAAAGTCTCACC |
| | | AGGCTTTTTTTTTCCTTCATAATGCACTTTGGAGAAGCAGCA |
| | | TCTTCCAGAATAAAGTCATGGTAGGGGAGCTTGGGGTCAT |
| | | GGCAAACTGTCTAA (A9) |
| BD_004 | 76 | ACAGATCTCAGGTCCCACACCCTTAAGAGAAGAAAACTCAC |
| (MSI-H) | | CTTCTGAGAAGATGATGTTGTCATTGCACTCATCAGAGCTA |
| | | CAGGAACACATGAAGAAAGTCTCACCAGGCTTTTTTTTTCC |
| | | TTCATAATGCACTTTGGAAAAGCAGCATCTTCCAAAATAAAG |
| | | TCATGGTAGGGGAGCTTGGGGTCATGGCA (A9) |
| BD_005 | 77 | TGGCACAGATCTCAGGTCCCACACCCTTAAGAGAAGAAAA |
| (MSI-H) | | CTCACCTTCTGAGAAGATGATGTTGTCATTGCACTCATCAG |
| | | AGCTACAGGAACACATGAAGAAAGTCTCACCAGGCTTTTTT |
| | | TTTCCTTCATAATGCACTTTGGAGAAGCA (A9) |
| BD_006 | 78 | ATTGGCACAGATCTCAGGTCCCACACCCTTAAGAGAAGAAA |
| (MSS) | | ACTCACCTTCTGAGAAGATGATGTTGTCATTGCACTCATCA |
| | | GAGCTACAGGAACACATGAAGAAAGTCTCACCAGGCTTTTT |
| | | TTTTTCCTTCAA (A10) |
| BD_007 | 79 | TGGCACAGATCTCAGGTCCCACACCCTTAAGAGAAGAAAA |
| (MSS) | | CTCACCTTCTGAGAAGATGATGTTGTCATTGCACTCATCAG |
| | | AGCTACAGGAACACATGAAGAAAGTCTCACCAGGCTTTTTT |
| | | TTTTCCTTCATAAG (A10) |
| BD_008 | 80 | ACAGATCTCAGGTCCCACACCCTTAAGAGAAGAAAACTCAC |
| (MSS) | | CTTCTGAGAAGATGATGTTGTCATTGCACTCATCAGAGCTA |
| | | CAGGAACACATGAAGAAAGTCTCACCAGGCTTTTTTTTTTC |
| | | CTTCTTA (A10) |
| BD_009 | 81 | TGGCACAGATCTCAGGTCCCACACCCTTAAGAGAAGAAAA |
| (MSS) | | CTCACCTTCTGAGAAGATGATGTTGTCATTGCACTCATCAG |
| | | AGCTACAGGAACACATGAAGAAAGTCTCACCAGGCTTTTTT |
| | | TTTTCCTTCATAA (A10) |
| BD_010 | 82 | AGATCTCAGGTCCCACACCCTTAAGAGAAGAAAACTCACCT |
| (MSS) | | TCTGAGAAGATGATGTTGTCATTGCACTCATCAGAGCTACA |
| | | GGAACACATGAAGAAAGTCTCACCAGGCTTTTTTTTTTCCTT |
| | | CATAA (A10) |

### Example VIII—Detection of TGFβR2 a10→a9 Frameshift Mutation at Different Annealing Temperatures

### i) Temperature Gradient PCR 1 (Parameter Adjustment)

This experiment was for the purpose of establishing a range of temperatures at which the primer pair is effective in annealing only to the mutant DNA template, and not the wild-type DNA template.

Synthetic DNA (mutant (F1) and wildtype (F2)) was used as the template for PCR, with primers P2 and P4. The PCR conditions are shown in Tables 12-14, and the PCR tube layout and annealing temperature used for each tube is set out in Table 15. The annealing temperature ranged from 54° C. to 60° C.

TABLE 12
Tubes 1-8 PCR master mix

| PCR master mix | 50 μl reaction | |
|---|---|---|
| # of samples | 8 | |
| 10x Key buffer, μL | 40 | |
| 10 mM dNTPs, μL | 8 | |
| FW primer, μL | 8 | P2 |
| REV primer, μL | 8 | P4 |
| Template DNA, μL | 8 | F1, 0.2 ng/μL |
| Taq-polymerase, μL | 2 | |
| ddH2O, μL | 326 | |
| Total, μL | 400 | |
| WO Primer, μL | 400 | |
| Aliquot each tube, μL | 50 | |

TABLE 13
Tubes 9-16 PCR master mix

| PCR master mix | 50 μl reaction | |
|---|---|---|
| # of samples | 8 | |
| 10x Key buffer, μL | 40 | |
| 10 mM dNTPs, μL | 8 | |
| FW primer, μL | 8 | P2 |
| REV primer, μL | 8 | P1 |
| Template DNA, μL | 8 | F2, 0.2 ng/μL |
| Taq-polymerase, μL | 2 | |
| ddH2O, μL | 326 | |
| Total, μL | 400 | |
| WO Primer, μL | 400 | |
| Aliquot each tube, μL | 50 | |

TABLE 14
Thermocycling conditions
Thermocycling conditions

| | | |
|---|---|---|
| Step | Temperature | Time |

| | | | | |
|---|---|---|---|---|
| Initial denaturation | 95° | C. | 2 | min. |
| Denaturation (30 cycles) | 95° | C. | 15 | sec. |
| Annealing (30 cycles) | 54-60° | C. | 15 | sec. |
| Extension (30 cycles) | 68° | C. | 45 | sec. |
| Final extension | 68° | C. | 5 | min. |

| | | | |
|---|---|---|---|
| Hold | 4° | C. | — |

TABLE 15
PCR tube layout and annealing temperature

| Tube | Primer | DNA fragment | Buffer | Length | Temp | |
|---|---|---|---|---|---|---|
| 1 | P4 | F1, mutant | Key | 102bp | 60° | C. |
| 2 | P4 | F1, mutant | Key | 102bp | 59.7° | C. |
| 3 | P4 | F1, mutant | Key | 102bp | 59° | C. |
| 4 | P4 | F1, mutant | Key | 102bp | 57.8° | C. |
| 5 | P4 | F1, mutant | Key | 102bp | 56.4° | C. |
| 6 | P4 | F1, mutant | Key | 102bp | 55.2° | C. |
| 7 | P4 | F1, mutant | Key | 102bp | 54.4° | C. |
| 8 | P4 | F1, mutant | Key | 102bp | 54° | C. |
| 9 | P4 | F2, wildtype | Key | 102bp | 60° | C. |
| 10 | P4 | F2, wildtype | Key | 102bp | 59.7° | C. |
| 11 | P4 | F2, wildtype | Key | 102bp | 59° | C. |
| 12 | P4 | F2, wildtype | Key | 102bp | 57.8° | C. |
| 13 | P4 | F2, wildtype | Key | 102bp | 56.4° | C. |
| 14 | P4 | F2, wildtype | Key | 102bp | 55.2° | C. |
| 15 | P4 | F2, wildtype | Key | 102bp | 54.4° | C. |
| 16 | P4 | F2, wildtype | Key | 102bp | 54° | C. |

Key Buffer

### Key Buffer

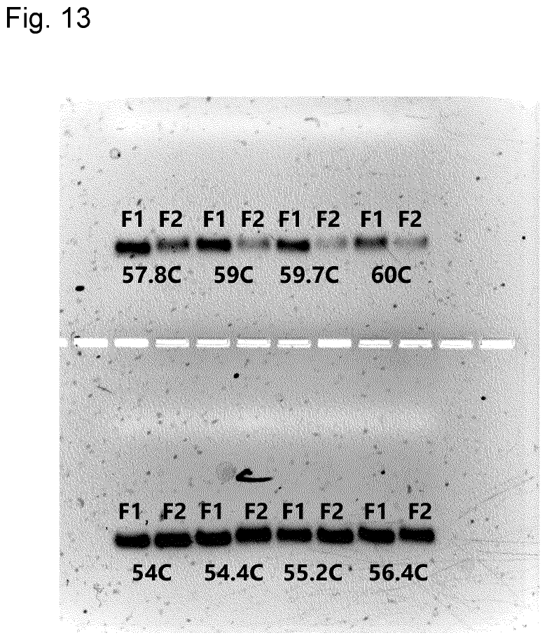

FIG. 13 shows the electrophoresis gels of the PCR products. It is clear that the primer-wildtype template interaction is most weakened at temperatures ranging from approximately 57.8° C. and upwards in comparison with primer-mutant template. In addition, at approximately 59° C., the primer-wildtype template interaction is more or less completely disrupted. Primer-mutant template interaction remains strong, and the primer still has high affinity even at high temperatures.

### ii) Temperature Gradient PCR 2 (Parameter Adjustment)

This experiment was carried out in order to further establish a range of temperatures where the primer pair is effective in annealing only to the mutant DNA template.

As with part i) above, synthetic DNA (mutant (F1; SEQ ID NO: 4) and wildtype (F2; SEQ ID NO: 3)) was used as the template for PCR, with primers P2 and P4. The PCR conditions are shown in Tables 16-18, and the PCR tube layout and annealing temperature used for each tube is set out in Table 19. The annealing temperature ranged from 57° C. to 62° C.

TABLE 16
Tues 1-8 PCR master mix

| PCR master mix | 50 μl reaction | |
|---|---|---|
| # of samples | 8 | |
| 10x Key buffer, μL | 40 | |
| 10 mM dNTPs, μL | 8 | |
| FW primer, μL | 8 | P2 |
| REV primer, μL | 8 | P4 |
| Template DNA, μL | 8 | F1, 0.2 ng/μL |
| Taq-polymerase, μL | 2 | |
| ddH2O, μL | 326 | |
| Total, μL | 400 | |
| WO Primer, μL | 400 | |
| Aliquot each tube, μL | 50 | |

TABLE 17
Tubes 1-9 PCR master mix

| PCR master mix | 50 μl reaction | |
|---|---|---|
| # of samples | 8 | |
| 10x Key buffer, μL | 40 | |
| 10 mM dNTPs, μL | 8 | |
| FW primer, μL | 8 | P2 |
| REV primer, μL | 8 | P1 |
| Template DNA, μL | 8 | F2, 0.2 ng/μL |
| Taq-polymerase, μL | 2 | |
| ddH2O, μL | 326 | |
| Total, μL | 400 | |
| WO Primer, μL | 400 | |
| Aliquot each tube, μL | 50 | |

TABLE 18
Thermocycling conditions
Thermocycling conditions

| | | |
|---|---|---|
| Step | Temperature | Time |

| | | | | |
|---|---|---|---|---|
| Initial denaturation | 95° | C. | 2 | min. |
| Denaturation (30 cycles) | 95° | C. | 15 | sec. |
| Annealing (30 cycles) | 57-62° | C. | 15 | sec. |
| Extension (30 cycles) | 68° | C. | 45 | sec. |
| Final extension | 68° | C. | 5 | min. |

| | | | |
|---|---|---|---|
| Hold | 4° | C. | — |

TABLE 19
PCR tube layout and annealing temperature

| Tube | Primer | DNA fragment | Buffer | Length | Temp | |
|---|---|---|---|---|---|---|
| 1 | P4 | F1, mutant | Key | 102bp | 62° | C. |
| 2 | P4 | F1, mutant | Key | 102bp | 61.7° | C. |
| 3 | P4 | F1, mutant | Key | 102bp | 61.2° | C. |
| 4 | P4 | F1, mutant | Key | 102bp | 60.2° | C. |
| 5 | P4 | F1, mutant | Key | 102bp | 59° | C. |
| 6 | P4 | F1, mutant | Key | 102bp | 58° | C. |
| 7 | P4 | F1, mutant | Key | 102bp | 57.3° | C. |
| 8 | P4 | F1, mutant | Key | 102bp | 57° | C. |
| 9 | P4 | F2, wildtype | Key | 102bp | 62° | C. |
| 10 | P4 | F2, wildtype | Key | 102bp | 61.7° | C. |
| 11 | P4 | F2, wildtype | Key | 102bp | 61.2° | C. |
| 12 | P4 | F2, wildtype | Key | 102bp | 60.2° | C. |
| 13 | P4 | F2, wildtype | Key | 102bp | 59° | C. |
| 14 | P4 | F2, wildtype | Key | 102bp | 58° | C. |
| 15 | P4 | F2, wildtype | Key | 102bp | 57.3° | C. |
| 16 | P4 | F2, wildtype | Key | 102bp | 57° | C. |

Key Buffer

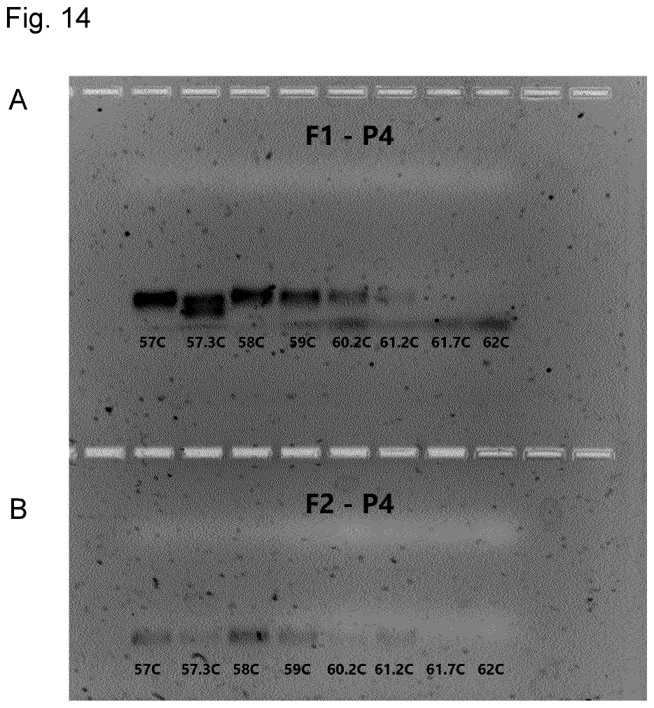

FIG. 14 shows the electrophoresis gels of the PCR products, and shows that even at temperatures lower than 57.8° C. mentioned in part i) above, the primer-wildtype template interaction is disrupted and the affinity of the primer is close to non-existent. In particular, FIG. 14 shows that the primer-wildtype template interaction is disrupted at 57° C. However, the primer affinity towards the mutant template remains strong in comparison under the same conditions.

### Example IX—Detection of TGFβR2 a10→a9 Frameshift Mutation Using Standard Taq (KCl) Buffer

This experiment was conducted to assess the efficacy of the primers in PCR mixes comprising standard Taq buffer (KCl). Synthetic DNA (mutant (F1) and wildtype (F2)) was used as the template for PCR. Primers P1 and P4 were each used with primer P2. Tables 20 and 21 set out the PCR conditions, and the primers and buffer used for each tube is set out in Table 22.

TABLE 20
PCR conditions for standard Taq buffer

| PCR master mix (Taq) | 50 μl reaction | |
|---|---|---|
| # of samples | 4 | |
| Taq buffer 10×, μL | 20 | |
| 10 mM dNTPs, μL | 4 | |
| FW primer, μL | 4 | P2 |
| REV primer, μL | 4 | Added separately |
| Template DNA, μL | 4 | Added seperatley |
| Taq-Polymerase, μL | 1 | |
| ddH2O, μL | 163 | |
| Total, μL | 200 | |
| Total master mix, μL | 192 | |
| Aliquot each tube, μL | 48 | |

TABLE 21
Thermocycling conditions
Thermocycling conditions

| Step | Temperature | Time | |
|---|---|---|---|
| Initial denaturation | 95° C. | 30 | sec. |
| Denaturation (25 cycles) | 95° C. | 15 | sec. |
| Annealing (25 cycles) | 58° C. | 15 | sec. |
| Extension (25 cycles) | 68° C. | 45 | sec. |
| Final extension | 68° C. | 5 | min. |

| | | |
|---|---|---|
| Hold | 4° C. | — |

TABLE 22
Tube set-up

| Tube | Primer | Template | Volume | Buffer |
|---|---|---|---|---|
| 1 | P1 | F1 | 1 μL | Taq |
| 2 | P4 | F1 | 1 μL | Taq |
| 3 | P1 | F2 | 1 μL | Taq |
| 4 | P4 | F2 | 1 μL | Taq |

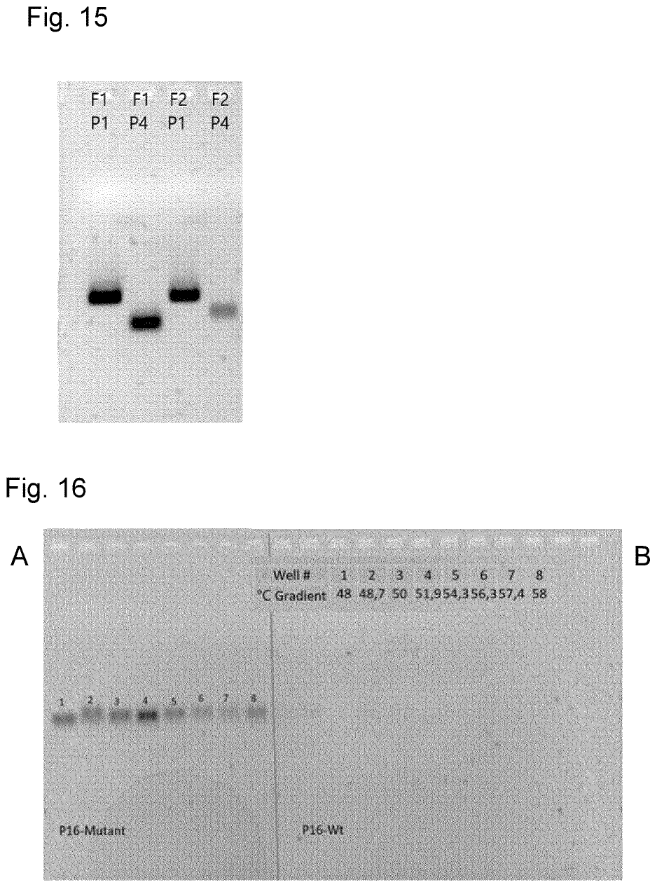

FIG. 15 shows the electrophoresis gels of the PCR products, and shows that, for a high, uniform, temperature across the thermocycler the standard Taq buffer is stable and produced conditions which retained primer-mutant template affinity. Primer-wild type template affinity is lost, and interaction is disrupted to a point where minimal amount of fragments are produced.

### Example X—Detection of ASTE1 a11→a10 Frameshift Mutations

A panel of PCR primers for detection of mutated ASTE1 was designed in a similar way to the TGFβR2 primers of Example I.

Primers P11 and P12 are positive control forward and reverse primers, respectively, which anneal to both the wild type and the mutant ASTE1 sequence. These primers detect the presence of ASTE1 regardless of the presence or absence of the a11-to-a10 frameshift mutation (i.e. P11 and P12 are not mutant-specific), and ensure reliable results and that all criteria to run the PCR are met (no signal from the control=unsuccessful PCR).

The ASTE1 mutant primers were designed with mutant variant ASTE1 as the primary target, and each primer harbours the a10 microsatellite sequence. As with the TGFβR2 primers in Examples I and II, the shorter 10a sequence, rather than the wild type 11a sequence, introduces a mismatch between the wild type ASTE1 and the 3′ end of the primers. By introducing one to four mismatches (randomly selected nucleotide substitutions) in the primers, 3′ of the microsatellite, the mismatch repulsion between the primers and the wild type ASTE1 sequence is reinforced.

ASTE1 primers P16 and P24 were tested using a similar protocol to Example VIII, to establish a range of temperatures at which the primers are effective in annealing only to the mutant DNA template, and not the wild-type DNA template. Positive control reverse primer P12 was also used at a range of temperatures. Primer P11 was used as the positive primer in all experiments. Synthetic ASTE1 DNA fragments comprising either the wild type (a11) or the mutant (a10) microsatellites were used for the PCR set up (referred to as F4 and F3, respectively; SEQ ID NOs: 7 and 8, respectively).

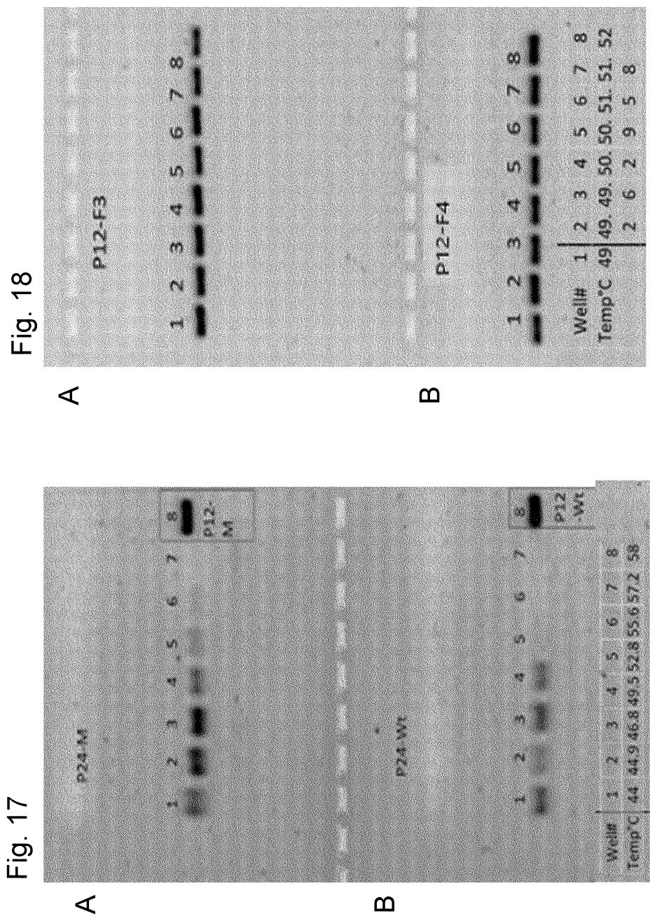

The PCR master mix used for each PCR is shown in Table 23 below, while the PCR conditions are shown in Table 24. A different annealing temperature was used for each PCR, as shown in Tables 25-27 and FIGS. 16-18. Standard (KCl) buffer was used for all of the PCRs. P11 was used as the forward primer for all PCRs.

TABLE 23
PCR master mix

| PCR master mix (Taq) | 50 μl reaction | |
|---|---|---|
| # of samples | 16 | |
| Taq buffer 10×, μL | 80 | |
| 10 mM dNTPs, μL | 16 | |
| FW primer, μL | 16 | P11 |
| REV primer, μL | 16 | Added separately |
| Template DNA, μL | 16 | Added separately |
| Taq-Polymerase, μL | 4 | |
| ddH2O, μL | 652 | |
| Total, μL | 800 | |
| Total master mix, μL | 768 | |
| Aliquot each tube, μL | 48 | |

TABLE 24
Thermocycling conditions
Thermocycling conditions

| Step | Temperature | Time | |
|---|---|---|---|
| Initial denaturation | 95° C. | 30 | sec. |
| Denaturation (25 cycles) | 95° C. | 15 | sec. |
| Annealing (25 cycles) | Varying | 15 | sec. |
| Extension (25 cycles) | 68° C. | 45 | sec. |
| Final extension | 68° C. | 5 | min. |

| | | |
|---|---|---|
| Hold | 4° C. | — |

TABLE 25
PCR annealing temperature for primer P16

| P16-mutant (F3) well no. | P16-WT (F4) well no. | Annealing temp. (° C.) |
|---|---|---|
| 1 | 1 | 48 |
| 2 | 2 | 48.7 |
| 3 | 3 | 50 |
| 4 | 4 | 51.9 |
| 5 | 5 | 54.3 |
| 6 | 6 | 56.3 |
| 7 | 7 | 57.4 |
| 8 | 8 | 58 |

TABLE 26
PCR annealing temperature for primer P24

| P24-M (F3) well no. | P24-Wt (F4) well no. | Annealing temp. (° C.) |
|---|---|---|
| 1 | 1 | 44 |
| 2 | 2 | 44.9 |
| 3 | 3 | 46.8 |
| 4 | 4 | 49.5 |
| 5 | 5 | 52.8 |
| 6 | 6 | 55.6 |
| 7 | 7 | 57.2 |
| 8 | 8 | 58 |

TABLE 27
PCR annealing temperature for primer P12

| P12-F3 well no. | P12-F4 well no. | Annealing temp. (° C.) |
|---|---|---|
| 1 | 1 | 49 |
| 2 | 2 | 49.2 |
| 3 | 3 | 49.6 |
| 4 | 4 | 50.2 |
| 5 | 5 | 50.9 |
| 6 | 6 | 51.5 |
| 7 | 7 | 51.8 |
| 8 | 8 | 52 |

### Results

FIGS. 16-18 show the results of these PCRs using different annealing temperatures. In particular, these Figures show that the primers designed based on a10 mutant ASTE1 (i.e. P16 and P24) are specific for a10 mutant ASTE1 and produce much lower, or no, PCR products when ASTE1 wild type is the template. FIG. 18 shows that P12 can be used to amplify both wild type and a10 mutant ASTE1, such that it can be used as a positive control primer.

### Example XI—Detection of ASTE1 a11→a10 Frameshift Mutations

A second panel of PCR primers for detection of frameshift mutated ASTE1 was designed in a similar way to the ASTE1 primers of EXAMPLE X.

ASTE1 primers P13, P23, P39 and P31 were tested using a similar protocol to Example VIII and Example X, to establish a range of temperatures at which the primers are effective in annealing only to the frameshift mutant DNA template, and not the wild-type DNA template. Positive control reverse primer P12 was also used at a range of temperatures. Primer P12 was used in all experiments. Synthetic ASTE1 DNA fragments comprising either the wild type (a11) or the mutant (a10) microsatellites were used for the PCR set up (referred to as F4 and F3, respectively; SEQ ID NOs: 7 and 8, respectively).

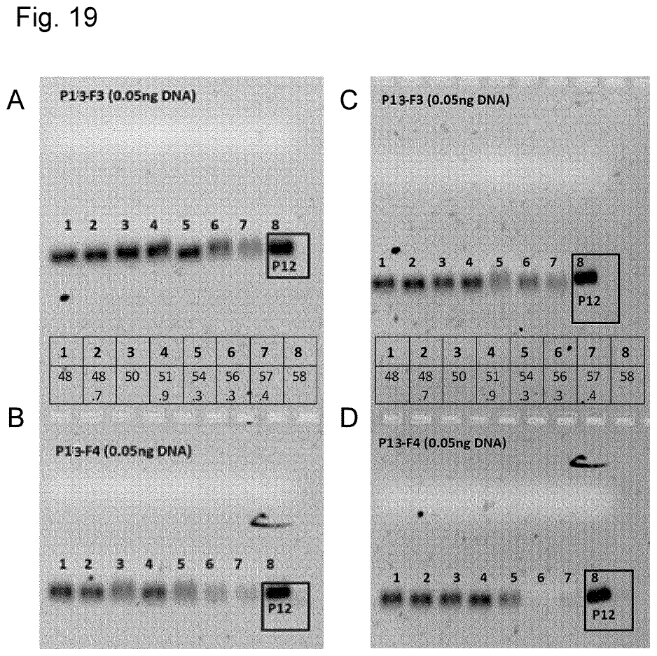

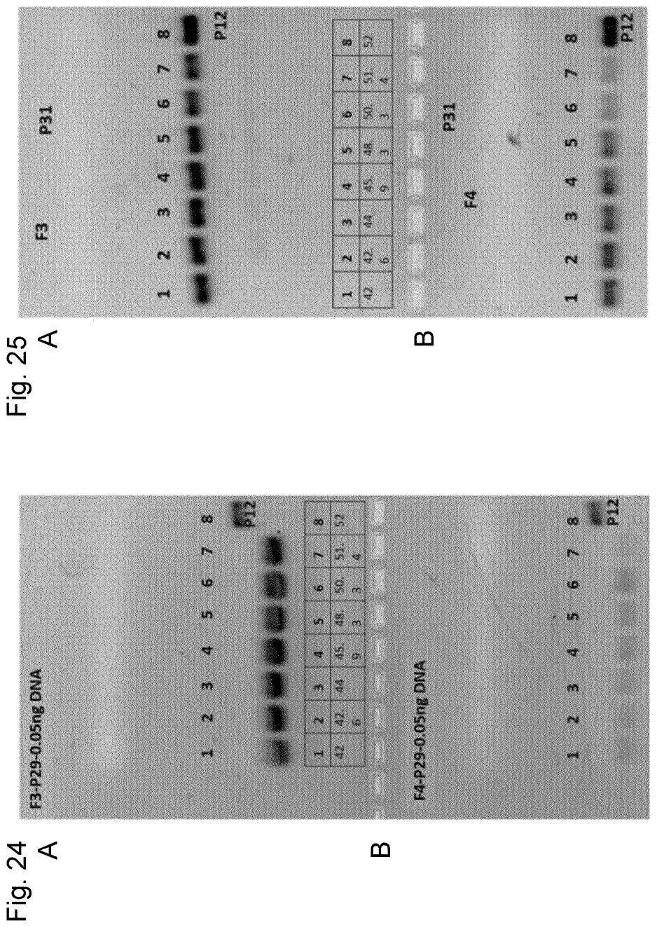

The PCR master mix used for each PCR is shown in Table 28 below, while the PCR conditions are shown in Table 29. A different annealing temperature was used for each PCR, as shown in Tables 30-36 and FIGS. 19-26. ThermoPol® buffer was used for all of the PCRs except for the PCR reaction shown in Table 34 and FIG. 24, in which the ThermoPol® buffer was replaced with Standard Taq (KCl) buffer. P11 was used as the positive control forward primer for all PCRs.

TABLE 28
PCR master mix

| PCR master mix (Taq) | 25 μl reaction | |
|---|---|---|
| # of samples | 18 | |
| 10× buffer, μL | 45 | |
| 10 mM dNTPs, L | 9 | |
| 10 μM FW primer, μL | 9 | P11 |
| 10 μM REV primer, μL | 9 | Added separately |
| Template DNA, μL | 3.6 to 9 | Added separately |
| Taq-polymerase, μL | 2.25 | Added separately |
| ddH2O, μL | 366.75 to 372.15 | |
| Total, μL | 450 | |
| W\O Primer, μL | 432 | |
| Aliquot each tube, μL | 25 | |

TABLE 29
Thermocycling conditions
Thermocycling conditions

| Step | Temperature | Time | |
|---|---|---|---|
| Initial denaturation | 95° C. | 2 | min. |
| Denaturation (25 cycles) | 95° C. | 15 | sec. |
| Annealing (25 cycles) | Varying | 15 | sec. |
| Extension (25 cycles) | 68° C. | 45 | sec. |
| Final extension | 68° C. | 5 | min. |

| | | |
|---|---|---|
| Hold | 4° C. | — |

TABLE 30
PCR annealing temperature for primer P13

| P13-mutant (F3) well no. | P13-WT (F4) well no. | Annealing temp. (° C.) |
|---|---|---|
| 1 | 1 | 48 |
| 2 | 2 | 48.7 |
| 3 | 3 | 50 |
| 4 | 4 | 51.9 |
| 5 | 5 | 54.3 |
| 6 | 6 | 56.3 |
| 7 | 7 | 57.4 |
| P12-mutant (F3) well no. | P12-WT (F4) well no. | Annealing temp. (° C.) |
| 8 - control | 8 - control | 58 |

TABLE 31
PCR annealing temperature for primer P13

| P13-mutant (F3) well no. | P13-WT (F4) well no. | Annealing temp. (° C.) |
|---|---|---|
| 1 | 1 | 54 |
| 2 | 2 | 54.3 |
| 3 | 3 | 54.8 |
| 4 | 4 | 55.6 |
| 5 | 5 | 56.6 |
| 6 | 6 | 57.3 |
| 7 | 7 | 57.8 |
| P12-mutant (F3) well no. | P12-WT (F4) well no. | Annealing temp. (° C.) |
| 8 - control | 8 - control | 58 |

TABLE 32
PCR annealing temperature for primer P23

| P23-mutant (F3) well no. | P23-WT (F4) well no. | Annealing temp. (° C.) |
|---|---|---|
| 1 | 1 | 48 |
| 2 | 2 | 48.7 |
| 3 | 3 | 50 |
| 4 | 4 | 51.9 |
| 5 | 5 | 54.3 |
| 6 | 6 | 56.3 |
| 7 | 7 | 57.4 |
| P12-mutant (F3) well no. | P12-WT (F4) well no. | Annealing temp. (° C.) |
| 8 - control | 8 - control | 58 |

TABLE 33
PCR annealing temperature for primer P23

| P23-mutant (F3) well no. | P23-WT (F4) well no. | Annealing temp. (° C.) |
|---|---|---|
| 1 | 1 | 54 |
| 2 | 2 | 54.3 |
| 3 | 3 | 54.8 |
| 4 | 4 | 55.6 |
| 5 | 5 | 56.6 |
| 6 | 6 | 57.3 |
| 7 | 7 | 57.8 |
| P12-mutant (F3) well no. | P12-WT (F4) well no. | Annealing temp. (° C.) |
| 8 - control | 8 - control | 58 |

TABLE 34
PCR annealing temperature for primer P23 in Standard Taq buffer

| P23-mutant (F3) well no. | P23-WT (F4) well no. | Annealing temp. (° C.) |
|---|---|---|
| 1 | 1 | 54 |
| 2 | 2 | 54.3 |
| 3 | 3 | 54.8 |
| 4 | 4 | 55.6 |
| 5 | 5 | 56.6 |
| 6 | 6 | 57.3 |
| 7 | 7 | 57.8 |
| P12-mutant (F3) well no. | P12-WT (F4) well no. | Annealing temp. (° C.) |
| 8 - control | 8 - control | 58 |

TABLE 35
PCR annealing temperature for primer P29

| P29-mutant (F3) well no. | P29-WT (F4) well no. | Annealing temp. (° C.) |
|---|---|---|
| 1 | 1 | 42 |
| 2 | 2 | 42.6 |
| 3 | 3 | 44 |
| 4 | 4 | 45.9 |
| 5 | 5 | 49.3 |
| 6 | 6 | 50.3 |
| 7 | 7 | 51.4 |
| P12-mutant (F3) well no. | P12-WT (F4) well no. | Annealing temp. (° C.) |
| 8 - control | 8 - control | 52 |

TABLE 36
PCR annealing temperature for primer P31

| P31-mutant (F3) well no. | P31-WT (F4) well no. | Annealing temp. (° C.) |
|---|---|---|
| 1 | 1 | 42 |
| 2 | 2 | 42.6 |
| 3 | 3 | 44 |
| 4 | 4 | 45.9 |
| 5 | 5 | 49.3 |
| 6 | 6 | 50.3 |
| 7 | 7 | 51.4 |
| P12-mutant (F3) well no. | P12-WT (F4) well no. | Annealing temp. (° C.) |
| 8 - control | 8 - control | 52 |

### Results

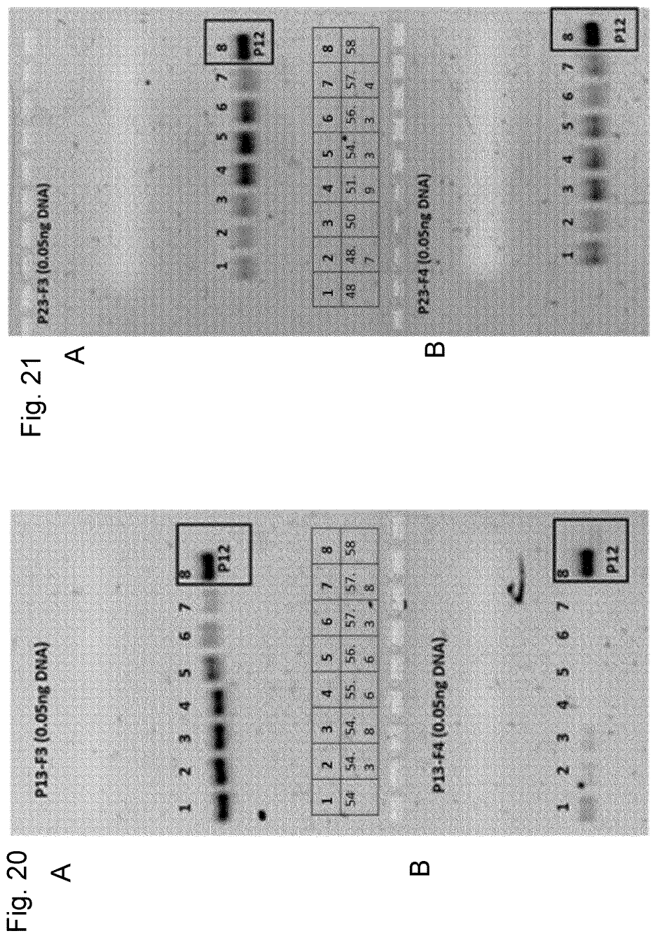

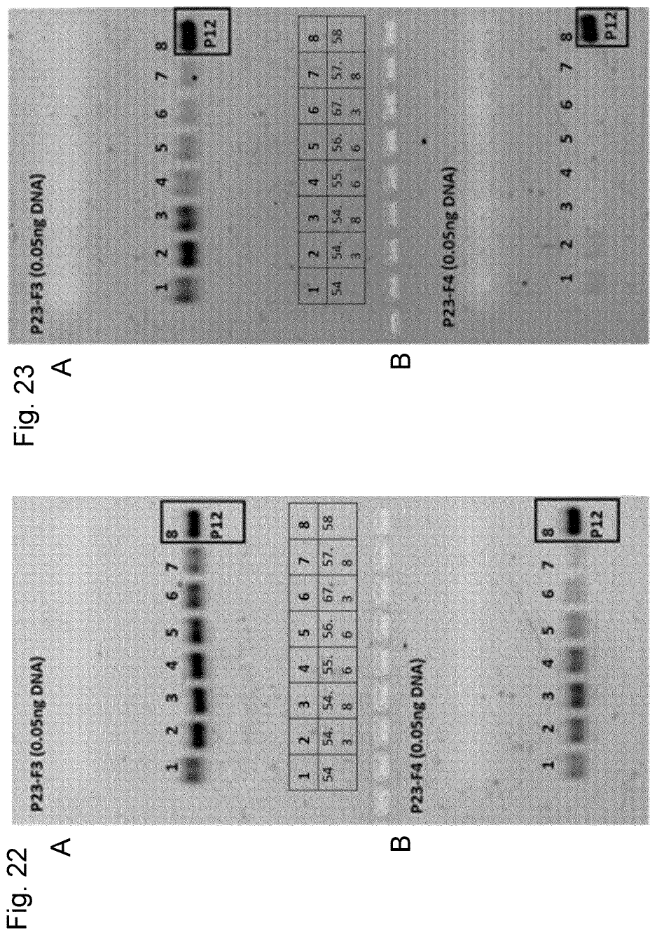

FIGS. 19-25 show the results of these PCRs using different annealing temperatures. In particular, these Figures show that the primers designed based on a10 mutant ASTE1 (i.e. P13, P23, P29 and P31) are specific for a10 mutant ASTE1 and produce much lower, or no, PCR products when ASTE1 wild type is the template. In particular, FIGS. 19 and 20 show the optimal temperature range for the P13 primer to be 52° C. to 56° C., FIGS. 21 and 22 show the optimal temperature range for the P23 primer to be 54° C. to 58° C., and FIG. 24 shows the optimal temperature range for the P29 primer to be 42° C. to 52° C., and FIG. 25 shows the optimal temperature range for the P29 primer to be 50.3° C. to 52° C., in these conditions.

In FIGS. 19-24, the PCR reaction mixture contained the DNA template at 0.05 ng/μL, whilst in FIG. 25, the DNA template concentration was 0.2 ng/μL. This shows that a low final concentration of template DNA is sufficient for the PCR reaction. This is beneficial as it shows that that a lower amount of template DNA is required for the PCT reaction than previously thought.

FIGS. 22 and 23 further show the different binding properties of the P23 primer on a10 mutant ASTE1 and a11 wild type ASTE1 when the buffer was replaced from the stabilizing ThermoPol® buffer to the destabilizing Standard Taq buffer, respectively. FIG. 23 shows that the overall signal strength of the result is decreased due to the destabilizing effect of the Standard Taq buffer. FIG. 22 shows the optimal temperature range for the P23 primer in ThermoPol® buffer to be 54° C. to 58° C., whilst FIG. 23 shows the optimal temperature range for the P23 primer in standard Taq buffer to be 54° C. to 55° C. This demonstrates that the optimal annealing temperature of the P23 primer differs depending on the buffer used.

### Example XII—Detection of TAF1β a11→a10 Frameshift Mutations

A panel of PCR primers for detection of frameshift mutated TAF1β was designed in a similar way to the TGFβR2 primers of Example I.

Primers P37 and P38 are positive control forward and reverse primers, respectively, which anneal to both the wild type and the mutant TAF1β sequence. These primers detect the presence of TAF1β regardless of the presence or absence of the a11-to-a10 frameshift mutation (i.e. P37 and P38 are not mutant-specific), and ensure reliable results and that all criteria to run the PCR are met (no signal from the control=unsuccessful PCR).

The TAF1β frameshift mutant primers were designed with a10 frameshift mutant TAF1β as the primary target, and each primer harbours the a10 microsatellite sequence. As with the TGFβR2 primers in Examples I and II, the shorter a10 sequence, rather than the wild type a11 sequence, introduces a mismatch between the wild type TAF1β and the 3′ end of the primers. By introducing one to four mismatches (randomly selected nucleotide substitutions) in the primers, 3′ of the microsatellite, the mismatch repulsion between the primers and the wild type TAF1β sequence is reinforced.

TAF1β primers P42 and P43 were tested using a similar protocol to Example VIII, to establish whether the primers are effective in annealing only to the mutant DNA template, and not the wild-type DNA template, at 54° C. Positive control forward primer P37 was also used at a range of temperatures. Primer P37 was used in all experiments. Synthetic TAF1β DNA fragments comprising either the wild type (a11) or the mutant (a10) microsatellite were used for the PCR set up (referred to as F6 and F5, respectively; SEQ ID NOs: 11 and 12, respectively).

The PCR master mix used for each PCR is shown in Table 37 below, while the PCR conditions are shown in Table 38. The results are shown in Table 39 and FIG. 26. Standard (KCl) buffer was used for all of the PCRs. P38 was used as the positive control reverse primer for all PCRs.

TABLE 37
PCR master mix

| PCR master mix (Taq) | 25 μl reaction | |
|---|---|---|
| # of samples | 7 | |
| 10× buffer, μL | 17.5 | |
| 10 mM dNTPs, μL | 3.5 | |
| FW primer, μL | 3.5 | P38 |
| REV primer, μL | 3.5 | Added separately |
| Template DNA, μL | 14 | Added separately |
| Taq-polymerase, μL | 0.875 | |
| ddH2O, μL | 132.125 | |
| Total, μL | 175 | |
| W\O Primer, μL | 171.5 | |
| Aliquot each tube, μL | 24.5 | |

TABLE 38
Thermocycling conditions
Thermocycling conditions

| Step | Temperature | Time | |
|---|---|---|---|
| Initial denaturation | 95° C. | 30 | sec. |
| Denaturation (25 cycles) | 95° C. | 15 | sec. |
| Annealing (25 cycles) | 54° C. | 15 | sec. |
| Extension (25 cycles) | 68° C. | 45 | sec. |
| Final extension | 68° C. | 5 | min. |

| | | |
|---|---|---|
| Hold | 4° C. | — |

TABLE 39
Gel for PCR for primers P42 and P43

| Wells | Primer | Template |
|---|---|---|
| 1 | P42 | F5 |
| 2 | P42 | F6 |
| 3 | — | H2O |
| 4 | P43 | F5 |
| 5 | P43 | F6 |
| 6 | — | H2O |
| 7 | — | H2O |
| 8 | P37 | F5 |
| 9 | — | H2O |
| 10 | P37 | F6 |

### Results

FIG. 26 shows that the primers designed based on the a10 frameshift mutant TAF1β (i.e. P42 and P43) are specific for a10 frameshift mutant TAF1β. In particular, FIG. 26 shows that P42 and P43 produce much lower, or no, PCR products using TAF1β wild type as the template compared to using the a10 frameshift mutant TAF1β as the template. FIG. 26 further confirms that P37 can be used to amplify both wild type and a10 mutant TAF1β, such that it can be used as a positive control primer.

### Example XIII—Detection of ACVR2A a8→a7 Frameshift Mutations

PCR primers for detection of exon 10 frameshift mutated ACVR2A was designed in a similar way to the TGFβR2, ASTE1 and TAF1β primers described above.

P.ACV.1 and P.ACV.2 are positive control reverse and forward primers, respectively, which anneal to both the wild type and the mutant ACVR2A sequence. These primers detect the presence of ACVR2A regardless of the presence or absence of the a8-to-a7 frameshift mutation (i.e. P.ACV.1 and P.ACV.2 are note mutant-specific), and ensure reliable results and that all criteria to run the PCR are met (no signal from the control=unsuccessful PCR).

The ACVR2A frameshift mutant primers were designed with exon 10 a7 frameshift mutant ACVR2A as the primary target, and each primer harbours the a7 microsatellite sequence. As with the TGFβR2 primers in Examples I and II, the shorter a7 sequence, rather than the wild type a8 sequence, introduces a mismatch between the wild type ACVR2A and the 3′ end of the primers. By introducing one to four mismatches (randomly selected nucleotide substitutions) in the primers, 3′ of the microsatellite, the mismatch repulsion between the primers and the wild type ACVR2A sequence is reinforced.

Synthetic fragments of a8 wild-type ACVR2A and exon 10 a7 mutant ACVR2A were used as template DNA (SEQ ID NO: 96 and 97). The PCR master mix content and the thermocycler conditions used in this Example are set out in Tables 40 and 41 below. The buffer used was Key buffer.

TABLE 40
PCR master mix

| PCR master mix | 25 μl reaction |
|---|---|
| # of samples | 1 |
| 10× buffer, μL | 2.5 |
| 10 mM dNTPs, μL | 0.5 |
| FW primer, μL | 0.5 |
| REV primer, μL | 0.5 |
| Template DNA, μL | 2 |
| Taq-polymerase, μL | 0.25 |
| ddH2O, μL | 18.75 |

TABLE 41
Thermocycling conditions
Thermocycling conditions

| | | |
|---|---|---|
| Step | Temperature | Time |

| | | | | |
|---|---|---|---|---|
| Initial denaturation | 95° | C. | 30 | sec. |
| Denaturation (25 cycles) | 95° | C. | 15 | sec. |
| Annealing (25 cycles) | 50-60° | C. | 15 | sec. |
| Extension (25 cycles) | 68° | C. | 45 | sec. |
| Final extension | 68° | C. | 5 | min. |

| | | | |
|---|---|---|---|
| Hold | 4° | C. | — |

To perform a PCR run, reagents were assembled carefully on ice after thawing on the lab bench, then briefly spun down before being placed in the thermocycler and run on the program described in Table 41. A temperature gradient was run with each primer, as shown in Table 41. Autoclaved, unused tips were used throughout all experiments.

1.8% agarose gel was cast 30 minutes before PCR completion. 8.5 μL sample was stained with 1.5 μL DNA-dye NonTox and loaded into wells of the gel. Electrophoresis was performed at 90V for 60 minutes, and the gel was subsequently imaged using Amersham Imager 600.

Both P.ACV.21 and P.ACV.22 show excellent affinity towards the exon 10 a7 ACVR2A mutant template DNA (SEQ ID NO: 97), with strong amplicons produced at all temperatures, while no or very weak amplicons are generated with the a8 wild-type ACVR2A fragment (SEQ ID NO: 96) (FIG. 27).

### Example XIV—Detection of TAF1β a11→a10 Frameshift Mutations

Primers P42 and P43 (SEQ ID NOs: 55 and 56) targeting a10 frameshift mutated TAF1β were tested with synthetic a10 mutant TAF1β template DNA (SEQ ID NO: 12) and synthetic wild type a11 TAF1β template DNA (SEQ ID NO: 11), at an annealing temperature of 56° C. In particular, the primers were tested with the same parameters as have been used for primers targeting frameshift mutated TGFβR2 above, namely, an annealing time of 15 seconds, an annealing temperature of 56° C. and a buffer containing ammonium sulphate (i.e. Key buffer).

The PCR master mix contents and the thermocycling conditions used are shown in Tables 43 and 44 below. The buffer used was Key buffer.

TABLE 43
PCR master mix

| PCR master mix | 25 μl reaction |
|---|---|
| # of samples | 5 |
| 10× buffer, μL | 12.5 |
| 10 mM dNTPs, μL | 2.5 |
| FW primer, μL | 2.5 |
| REV primer, μL | 2.5 |
| Template DNA, μL | 15 |
| Taq-polymerase, μL | 0.6 |
| ddH2O, μL | 89.3 |

TABLE 44
Thermocycling conditions
Thermocycling conditions

| Step | Temperature | Time | |
|---|---|---|---|
| Initial denaturation | 95° C. | 30 | sec. |
| Denaturation (25 cycles) | 95° C. | 15 | sec. |
| Annealing (25 cycles) | 56° C. | 15 | sec. |
| Extension (25 cycles) | 68° C. | 45 | sec. |
| Final extension | 68° C. | 5 | min. |

| | | |
|---|---|---|
| Hold | 4° C. | — |

After thawing on the lab bench, reagents were assembled carefully on ice, then briefly spun down before being placed in the thermocycler. Autoclaved, unused tips were used throughout the experiment.

1.8% agarose gel was cast 30 min before PCR completion. Samples were stained with DNA-dye NonTox and loaded into wells of the gel.

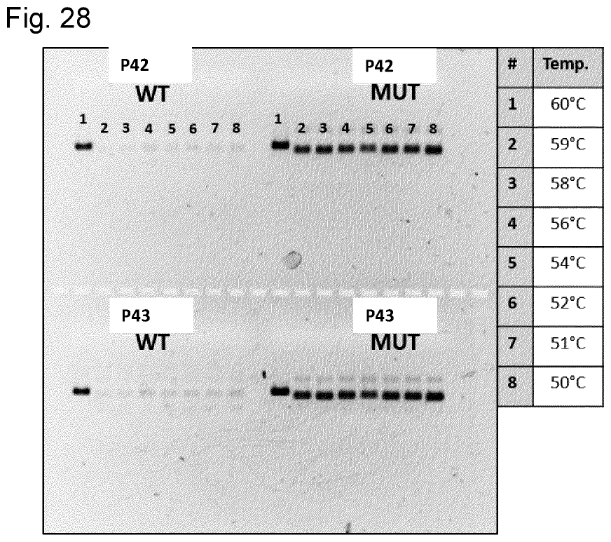

Under the same conditions as the previously designed TGFβR2 primers, P42 and P43 had no noticeable affinity for the undesired wild type TAF1β template DNA, with a high degree of variability in terms of temperature conditions. On the other hand, P42 and P43 were able to to anneal and amplify the a10 frameshift mutated TAF1β template DNA (FIG. 28). Thus, P42 and P43 demonstrate a high degree of selectivity and sensitivity towards a10 frameshift mutated TAF1β while having a low affinity for the wild-type sequence.

### Example XV—Detection of ASTE1 a11→a10 Frameshift Mutations

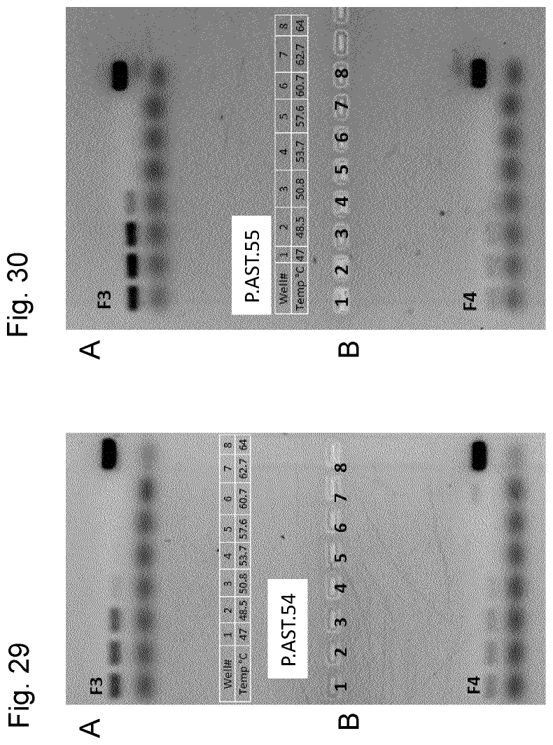

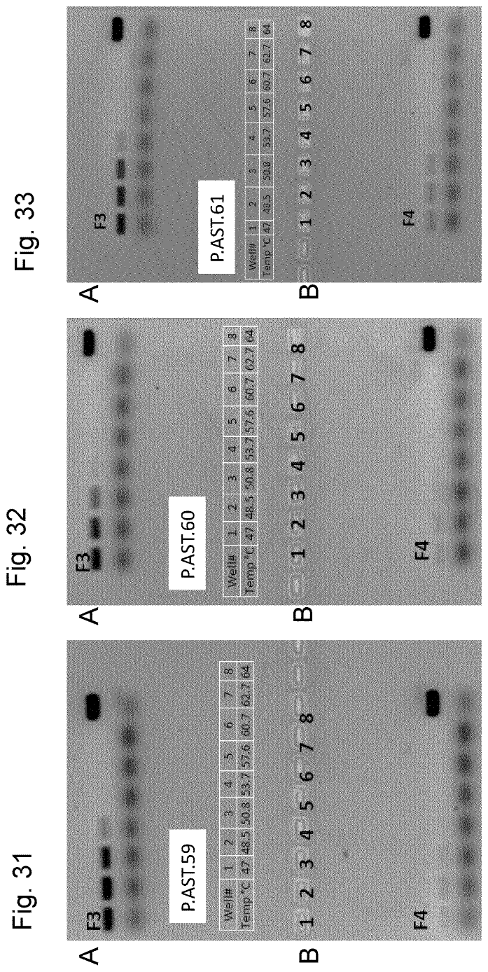

Primers pairs P.AST.54-P11 (SEQ ID NOs: 84 and 29), P.AST.55-P11 (SEQ ID NOs: 85 and 29), P.AST.59-P11 (SEQ ID NOs: 86 and 29), P.AST.60-P11 (SEQ ID NOs: 87 and 29), and P.AST.61-P11 (SEQ ID NOs: 88 and 29) targeting a10 frameshift mutated ASTE1 were tested with a synthetic a10 mutant ASTE1 template DNA (SEQ ID NO: 8) and a synthetic wild-type ASTE1 template DNA (SEQ ID NO: 7) in the temperature range 47-62.7° C., using a buffer containing (NH₄)₂SO₄ and 20 mM MgCl₂. P.AST.2 (SEQ ID NO: 102) was used as a positive control reverse primer, and the P.AST.2+P11 primer pair is shown in lane 8 of each of FIGS. 29 to 33. All primer pairs have a calculated Tₘ (melting temperature) with Taq polymerase in the annealing step of 48° C., with the exception of P.AST.60, which has a Tₘ of 46° C. Therefore, the experiments were performed with a temperature gradient ranging from 47-62.7° C. with the intention of seeing when the primers would destabilise. The reason for not setting a lower temperature than 47° C. is that a lower temperature step for the annealing will induce non-specific binding.

The PCR master mix contents, the thermocycling conditions and the annealing step temperatures used are shown in Tables 45 to 47 below. The buffer used was Taq buffer.

TABLE 45
PCR master mix

| | | | Volume, μl | Volume, μl |
|---|---|---|---|---|
| PCR Master mix | Volume, μl | Volume, μl | (28 reactions, | (28 reactions, |
| (25 μl reaction) | (56 reactions) | (28 reactions) | 195 μl) | 97.5 μl) |
| ddH₂O | 1141 | | | |
| 10 mM dNTPs | 28 | | | |
| Fwd. primer (P11) | 28 | | | |
| 10× Buffer, μL | 140 | | | |
| | Mix gently, split | | | |
| | into two vials | | | |
| | a 668.5 | | | |
| DNA Template | | 14 | | |
| (F3/F4), 0.2^{ng}/ₚₗ | | | | |
| | | Mix gently, split | | |
| | | into four vials | | |
| | | (1 × 97.5, | | |
| | | 3 × 195) | | |
| REV primer, μL | | | 4 (P.AST.54, | 2 (P.AST.2) |
| | | | P.AST.55, | |
| | | | P.AST.59, | |
| | | | P.AST.60, | |
| | | | P.AST.61) | |
| Taq-polymerase | | | 1 | 0.5 |
| Aliquote each | | | 25 | 25 |
| tube | | | | |

TABLE 46
Thermocycling conditions

| | | |
|---|---|---|
| Step | Temperature | Time |

| | | | | |
|---|---|---|---|---|
| 1) Initial denaturation | 95° | C. | 2 | min. |
| 2) Denaturation (25 | 95° | C. | 15 | sec. |
| cycles) | | | | |
| 3) Annealing (25 cycles) | X° | C. | 15 | sec. |
| 4) Extension (25 cycles) | 68° | C. | 45 | sec. |
| 5) Final extension | 68° | C. | 5 | min. |

| | | | |
|---|---|---|---|
| 6) Hold | 4° | C. | — |

TABLE 47
Annealing step temperature

| Well | 1 | 2 | 3 | 4 | 5 | 6 | 7 | 8 |
|---|---|---|---|---|---|---|---|---|
| Temperature | 47 | 48.5 | 50.8 | 53.7 | 57.6 | 60.7 | 62.7 | 64 |
| (° C.) | | | | | | | | |

All reagents, except Taq DNA polymerase, were thawed on the lab bench and then assembled on ice. Before the samples was placed in the thermocycler, they were spun down quickly (2 sec.). Autoclaved, unused pipette tips were used throughout the experiment. 100 ml 1.8% Agarose gel was stained with 10 μl GelRed/GelGreen DNA dye and left on the bench for 50 minutes. Samples were loaded by mixing in Blue loading buffer before loading 10 μl samples. Electrophoresis was performed at 90 V for 60 min. The gel was imaged using an Amersham Imager 600.

The results are shown in FIGS. 29 to 33. The buffer used for the PCR proves to greatly enhance the binding properties of primers P.AST.54, P.AST.55, P.AST.59, P.AST.60 and P.AST.61 to the mutated DNA template in the temperature range 47-62.7° C. The properties of the buffer greatly enhance the ability of P.AST.54, P.AST.55, P.AST.59, P.AST.60 and P.AST.61 to bind to the mutated DNA template over the wild-type DNA template in the temperature range 47-62.7° C.

### Example XVI—Testing of ASTE1 10a Frameshift Primers P.AST.59, P.AST.60 and P.AST.61

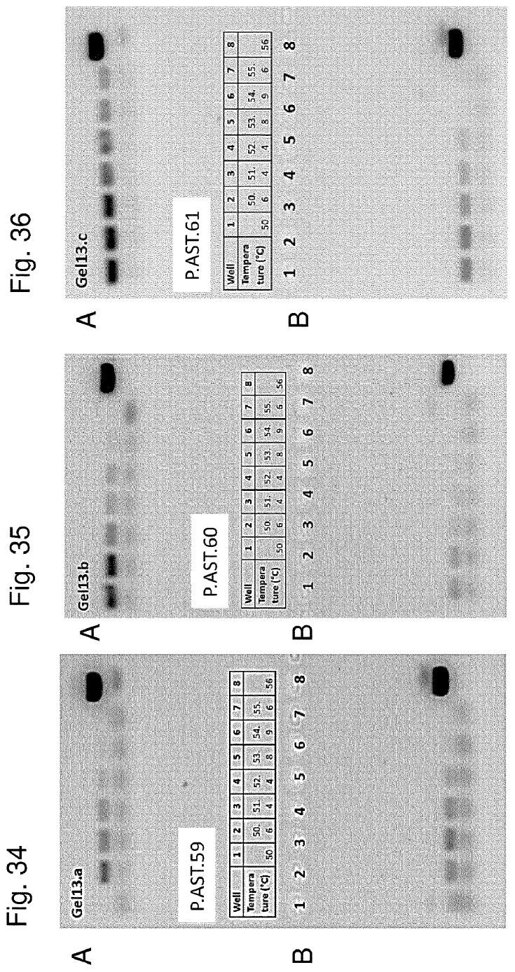

Primer pairs P.AST.59-P11 (SEQ ID NOs: 86 and 29), P.AST.60-P11 (SEQ ID NOs: 87 and 29), and P.AST.61-P11 (SEQ ID NOs: 88 and 29) were tested against synthetic a10 mutant ASTE1 template DNA (SEQ ID NO: 8) and a synthetic wild-type ASTE1 template DNA (SEQ ID NO: 7) in a smaller temperature range (50-55.6° C.), using a buffer containing (NH₄)₂SO₄ and 20 mM MgCl₂, with an increase of amplification cycles from 25 to 30. P.AST.2 (SEQ ID NO: 102) was used as a control primer in combination with P11. The control is shown in lane 8 of each of FIGS. 34 to 36. The initial experiments aimed to investigate primer functionality under standard PCR conditions, in terms of annealing temperature and time, buffers, and polymerase. Based on previous experiments, a higher shift in affinity was observed with these primers for 25 amplification cycles. Therefore, the experiments were performed with a 5 amplification cycle increase (30 cycles in total) to see if the primers that already annealed to the 10a ASTE1 template DNA would give a higher amplicon yield when allowed more cycles. A temperature gradient ranging from 50-55.6° C. was chosen with the intention of seeing when the primers would create the biggest shift between the DNA templates.

The PCR master mix contents, the thermocycling conditions and the annealing step temperatures used are shown in Tables 48 to 50 below. The buffer used was a buffer containing (NH₄)₂SO₄ and 20 mM MgCl₂.

TABLE 48
PCR master mix

| | | | Volume, μl | Volume, μl |
|---|---|---|---|---|
| PCR Master mix | Volume, μl | Volume, μl | (28 reactions, | (28 reactions, |
| (25 μl reaction) | (56 reactions) | (28 reactions) | 195 μl) | 97.5 μl) |
| ddH₂O | 1141 | | | |
| 10 mM dNTPs | 28 | | | |
| Fwd. primer (P11) | 28 | | | |
| 10× Buffer, μL | 140 | | | |
| | Mix gently, split | | | |
| | into two vials | | | |
| | a 668.5 | | | |
| DNA Template | | 14 | | |
| (F3/F4), 0.2 ng/μl | | | | |
| | | Mix gently, split | | |
| | | into four vials | | |
| | | (1 × 97.5, | | |
| | | 3 × 195) | | |
| REV primer, μL | | | 4 (P.AST.59, | 2 (P.AST.2) |
| | | | P.AST.60, | |
| | | | P.AST.61) | |
| Taq-polymerase | | | 1 | 0.5 |
| Aliquote each | | | 25 | 25 |
| tube | | | | |

TABLE 49
Thermocycler conditions

| | | |
|---|---|---|
| Step | Temperature | Time |

| | | | | |
|---|---|---|---|---|
| 1) Initial denaturation | 95° | C. | 2 | min. |
| 2) Denaturation (30 cycles) | 95° | C. | 15 | sec. |
| 3) Annealing (30 cycles) | X° | C. | 15 | sec. |
| 4) Extension (30 cycles) | 68° | C. | 45 | sec. |
| 5) Final extension | 68° | C. | 5 | min. |

| | | | |
|---|---|---|---|
| 6) Hold | 4° | C. | — |

TABLE 50
Annealing step temperature

| Well | 1 | 2 | 3 | 4 | 5 | 6 | 7 | 8 |
|---|---|---|---|---|---|---|---|---|
| Temperature | 50 | 50.6 | 51.4 | 52.4 | 53.8 | 54.9 | 55.6 | 56 |
| (° C.) | | | | | | | | |

All reagents, except Taq DNA polymerase, were thawed on the lab bench and then assembled on ice. Samples were spun down for 2 seconds before being placed in the thermocycler. Autoclaved, unused pipettes tips were used throughout the whole assembly. 100 ml 1.8% Agarose gel was prepared and stained with 10 μl GelRed DNA dye and left on the bench for 50 minutes. Samples were loaded by mixing in Blue loading buffer before loading 10 μl samples. Electrophoresis was performed at 90V for 60 min. The gel was imaged using an Amersham Imager 600. All experiments were conducted in an identical manner.

Results are shown in FIGS. 34 to 36. When the number of amplification cycles was increased from 25 to 30, the signal strength of P.AST.60 (FIG. 35) and P.AST.61 (FIG. 36) increase and are stronger compared to the control. The overall increase of the signal from the primers that is observed when compared to the control primer pairs (P.AST.2+P11) under the same conditions is due to the increasing of the amplification cycles.

### Example XVII—Testing of ASTE1 10a Frameshift Primers P.AST.65, P.AST.67 and P.AST.79

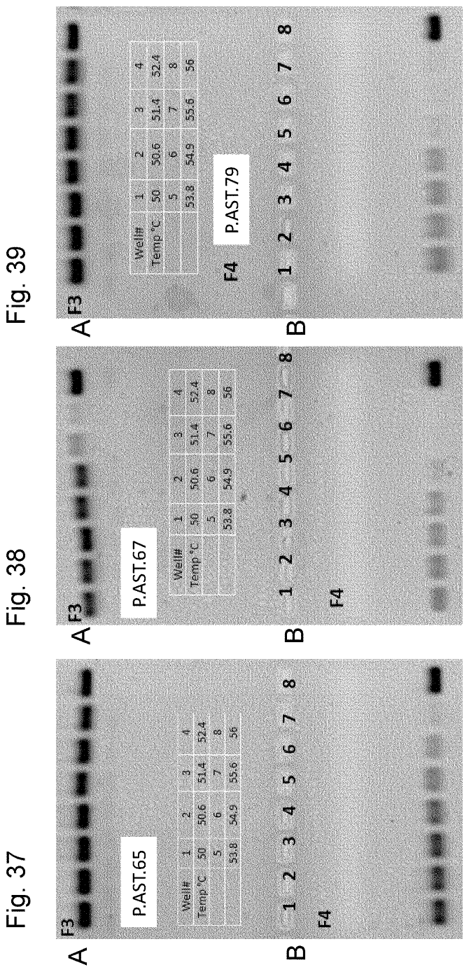

This experiment was carried out according to the method of Example XVI. The primers P.AST.65 (SEQ ID NO: 98), P.AST.67 (SEQ ID NO: 99), and P.AST.79 (SEQ ID NO: 101), each in a pair with P11 (SEQ ID NO: 29), were tested against synthetic a10 mutant ASTE1 template DNA (SEQ ID NO: 8) and a synthetic wild-type ASTE1 template DNA (SEQ ID NO: 7), using a buffer containing (NH₄)₂SO₄ and 20 mM MgCl₂. P.AST.2 was used as a positive control reverse primer (SEQ ID NO: 102), and the results of the control P11+P.AST.2 primer pair is shown in lane 8 of FIGS. 37 to 39. All primer pairs have a calculated Tₘ (melting temperature) with Taq polymerase in the annealing step of 48° C. The number of amplification cycles was increased from 25 to 30.

The PCR master mix contents, the thermocycling conditions and the annealing step temperatures used are shown in Tables 51 to 53 below. The buffer used was a buffer containing (NH₄)₂SO₄ and 20 mM MgCl₂.

TABLE 51
PCR master mix

| | | | Volume, μl | Volume, μl |
|---|---|---|---|---|
| PCR Master mix | Volume, μl | Volume, μl | (28 reactions, | (28 reactions, |
| (25 μl reaction) | (56 reactions) | (28 reactions) | 195 μl) | 97.5 μl) |
| ddH₂O | 1141 | | | |
| 10 mM dNTPs | 28 | | | |
| Fwd. primer (P11) | 28 | | | |
| 10× Buffer, μL | 140 | | | |
| | Mix gently, split | | | |
| | into two vials | | | |
| | a 668.5 | | | |
| DNA Template | | 14 | | |
| (F3/F4), 0.2 ng/μl | | | | |
| | | Mix gently, split | | |
| | | into four vials | | |
| | | (1 × 97.5, | | |
| | | 3 × 195) | | |
| REV primer, μL | | | 4 (P.AST.65, | 2 (P.AST.2) |
| | | | P.AST.67, | |
| | | | P.AST.79) | |
| Taq-polymerase | | | 1 | 0.5 |
| Aliquote each | | | 25 | 25 |
| tube | | | | |

TABLE 52
Thermocycler conditions

| | | |
|---|---|---|
| Step | Temperature | Time |

| | | | | |
|---|---|---|---|---|
| 1) Initial denaturation | 95° | C. | 2 | min. |
| 2) Denaturation (30 cycles) | 95° | C. | 15 | sec. |
| 3) Annealing (30 cycles) | X° | C. | 15 | sec. |
| 4) Extension (30 cycles) | 68° | C. | 45 | sec. |
| 5) Final extension | 68° | C. | 5 | min. |

| | | | |
|---|---|---|---|
| 6) Hold | 4° | C. | — |

TABLE 53
Annealing step temperature

| Well | 1 | 2 | 3 | 4 | 5 | 6 | 7 | 8 |
|---|---|---|---|---|---|---|---|---|
| Temperature | 50 | 50.6 | 51.4 | 52.4 | 53.8 | 54.9 | 55.6 | 56 |
| (° C.) | | | | | | | | |

All reagents, except Taq DNA polymerase, were thawed on the lab bench and then assembled on ice. Samples were spun down for 2 seconds before being placed in the thermocycler. Autoclaved, unused pipettes tips were used throughout the whole assembly. 100 ml 1.8% Agarose gel was left on the bench for 50 minutes. Samples were loaded by mixing in Blue loading buffer before loading 10 μl samples. Electrophoresis was performed at 90 V for 60 min. The picture of the gel was taken by Amersham Imager 600. All experiments were conducted in an identical manner

Results are shown in FIGS. 37 to 39. P.AST.65 (FIG. 37), P.AST.67 (FIG. 38) and P.AST.79 (FIG. 39) show affinity towards the mutated 10a ASTE1 template DNA and not the wild-type 11a ASTE1 template DNA.

### REFERENCES

Cortes-Ciriano, I; Lee, S; Park W Y; Kim T M; Park; P J et al. (6 Jun. 2017) “A molecular portrait of microsatellite instability across multiple cancers”. Nature Communications. 8 (15180): 1-12.
Iannuzzi, M C; Stern, R C; Collins, F S; Hon, C T; Hidaka, N; Strong, T; Becker, L; Drumm, M L; White, M B; Gerrard, B (February 1991). “Two frameshift mutations in the cystic fibrosis gene”. American Journal of Human Genetics. 48 (2): 227-31. PMC 1683026. PMID 1990834.
Maby, P; Tougeron, D; Hamieh, M; et al (Sep. 1 2015). “Correlation between Density of CD8+ T-cell Infiltrate in Microsatellite Unstable Colorectal Cancers and Frameshift Mutations: A Rationale for Personalized Immunotherapy”. Cancer Research. 75 (17): 3446-3455.
Ogura Y, Bonen D K, Inohara N, Nicolae D L, Chen F F, Ramos R, Britton H, Moran T, Karaliuskas R, Duerr R H, Achkar J P, Brant S R, Bayless T M, Kirschner B S, Hanauer S B, Nuñez G, Cho J H (May 31, 2001). “A frameshift mutation in NOD2 associated with susceptibility to Crohn's disease” (PDF). Nature. 411 (6837): 603-6. doi:10.1038/35079114. hdl:2027.42/62856. PMID 11385577. S2CID 205017657.
Allawi H T, SantaLucia J (1997) Thermodynamics and NMR of internal G-T mismatches in DNA. Biochemistry 36 (34), p 10581-10594.

TABLE 54
Sequences

| | SEQ ID | 5′-3′ | | |
|---|---|---|---|---|
| Primer | NO: | Direction | Sequence | Target |
| P1 | 13 | Reverse | CAGTGTCATAGAGCACATTCCT | TGFβR2 |
| P2 | 14 | Forward | AGACAGTTTGCCATGACCC | TGFβR2 |
| Pwt | 15 | Reverse | GTCTCACCAGGCTTTTTTTTTCC | TGFβR2 |
| Pwt2 | 16 | Reverse | CACCAGGCTTTTTTTTTCCTTC | TGFβR2 |
| P4 | 17 | Reverse | GTCTCACCAGGCTTTTTTTTTCG | TGFβR2 |
| P4.1 | 18 | Reverse | GTCTCACCAGGCTTTTTTTTCCG | TGFβR2 |
| P4.2 | 19 | Reverse | GTCTCACCAGGCTTTTTTTACCG | TGFβR2 |
| P5 | 20 | Reverse | GTCTCACCAGGCTTTTTTTTTCC | TGFβR2 |
| P6 | 21 | Reverse | GTCTCACCAGGCTTTTTTTTACG | TGFβR2 |
| P10 | 22 | Reverse | CACCAGGCTTTTTTTATCGTTC | TGFβR2 |
| P10.1 | 23 | Reverse | CACCAGGCTTTTTTTATCGTCC | TGFβR2 |
| P4x | 24 | Reverse | GTCTCACCAGGCTTTTTTTTTCX₁ | TGFβR2 |
| P4.1x/P6x | 25 | Reverse | GTCTCACCAGGCTTTTTTTTX₂CX₃ | TGFβR2 |
| P4.2x | 26 | Reverse | GTCTCACCAGGCTTTTTTTX₄X₅CX₆ | TGFβR2 |
| P10x | 27 | Reverse | CACCAGGCTTTTTTTX₇TCX₈TTC | TGFβR2 |
| P10.1x | 28 | Reverse | CACCAGGCTTTTTTTX₉TCX₁₀TX₁₁C | TGFβR2 |
| P11 | 29 | Forward | ATGTCCTGAGGCTAAGC | ASTE1 |
| P12 | 30 | Reverse | CACTGCACACACCAAGT | ASTE1 |
| P12.1 | 31 | Reverse | CACTGCACACACCAAGTG | ASTE1 |
| Pwt3 | 32 | Reverse | GTTTCTTCTGCCTTTTTTTTTTG | ASTE1 |
| Pwt4 | 33 | Reverse | CTTTTTTTTTTGAATTTGATCTAC | ASTE1 |
| P13 | 34 | Reverse | GTTTCTTCTGCCTTTTTTTATCG | ASTE1 |
| P14 | 35 | Reverse | GTTTCTTCTGCCTTTTTTTGTCG | ASTE1 |
| P15 | 36 | Reverse | GTTTCTTCTGCCTTTTTTTTACG | ASTE1 |
| P16 | 37 | Reverse | GTTTCTTCTGCCTTTTTTAATCG | ASTE1 |
| P17 | 38 | Reverse | GTTTCTTCTGCCTTTTTTACTCG | ASTE1 |
| P23 | 39 | Reverse | GTTTCTTCTGCCTTTTTTTAGTG | ASTE1 |
| P24 | 40 | Reverse | CTTTTTTTTCTGAATGTGATCTCC | ASTE1 |
| P29 | 41 | Reverse | GTTTCTTCTGCCTTTTTTATACG | ASTE1 |
| P31 | 42 | Reverse | GTTTCTTCTGCCTTTTTAATTCG | ASTE1 |
| P13x/P14x | 43 | Reverse | GTTTCTTCTGCCTTTTTTTX₁₂TX₁₃G | ASTE1 |
| P15x | 44 | Reverse | GTTTCTTCTGCCTTTTTTTTX₁₄X₁₅G | ASTE1 |
| P16x/P17x | 45 | Reverse | GTTTCTTCTGCCTTTTTTX₁₆X₁₇TX₁₈G | ASTE1 |
| P23x | 46 | Reverse | GTTTCTTCTGCCTTTTTTTX₁₉X₂₀TG | ASTE1 |
| P24x | 47 | Reverse | CTTTTTTTTX₂₁TGAATX₂₁TGATCTX₂₃C | ASTE1 |
| P29x | 48 | Reverse | GTTTCTTCTGCCTTTTTTX₂₄TX₂₅X₂₆G | ASTE1 |
| P31x | 49 | Reverse | GTTTCTTCTGCCTTTTTX₂₇X₂₈TTX₂₉G | ASTE1 |
| P37 | 50 | Forward | TGCACTTCTTGCCACAATG | TAF1β |
| P38 | 51 | Reverse | AGGCTTCTGCTTGTTGATAAAG | TAF1β |
| P39 | 52 | Forward | CCGGGGGCTTAAAAAAAAAAC | TAF1β |
| P40 | 53 | Forward | CCGGGGGCTTAAAAAAAAATC | TAF1β |
| P41 | 54 | Forward | CCGGGGGCTTAAAAAAAAAGC | TAF1β |
| P42 | 55 | Forward | CCGGGGGCTTAAAAAAAATTC | TAF1β |
| P43 | 56 | Forward | CCGGGGGCTTAAAAAAAAGTC | TAF1β |
| P44 | 57 | Forward | CCGGGGGCTTAAAAAATATTC | TAF1β |
| P45 | 58 | Forward | CCGGGGGCTTAAAAAAGATTC | TAF1β |
| P46 | 59 | Forward | CCGGGGGCTTAAAAAAGAGTC | TAF1β |
| P40x/P41x | 60 | Forward | CCGGGGGCTTAAAAAAAAAX₃₀C | TAF1β |
| P42x/P43x | 61 | Forward | CCGGGGGCTTAAAAAAAAX₃₁X₃₂C | TAF1β |
| P44x/P45x/ | 62 | Forward | CCGGGGGCTTAAAAAAX₃₃AX₃₄X₃₅C | TAF1β |
| P46x | | | | |
| P.AST.X1 | 83 | Reverse | GTTTCTTCTGCCTTTTTTX₃₆X₃₇X₃₈TG | ASTE1 |
| P.AST.54 | 84 | Reverse | GTTTCTTCTGCCTTTTTTGATGG | ASTE1 |
| P.AST.55 | 85 | Reverse | GTTTCTTCTGCCTTTTTTCATCG | ASTE1 |
| P.AST.59 | 86 | Reverse | GTTTCTTCTGCCTTTTTTAGGTG | ASTE1 |
| P.AST.60 | 87 | Reverse | GTTTCTTCTGCCTTTTTTAAGTG | ASTE1 |
| P.AST.61 | 88 | Reverse | GTTTCTTCTGCCTTTTTTACCTG | ASTE1 |
| P.AST.65 | 98 | Reverse | GTTTCTTCTGCCTTTTTTAGCTG | ASTE1 |
| P.AST.67 | 99 | Reverse | GTTTCTTCTGCCTTTTTTGGATG | ASTE1 |
| P.AST.X2 | 100 | Reverse | GTTTCTTCTGCCTTTTTX₄₂X₄₃X₄₄TTG | ASTE1 |
| P.AST.79 | 101 | Reverse | GTTTCTTCTGCCTTTTTACCTTG | ASTE1 |
| P.AST.2 | 102 | Reverse | ACTTGGTGTGTGCAGTG | ASTE1 |
| P.ACV.X | 89 | Forward | AGTTGTTGTGCATAAAAX₃₉X₄₀X₄₁GA | ACVR2A |
| P.ACV.21 | 90 | Forward | AGTTGTTGTGCATAAAATTTGA | ACVR2A |
| P.ACV.22 | 91 | Forward | AGTTGTTGTGCATAAAACCCGA | ACVR2A |
| P.ACV.1 | 92 | Reverse | GAATATCCTGTGTGAAGATCAC | ACVR2A |
| P.ACV.2 | 93 | Forward | GTTCCTTATGTCCTCTGTGC | ACVR2A |

| | |
|---|---|
| wild type TGFβR2(a10) | 5′-GGAAAAAAAAAAGCCTGGTGAGAC-3′ |
| microsatellite target | or |
| (SEQ ID NO: 63 and SEQ ID NO: 64, | 5′-GAAGGAAAAAAAAAAGCCTGGTG-3′ |
| respectively) | |
| mutant TGFβR2(a9) | 5′-GGAAAAAAAAAGCCTGGTGAGAC-3′ |
| microsatellite target | or |
| (SEQ ID NO: 65 and SEQ ID NO: 66, | 5′-GAAGGAAAAAAAAAGCCTGGTG-3′ |
| respectively) | |
| wild type ASTE1 (a11) | 5′-CAAAAAAAAAAAGGCAGAAGAAAC-3′ |
| microsatellite target | or |
| (SEQ ID NO: 67 and SEQ ID NO: 68, | 5′-GTAGATCAAATTCAAAAAAAAAAAG-3′ |
| respectively) | |
| mutant ASTE1 (a10) | 5′-CAAAAAAAAAAGGCAGAAGAAAC-3′ |
| microsatellite target | or |
| (SEQ ID NO: 69 and SEQ ID NO: 70, | 5′-GTAGATCAAATTCAAAAAAAAAAG-3′ |
| respectively) | |
| wild type TAF1β (a11) | 3′-GGCCCCCGAATTTTTTTTTTTG-5′ |
| microsatellite target | |
| (SEQ ID NO: 71) | |
| mutant TAF1β (a10) | 3′-GGCCCCCGAATTTTTTTTTTG-5′ |
| microsatellite target | |
| (SEQ ID NO: 72) | |

(bold underlined bases show mismatches to target sequence)

-wild type (a10) TGFβR2 full length;
SEQ ID NO: 1
ATGGGTCGGGGGCTGCTCAGGGGCCTGTGGCCGCTGCACATCGTCCTGTGGACGCGTAT
CGCCAGCACGATCCCACCGCACGTTCAGAAGTCGGATGTGGAAATGGAGGCCCAGAAAG
ATGAAATCATCTGCCCCAGCTGTAATAGGACTGCCCATCCACTGAGACATATTAATAACGA
CATGATAGTCACTGACAACAACGGTGCAGTCAAGTTTCCACAACTGTGTAAATTTTGTGATG
TGAGATTTTCCACCTGTGACAACCAGAAATCCTGCATGAGCAACTGCAGCATCACCTCCAT
CTGTGAGAAGCCACAGGAAGTCTGTGTGGCTGTATGGAGAAAGAATGACGAGAACATAAC
ACTAGAGACAGTTTGCCATGACCCCAAGCTCCCCTACCATGACTTTATTCTGGAAGATGCT
GCTTCTCCAAAGTGCATTATGAAGGAAAAAAAAAAGCCTGGTGAGACTTTCTTCATGTGTT
CCTGTAGCTCTGATGAGTGCAATGACAACATCATCTTCTCAGAAGAATATAACACCAGCAAT
CCTGACTTGTTGCTAGTCATATTTCAAGTGACAGGCATCAGCCTCCTGCCACCACTGGGAG
TTGCCATATCTGTCATCATCATCTTCTACTGCTACCGCGTTAACCGGCAGCAGAAGCTGAG
TTCAACCTGGGAAACCGGCAAGACGCGGAAGCTCATGGAGTTCAGCGAGCACTGTGCCAT
CATCCTGGAAGATGACCGCTCTGACATCAGCTCCACGTGTGCCAACAACATCAACCACAAC
ACAGAGCTGCTGCCCATTGAGCTGGACACCCTGGTGGGGAAAGGTCGCTTTGCTGAGGTC
TATAAGGCCAAGCTGAAGCAGAACACTTCAGAGCAGTTTGAGACAGTGGCAGTCAAGATCT
TTCCCTATGAGGAGTATGCCTCTTGGAAGACAGAGAAGGACATCTTCTCAGACATCAATCT
GAAGCATGAGAACATACTCCAGTTCCTGACGGCTGAGGAGCGGAAGACGGAGTTGGGGA
AACAATACTGGCTGATCACCGCCTTCCACGCCAAGGGCAACCTACAGGAGTACCTGACGC
GGCATGTCATCAGCTGGGAGGACCTGCGCAAGCTGGGCAGCTCCCTCGCCCGGGGGATT
GCTCACCTCCACAGTGATCACACTCCATGTGGGAGGCCCAAGATGCCCATCGTGCACAGG
GACCTCAAGAGCTCCAATATCCTCGTGAAGAACGACCTAACCTGCTGCCTGTGTGACTTTG
GGCTTTCCCTGCGTCTGGACCCTACTCTGTCTGTGGATGACCTGGCTAACAGTGGGCAGG
TGGGAACTGCAAGATACATGGCTCCAGAAGTCCTAGAATCCAGGATGAATTTGGAGAATGT
TGAGTCCTTCAAGCAGACCGATGTCTACTCCATGGCTCTGGTGCTCTGGGAAATGACATCT
CGCTGTAATGCAGTGGGAGAAGTAAAAGATTATGAGCCTCCATTTGGTTCCAAGGTGCGG
GAGCACCCCTGTGTCGAAAGCATGAAGGACAACGTGTTGAGAGATCGAGGGCGACCAGAA
ATTCCCAGCTTCTGGCTCAACCACCAGGGCATCCAGATGGTGTGTGAGACGTTGACTGAG
TGCTGGGACCACGACCCAGAGGCCCGTCTCACAGCCCAGTGTGTGGCAGAACGCTTCAG
TGAGCTGGAGCATCTGGACAGGCTCTCGGGGAGGAGCTGCTCGGAGGAGAAGATTCCTG
AAGACGGCTCCCTAAACACTACCAAATAG
-mutant TGFβR2 (a9) full length;
SEQ ID NO: 2
ATGGGTCGGGGGCTGCTCAGGGGCCTGTGGCCGCTGCACATCGTCCTGTGGACGCGTAT
CGCCAGCACGATCCCACCGCACGTTCAGAAGTCGGATGTGGAAATGGAGGCCCAGAAAG
ATGAAATCATCTGCCCCAGCTGTAATAGGACTGCCCATCCACTGAGACATATTAATAACGA
CATGATAGTCACTGACAACAACGGTGCAGTCAAGTTTCCACAACTGTGTAAATTTTGTGATG
TGAGATTTTCCACCTGTGACAACCAGAAATCCTGCATGAGCAACTGCAGCATCACCTCCAT
CTGTGAGAAGCCACAGGAAGTCTGTGTGGCTGTATGGAGAAAGAATGACGAGAACATAAC
ACTAGAGACAGTTTGCCATGACCCCAAGCTCCCCTACCATGACTTTATTCTGGAAGATGCT
GCTTCTCCAAAGTGCATTATGAAGGAAAAAAAAAGCCTGGTGAGACTTTCTTCATGTGTTC
CTGTAGCTCTGATGAGTGCAATGACAACATCATCTTCTCAGAAGAATATAACACCAGCAATC
CTGACTTGTTGCTAG
-wild type TGFβR2 (a10) fragment (F2);
SEQ ID NO: 3
TGCGAATGCTGGAGAACAGGAACCAGCTGCCGTTGTTAGGAACAACTTCATGAAGGAAAA
GTATTCCAGATTGCCTTTCTGTCTGGAGGCCATATTATTCATTTATTCTCTTTCTCTCTCTCC
CTCTCCCCTCGCTTCCAATGAATCTCTTCACTCTAGGAGAAAGAATGACGAGAACATAACA
CTAGAGACAGTTTGCCATGACCCCAAGCTCCCCTACCATGACTTTATTCTGGAAGATGCTG
CTTCTCCAAAGTGCATTATGAAGGAAAAAAAAAAGCCTGGTGAGACTTTCTTCATGTGTTC
CTGTAGCTCTGATGAGTGCAATGACAACATCATCTTCTCAGAAGGTGAGTTTTCTTCTCTTA
AGGGTGTGGGACCTGAGATCTGTGCCAATTTTTTGTATCCTTGGTCTGCAGTGTCATAGAG
CACATTCCTCCTGTGGTGGATTGCATAC
-mutant TGFβR2 (a9) fragment (F1);
SEQ ID NO: 4
TGCGAATGCTGGAGAACAGGAACCAGCTGCCGTTGTTAGGAACAACTTCATGAAGGAAAA
GTATTCCAGATTGCCTTTCTGTCTGGAGGCCATATTATTCATTTATTCTCTTTCTCTCTCTCC
CTCTCCCCTCGCTTCCAATGAATCTCTTCACTCTAGGAGAAAGAATGACGAGAACATAACA
CTAGAGACAGTTTGCCATGACCCCAAGCTCCCCTACCATGACTTTATTCTGGAAGATGCTG
CTTCTCCAAAGTGCATTATGAAGGAAAAAAAAAGCCTGGTGAGACTTTCTTCATGTGTTCC
TGTAGCTCTGATGAGTGCAATGACAACATCATCTTCTCAGAAGGTGAGTTTTCTTCTCTTAA
GGGTGTGGGACCTGAGATCTGTGCCAATTTTTTGTATCCTTGGTCTGCAGTGTCATAGAGC
ACATTCCTCCTGTGGTGGATTGCATAC
-wild type ASTE1 (a11) full length;
SEQ ID NO: 5
ATGGCCCATTCCCTTTCTGTTGGTGGGAGTGGGTACGTATGTCCCTTACTCATCCGGGAAG
TATTCATACAGGTTTTGATCAAGCTGCGGGTGTGTTTTGTCCAGTGCTTTTCAGAAGCAGAT
CGGGACATTATGACACTTGCTAACCATTGGAATTGCCCTGTGTTATCATCAGATAGTGACTT
TTGCATTTTTGACCTGAAAACTGGGTTTTGCCCATTGAATAGCTTTCAGTGGAGAAATATGA
ACACTATTAAGGGCACACAAAACTATATCCCTGCCAAATGCTTTTCCCTTGATGCATTCTGC
CATCACTTCAGCAATATGAATAAAGCTCTACTACCTCTCTTTGCGGTGCTATGTGGAAATGA
CCATGTTAATCTACCCATCATGGAGACATTCTTAAGTAAAGCGCGTCTTCCTCTTGGAGCTA
CCAGTTCTAAAGGGAGGAGACACCACCGAATCCTGGGACTTCTGAATTGGTTGTCTCATTT
TGCCAACCCTACCGAAGCACTAGATAATGTTCTGAAATACCTCCCAAAAAAGGATCGAGAA
AATGTTAAGGAACTTCTCTGCTGTTCCATGGAAGAATACCAACAGTCCCAGGTGAAGCTAC
AGGACTTCTTCCAGTGTGGTACTTATGTCTGTCCAGATGCCTTGAATCTTGGTTTACCAGAA
TGGGTATTAGTGGCTTTAGCTAAAGGCCAGCTATCTCCTTTCATCAGTGATGCTTTGGTCCT
AAGACGGACCATTCTTCCCACACAGGTGGAAAACATGCAGCAACCAAATGCCCACAGAATA
TCTCAGCCCATCAGGCAAATCATCTATGGGCTTCTTTTAAATGCCTCACCACATCTGGACAA
GACATCCTGGAATGCATTGCCTCCTCAGCCTCTAGCTTTCAGTGAAGTGGAAAGGATTAAT
AAAAATATCAGAACCTCAATCATTGATGCAGTAGAACTGGCCAAGGATCATTCTGACTTAAG
CAGATTGACTGAGCTCTCCTTGAGGAGGCGGCAGATGCTTCTGTTAGAAACCCTGAAGGT
GAAACAGACCATTCTGGAGCCAATCCCTACTTCACTGAAGTTGCCCATTGCTGTCAGTTGC
TACTGGTTGCAGCACACCGAGACCAAAGCAAAGCTACATCATCTACAATCCTTACTGCTCA
CAATGCTAGTGGGGCCCTTGATTGCCATAATCAACAGCCCTGGTAAGGAAGAGCTGCAGG
AAGATGGTGCTAAGATGTTGTATGCAGAGTTCCAAAGAGTGAAGGCGCAGACACGGCTGG
GCACAAGACTGGACTTAGACACAGCTCACATCTTCTGTCAGTGGCAGTCCTGTCTCCAGAT
GGGGATGTATCTCAACCAGCTGCTGTCCACTCCTCTCCCAGAGCCAGACCTAACTCGACT
GTACAGTGGAAGCCTGGTGCACGGACTATGCCAGCAACTGCTAGCATCGACCTCTGTAGA
AAGTCTCCTGAGCATATGTCCTGAGGCTAAGCAACTTTATGAATATCTATTCAATGCCACAA
GGTCATATGCCCCCGCTGAAATATTCCTACCAAAAGGTAGATCAAATTCAAAAAAAAAAAG
GCAGAAGAAACAGAATACCAGCTGTTCTAAGAACAGAGGGAGAACCACTGCACACACCAA
GTGTTGGTATGAGGGAAACAACCGGTTTGGGTTGTTAATGGTTGAAAACTTAGAGGAACAT
AGTGAGGCCTCCAACATTGAATAA
-mutant ASTE1 (a10) full length;
SEQ ID NO: 6
ATGGCCCATTCCCTTTCTGTTGGTGGGAGTGGGTACGTATGTCCCTTACTCATCCGGGAAG
TATTCATACAGGTTTTGATCAAGCTGCGGGTGTGTTTTGTCCAGTGCTTTTCAGAAGCAGAT
CGGGACATTATGACACTTGCTAACCATTGGAATTGCCCTGTGTTATCATCAGATAGTGACTT
TTGCATTTTTGACCTGAAAACTGGGTTTTGCCCATTGAATAGCTTTCAGTGGAGAAATATGA
ACACTATTAAGGGCACACAAAACTATATCCCTGCCAAATGCTTTTCCCTTGATGCATTCTGC
CATCACTTCAGCAATATGAATAAAGCTCTACTACCTCTCTTTGCGGTGCTATGTGGAAATGA
CCATGTTAATCTACCCATCATGGAGACATTCTTAAGTAAAGCGCGTCTTCCTCTTGGAGCTA
CCAGTTCTAAAGGGAGGAGACACCACCGAATCCTGGGACTTCTGAATTGGTTGTCTCATTT
TGCCAACCCTACCGAAGCACTAGATAATGTTCTGAAATACCTCCCAAAAAAGGATCGAGAA
AATGTTAAGGAACTTCTCTGCTGTTCCATGGAAGAATACCAACAGTCCCAGGTGAAGCTAC
AGGACTTCTTCCAGTGTGGTACTTATGTCTGTCCAGATGCCTTGAATCTTGGTTTACCAGAA
TGGGTATTAGTGGCTTTAGCTAAAGGCCAGCTATCTCCTTTCATCAGTGATGCTTTGGTCCT
AAGACGGACCATTCTTCCCACACAGGTGGAAAACATGCAGCAACCAAATGCCCACAGAATA
TCTCAGCCCATCAGGCAAATCATCTATGGGCTTCTTTTAAATGCCTCACCACATCTGGACAA
GACATCCTGGAATGCATTGCCTCCTCAGCCTCTAGCTTTCAGTGAAGTGGAAAGGATTAAT
AAAAATATCAGAACCTCAATCATTGATGCAGTAGAACTGGCCAAGGATCATTCTGACTTAAG
CAGATTGACTGAGCTCTCCTTGAGGAGGCGGCAGATGCTTCTGTTAGAAACCCTGAAGGT
GAAACAGACCATTCTGGAGCCAATCCCTACTTCACTGAAGTTGCCCATTGCTGTCAGTTGC
TACTGGTTGCAGCACACCGAGACCAAAGCAAAGCTACATCATCTACAATCCTTACTGCTCA
CAATGCTAGTGGGGCCCTTGATTGCCATAATCAACAGCCCTGGTAAGGAAGAGCTGCAGG
AAGATGGTGCTAAGATGTTGTATGCAGAGTTCCAAAGAGTGAAGGCGCAGACACGGCTGG
GCACAAGACTGGACTTAGACACAGCTCACATCTTCTGTCAGTGGCAGTCCTGTCTCCAGAT
GGGGATGTATCTCAACCAGCTGCTGTCCACTCCTCTCCCAGAGCCAGACCTAACTCGACT
GTACAGTGGAAGCCTGGTGCACGGACTATGCCAGCAACTGCTAGCATCGACCTCTGTAGA
AAGTCTCCTGAGCATATGTCCTGAGGCTAAGCAACTTTATGAATATCTATTCAATGCCACAA
GGTCATATGCCCCCGCTGAAATATTCCTACCAAAAGGTAGATCAAATTCAAAAAAAAAAGG
CAGAAGAAACAGAATACCAGCTGTTCTAAGAACAGAGGGAGAACCACTGCACACACCAAG
TGTTGGTATGAGGGAAACAACCGGTTTGGGTTGTTAA
-wild type ASTE1 (a11) fragment (F4);
SEQ ID NO: 7
AGAGTTCCAAAGAGTGAAGGCGCAGACACGGCTGGGCACAAGACTGGACTTAGACACAGC
TCACATCTTCTGTCAGTGGCAGTCCTGTCTCCAGATGGGGATGTATCTCAACCAGCTGCTG
TCCACTCCTCTCCCAGAGCCAGACCTAACTCGACTGTACAGTGGAAGCCTGGTGCACGGA
CTATGCCAGCAACTGCTAGCATCGACCTCTGTAGAAAGTCTCCTGAGCATATGTCCTGAGG
CTAAGCAACTTTATGAATATCTATTCAATGCCACAAGGTCATATGCCCCCGCTGAAATATTC
CTACCAAAAGGTAGATCAAATTCAAAAAAAAAAAGGCAGAAGAAACAGAATACCAGCTGTT
CTAAGAACAGAGGGAGAACCACTGCACACACCAAGTGTTGGTATGAGGGAAACAACCGGT
TTGGGTTGTTAATGGTTGAAAACTTAGAGGAACATAGTGAGGCCTCCAACATTGAATAAAAC
TCAGTTTG
-mutant ASTE1 (a10) fragment (F3);
SEQ ID NO: 8
AGAGTTCCAAAGAGTGAAGGCGCAGACACGGCTGGGCACAAGACTGGACTTAGACACAGC
TCACATCTTCTGTCAGTGGCAGTCCTGTCTCCAGATGGGGATGTATCTCAACCAGCTGCTG
TCCACTCCTCTCCCAGAGCCAGACCTAACTCGACTGTACAGTGGAAGCCTGGTGCACGGA
CTATGCCAGCAACTGCTAGCATCGACCTCTGTAGAAAGTCTCCTGAGCATATGTCCTGAGG
CTAAGCAACTTTATGAATATCTATTCAATGCCACAAGGTCATATGCCCCCGCTGAAATATTC
CTACCAAAAGGTAGATCAAATTCAAAAAAAAAAGGCAGAAGAAACAGAATACCAGCTGTTC
TAAGAACAGAGGGAGAACCACTGCACACACCAAGTGTTGGTATGAGGGAAACAACCGGTT
TGGGTTGTTAATGGTTGAAAACTTAGAGGAACATAGTGAGGCCTCCAACATTGAATAAAACT
CAGTTTG
-wild type TAF1β (a11) full length;
SEQ ID NO: 9
ATGGACCTCGAGGAGGCGGAAGAGTTTAAAGAACGCTGTACTCAGTGTGCTGCTGTCTCA
TGGGGTCTTACTGATGAAGGCAAATATTATTGCACTTCTTGCCACAATGTTACAGAGAGATA
TCAGGAAGTTACAAACACTGATCTTATTCCTAATACCCAAATAAAAGCCCTCAACCGGGGG
CTTAAAAAAAAAAACAATACTGAAAAAGGCTGGGATTGGTATGTGTGTGAAGGTTTCCAGT
ATATTCTTTATCAACAAGCAGAAGCCTTAAAGAACCTTGGAGTAGGCCCAGAGTTAAAGAA
CGATGTTTTACATAATTTTTGGAAGCGCTACCTTCAGAAGAGCAAGCAGGCATATTGTAAGA
ACCCAGTTTATACCACTGGAAGGAAACCTACGGTATTAGAAGATAATCTAAGTCATTCAGAC
TGGGCTAGTGAGCCTGAGCTGCTAAGTGATGTCAGCTGTCCTCCTTTTCTTGAAAGTGGAG
CGGAGTCTCAGTCTGACATCCACACTCGAAAACCTTTCCCCGTCAGCAAAGCATCACAATC
AGAAACGTCTGTCTGCTCTGGATCTCTGGATGGAGTTGAATACTCACAACGAAAGGAGAAG
GGAATCGTGAAGATGACCATGCCACAGACACTTGCCTTCTGTTATCTGTCCTTACTTTGGC
AGAGAGAAGCAATAACACTTTCAGATCTTTTGAGGTTTGTTGAAGAGGACCATATTCCTTAC
ATAAATGCTTTTCAGCATTTTCCAGAACAGATGAAATTATATGGACGTGACAGAGGAATCTT
TGGTATAGAGTCTTGGCCTGACTACGAGGACATCTACAAAAAAACAGTAGAAGTTGGAACA
TTTTTAGATTTGCCTCGTTTTCCAGACATAACTGAAGACTGCTATCTTCATCCCAACATACTG
TGTATGAAATACTTGATGGAAGTCAACCTCCCTGATGAAATGCATAGCTTAACTTGCCACGT
GGTAAAAATGACTGGAATGGGAGAAGTGGATTTTCTGACATTTGATCCTATAGCTAAAATG
GCAAAAACTGTTAAGTACGATGTACAAGCTGTAGCTATCATTGTGGTGGTATTGAAACTGCT
CTTTCTATTGGATGACAGTTTCGAGTGGTCTTTGTCTAATCTTGCTGAAAAGCATAATGAAA
AGAACAAAAAAGATAAGCCATGGTTTGATTTCAGAAAGTGGTACCAAATTATGAAGAAAGCT
TTTGATGAGAAAAAACAAAAATGGGAAGAAGCAAGGGCCAAGTACCTGTGGAAAAGTGAAA
AGCCACTCTACTACTCATTTGTCGACAAACCAGTAGCATATAAAAAAAGAGAAATGGTGGT
GAATCTACAGAAACAATTTAGCACACTGGTCGAGTCAACAGCAACTGCTGGAAAAAAAAGC
CCTTCAAGTTTTCAGTTCAACTGGACTGAAGAGGACACTGATAGAACGTGTTTCCATGGAC
ACAGCCTTCAGGGAGTCCTGAAAGAGAAAGGCCAATCACTGCTGACTAAGAATTCATTATA
TTGGCTTAGTACACAGAAATTCTGCAGATGCTATTGTACACATGTGACAACCTATGAAGAAT
CAAATTATTCTCTGAGTTATCAGTTTATACTAAATCTCTTCTCCTTCCTGCTCAGAATAAAGA
CTTCCCTTCTCCATGAAGAAGTGAGCTTAGTTGAGAAGAAACTTTTTGAGAAAAAATACAGT
GTAAAAAGAAAGAAATCAAGATCCAAGAAAGTGAGACGACATTGA
-mutant TAF1β (a10) full length;
SEQ ID NO: 10
ATGGACCTCGAGGAGGCGGAAGAGTTTAAAGAACGCTGTACTCAGTGTGCTGCTGTCTCA
TGGGGTCTTACTGATGAAGGCAAATATTATTGCACTTCTTGCCACAATGTTACAGAGAGATA
TCAGGAAGTTACAAACACTGATCTTATTCCTAATACCCAAATAAAAGCCCTCAACCGGGGG
CTTAAAAAAAAAACAATACTGAAAAAGGCTGGGATTGGTATGTGTGTGAAGGTTTCCAGTA
TATTCTTTATCAACAAGCAGAAGCCTTAAAGAACCTTGGAGTAGGCCCAGAGTTAAAGAAC
GATGTTTTACATAATTTTTGGAAGCGCTACCTTCAGAAGAGCAAGCAGGCATATTGTAAGAA
CCCAGTTTATACCACTGGAAGGAAACCTACGGTATTAGAAGATAATCTAAGTCATTCAGACT
GGGCTAGTGAGCCTGAGCTGCTAAGTGATGTCAGCTGTCCTCCTTTTCTTGAAAGTGGAGC
GGAGTCTCAGTCTGACATCCACACTCGAAAACCTTTCCCCGTCAGCAAAGCATCACAATCA
GAAACGTCTGTCTGCTCTGGATCTCTGGATGGAGTTGAATACTCACAACGAAAGGAGAAGG
GAATCGTGAAGATGACCATGCCACAGACACTTGCCTTCTGTTATCTGTCCTTACTTTGGCA
GAGAGAAGCAATAACACTTTCAGATCTTTTGAGGTTTGTTGAAGAGGACCATATTCCTTACA
TAAATGCTTTTCAGCATTTTCCAGAACAGATGAAATTATATGGACGTGACAGAGGAATCTTT
GGTATAGAGTCTTGGCCTGACTACGAGGACATCTACAAAAAAACAGTAGAAGTTGGAACAT
TTTTAGATTTGCCTCGTTTTCCAGACATAACTGAAGACTGCTATCTTCATCCCAACATACTGT
GTATGAAATACTTGATGGAAGTCAACCTCCCTGATGAAATGCATAGCTTAACTTGCCACGT
GGTAAAAATGACTGGAATGGGAGAAGTGGATTTTCTGACATTTGATCCTATAGCTAAAATG
GCAAAAACTGTTAAGTACGATGTACAAGCTGTAGCTATCATTGTGGTGGTATTGAAACTGCT
CTTTCTATTGGATGACAGTTTCGAGTGGTCTTTGTCTAATCTTGCTGAAAAGCATAATGAAA
AGAACAAAAAAGATAAGCCATGGTTTGATTTCAGAAAGTGGTACCAAATTATGAAGAAAGCT
TTTGATGAGAAAAAACAAAAATGGGAAGAAGCAAGGGCCAAGTACCTGTGGAAAAGTGAAA
AGCCACTCTACTACTCATTTGTCGACAAACCAGTAGCATATAAAAAAAGAGAAATGGTGGT
GAATCTACAGAAACAATTTAGCACACTGGTCGAGTCAACAGCAACTGCTGGAAAAAAAAGC
CCTTCAAGTTTTCAGTTCAACTGGACTGAAGAGGACACTGATAGAACGTGTTTCCATGGAC
ACAGCCTTCAGGGAGTCCTGAAAGAGAAAGGCCAATCACTGCTGACTAAGAATTCATTATA
TTGGCTTAGTACACAGAAATTCTGCAGATGCTATTGTACACATGTGACAACCTATGAAGAAT
CAAATTATTCTCTGAGTTATCAGTTTATACTAAATCTCTTCTCCTTCCTGCTCAGAATAAAGA
CTTCCCTTCTCCATGAAGAAGTGAGCTTAGTTGAGAAGAAACTTTTTGAGAAAAAATACAGT
GTAAAAAGAAAGAAATCAAGATCCAAGAAAGTGAGACGACATTGA
-wild type TAF1β (a11) fragment (F6);
SEQ ID NO: 11
CTTTCCCGGAAGCTGCGCTCGCTACCCGGGTAACGGGTCCCGGCTGTGGAAGCTCCCGC
GGCGCCGCGATGGACCTCGAGGAGGCGGAAGAGTTTAAAGAACGCTGTACTCAGTGTGC
TGCTGTCTCATGGGGTCTTACTGATGAAGGCAAATATTATTGCACTTCTTGCCACAATGTTA
CAGAGAGATATCAGGAAGTTACAAACACTGATCTTATTCCTAATACCCAAATAAAAGCCCTC
AACCGGGGGCTTAAAAAAAAAAACAATACTGAAAAAGGCTGGGATTGGTATGTGTGTGAA
GGTTTCCAGTATATTCTTTATCAACAAGCAGAAGCCTTAAAGAACCTTGGAGTAGGCCCAG
AGTTAAAGAACGATGTTTTACATAATTTTTGGAAGCGCTACCTTCAGAAGAGCAAGCAGGC
ATATTGTAAGAACCCAGTTTATACCACTGGAAGGAAACCTACGGTATTAGAAGATAATCTAA
GTCATTCAGACTG
-mutant TAF1ß (a10) fragment (F5);
SEQ ID NO: 12
CTTTCCCGGAAGCTGCGCTCGCTACCCGGGTAACGGGTCCCGGCTGTGGAAGCTCCCGC
GGCGCCGCGATGGACCTCGAGGAGGCGGAAGAGTTTAAAGAACGCTGTACTCAGTGTGC
TGCTGTCTCATGGGGTCTTACTGATGAAGGCAAATATTATTGCACTTCTTGCCACAATGTTA
CAGAGAGATATCAGGAAGTTACAAACACTGATCTTATTCCTAATACCCAAATAAAAGCCCTC
AACCGGGGGCTTAAAAAAAAAACAATACTGAAAAAGGCTGGGATTGGTATGTGTGTGAAG
GTTTCCAGTATATTCTTTATCAACAAGCAGAAGCCTTAAAGAACCTTGGAGTAGGCCCAGA
GTTAAAGAACGATGTTTTACATAATTTTTGGAAGCGCTACCTTCAGAAGAGCAAGCAGGCAT
ATTGTAAGAACCCAGTTTATACCACTGGAAGGAAACCTACGGTATTAGAAGATAATCTAAGT
CATTCAGACTG
-wild type (a8) ACVR2A full length
SEQ ID NO: 94
GTCTGGGCTTCCGAATATGTTTTATGACGGTTGATTTTACACCAGGAGGTTTGTCTCCGAG
GAAGACCCAGGGAACTGGATATCTAGCGAGAACTTCCTCCGGATTCCCCGGCGCCTCGGG
AAAATGGGAGCTGCTGCAAAGTTGGCGTTTGCCGTCTTTCTTATCTCCTGTTCTTCAGGTG
CTATACTTGGTAGATCAGAAACTCAGGAGTGTCTTTTCTTTAATGCTAATTGGGAAAAAGAC
AGAACCAATCAAACTGGTGTTGAACCGTGTTATGGTGACAAAGATAAACGGCGGCATTGTT
TTGCTACCTGGAAGAATATTTCTGGTTCCATTGAAATAGTGAAACAAGGTTGTTGGCTGGAT
GATATCAACTGCTATGACAGGACTGATTGTGTAGAAAAAAAAGACAGCCCTGAAGTATATTT
TTGTTGCTGTGAGGGCAATATGTGTAATGAAAAGTTTTCTTATTTTCCGGAGATGGAAGTCA
CACAGCCCACTTCAAATCCAGTTACACCTAAGCCACCCTATTACAACATCCTGCTCTATTCC
TTGGTGCCACTTATGTTAATTGCGGGGATTGTCATTTGTGCATTTTGGGTGTACAGGCATCA
CAAGATGGCCTACCCTCCTGTACTTGTTCCAACTCAAGACCCAGGACCACCCCCACCTTCT
CCATTACTAGGTTTGAAACCACTGCAGTTATTAGAAGTGAAAGCAAGGGGAAGATTTGGTT
GTGTCTGGAAAGCCCAGTTGCTTAACGAATATGTGGCTGTCAAAATATTTCCAATACAGGA
CAAACAGTCATGGCAAAATGAATACGAAGTCTACAGTTTGCCTGGAATGAAGCATGAGAAC
ATATTACAGTTCATTGGTGCAGAAAAACGAGGCACCAGTGTTGATGTGGATCTTTGGCTGA
TCACAGCATTTCATGAAAAGGGTTCACTATCAGACTTTCTTAAGGCTAATGTGGTCTCTTGG
AATGAACTGTGTCATATTGCAGAAACCATGGCTAGAGGATTGGCATATTTACATGAGGATAT
ACCTGGCCTAAAAGATGGCCACAAACCTGCCATATCTCACAGGGACATCAAAAGTAAAAAT
GTGCTGTTGAAAAACAACCTGACAGCTTGCATTGCTGACTTTGGGTTGGCCTTAAAATTTGA
GGCTGGCAAGTCTGCAGGCGATACCCATGGACAGGTTGGTACCCGGAGGTACATGGCTC
CAGAGGTATTAGAGGGTGCTATAAACTTCCAAAGGGATGCATTTTTGAGGATAGATATGTAT
GCCATGGGATTAGTCCTATGGGAACTGGCTTCTCGCTGTACTGCTGCAGATGGACCTGTA
GATGAATACATGTTGCCATTTGAGGAGGAAATTGGCCAGCATCCATCTCTTGAAGACATGC
AGGAAGTTGTTGTGCATAAAAAAAAGAGGCCTGTTTTAAGAGATTATTGGCAGAAACATGC
TGGAATGGCAATGCTCTGTGAAACCATTGAAGAATGTTGGGATCACGACGCAGAAGCCAG
GTTATCAGCTGGATGTGTAGGTGAAAGAATTACCCAGATGCAGAGACTAACAAATATTATTA
CCACAGAGGACATTGTAACAGTGGTCACAATGGTGACAAATGTTGACTTTCCTCCCAAAGA
ATCTAGTCTATGATGGTTGCGCCATCTGTGCACACTAAGAAATGGGACTCTGAACTGGAGC
TGCTAAGCTAAAGAAACTGCTTACAGTTTATTTTCTGTGTAAAATGAGTAGGATGTCTCTTG
GAAATGTTAAGAAAGAAGACCCTTTGTTGAAAAATGTTGCTCTGGGAGACTTACTGCATTGC
CGACAGCACAGATGTGAAGGACATGAGACTAAGAGAAACCTTGCAAACTCTATAAAGAAAC
TTTTGAAAAAGTGTACATGAAGAATGTAGCCCTCTCCAAATCAAGGATCTTTTGGACCTGG
CTAATGGAGTGTTTGAAAACTGACATCAGATTTCTTAATGTCTGTCAGAAGACACTAATTCC
TTAAATGAACTACTGCTATTTTTTTTAAATCAAAAACTTTTCATTTCAGATTTTAAAAAGGGTA
ACTTGTTTTTATTGCATTTGCTGTTGTTTCTATAAATGACTATTGTAATGCCAATATGACACA
GCTTGTGAATGTTTAGTGTGCTGCTGTTCTGTGTACATAAAGTCATCAAAGTGGGGTACAG
TAAAGAGGCTTCCAAGCATTACTTTAACCTCCCTCAACAAGGTATACCTCAGTTCCACGGTT
GCTAAATTATAAAATTGAAAACACTAACAAAATTTGAATAATAAATCGATCCATGTTTTGTAA
CAAATTCACTGTGTTATTTAAGGAAAAAAAGGTAAGCTATGCTTAGTGCCAACAATAAGTGG
CCATTCGTAAAGCAGTGTTTTAGCATTTCTTGTGCTGGCTTGTAATGTAGGGAAAAAAAGTG
CTGTTTTTTGAAAAGATGGTGTCATTTCCCCCTTCTTCCCATGTTTTAAAGCCCCATCTTATA
TCCAGTTCCCAAAATTTGCATACTTACCTAAGTATTTTTTTTAGGTGTGCTGTGTTTGGGGA
ATATTTGAAAATTTAAAGCATGATTTAAAATTTTTTAAAGTGAGCTGTGACACTGGAAAGCTC
TTCATTTTATCTTTTAAAATAGAGTTTTTTCTATTTATATATGTAAAATTGTAGTGTATTTCTTT
TCACCAAACAGTGTGTGGGACATTCTTTATCACTGTTTTAGGATCACCTCAGGAAGTGTCGT
TACCCAGAATTCCCCACTGTCTGCTATGAGACTTGTAACTTTATCACTATACTTCTGCTTGG
TGCCATCTTGTCAGAGTAATATTTGATGTCTGTGATATGTAAAGAATTATCCTAGGATAAAG
ATATTAAACTTTAAGCAGATTTCAGATGTTACTGCTTTAAAACAAATCAGGGATAACAAATTA
AACGTATAACTTAAAATATGCAATGACATTTAGAGGTAACCAATGTTGATATAGGTAGCATA
GCCTAGCCTCCTCCCCAAAATTGCTTTTACAACTAACACTGATACTAATTTAGGATAGTTCA
TGCCTTATCCTTGCTAAGAAAATGGAATTGATGGTAGGCAGGTGCTAAAGTGCTTTTCAAAA
CAATATTACGTTAGAATACAATTGGATTCTTCCTCAAATTTATACAGGCCAAAAAGTAAAACA
TTAATTTTCTGAATTTCCAGATTACCAATCAATTAATCAACAAATAGCCAGTATTATGCTGTG
TATTTCTGTCAGGTCATTTTAAAATCCATGTTAATTTTATAAAAGAATTTTTTACATGTCACTG
TCAGGAGCTCACTGTGAATGTGTTGTCTTCAAATGGTTATTTAACCACACAGTACACTACAT
TTTACATATATGTACGTAATCTCTGGGAATAGTAAATTAATTATGTTATTTATAAACAATACAT
AGGTCAACAGACTTTAAGCAGGGAGGAAAAGAAGAGTAATAGCGTCTGTGTGCTGCAGAC
CATTCAGAACTGTCACGTGTGTCCCCATGGTCTCATTCATTGTATTCCTAGCAATTCCCTTT
TCAATGTTGAGTTCACCTCTTTATTTCACAAAGTACTTGGTCTCTCAATTTCTTGATCTGGTT
TTGCTTCCATTTAAAAACTAATCAAGAAGGGAAAATATTGAGAATGTGCATACAAGAAAATC
ATTAATTTCCTGAAGATGAATTTCTACCTGTTGTGAACATTTAACTTTCTTTTTAAAAGTTAAA
CAAAAATAAACAAGGGATATTATGATGAATGTTTGGCTTATGTGAGTACTAGAGATAAAATT
TTTAAACCCAGTTATTCACAATATAAAATGTTTTCAAGTTAGAAAAAATTTTTAGAAATCCTG
GGTATTGTATTTAACTGTAGCTAACCAATTTTAAAACTTGTATTCTTTTGAGAACTATTATTAA
TAGAAAAACTTTTTATAAGCAGTAAAATAAGAATGTTCCAGTGACTACCTGTCCTTATACCTA
GTCTTGTTAAAACTTTCTTTTGCAGGGTATTTAGTGTTTGGTTTACAGTCAGTGCAGAGTGG
GCAAGTTAACAGAAAGTTTGAGCTAGAGATACTGGAAAAAAAAAAGATCAAAGAATGAGAA
AAATGGTGATCCATTTTGGGGCAAACTGAGACCCCCCAAATAACTCTTTCCTCATGTGTATG
GTGCTCCTCATGACTCGTCTTGTATTTTGCCTTTCTGATACCCATCAGAACTGCTGCTGCTC
TAACTTATACTCTTTACCTTGCCCAGATCTCCGCGTAAGGAATGCTTTATGATCAACTTGCC
ATAGGACTGATGGATTAACCAGTGTTCGGCTTTATTTGAAGTCTATGCCCTGCACAGCTCTT
GTATGTATTTTAGATGCTAGAAGTTTTTTTAGCATGTGATGTGTGATTCTTGTTTGAATTCTA
GGTACCTTGTGAATTCCAGAAAAAGAGACTGTGCTTCACGATTGTTAGTCCCATGAACTTG
CACTATCTATCTTTCATGGTGATGTTTTGAAAATACAATCAGGAAAAAACCCAACACCTTTG
GAATTTAAAATAGAATCATATCATGAAATTTAAAAAGAATCTCTTCTGTTGCATTTCCTCACC
CCTAAGTAACAGCTACATTTAAGTAAAATGCAGGTGGTAGGGGAAAAAAAACCATGGCGAG
ATGGTGGTTTAGTGGAATAAACTGATTACTGGTTTTTTTGTTTTTTTTTTTTTTTTTAAAGAAA
GAAGCTTCATCACAGATACTTTCCAGTTTCTCTTTTATACTTTTTTGAAAGATTACTTTTTAG
GAACATTTGGTATGATATGCATAAAATTATTTATCCATTTATGGGCAAAATGATACAAGTAGC
ATCTTGATTGAACATCATTTACCTCAGATATTCAACCAGCAGTACGTTTTTTATGCAGTCTCA
ACCCATATCCCATTTGTTACCTCTCAGAATATTGGTAAGCAGTTATTTTCGCTTTACTCTGTA
TTTCTTGTGTTTTGGGCACAGGTTATTGTACTACTGTCAAATCGTACTTGCTATTTTTTCTGC
AAGTATTTAACAGAAAGCTTAAAATCCCCATAAAACCCCACCTTGGATAAGTGATTGTTAAA
TATTGTACAAATAAAATGTATGCTATCCCCATTCCATCCCCAAGTTAAATAAAAAAATGAATA
CGG
-mutant (a7) exon 10 ACVR2A full length
SEQ ID NO: 95
GTCTGGGCTTCCGAATATGTTTTATGACGGTTGATTTTACACCAGGAGGTTTGTCTCCGAG
GAAGACCCAGGGAACTGGATATCTAGCGAGAACTTCCTCCGGATTCCCCGGCGCCTCGGG
AAAATGGGAGCTGCTGCAAAGTTGGCGTTTGCCGTCTTTCTTATCTCCTGTTCTTCAGGTG
CTATACTTGGTAGATCAGAAACTCAGGAGTGTCTTTTCTTTAATGCTAATTGGGAAAAAGAC
AGAACCAATCAAACTGGTGTTGAACCGTGTTATGGTGACAAAGATAAACGGCGGCATTGTT
TTGCTACCTGGAAGAATATTTCTGGTTCCATTGAAATAGTGAAACAAGGTTGTTGGCTGGAT
GATATCAACTGCTATGACAGGACTGATTGTGTAGAAAAAAAAGACAGCCCTGAAGTATATTT
TTGTTGCTGTGAGGGCAATATGTGTAATGAAAAGTTTTCTTATTTTCCGGAGATGGAAGTCA
CACAGCCCACTTCAAATCCAGTTACACCTAAGCCACCCTATTACAACATCCTGCTCTATTCC
TTGGTGCCACTTATGTTAATTGCGGGGATTGTCATTTGTGCATTTTGGGTGTACAGGCATCA
CAAGATGGCCTACCCTCCTGTACTTGTTCCAACTCAAGACCCAGGACCACCCCCACCTTCT
CCATTACTAGGTTTGAAACCACTGCAGTTATTAGAAGTGAAAGCAAGGGGAAGATTTGGTT
GTGTCTGGAAAGCCCAGTTGCTTAACGAATATGTGGCTGTCAAAATATTTCCAATACAGGA
CAAACAGTCATGGCAAAATGAATACGAAGTCTACAGTTTGCCTGGAATGAAGCATGAGAAC
ATATTACAGTTCATTGGTGCAGAAAAACGAGGCACCAGTGTTGATGTGGATCTTTGGCTGA
TCACAGCATTTCATGAAAAGGGTTCACTATCAGACTTTCTTAAGGCTAATGTGGTCTCTTGG
AATGAACTGTGTCATATTGCAGAAACCATGGCTAGAGGATTGGCATATTTACATGAGGATAT
ACCTGGCCTAAAAGATGGCCACAAACCTGCCATATCTCACAGGGACATCAAAAGTAAAAAT
GTGCTGTTGAAAAACAACCTGACAGCTTGCATTGCTGACTTTGGGTTGGCCTTAAAATTTGA
GGCTGGCAAGTCTGCAGGCGATACCCATGGACAGGTTGGTACCCGGAGGTACATGGCTC
CAGAGGTATTAGAGGGTGCTATAAACTTCCAAAGGGATGCATTTTTGAGGATAGATATGTAT
GCCATGGGATTAGTCCTATGGGAACTGGCTTCTCGCTGTACTGCTGCAGATGGACCTGTA
GATGAATACATGTTGCCATTTGAGGAGGAAATTGGCCAGCATCCATCTCTTGAAGACATGC
AGGAAGTTGTTGTGCATAAAAAAAGAGGCCTGTTTTAAGAGATTATTGGCAGAAACATGC
TGGAATGGCAATGCTCTGTGAAACCATTGAAGAATGTTGGGATCACGACGCAGAAGCCAG
GTTATCAGCTGGATGTGTAGGTGAAAGAATTACCCAGATGCAGAGACTAACAAATATTATTA
CCACAGAGGACATTGTAACAGTGGTCACAATGGTGACAAATGTTGACTTTCCTCCCAAAGA
ATCTAGTCTATGATGGTTGCGCCATCTGTGCACACTAAGAAATGGGACTCTGAACTGGAGC
TGCTAAGCTAAAGAAACTGCTTACAGTTTATTTTCTGTGTAAAATGAGTAGGATGTCTCTTG
GAAATGTTAAGAAAGAAGACCCTTTGTTGAAAAATGTTGCTCTGGGAGACTTACTGCATTGC
CGACAGCACAGATGTGAAGGACATGAGACTAAGAGAAACCTTGCAAACTCTATAAAGAAAC
TTTTGAAAAAGTGTACATGAAGAATGTAGCCCTCTCCAAATCAAGGATCTTTTGGACCTGG
CTAATGGAGTGTTTGAAAACTGACATCAGATTTCTTAATGTCTGTCAGAAGACACTAATTCC
TTAAATGAACTACTGCTATTTTTTTTAAATCAAAAACTTTTCATTTCAGATTTTAAAAAGGGTA
ACTTGTTTTTATTGCATTTGCTGTTGTTTCTATAAATGACTATTGTAATGCCAATATGACACA
GCTTGTGAATGTTTAGTGTGCTGCTGTTCTGTGTACATAAAGTCATCAAAGTGGGGTACAG
TAAAGAGGCTTCCAAGCATTACTTTAACCTCCCTCAACAAGGTATACCTCAGTTCCACGGTT
GCTAAATTATAAAATTGAAAACACTAACAAAATTTGAATAATAAATCGATCCATGTTTTGTAA
CAAATTCACTGTGTTATTTAAGGAAAAAAAGGTAAGCTATGCTTAGTGCCAACAATAAGTGG
CCATTCGTAAAGCAGTGTTTTAGCATTTCTTGTGCTGGCTTGTAATGTAGGGAAAAAAAGTG
CTGTTTTTTGAAAAGATGGTGTCATTTCCCCCTTCTTCCCATGTTTTAAAGCCCCATCTTATA
TCCAGTTCCCAAAATTTGCATACTTACCTAAGTATTTTTTTTAGGTGTGCTGTGTTTGGGGA
ATATTTGAAAATTTAAAGCATGATTTAAAATTTTTTAAAGTGAGCTGTGACACTGGAAAGCTC
TTCATTTTATCTTTTAAAATAGAGTTTTTTCTATTTATATATGTAAAATTGTAGTGTATTTCTTT
TCACCAAACAGTGTGTGGGACATTCTTTATCACTGTTTTAGGATCACCTCAGGAAGTGTCGT
TACCCAGAATTCCCCACTGTCTGCTATGAGACTTGTAACTTTATCACTATACTTCTGCTTGG
TGCCATCTTGTCAGAGTAATATTTGATGTCTGTGATATGTAAAGAATTATCCTAGGATAAAG
ATATTAAACTTTAAGCAGATTTCAGATGTTACTGCTTTAAAACAAATCAGGGATAACAAATTA
AACGTATAACTTAAAATATGCAATGACATTTAGAGGTAACCAATGTTGATATAGGTAGCATA
GCCTAGCCTCCTCCCCAAAATTGCTTTTACAACTAACACTGATACTAATTTAGGATAGTTCA
TGCCTTATCCTTGCTAAGAAAATGGAATTGATGGTAGGCAGGTGCTAAAGTGCTTTTCAAAA
CAATATTACGTTAGAATACAATTGGATTCTTCCTCAAATTTATACAGGCCAAAAAGTAAAACA
TTAATTTTCTGAATTTCCAGATTACCAATCAATTAATCAACAAATAGCCAGTATTATGCTGTG
TATTTCTGTCAGGTCATTTTAAAATCCATGTTAATTTTATAAAAGAATTTTTTACATGTCACTG
TCAGGAGCTCACTGTGAATGTGTTGTCTTCAAATGGTTATTTAACCACACAGTACACTACAT
TTTACATATATGTACGTAATCTCTGGGAATAGTAAATTAATTATGTTATTTATAAACAATACAT
AGGTCAACAGACTTTAAGCAGGGAGGAAAAGAAGAGTAATAGCGTCTGTGTGCTGCAGAC
CATTCAGAACTGTCACGTGTGTCCCCATGGTCTCATTCATTGTATTCCTAGCAATTCCCTTT
TCAATGTTGAGTTCACCTCTTTATTTCACAAAGTACTTGGTCTCTCAATTTCTTGATCTGGTT
TTGCTTCCATTTAAAAACTAATCAAGAAGGGAAAATATTGAGAATGTGCATACAAGAAAATC
ATTAATTTCCTGAAGATGAATTTCTACCTGTTGTGAACATTTAACTTTCTTTTTAAAAGTTAAA
CAAAAATAAACAAGGGATATTATGATGAATGTTTGGCTTATGTGAGTACTAGAGATAAAATT
TTTAAACCCAGTTATTCACAATATAAAATGTTTTCAAGTTAGAAAAAATTTTTAGAAATCCTG
GGTATTGTATTTAACTGTAGCTAACCAATTTTAAAACTTGTATTCTTTTGAGAACTATTATTAA
TAGAAAAACTTTTTATAAGCAGTAAAATAAGAATGTTCCAGTGACTACCTGTCCTTATACCTA
GTCTTGTTAAAACTTTCTTTTGCAGGGTATTTAGTGTTTGGTTTACAGTCAGTGCAGAGTGG
GCAAGTTAACAGAAAGTTTGAGCTAGAGATACTGGAAAAAAAAAAGATCAAAGAATGAGAA
AAATGGTGATCCATTTTGGGGCAAACTGAGACCCCCCAAATAACTCTTTCCTCATGTGTATG
GTGCTCCTCATGACTCGTCTTGTATTTTGCCTTTCTGATACCCATCAGAACTGCTGCTGCTC
TAACTTATACTCTTTACCTTGCCCAGATCTCCGCGTAAGGAATGCTTTATGATCAACTTGCC
ATAGGACTGATGGATTAACCAGTGTTCGGCTTTATTTGAAGTCTATGCCCTGCACAGCTCTT
GTATGTATTTTAGATGCTAGAAGTTTTTTTAGCATGTGATGTGTGATTCTTGTTTGAATTCTA
GGTACCTTGTGAATTCCAGAAAAAGAGACTGTGCTTCACGATTGTTAGTCCCATGAACTTG
CACTATCTATCTTTCATGGTGATGTTTTGAAAATACAATCAGGAAAAAACCCAACACCTTTG
GAATTTAAAATAGAATCATATCATGAAATTTAAAAAGAATCTCTTCTGTTGCATTTCCTCACC
CCTAAGTAACAGCTACATTTAAGTAAAATGCAGGTGGTAGGGGAAAAAAAACCATGGCGAG
ATGGTGGTTTAGTGGAATAAACTGATTACTGGTTTTTTTGTTTTTTTTTTTTTTTTTAAAGAAA
GAAGCTTCATCACAGATACTTTCCAGTTTCTCTTTTATACTTTTTTGAAAGATTACTTTTTAG
GAACATTTGGTATGATATGCATAAAATTATTTATCCATTTATGGGCAAAATGATACAAGTAGC
ATCTTGATTGAACATCATTTACCTCAGATATTCAACCAGCAGTACGTTTTTTATGCAGTCTCA
ACCCATATCCCATTTGTTACCTCTCAGAATATTGGTAAGCAGTTATTTTCGCTTTACTCTGTA
TTTCTTGTGTTTTGGGCACAGGTTATTGTACTACTGTCAAATCGTACTTGCTATTTTTTCTGC
AAGTATTTAACAGAAAGCTTAAAATCCCCATAAAACCCCACCTTGGATAAGTGATTGTTAAA
TATTGTACAAATAAAATGTATGCTATCCCCATTCCATCCCCAAGTTAAATAAAAAAATGAATA
CGG
-wild type ACVR2A (a8) fragment
SEQ ID NO: 96
CATGGTTATAATAGATTGAACCTAGAGCCTCCAGAGCTGGAAGCACCACTGTTAACATTCT
GATCTGAATTGTAAGGAATGGGTACCTTAAAGGGATATTATATAACGTTAATTTACAAATACT
GATTGTTCCTTATGTCCTCTGTGCAGATGGAAATAGGCATCCTTTATACCTAGATAAGAAAG
CCCCTTATGCAATCTTTAAGGGAATTACATGCCAAATTATAGGCCTTTTCATTTCCCATACAT
TAGTTTGGTCACACTGTGGTATAAGTACAGTTGAGAGTCTGTTTCTCTTCTGTCCTCATAGC
ATGTAAACAGTTGGGAATAGGTGACAGAGTATATTTTAGAAAGTTTGTACCAGTTTGAAAGT
CAGGAGGATTTTAATGAAAATGATTTATTTTACTTTTCTTACTTTTCAGGACCTGTAGATGAA
TACATGTTGCCATTTGAGGAGGAAATTGGCCAGCATCCATCTCTTGAAGACATGCAGGAAG
TTGTTGTGCATAAAAAAAAGAGGCCTGTTTTAAGAGATTATTGGCAGAAACATGCTGTAAG
TTATCCAGTTAGCTTTTCATTTGAAATTCCAATAAAACACTTTTCAGAGGAATTATTTATCTCT
GCACATTTCTCTTTCTTCTGCAAGTATTTTCTGGAAGGTGATCTTCACACAGGATATTCTAG
AGTTCTAGAGGCAGAATTAGGGCTATGTCTGTATACCCCTGAAGGTGATTGTAAAGTAATA
GAGCTTTAGAGGGCTTTTGTCTCAATGGTCCTGTGCAGAAGATTGTGTCATCTATTTAGAAA
GTTTCCCAGGGAAAAGGCATGCCAGACTCGGAAACTGTGGATAGTTGGGTAACTTTGCTG
ATGACCACTTCCAATATGACAACATTTTAAAAAGTTTATGAAGCACCATGTTTTCTCCTCTCC
ATGGGAGTTTGTTGTAGCCTTTTAATTTCGGCTTAGACTTTCAAGTCTTAATAGTGCTTTAA
-mutant ACVR2A (a7) fragment
SEQ ID NO: 97
CATGGTTATAATAGATTGAACCTAGAGCCTCCAGAGCTGGAAGCACCACTGTTAACATTCT
GATCTGAATTGTAAGGAATGGGTACCTTAAAGGGATATTATATAACGTTAATTTACAAATACT
GATTGTTCCTTATGTCCTCTGTGCAGATGGAAATAGGCATCCTTTATACCTAGATAAGAAAG
CCCCTTATGCAATCTTTAAGGGAATTACATGCCAAATTATAGGCCTTTTCATTTCCCATACAT
TAGTTTGGTCACACTGTGGTATAAGTACAGTTGAGAGTCTGTTTCTCTTCTGTCCTCATAGC
ATGTAAACAGTTGGGAATAGGTGACAGAGTATATTTTAGAAAGTTTGTACCAGTTTGAAAGT
CAGGAGGATTTTAATGAAAATGATTTATTTTACTTTTCTTACTTTTCAGGACCTGTAGATGAA
TACATGTTGCCATTTGAGGAGGAAATTGGCCAGCATCCATCTCTTGAAGACATGCAGGAAG
TTGTTGTGCATAAAAAAAGAGGCCTGTTTTAAGAGATTATTGGCAGAAACATGCTGTAAGT
TATCCAGTTAGCTTTTCATTTGAAATTCCAATAAAACACTTTTCAGAGGAATTATTTATCTCT
GCACATTTCTCTTTCTTCTGCAAGTATTTTCTGGAAGGTGATCTTCACACAGGATATTCTAG
AGTTCTAGAGGCAGAATTAGGGCTATGTCTGTATACCCCTGAAGGTGATTGTAAAGTAATA
GAGCTTTAGAGGGCTTTTGTCTCAATGGTCCTGTGCAGAAGATTGTGTCATCTATTTAGAAA
GTTTCCCAGGGAAAAGGCATGCCAGACTCGGAAACTGTGGATAGTTGGGTAACTTTGCTG
ATGACCACTTCCAATATGACAACATTTTAAAAAGTTTATGAAGCACCATGTTTTCTCCTCTCC
ATGGGAGTTTGTTGTAGCCTTTTAATTTCGGCTTAGACTTTCAAGTCTTAATAGTGCTTTAA

1. A primer for detection of a mutation in a microsatellite contained in a target sequence of a double stranded DNA molecule, wherein the mutation is a frameshift of the microsatellite as compared with the corresponding wild type microsatellite sequence, and wherein the primer comprises a region of at least 10 nucleotides that is complementary to the target sequence of the antisense or the sense strand of the DNA molecule containing the microsatellite having a frameshift mutation, except that the primer includes between one and four nucleotides which are mismatched to the target sequence containing the mutation in the microsatellite and which are also mismatched to a corresponding sequence containing the wild type microsatellite, wherein the primer is configured to anneal across the length of the microsatellite having a frameshift mutation and to anneal to at least one nucleotide flanking the 5′ end of the microsatellite having a frameshift mutation and at least one nucleotide flanking the 3′ end of the microsatellite having a frameshift mutation.

2. The primer of claim 1, wherein the primer consists of between 16 and 30 nucleotides.

3. The primer of claim 1 or 2, wherein at least one mismatched nucleotide is located in a position of the primer that is configured to anneal 3′ downstream of the microsatellite or within the microsatellite, preferably wherein at least one mismatched nucleotide is within five nucleotides 5′ upstream of the 3′ end of the microsatellite or within 12 nucleotides 3′ downstream of the 3′ end of the microsatellite.

4. The primer of any one of claims 1 to 3, wherein the target sequence is in a TGFβR2 gene, an ASTE1 gene, a TAF1β gene or a ACVR2A gene, preferably a ASTE1 gene, a TAF1β gene or a ACVR2A gene.

5. The primer of any one of claims 1 to 4, wherein the first nucleotide 3′ downstream of the 3′ end of the microsatellite is not a mismatched nucleotide.

6. The primer of any one of claims 1 to 5, wherein the primer comprises between 1 and 13, or between 1 and 12 nucleotides, flanking the 5′ end of the microsatellite.

7. The primer of claim 6, wherein the primer comprises 9, 10, 11, 12 or 13 nucleotides, preferably 10, 12 or 13 nucleotides, flanking the 5′ end of the microsatellite.

8. The primer of any one of claims 1 to 7, wherein the primer comprises between 1 and 13 nucleotides flanking the 3′ end of the microsatellite.

9. The primer of any one of claims 1 to 8, wherein all of the at least one mismatched nucleotides are within the microsatellite.

10. The primer of any one of claims 1 to 9, wherein at least one mismatched nucleotide is located at the first nucleotide 5′ upstream of the 3′ end of the microsatellite and/or at the second nucleotide 5′ upstream of the 3′ end of the microsatellite and/or at the third nucleotide 5′ upstream of the 3′ end of the microsatellite.

11. The primer of any one of claims 1 to 10, wherein at least one mismatched nucleotide is:
a substitution of a thymine (T) with an adenine (A), a substitution of a thymine (T) with a cytosine (C), or a substitution of a cytosine (C) with a guanine (G);
a substitution of a thymine (T) with a cytosine (C), a substitution of a thymine (T) with an adenine (A), a substitution of a thymine (T) with a guanine (G), or a substitution of an adenine (A) with a cytosine (C); or
a substitution of an adenine (A) with a guanine (G), a substitution of an adenine (A) with a thymine (T), or a substitution of an adenine (A) with a cytosine (C).

12. The primer of claim 10 or 11, wherein at least one mismatched nucleotide is located at the first nucleotide 5′ upstream of the 3′ end of the microsatellite and at the second nucleotide 5′ upstream of the 3′ end of the microsatellite, wherein the at least one mismatched nucleotide located at the first nucleotide 5′ upstream of the 3′ end of the microsatellite is a substitution of an adenine (A) with a thymine (T) and the at least one mismatched nucleotide located at the second nucleotide 5′ upstream of the 3′ end of the microsatellite is a substitution of an adenine (A) with a guanine (G) or thymine (T).

13. The primer of any one of claims 1 to 11, wherein at least one mismatched nucleotide is located at:
the first, second or third nucleotide 5′ upstream of the 3′ end of the microsatellite, and
wherein the at least one mismatched nucleotide is a substitution of a thymine (T) with a guanine (G) or a cytosine (C).

14. The primer of any one of claim 1 to 11 or 13, wherein the primer has:
a mismatched nucleotide at the second nucleotide 5′ upstream of the 3′ end of the microsatellite and at the third nucleotide 5′ upstream of the 3′ end of the microsatellite;
a mismatched nucleotide at the first nucleotide 5′ upstream of the 3′ end of the microsatellite, at the second nucleotide 5′ upstream of the 3′ end of the microsatellite and at the fourth nucleotide 5′ upstream of the 3′ end of the microsatellite;
a mismatched nucleotide at the first nucleotide 5′ upstream of the 3′ end of the microsatellite, at the fourth nucleotide 5′ upstream of the 3′ end of the microsatellite and at the fifth nucleotide 5′ upstream of the 3′ end of the microsatellite;
a mismatched nucleotide at the first nucleotide 5′ upstream of the 3′ end of the microsatellite, at the third nucleotide 5′ upstream of the 3′ end of the microsatellite and at the fourth nucleotide 5′ upstream of the 3′ end of the microsatellite;
a mismatched nucleotide at the second nucleotide 5′ upstream of the 3′ end of the microsatellite, at the third nucleotide 5′ upstream of the 3′ end of the microsatellite and at the fourth nucleotide 5′ upstream of the 3′ end of the microsatellite; or
a mismatched nucleotide at the third 5′ upstream of the 3′ end of the microsatellite, at the fourth nucleotide 5′ upstream of the 3′ end of the microsatellite and at the fifth nucleotide 5′ upstream of the 3′ end of the microsatellite;
and wherein the frameshift mutation is preferably in a microsatellite in ASTE1.

15. The primer of any one of claims 1 to 12, wherein the primer has:
a mismatched nucleotide at the first nucleotide 5′ upstream of the 3′ end of the microsatellite;
a mismatched nucleotide at the first nucleotide 5′ upstream of the 3′ end of the microsatellite and at the second nucleotide 5′ upstream of the 3′ end of the microsatellite; or
a mismatched nucleotide at the first nucleotide 5′ upstream of the 3′ end of the microsatellite, at the second nucleotide 5′ upstream of the 3′ end of the microsatellite, and at the fourth nucleotide 5′ upstream of the 3′ end of the microsatellite;
and wherein the frameshift mutation is preferably in a microsatellite in TAF1β.

16. The primer of any one of claims 1 to 11, wherein the primer has a mismatched nucleotide at the first nucleotide 5′ upstream of the 3′ end of the microsatellite, at the second nucleotide 5′ upstream of the 3′ end of the microsatellite and at the third nucleotide 5′ upstream of the 3′ end of the microsatellite, and wherein the frameshift mutation is preferably in a microsatellite in ACVR2A.

17. The primer of any one of claims 1 to 16, wherein the primer includes between one and three nucleotides which are mismatched to the target sequence containing the mutation in the microsatellite and which are also mismatched to a corresponding sequence containing the wild type microsatellite.

18. A primer for DNA amplification comprising:
the sequence defined by any one of SEQ ID NO: 24 to 28 or;
the sequence defined by any one of SEQ ID NO: 45, 47, 83 or 100;
the sequence defined by any one of SEQ ID NO: 43, 44, 46, 48 or 49; or
the sequence defined by any one of SEQ ID NO: 60 to 62;
the sequence defined by SEQ ID NO: 89;
and wherein X defines a substitution to any nucleotide which is mismatched to the corresponding nucleotide in the target sequence and the wild type sequence.

19. A primer for DNA amplification comprising:
the sequence defined by any one of SEQ ID NO: 17 to 19 or 21 to 23;
the sequence defined by any one of SEQ ID NO: 34 to 38 or 40;
the sequence defined by any one of SEQ ID NO: 39, 41, 42, 84-88, 98, 99 or 101;
the sequence defined by any one of SEQ ID NO: 53 to 59; or
the sequence defined by SEQ ID NO: 90 or 91.

20. A kit for detection of a mutation in a microsatellite contained in a target sequence of a double stranded DNA molecule, wherein the mutation is a frameshift of the microsatellite as compared with the corresponding wild type microsatellite sequence, and wherein the kit comprises a first primer according to any one of claims 1 to 19, and optionally a second primer, wherein the second primer is configured to anneal to the target sequence 3′ downstream of the microsatellite on the opposite strand of the DNA molecule to the strand on which the first primer is configured to anneal.

21. The kit of claim 20, wherein the kit comprises the second primer and the kit further comprises:
a third primer, wherein the third primer is configured to anneal 5′ upstream of the region to which the second primer is configured to anneal, and wherein the third primer is configured to anneal to the same strand as the first primer and the opposite strand to the second primer, or
another control primer pair, preferably wherein the sequence containing the wild type microsatellite consists of the sequence defined in SEQ ID NO: 3, SEQ ID NO: 7, SEQ ID NO: 11 or SEQ ID NO: 96, and preferably the third primer comprises the sequence defined by SEQ ID NO: 13, SEQ ID NO: 30, SEQ ID NO: 31, SEQ ID NO: 50 or SEQ ID NO: 93.

22. A method for detecting a mutation in a microsatellite contained in a target sequence of a double-stranded DNA molecule, wherein the mutation is a frameshift of the microsatellite as compared with the corresponding wild type microsatellite sequence, and the method comprises:
a) providing a first aliquot of a sample comprising human DNA,
b) adding to the first aliquot the necessary components for DNA amplification and a first primer to form a first reaction mix,
wherein the first primer is suitable for detection of a mutation in a microsatellite contained in a target sequence of a double stranded DNA molecule and comprises a region of nucleotides that is complementary to the target sequence containing the microsatellite having a frameshift mutation except for between one and four nucleotides which are mismatched to the target sequence containing the microsatellite having a frameshift mutation and which are also mismatched to a corresponding sequence containing the wild type microsatellite, and wherein the first primer is configured to anneal across the length of the microsatellite having a frameshift mutation and to anneal to at least one nucleotide flanking the 3′ end of the microsatellite having a frameshift mutation and at least one nucleotide flanking the 5′ end of the microsatellite having a frameshift mutation,
c) carrying out DNA amplification on the first reaction mix,
d) detecting the presence of the frameshift mutation in the sample when an amplification product is produced from the DNA amplification of the first reaction mix.

23. The method of claim 22, for detecting a mutation in a microsatellite contained in a target sequence of a double stranded DNA molecule, wherein the mutation is a frameshift of the microsatellite as compared with the corresponding wild type microsatellite sequence, and the method comprising:
a) providing a first aliquot of a sample comprising human DNA,
b) adding to the first aliquot the necessary components for DNA amplification, a first primer and a second primer;
wherein the first primer is suitable for detection of a mutation in a microsatellite contained in a target sequence of a double stranded DNA molecule and comprises a region of nucleotides that is complementary to the target sequence containing the microsatellite having a frameshift mutation except for between one and four nucleotides which are mismatched to the target sequence containing the microsatellite having a frameshift mutation and which are also mismatched to a corresponding sequence containing the wild type microsatellite and wherein the first primer is configured to anneal across the length of the microsatellite having a frameshift mutation and to anneal to at least one nucleotide flanking the 3′ end of the microsatellite having a frameshift mutation and at least one nucleotide flanking the 5′ end of the microsatellite having a frameshift mutation;
and wherein the second primer is configured to anneal to the target sequence 3′ downstream of the microsatellite, to form a first reaction mix;
wherein the first primer is configured to anneal to the sense strand of the DNA molecule and the second primer is configured to anneal to the antisense strand of the DNA molecule, or the first primer is configured to anneal to the antisense strand of the DNA molecule and the second primer is configured to anneal to the sense strand of the DNA molecule,
c) carrying out DNA amplification on the first reaction mix,
d) detecting the presence of the frameshift mutation in the sample when an amplification product is produced from the DNA amplification of the first reaction mix.

24. The method of claim 24, further comprising:
a) also adding to the first aliquot at step b) of the method either:
I) the second primer and a third primer, wherein the third primer is configured to anneal 5′ upstream of the region to which the second primer is configured to anneal, and wherein the third primer is configured to anneal to the same strand as the first primer and the opposite strand to the second primer or
II) a control primer pair, to form the first reaction mix,
and detecting that DNA amplification has been carried out successfully when an amplification product is produced from the DNA amplification using the second and third primers or the control primer pair; or
b) providing a second aliquot of the sample comprising human DNA and adding to the second aliquot the necessary components for DNA amplification and either:
I) the second primer and a third primer, wherein the third primer is configured to anneal 5′ upstream of the region to which the second primer is configured to anneal, and wherein the third primer is configured to anneal to the same strand as the first primer and the opposite strand to the second primer or
II) a control primer pair, to form a second reaction mix, and
carrying out DNA amplification on the second reaction mix, and detecting that DNA amplification has been carried out successfully when an amplification product is produced from the DNA amplification of the second reaction mix.

25. The method of any one of claims 21 to 23, wherein DNA amplification is polymerase chain reaction (PCR), wherein the PCR comprises a plurality of cycles of denaturation, annealing and extension.

26. The method of claim 25, wherein step d) further comprises running the product of the PCR reaction on a gel and visualising a band to confirm that DNA amplification has been successful, preferably wherein the method further comprises step f) cutting out the band for DNA sequencing.

27. The kit of claim 20 or 21, or the method of any one of claims 22 to 26, wherein the frameshift mutation is in a microsatellite in a TGFβR2 gene, an ASTE1 gene, a TAF1β gene or an AVCR2A gene, preferably an ASTE1 gene, a TAF1β gene or an ACVR2A gene.

28. The kit of claim 20 or 21, or the method of any one of claims 22 to 26, wherein:
the frameshift mutation is in a microsatellite in the TGFβR2 gene and the target sequence comprises the sequence according to residues 270 to 278 of SEQ ID NO: 3, and wherein the first primer comprises the sequence defined by any one of SEQ ID NO: 24 to 28 and/or the second primer comprises the sequence defined by SEQ ID NO: 14; or
the frameshift mutation is in a microsatellite in the ASTE1 gene and the target sequence comprises the sequence according to residues 328 to 337 of SEQ ID NO: 7, and wherein the first primer comprises the sequence defined by SEQ ID NO: 45, 47, 83 or 100, and/or, when present, the second primer comprises the sequence defined by SEQ ID NO: 29; or
the frameshift mutation is in a microsatellite in the ASTE1 gene and the target sequence comprises the sequence according to residues 328 to 337 of SEQ ID NO: 7, and wherein the first primer comprises the sequence defined by SEQ ID NO: 43, 44, 46, 48 or 49, and/or, when present, the second primer comprises the sequence defined by SEQ ID NO: 29;
the frameshift mutation is in a microsatellite in the TAF1β gene and the target sequence comprises the sequence according to residues 255 to 264 of SEQ ID NO: 11, and wherein the first primer comprises the sequence defined by any one of SEQ ID NOS: 60 to 62, and/or, when present, the second primer comprises the sequence defined by SEQ ID NO: 51; or
the frameshift mutation is in a microsatellite in the ACVR2A gene and the target sequence comprises the sequence according to residues 509 to 515 of SEQ ID NO: 96, and wherein the first primer comprises the sequence defined by SEQ ID NO: 89, and/or, when present, the second primer comprises the sequence defined by SEQ ID NO: 92;
and wherein X defines a substitution to any nucleotide which is mismatched to the corresponding nucleotide in the target sequence and the wild type sequence.

29. The kit of claim 20 or 21, or the method of any one of claims 22 to 26, wherein:
the frameshift mutation is in a microsatellite in the TGFβR2 gene and the target sequence comprises the sequence according to residues 270 to 278 of SEQ ID NO: 3, and wherein the first primer comprises the sequence defined by any one of SEQ ID NO: 17 to 19 or 21 to 23 and/or, when present, the second primer comprises the sequence defined by SEQ ID NO: 14; or
the frameshift mutation is in a microsatellite in the ASTE1 gene and the target sequence comprises the sequence according to residues 328 to 337 of SEQ ID NO: 7, and wherein the first primer comprises the sequence defined by SEQ ID NO: 34 to 38 or 40, and/or, when present, the second primer comprises the sequence defined by SEQ ID NO: 29; or
the frameshift mutation is in a microsatellite in the ASTE1 gene and the target sequence comprises the sequence according to residues 328 to 337 of SEQ ID NO: 7, and wherein the first primer comprises the sequence defined by any one of SEQ ID NO: 39, 41, 42, 84 to 88, 98, 99 and 101 and/or, when present, the second primer comprises the sequence defined by SEQ ID NO: 29;
the frameshift mutation is in a microsatellite in the TAF1β gene and the target sequence comprises the sequence according to residues 255 to 264 of SEQ ID NO: 11, and wherein the first primer comprises the sequence defined by any one of SEQ ID NOS: 53 to 59, and/or, when present, the second primer comprises the sequence defined by SEQ ID NO: 51; or
the frameshift mutation is in a microsatellite in the ACVR2A gene and the target sequence comprises the sequence according to residues 509 to 515 of SEQ ID NO: 96, and wherein the first primer comprises the sequence defined by SEQ ID NO: 90 or 91, and/or, when present, the second primer comprises the sequence defined by SEQ ID NO: 92.

30. The method of claim 24, wherein:
the microsatellite is in a TGFβR2 gene, the sequence comprising the wild type microsatellite comprises the sequence according to SEQ ID NO: 3, and wherein the third primer comprises the sequence defined by SEQ ID NO: 13; or
the microsatellite is in an ASTE1 gene, the sequence comprising the wild type microsatellite comprises the sequence according to SEQ ID NO: 7, and wherein the third primer comprises the sequence defined by SEQ ID NO: 30 or SEQ ID NO: 31;
the microsatellite is in an TAF1β gene, the sequence comprising the wild type microsatellite comprises the sequence according to SEQ ID NO: 11, and wherein the third primer comprises the sequence defined by SEQ ID NO: 50; or
the microsatellite is in an ACVR2A gene, the sequence comprising the wild type microsatellite comprises the sequence of SEQ ID NO: 96, and wherein the third primer comprises the sequence defined by SEQ ID NO: 93.

31. A method of diagnosing a disease associated with a frameshift mutation in a microsatellite, comprising carrying out the method according to any one of claims 22 to 30.

32. The method of any one of claims claims 22 to 26, further comprising determining that a patient suffering from a disease or disorder associated with a frameshift mutation is suitable for a treatment targeting said frameshift mutation if the frameshift mutation is detected in step d) in a sample from the patient, preferably wherein the disease or disorder associated with a frameshift mutation is a cancer, and more preferably wherein:
the cancer is colorectal cancer or gastric cancer, and the treatment targeting the frameshift mutation is FMPV-1 or FMPV-3, and the frameshift mutation is in a microsatellite in the TGFβR2 gene and comprises the sequence according to residues 270 to 278 of SEQ ID NO: 4, or
the cancer is endometrial cancer or gastric cancer and the treatment targeting the frameshift mutation is FMPV-2 or FMPV-3, and the frameshift mutation is in a microsatellite in the ASTE1 gene and comprises the sequence according to residues 328 to 337 of SEQ ID NO: 8,
the cancer is endometrial cancer or gastric cancer and the treatment targeting the frameshift mutation is an immunogenic fragment of the TAF1β-1a frameshift mutant protein or FMPV-3, and the frameshift mutation is in a microsatellite in the TAF1β gene and comprises the sequence according to residues 255 to 264 of SEQ ID NO: 12, or
the cancer is colon adenocarcinoma, stomach adenocarcinoma or uterine corpus endometrial cancer, and the frameshift mutation is in a microsatellite in the ACVR2A gene and comprises the sequence according to residues 509 to 515 of SEQ ID NO: 97.

33. The method of claim 32, wherein:
the microsatellite is in a TGFβR2 gene, the sequence comprising the wild type microsatellite comprises the sequence according to SEQ ID NO: 3, and wherein the third primer comprises the sequence defined by SEQ ID NO: 13, preferably wherein the first primer comprises the sequence defined by any one of SEQ ID NO: 24 to 28, and/or, when present, the second primer comprises the sequence defined by SEQ ID NO: 14; or
the microsatellite is in a ASTE1 gene, the sequence comprising the wild type microsatellite comprises the sequence according to SEQ ID NO: 7, and wherein the third primer comprises the sequence defined by SEQ ID NO: 30 or SEQ ID NO: 31, preferably wherein the first primer comprises the sequence defined by SEQ ID NO: 45, 47, 83 or 100 and/or, when present the second primer comprises the sequence defined by SEQ ID NO: 29; or
the microsatellite is in a ASTE1 gene, the sequence comprising the wild type microsatellite comprises the sequence according to SEQ ID NO: 7, and wherein the third primer comprises the sequence defined by SEQ ID NO: 30 or SEQ ID NO: 31, preferably wherein the first primer comprises the sequence defined by SEQ ID NO: 43, 44, 46, 48 or 49 and/or when present, the second primer comprises the sequence defined by SEQ ID NO: 29;
the microsatellite is in a TAF1β gene, the sequence comprising the wild type microsatellite comprises the sequence according to SEQ ID NO: 11, and wherein the third primer comprises the sequence defined by SEQ ID NO: 50, preferably wherein the first primer comprises the sequence defined by any one of SEQ ID NOS: 60 to 62 and/or, when present, the second primer comprises the sequence defined by SEQ ID NO: 51; or
the frameshift mutation is in a microsatellite in the ACVR2A gene and the target sequence comprises the sequence according to residues 509 to 515 of SEQ ID NO: 96, and wherein the first primer comprises the sequence defined by SEQ ID NO: 89, and/or, when present, the second primer comprises the sequence defined by SEQ ID NO: 92;
and wherein X defines a substitution to any nucleotide which is mismatched to the corresponding nucleotide in the target sequence and the wild type sequence.

34. The method of claim 32, wherein:
the microsatellite is in a TGFβR2 gene, the sequence comprising the wild type microsatellite comprises the sequence according to SEQ ID NO: 3, and wherein the third primer comprises the sequence defined by SEQ ID NO: 13, preferably wherein the first primer comprises the sequence defined by any one of SEQ ID NO: 17 to 19 and 21 to 23, and/or, when present, the second primer comprises the sequence defined by SEQ ID NO: 14; or
the microsatellite is in a ASTE1 gene, the sequence comprising the wild type microsatellite comprises the sequence according to SEQ ID NO: 7, and wherein the third primer comprises the sequence defined by SEQ ID NO: 30 or SEQ ID NO: 31, preferably wherein the first primer comprises the sequence defined by SEQ ID NO: 34 to 38 or 40 and/or, when present, the second primer comprises the sequence defined by SEQ ID NO: 29; or
the microsatellite is in a ASTE1 gene, the sequence comprising the wild type microsatellite comprises the sequence according to SEQ ID NO: 7, and wherein the third primer comprises the sequence defined by SEQ ID NO: 30 or SEQ ID NO: 31, preferably wherein the first primer comprises the sequence defined by any one of SEQ ID NOs: 39, 41, 42, 84-88, 98, 99 and 101 and/or, when present, the second primer comprises the sequence defined by SEQ ID NO: 29;
the microsatellite is in a TAF1β gene, the sequence comprising the wild type microsatellite comprises the sequence according to SEQ ID NO: 11, and wherein the third primer comprises the sequence defined by SEQ ID NO: 50, preferably wherein the first primer comprises the sequence defined by any one of SEQ ID NOS: 53 to 59 and/or, when present, the second primer comprises the sequence defined by SEQ ID NO: 51; or
the microsatellite is in an ACVR2A gene, the sequence comprising the wild type microsatellite comprises the sequence according to SEQ ID NO: 96, and wherein the third primer comprises the sequence defined by SEQ ID NO: 93, preferably wherein the first primer comprises the sequence defined by SEQ ID NO: 90 or 91 and/or, when present, the second primer comprises the sequence defined by SEQ ID NO: 92.

35. The method of any one of claims 22 to 34, wherein the sample is a liquid biopsy comprising cell free DNA, preferably wherein the liquid biopsy is plasma.

36. The method of any one of claims 22 to 35, wherein the DNA amplification is PCR, and the PCR is carried out in high stringency conditions, optionally wherein the high stringency conditions comprise at least one of:
a) carrying out the annealing step of PCR at a temperature that is at least 1° C., 2° C., 3° C., 4° C., 5° C., 6° C., 7° C., 8° C., 9° C. or 10° C. higher than the recommended annealing temperature of the reaction;
b) carrying out the annealing step of PCR for only 30 seconds, preferably 15 seconds, per cycle;
c) carrying out the DNA amplification in a buffer concentration that is less than 0.1×, 0.2×, 0.3×, 0.4×, 0.5×, 0.6× or 0.75×;
d) carrying out the DNA amplification in a buffer comprising ammonium ions; and
e) performing 30 or fewer, 25 or fewer, 20 or fewer, 15 or fewer, or 10 or fewer, cycles of PCR; and
f) carrying out the DNA amplification using a template DNA concentration of 0.2 ng or less, or 0.05 ng or less.
